(19) United States
(12) Patent Application Publication
(10) Pub. No.: US 2024/0156790 A1
(43) Pub. Date: May 16, 2024

(54) COMPOUNDS, COMPOSITIONS, AND METHODS FOR MODULATING FERROPTOSIS AND TREATING EXCITOTOXIC DISORDERS

(71) Applicant: THE TRUSTEES OF COLUMBIA UNIVERSITY IN THE CITY OF NEW YORK, New York, NY (US)

(72) Inventors: Brent R. STOCKWELL, New York, NY (US); Arie ZASK, New York, NY (US); Hui TAN, New York, NY (US); Jacob D. DANIELS, New York, NY (US)

(21) Appl. No.: 18/518,731

(22) Filed: Nov. 24, 2023

Related U.S. Application Data
(63) Continuation-in-part of application No. PCT/US2022/030843, filed on May 25, 2022.
Application No. PCT/US2022/030843 is a continuation of application No. 17/330,386, filed on May 25, 2021.
Application No. 17/330,386 is a continuation-in-part of application No. PCT/US2019/063640, filed on Nov. 27, 2019.
(60) Provisional application No. 62/771,841, filed on Nov. 27, 2018.

(51) Int. Cl. A61K 31/4245; A61K 31/166; A61K 31/245; A61K 31/421; A61K 31/44; A61K 31/4439; A61K 31/5375; A61P 31/06; A61P 39/06; C07C 229/60; C07C 233/65; C07D 213/74; C07D 213/80; C07D 263/32; C07D 271/10; C07D 295/13; C07D 413/04; C07D 413/12

(52) U.S. Cl. CPC A61K 31/4245; A61K 31/166; A61K 31/245; A61K 31/421; A61K 31/44; A61K 31/4439; A61K 31/5375; A61P 31/06; A61P 39/06; C07C 229/60; C07C 233/65; C07D 213/74; C07D 213/80; C07D 263/32; C07D 271/10; C07D 295/13; C07D 413/04; C07D 413/12

(57) ABSTRACT

The present disclosure provides, inter alia, a compound having the structure:

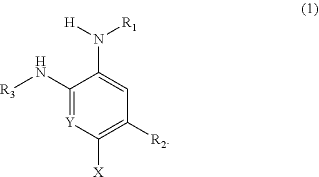

Also provided are compositions containing a pharmaceutically acceptable carrier and one or more compounds according to the present disclosure. Further provided are methods for treating or ameliorating the effects of an excitotoxic disorder in a subject, methods of modulating ferroptosis in a subject, methods of reducing reactive oxygen species (ROS) in a cell, methods for treating or ameliorating the effects of a neurodegenerative disease, methods for alleviating side effects in a subject undergoing radiotherapy and/or immunotherapy, and methods for treating or ameliorating the effects of an infection associated with ferroptosis in a subject.

### CROSS REFERENCE TO RELATED APPLICATIONS

The present application is a continuation-in-part of PCT international application no. PCT/US2022/030843, filed on May 25, 2022, which claims benefit to U.S. patent application Ser. No. 17/330,386, filed on May 25, 2021, which is a continuation-in-part of PCT international application no. PCT/US2019/063640, filed on Nov. 27, 2019, which claims benefit of U.S. Provisional Patent Application Ser. No. 62/771,841, filed on Nov. 27, 2018, which applications are incorporated by reference herein in their entireties.

### GOVERNMENT FUNDING

This disclosure was made with government support under grant nos. CA097061, CA209896 and NS109407, awarded by National Institutes of Health. The government has certain rights in the disclosure.

### FIELD OF DISCLOSURE

The present disclosure provides, inter alia, compounds having the structure:

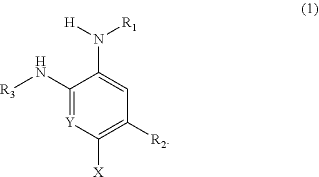

Also provided are pharmaceutical compositions containing the compounds of the present disclosure, as well as methods of using such compounds and compositions.

### BACKGROUND OF THE DISCLOSURE

Cell death is crucial for normal development, homeostasis and the prevention of hyper-proliferative diseases such as cancer (Fuchs and Steller, 2011; Thompson, 1995). It was once thought that almost all regulated cell death in mammalian cells resulted from the activation of caspase-dependent apoptosis (Fuchs and Steller, 2011; Thompson, 1995). More recently this view has been challenged by the discovery of several regulated non-apoptotic cell death pathways activated in specific disease states, including poly(ADP-ribose) polymerase-1 (PARP-1) and apoptosis inducing factor 1 (AIF1)-dependent parthanatos, caspase-1-dependent pyroptosis and receptor interacting protein kinase 1 (RIPK1)-dependent necroptosis (Bergsbaken et al., 2009; Christofferson and Yuan, 2010; Wang et al., 2009). It is believed that additional regulated forms of non-apoptotic cell death likely remain to be discovered that mediate cell death in other developmental or pathological circumstances.

The RAS family of small GTPases (HRAS, NRAS and KRAS) is mutated in about 30% of all cancers (Vigil et al., 2010). Finding compounds that are selectively lethal to RAS-mutant tumor cells is, therefore, a high priority. Two structurally unrelated small molecules, named erastin and RSL3, were previously identified. These molecules were selectively lethal to oncogenic RAS-mutant cell lines, and together, they were referred to as RAS-selective lethal (RSL) compounds (Dolma et al., 2003; Yang and Stockwell, 2008). Using affinity purification, voltage dependent anion channels 2 and 3 (VDAC2/3) were identified as direct targets of erastin (Yagoda et al., 2007), but not RSL3. ShRNA and cDNA overexpression studies demonstrated that VDAC2 and VDAC3 are necessary, but not sufficient, for erastin-induced death (Yagoda et al., 2007), indicating that additional unknown targets are required for this process.

The type of cell death activated by the RSLs has been enigmatic. Classic features of apoptosis, such as mitochondrial cytochrome c release, caspase activation and chromatin fragmentation, are not observed in RSL-treated cells (Dolma et al., 2003; Yagoda et al., 2007; Yang and Stockwell, 2008). RSL-induced death is, however, associated with increased levels of intracellular reactive oxygen species (ROS) and is prevented by iron chelation or genetic inhibition of cellular iron uptake (Yagoda et al., 2007; Yang and Stockwell, 2008). In a recent systematic study of various mechanistically unique lethal compounds, the prevention of cell death by iron chelation was a rare phenomenon (Wolpaw et al., 2011), suggesting that few triggers can access iron-dependent lethal mechanisms.

Accordingly, there is a need for the exploration of various pathways of regulated cell death, as well as for compositions and methods for preventing the occurrence of regulated cell death. This disclosure is directed to meeting these and other needs.

### SUMMARY OF THE DISCLOSURE

Without being bound to a particular theory, the inventors hypothesized that RSLs, such as erastin, activate a lethal pathway that is different from apoptosis, necrosis and other well-characterized types of regulated cell death. It was found that erastin-induced death involves a unique constellation of morphological, biochemical and genetic features, which led to the name “ferroptosis” as a description for this phenotype. Small molecule inhibitors of ferroptosis that prevent ferroptosis in cancer cells, as well as glutamate-induced cell death in postnatal rat brain slices have been identified and disclosed herein. The inventors have found an underlying similarity between diverse forms of iron-dependent, non-apoptotic death and that the manipulation of ferroptosis may be exploited to selectively destroy RAS-mutant tumor cells or to preserve neuronal cells exposed to specific oxidative conditions.

Accordingly, one embodiment of the present disclosure is a compound according to formula (1):

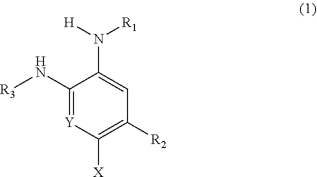

wherein:
R₁ is selected from the group consisting of H, alkyl, aryl, C₁₋₆alkyl-aryl, C₁₋₆alkyl-phenolyl, and C₃₋₁₀carbocycle, wherein each of the alkyl, aryl, C₁₋₆alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
R₃ is a C₃₋₁₂carbocycle, or a polyyne, wherein each of the C₃₋₁₂carbocycle and polyyne are optionally substituted with one or more atoms or groups; and
X is selected from the group consisting of H, optionally substituted alkyl, and halo;
Y is —CH or N;
or an N-oxide, crystalline form, hydrate, or a pharmaceutically acceptable salt thereof,
with the proviso that:
when R₁ and X are both H, Y is —CH and R₃ is

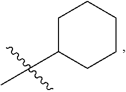 [SMILES: *C1CCCCC1]

R₂ cannot be

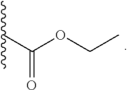 [SMILES: *C(=O)OCC]

Another embodiment of the present disclosure is a compound selected from the group consisting of:

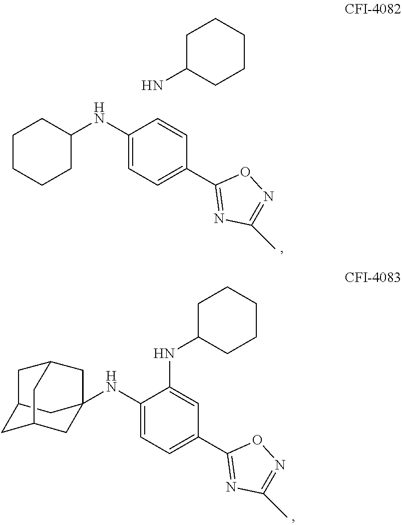

and combinations thereof, or an N-oxide, crystalline form, hydrate, or a pharmaceutically acceptable salt thereof.

Another embodiment of the present disclosure is a compound having the structure selected from the group consisting of:

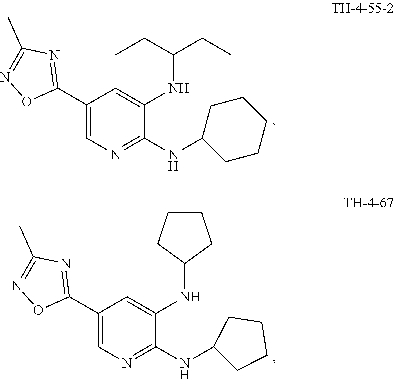 [SMILES: CCC(CC)Nc1cc(-c2nc(C)no2)cnc1NC1CCCCC1 ; Cc1noc(-c2cnc(NC3CCCC3)c(NC3CCCC3)c2)n1]

and combinations thereof, or an N-oxide, crystalline form, hydrate, or a pharmaceutically acceptable salt thereof.

Another embodiment of the present disclosure is a pharmaceutical composition. This pharmaceutical composition comprises a pharmaceutically acceptable carrier or diluent and one or more compounds according to formula (1):

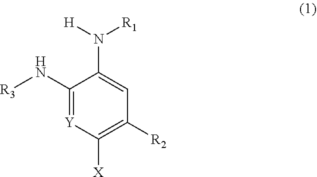

wherein:
R₁ is selected from the group consisting of H, alkyl, aryl, C₁₋₆alkyl-aryl, C₁₋₆alkyl-phenolyl, and C₃₋₁₀carbocycle, wherein each of the alkyl, aryl, C₁₋₆alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
R₃ is a C₃₋₁₂carbocycle, or a polyyne, wherein each of the C₃₋₁₂carbocycle and polyyne are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R₁ and X are both H, Y is —CH and R₃ is

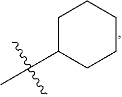 [SMILES: *C1CCCCC1]

R₂ cannot be

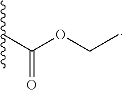 [SMILES: *C(=O)OCC]

A further embodiment of the present disclosure is a kit. This kit comprises a compound or a pharmaceutical composition according to the present disclosure with instructions for the use of the compound or the pharmaceutical composition, respectively.

Another embodiment of the present disclosure is a method for treating or ameliorating the effects of a disorder in a subject in need thereof. This method comprises administering to the subject an effective amount of one or more compounds having the structure of formula (1):

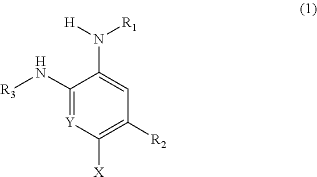

wherein:
R₁ is selected from the group consisting of H, alkyl, aryl, C₁₋₆alkyl-aryl, C₁₋₆alkyl-phenolyl, and C₃₋₁₀carbocycle, wherein each of the alkyl, aryl, C₁₋₆alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
R₃ is a C₃₋₁₂carbocycle, or a polyyne, wherein each of the C₃₋₁₂carbocycle and polyyne are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R₁ and X are both H, Y is —CH and R₃ is

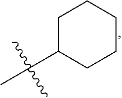 [SMILES: *C1CCCCC1]

R₂ cannot be

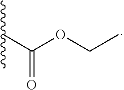 [SMILES: *C(=O)OCC]

An additional embodiment of the present disclosure is a method for treating or ameliorating the effects of a disorder in a subject in need thereof. This method comprises administering to the subject an effective amount of a pharmaceutical composition comprising a pharmaceutically acceptable carrier or diluent and one or more compounds having the structure of formula (1):

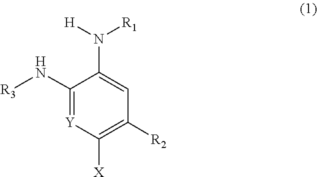

wherein:
R₁ is selected from the group consisting of H, alkyl, aryl, C₁₋₆alkyl-aryl, C₁₋₆alkyl-phenolyl, and C₃₋₁₀carbocycle, wherein each of the alkyl, aryl, C₁₋₆alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
R₃ is a C₃₋₁₂carbocycle, or a polyyne, wherein each of the C₃₋₁₂carbocycle and polyyne are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R₁ and X are both H, Y is —CH and R₃ is

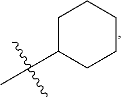 [SMILES: *C1CCCCC1]

R₂ cannot be

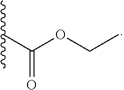 [SMILES: *C(=O)OCC]

Another embodiment of the present disclosure is a method of modulating ferroptosis in a subject in need thereof. This method comprises administering to the subject an effective amount of a ferroptosis inhibitor, which comprises one or more compounds having the structure of formula (1):

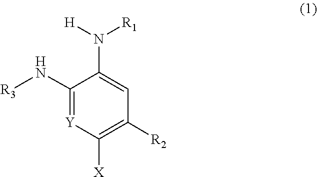

wherein:
R₁ is selected from the group consisting of H, alkyl, aryl, C₁₋₆alkyl-aryl, C₁₋₆alkyl-phenolyl, and C₃₋₁₀carbocycle, wherein each of the alkyl, aryl, C₁₋₆alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
R₃ is a C₃₋₁₂carbocycle, or a polyyne, wherein each of the C₃₋₁₂carbocycle and polyyne are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R₁ and X are both H, Y is —CH and R₃ is

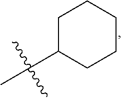 [SMILES: *C1CCCCC1]

R₂ cannot be

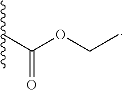 [SMILES: *C(=O)OCC]

A further embodiment of the present disclosure is a method of reducing reactive oxygen species (ROS) in a cell. This method comprises contacting a cell with a ferroptosis modulator, which comprises one or more compounds having the structure of formula (1):

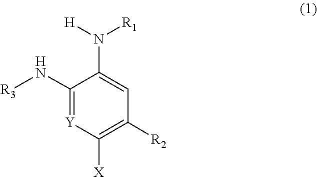

wherein:
R₁ is selected from the group consisting of H, alkyl, aryl, C₁₋₆alkyl-aryl, C₁₋₆alkyl-phenolyl, and C₃₋₁₀carbocycle, wherein each of the alkyl, aryl, C₁₋₆alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
R₃ is a C₃₋₁₂carbocycle, or a polyyne, wherein each of the C₃₋₁₂carbocycle and polyyne are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R₁ and X are both H, Y is —CH and R₃ is

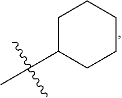 [SMILES: *C1CCCCC1]

R₂ cannot be

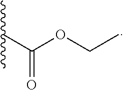 [SMILES: *C(=O)OCC]

An additional embodiment of the present disclosure is a method for treating or ameliorating the effects of a neurodegenerative disease in a subject in need thereof. This method comprises administering to the subject an effective amount of one or more compounds having the structure of formula (1):

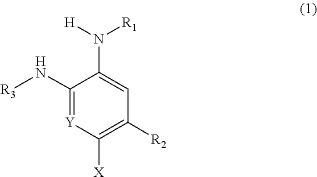

wherein:
R₁ is selected from the group consisting of H, alkyl, aryl, C₁₋₆alkyl-aryl, C₁₋₆alkyl-phenolyl, and C₃₋₁₀carbocycle, wherein each of the alkyl, aryl, C₁₋₆alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
R₃ is a C₃₋₁₂carbocycle, or a polyyne, wherein each of the C₃₋₁₂carbocycle and polyyne are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R₁ and X are both H, Y is —CH and R₃ is

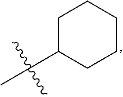 [SMILES: *C1CCCCC1]

R₂ cannot be

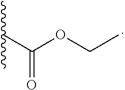 [SMILES: *C(=O)OCC]

A further embodiment of the present disclosure is a compound according to formula (2):

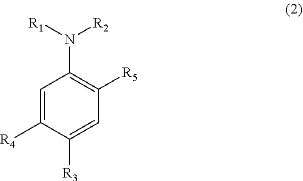

wherein:
R₁ and R₂ are independently selected from the group consisting of H, aryl, C₁₋₆alkyl-aryl, C₁₋₆alkyl-phenolyl, C₁₋₆alkyl-bicycle, and C₃₋₁₀carbocycle, wherein each of the aryl, C₁₋₆alkyl-aryl, C₁₋₆alkyl-phenolyl, C₁₋₆alkyl-bicycle, and C₃₋₁₀carbocycle are optionally substituted with one or more atoms or groups; or together, with the nitrogen attached, form a cyclic or bicyclic structure, wherein the cyclic or bicyclic structure is optionally substituted with one or more atoms or groups;
R₃ is selected from the group consisting of hydroxyl, alkoxy, and alcohol, wherein each of the hydroxyl, alkoxy, and alcohol are optionally substituted with one or more atoms or groups;
R₄ is selected from the group consisting of H, alkyl, and alkoxy; or together with R₃, form a ring structure, wherein the ring structure is optionally substituted with one or more atoms or groups; and
R₅ is selected from the group consisting of H, and alkoxy;
or an N-oxide, crystalline form, hydrate, or a pharmaceutically acceptable salt thereof.

Still another embodiment of the present disclosure is a compound according to formula (3):

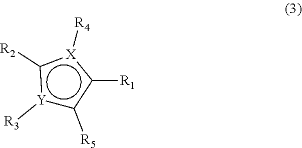

wherein:
X is selected from N, O, and S;
Y is C or N;
R₁ and R₅ are independently selected from the group consisting of H, alkenyl, ester, amino, and aryl, wherein each of the alkenyl, ester, amino, and aryl are optionally substituted with one or more atoms or groups; or together form a ring structure, wherein the ring structure is optionally substituted with one or more atoms or groups;
R₂ and R₃ together form a saturated or unsaturated ring structure, wherein the ring structure is optionally substituted with one or more atoms or groups; and
R₄ is selected from the group consisting of no atom, H, alkyl, alkenyl, and ketone;
or an N-oxide, crystalline form, hydrate, or a pharmaceutically acceptable salt thereof.

Another embodiment of the present disclosure is a compound selected from the group consisting of:

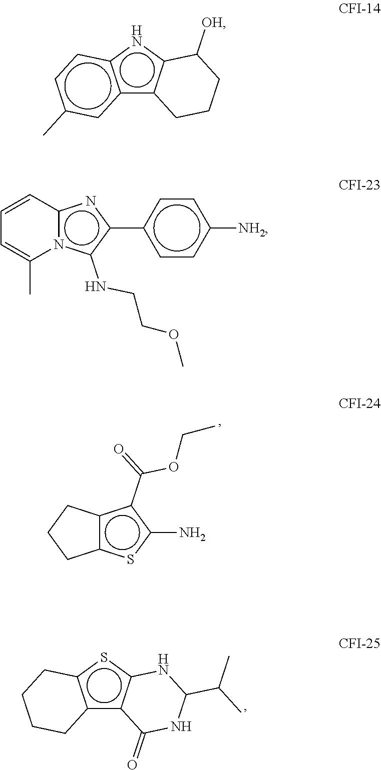

and combinations thereof, or an N-oxide, crystalline form, hydrate, or a pharmaceutically acceptable salt thereof.

Yet another embodiment of the present disclosure is a compound according to formula (4):

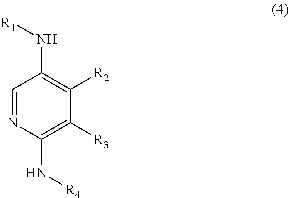

wherein:
R₁ and R₄ are independently selected from the group consisting of H, alkyl, aryl, C₁₋₆alkyl-aryl, C₁₋₆alkyl-phenolyl, C₃₋₁₂carbocycle, and polyyne, wherein each of the alkyl, aryl, C₁₋₆alkyl-aryl, C₁₋₆alkyl-phenolyl, C₃₋₁₂carbocycle, and polyyne are optionally substituted with one or more atoms or groups;
R₂ is selected from the group consisting of H, alkyl, aryl, and ether, wherein each of the alkyl, aryl, and ether are optionally substituted with one or more atoms or groups; and
R₃ is selected from the group consisting fo H, alkyl, aryl, an oxazole, an oxadiazole, an amide, an ether, or an ester, wherein each of the alkyl, aryl, oxazole, oxadiazole, amide, ether, and ester are optionally substituted with one or more atoms or groups;
or an N-oxide, crystalline form, hydrate, or a pharmaceutically acceptable salt thereof.

Another embodiment of the present disclosure is a pharmaceutical composition. This pharmaceutical composition comprises a pharmaceutically acceptable carrier or diluent and one or more compounds according to formula (2):

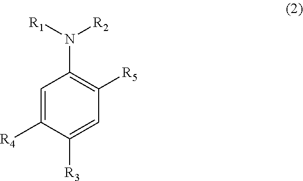

wherein:
R₁ and R₂ are independently selected from the group consisting of H, aryl, C₁₋₆alkyl-aryl, C₁₋₆alkyl-phenolyl, C₁₋₆alkyl-bicycle, and C₃₋₁₀carbocycle, wherein each of the aryl, C₁₋₆alkyl-aryl, C₁₋₆alkyl-phenolyl, C₁₋₆alkyl-bicycle, and C₃₋₁₀carbocycle are optionally substituted with one or more atoms or groups; or together, with the nitrogen attached, form a cyclic or bicyclic structure, wherein the cyclic or bicyclic structure is optionally substituted with one or more atoms or groups;
R₃ is selected from the group consisting of hydroxyl, alkoxy, and alcohol, wherein each of the hydroxyl, alkoxy, and alcohol are optionally substituted with one or more atoms or groups;
R₄ is selected from the group consisting of H, alkyl, and alkoxy; or together with R₃, form a ring structure, wherein the ring structure is optionally substituted with one or more atoms or groups; and
R₅ is selected from the group consisting of H, and alkoxy;
or an N-oxide, crystalline form, hydrate, or a pharmaceutically acceptable salt thereof.

Another embodiment of the present disclosure is a pharmaceutical composition. This pharmaceutical composition comprises a pharmaceutically acceptable carrier or diluent and one or more compounds according to formula (3):

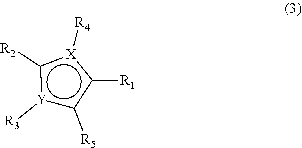

wherein:
X is selected from N, O, and S;
Y is C or N;
R₁ and R₅ are independently selected from the group consisting of H, alkenyl, ester, amino, and aryl, wherein each of the alkenyl, ester, amino, and aryl are optionally substituted with one or more atoms or groups; or together form a ring structure, wherein the ring structure is optionally substituted with one or more atoms or groups;
R₂ and R₃ together form a saturated or unsaturated ring structure, wherein the ring structure is optionally substituted with one or more atoms or groups; and
R₄ is selected from the group consisting of no atom, H, alkyl, alkenyl, and ketone;
or an N-oxide, crystalline form, hydrate, or a pharmaceutically acceptable salt thereof.

An additional embodiment of the present disclosure is a method for treating or ameliorating the effects of a neurodegenerative disease in a subject in need thereof. This method comprises administering to the subject an effective amount of a compound having the structure selected from the group consisting of:

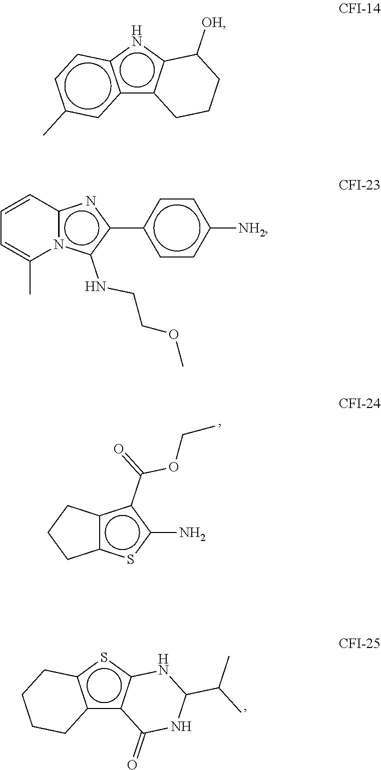

and combinations thereof, or an N-oxide, crystalline form, hydrate, or a pharmaceutically acceptable salt thereof.

An additional embodiment of the present disclosure is a method of modulating ferroptosis in a subject in need thereof. This method comprises administering to the subject an effective amount of a ferroptosis inhibitor, which comprises a compound having the structure selected from the group consisting of:

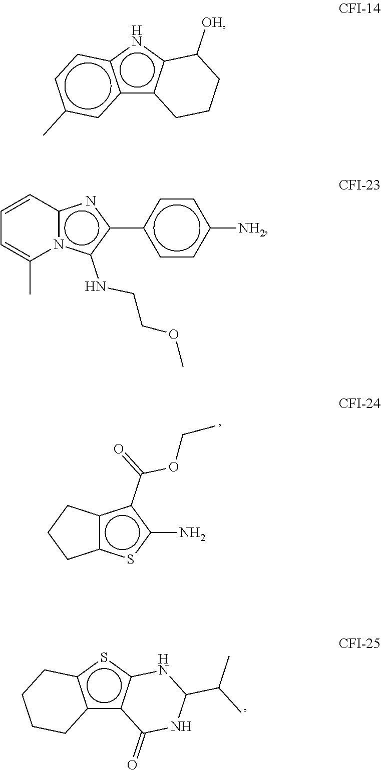

and combinations thereof, or an N-oxide, crystalline form, hydrate, or a pharmaceutically acceptable salt thereof.

An additional embodiment of the present disclosure is a method of reducing reactive oxygen species (ROS) in a cell. This method comprises contacting a cell with a ferroptosis modulator, which comprises a compound having the structure selected from the group consisting of:

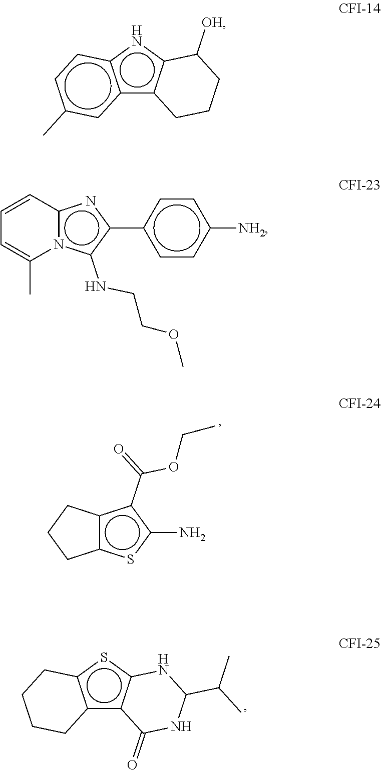

and combinations thereof, or an N-oxide, crystalline form, hydrate, or a pharmaceutically acceptable salt thereof.

An additional embodiment of the present disclosure is a method for treating or ameliorating the effects of a neurodegenerative disease in a subject in need thereof. This method comprises administering to the subject an effective amount of a compound having the structure selected from the group consisting of:

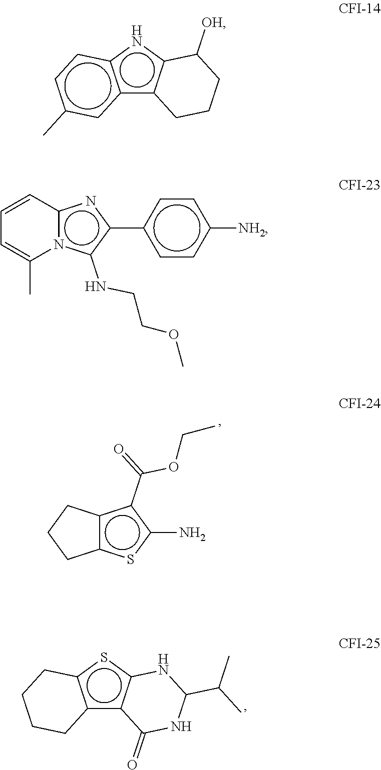

and combinations thereof, or an N-oxide, crystalline form, hydrate, or a pharmaceutically acceptable salt thereof.

Still another embodiment of the present disclosure is a method for alleviating side effects in a subject undergoing radiotherapy and/or immunotherapy, comprising administering to the subject an effective amount of one or more compounds disclosed herein.

A further embodiment of the present disclosure is a method for treating or ameliorating the effects of an infection associated with ferroptosis in a subject, comprising administering to the subject an effective amount of one or more compounds disclosed herein.

### BRIEF DESCRIPTION OF THE DRAWINGS

FIGS. 1A-1C show the biological activities of Ferrostatin-1 and analogs. FIG. 1A shows the dose-response relationship for inhibition of erastin (10 μM, 24 hours)-induced death in HT-1080 cells by Fer-1 and analogs. FIG. 1B shows the dose-response relationship for inhibition of IKE or RSL3-induced death in HT-1080 cells by Fer-1 and analogs. FIG. 1C shows the structure of various compounds listed in FIGS. 1A and 1B.

FIG. 2 shows the microsomal stability of Fer-1, CFI-102 and TH-2-9-1 in mouse.

FIG. 3 shows the metabolic stability of CFI-4082 in plasma, brain, liver and kidney.

FIG. 4 shows the structure of selected Fer-1 analogs further tested in Example 4.

FIG. 5A shows the dose-response curves of TH-2-9-1, TH-2-5, and Fer-1 at a concentration range from 20 μM-0 μM against 3 μM IKE and 0.2 μM RSL3.

FIG. 5B shows the dose-response curves of TH-2-9-1, TH-2-5, and Fer-1 at a concentration range from 10 μM-0 μM against 3 μM IKE and 0.2 μM RSL3.

FIG. 5C shows the dose-response curves of TH-2-9-1, TH-2-5, and Fer-1 at a concentration range from 1 μM-0 μM against 10 μM Erastin, 3 μM IKE and 0.2 μM RSL3. Asterisk (*) indicates standardized result.

FIG. 5D shows the dose-response curves of TH-2-9-1, TH-2-5, and Fer-1 at a concentration range from 1 μM-0 μM against 10 μM Erastin, 3 μM IKE and 0.2 μM RSL3, from a second set of experiments.

FIG. 6A shows the dose-response curves of CFI-102 and TH-2-30 at a concentration range from 10 μM-0 μM against 3 μM IKE and 0.2 μM RSL3.

FIG. 6B shows the dose-response curves of CFI-102 and TH-2-30 at a concentration range from 2.5 μM-0 μM against 3 μM IKE and 0.2 μM RSL3.

FIG. 6C shows the dose-response curves of CFI-102 and TH-2-30 at a concentration range from 5 μM-0 μM against 3 μM IKE and 0.2 μM RSL3. HT-1080 cells were incubated for 51 hours.

FIG. 7 shows the dose-response curves of CFI-102, TH-2-30, TH-2-9-1 and Fer-1 at a concentration range from 5 μM-0 μM against 3 μM IKE and 0.2 μM RSL3. HT-1080 cells were incubated for 49 hours.

FIG. 8 shows the structures of the optimized analogs and the corresponding inactive analogs.

FIG. 9 shows the structures and representative dose-response curves of active ferrostatins TH-2-31, TH-4-55-2, and TH-4-67 (N=3).

FIG. 10 shows the structures and dose-response curves of inactive controls TH-4-50-2, TH-4-46-2, and TH-4-58-2 (N=3).

FIG. 11A shows microsomal stability of 3 active analogs (n=2 wells/compound/experiment).

FIG. 11B shows plasma stability (mouse) curves of each optimized analogs (n=2 wells/compound/experiment, N=2).

FIG. 12 shows the mutagenic potential of selected optimized analogs was assessed using the Fluctuation AMES test.

FIG. 13 shows pharmacokinetics in plasma and brain of three active ferrostatins administered via IP, IV and PO.

FIG. 14 shows BBB permeabilities calculated as log₁₀(brain/plasma) values for each compound at each time point.

FIG. 15 shows brain concentration of each compound over time.

FIG. 16 shows Cₘₐₓ/IC₅₀ for brain and plasma of each optimized compounds and R.O.A.

FIG. 17A shows the effects of selected optimized ferrostatin analogs treatment on 3-NP-induced weight loss.

FIGS. 17B-17D show the effects of optimized ferrostatin analogs treatment on OpenField behavior at Day −5 (B), Day −2 (C) and Day 4 (D).

FIGS. 18A-18D show that the optimized ferrostatin analogs are well tolerated in symptomatic R6/2 mice. R6/2 mice at 10 weeks of age were dosed with vehicle or fifth generation analog at 20 mg/kg via intraperitoneal injection or oral gavage for one month and the change in body weight compared to the baseline calculated. FIG. 18A shows the mice survial rate via IP administration. FIG. 18B shows the mice survial rate via PO administration. FIG. 18C shows the mice weight loss via IP administration. FIG. 18D shows the mice weight loss via PO administration.

FIG. 19 shows the fourth generation ferrostatin scaffold. The ferrostatin molecular scaffold incorporates four sites for substitution.

FIGS. 20A-20C show that fourth generation ferrostatin analogs have differential stability in mouse liver microsomes and structural diversity. FIG. 20A shows that the microsomal stability of fourth generation ferrostatin analogs was assessed over a two-hour time period. FIG. 20B shows that the stability of fourth generation analogs was assessed in mouse plasma over a six-hour time period. FIG. 20C shows structures of fourth generation analogs.

FIGS. 21A-21D show that PHB4082 stably accumulates in mouse kidney with minimal accumulation observed in brain. C57BL/6 mice at ˜8 weeks of age were dosed with PHB4082 (same as CFI-4082) via intraperitoneal injection and the mice were sacrificed. FIG. 21A shows that the concentration of PHB4082 in murine plasma and brain was determined via UHPLC-MS. FIG. 21B shows that the log₁₀ ratio of the concentration of PHB4082 in brain over plasma was calculated at each time-point. FIG. 21C shows that the concentration of PHB4082 in plasma, brain, liver, and kidney was determined at each time-point. FIG. 21D shows that the effect of PHB4082 on glutathione adduct formation and mutagenesis potential were assessed. PHB4082 was positive in the GSH adduct formation assay; the common criteria for GSH reactivity is 200 pmol/hr/mg protein.

FIGS. 22A-22D show that PHB4082 ameliorated renal tubular ferroptosis triggered by ferric nitrilotriacetate (Fe-NTA). FIG. 22A shows that PHB4082/vehicle was injected IP at time point 0, and Fe-NTA was injected after 2 hrs. Then after 24 hrs, mice were sacrificed for analysis. FIG. 22B shows that the rats with vehicle formulation died of renal failure with a high ratio (76.9%) after Fe-NTA IP, whereas all of those with PHB4082 pretreatment survived 24 hrs after Fe-NTA administration. FIG. 22C shows that PHB4082 dramatically reduced the necrotized renal tubular area. FIG. 22D shows that PHB4082 pretreatment group showed less amounts of HNE adducts accumulated in proximal tubules and less signals for TRF1/CD71 in the proximal tubules.

FIG. 23 shows that fifth generation analogs have differential stability in mouse liver microsomes. The stability of four lead fifth-generation analogs and Fer-1 in mouse liver microsomes was assessed over a two-hour time period and the half-life period of them were calculated.

FIGS. 24A-24D show that lead fifth generation analogs have desirable in vitro properties. FIG. 24A shows that the stability of three fifth generation analogs in mouse liver microsomes was assessed over a two-hour time period. FIG. 24B shows that the stability of the lead analogs was assessed in mouse plasma over a four-hour time period. FIG. 24C shows structures of the lead analogs. FIG. 24D shows that the mutagenic potential of fifth generation analogs was assessed using the Fluctuation Ames test.

FIGS. 25A-25D show that fifth generation analogs have good in vivo pharmacokinetic properties. C57BL/6 mice were dosed with TH-2-31, TH-4-55-2, and TH-4-67 at 20 mg/kg via intraperitoneal, intravenous, and oral gavage routes of administration. The concentration of each analog in plasma (FIG. 25A) and brain (FIG. 25B) was determined over a 24-hour time period. FIG. 25C shows that the maximum concentration was determined for each analog and route of administration. FIG. 25D shows that the ratio of the concentration of analog in brain over plasma was determined for each route of administration.

FIGS. 26A-26D show that fifth generation analogs are well tolerated in symptomatic R6/2 mice, and TH-4-55-2 showed a significant protective effect. R6/2 mice at 10 weeks of age were dosed with vehicle or fifth generation analog at 20 mg/kg via intraperitoneal injection or oral gavage for one month and the change in body weight compared to the baseline calculated. FIG. 26A shows the weight change of R6/2 male mice group via PO administration over 30-day course. FIG. 26B shows the weight change of R6/2 female mice group via PO administration over 30-day course. FIG. 26C shows R6/2 mice survival rate over the 30-day course. FIG. 26D shows that R6/2 male mice groups treated by different lead compounds via PO administration over 20-day course were analyzed separately (On the 20^{th} day, the first mouse was sacrificed for losing weight over 20%). TH-4-55-2 showed a significant protective effect.

FIG. 27 shows the updated SAR reflecting fifth generation ferrostatins. Different substitution groups were adopted on the benzene core and pyridine core. Anti-ferroptotic activity increases when both amines are secondary amines. Electron-donating groups also increase potency. Bulky ester groups or oxadiazoles can provide greater stability.

FIGS. 28A-28D show the formulation without PEG developed for efficacy study and LC-MS analysis. FIG. 28A shows that multiple solvents were tested using Nephelometer. Higher NTU (nephelometric turbidity units) means poor solubility. The co-solvents without a measurement indicate a strong visual of immiscibility. FIG. 28B shows that five potential co-solvents were tested in two formulation combinations. Citric acid showed the lowest NTU and was most promising. FIG. 28C shows that the new formulation of three lead compounds was tested. FIG. 28D shows the concentration of TH-4-55-2 in brain samples of R6/2 mice.

FIGS. 29A-29B show weights and survival of R6/2 mice over 45-day dosing regimen of fifth-generation analogs. FIG. 29A shows that mice undergoing PO and drinking water (H2O) ferrostatin administration at 20 mg/kg were weighed daily, with percent change in body weight calculated from Day 0 baseline. FIG. 29B shows that survival was calculated as percent of total sample size for each treatment group.

FIG. 30 shows that R6/2 mice exhibited similar behavior across treatment conditions in the Rotarod behavioral task. Rotarod “Linear Incline” experiment was performed with 11- to 12-week-old R6/2 mice at Day 25 with a rotations per minute (RPM) ramp over a 300 s duration, start speed of 5 RPM, and max speed of 40 RPM. Latency to fall was recorded in seconds for each mouse. No significant differences were found among PO treatment groups; however, TH-4-55-2 showed consistently higher mean latency to fall across trials compared to control.

FIGS. 31A-31B show that R6/2 mice showed significant improvement to motor performance in the Catwalk Behavioral task for TH-4-67, partial improvement for TH-4-55-2, and mixed results for TH-2-31. Catwalk behavioral task was performed with 11- to 12-week-old R6/2 mice on Day 25; each mouse completed Catwalk runs until three coordinated runs were detected and verified. Runs and mice were paired by Regularity Index, a measure of interlimb coordination, to group runs and mice into most coordinated “Best” and least coordinated “Worst” runs and mice. FIG. 31A shows that R6/2 mouse gait assessed by Cadence shows improved motor performance with treatment of all ferrostatin analogs with the most coordinated runs and mice. FIG. 31B shows that R6/2 mouse gait assessed by Base of Support (BOS) shows significantly worse performance by TH-2-31 mice in both front and hind paws. TH-4-67 shows significant improvement of BOS in the hind paws of least coordinated mice.

FIG. 32 shows representation of In vivo pharmacokinetic mouse study of TH-4-55-2, TH-4-67, TH-2-31 in drinking water and PO. Depiction of the strategy involving the administration of ferrostatin analogs through drinking water and PO over a 45-day period in symptomatic male R6/2 mice. Subsequent to CO₂ euthanasia, plasma, brain, and liver samples were harvested.

FIG. 33 shows summary of all 19 fourth generation ferrostatins, related to FIGS. 20A-20C.

FIGS. 34A-34B show in vitro stability of fourth generation ferrostatins in mouse microsomes and plasma, related to FIGS. 20A-20C. FIG. 34A shows the stability of fourth generation analogs in mouse liver microsomes over a two-hour time period compared to fer-1. FIG. 34B shows the stability of fourth generation analogs in mouse plasma over a four-hour time period compared to fer-1.

FIG. 35 shows fifth generation analogs that incorporate a wide range of structural diversity, related to FIGS. 21A-21D.

FIGS. 36A-36B show that TH-2-31 has favorable pharmacokinetics in vivo, related to FIG. 23. C57BL/6 mice were dosed at 40 mg/kg IP and the concentrations of TH-2-31 were determined in plasma and brain (FIG. 36A), and the log₁₀ of the concentration of analog in brain over plasma calculated (FIG. 36B).

FIG. 37 shows that fifth generation analogs accumulate in brain and plasma at orders of magnitude higher than the IC₅₀ values, related to FIGS. 25A-25D.

FIGS. 38A-38C show that fifth generation analogs do not protect against 3-nitropropinoic acid-induced behavioral deficits. FIG. 38A shows the 3-NP efficacy study design. FIG. 38B shows the effects of fifth generation ferrostatin analog treatment on 3-NP-induced weight loss. FIG. 38C shows the effect of ferrostatin treatment on OpenField behavior at X₁ (i), X₂ (ii), and X₃ (iii).

FIGS. 39A-39D show that lead compounds are well tolerated over 30 days in wild-type and R6/2 mice, related to FIGS. 26A-26D. R6/2 mice and C57BL/6 wild type mice at 10 weeks of age were dosed with vehicle or fifth generation analog at 20 mg/kg and the change in body weight compared to the baseline calculated. FIG. 39A shows R6/2 male mice group via intraperitoneal injection administration over 30-day course. FIG. 39B shows R6/2 female mice group via IP administration over 30-day course. FIG. 39C shows C57BL/6 wild type mice group via IP administration over 30-day course. FIG. 39D shows C57BL/6 wild type mice group via oral gavage administration over 30-day course.

FIG. 40 shows that route of administration had a significant effect on survival. R6/2 mice at ˜6-7 weeks were dosed with vehicle or fifth generation analog at 20 mg/kg via drinking water or oral gavage over 45-day course. Cox regression analysis on survival data revealed a significant effect of route of administration on survival, with the drinking water group yielding a lower survival rate. Drug administration versus vehicle did not have a significant effect on survival.

FIG. 41 shows that regularity Index is an effective metric for pairing in Catwalk Gait analysis. Individual runs and mice were paired by regularity index. The runs for highest-scoring mice within each treatment group were paired sequentially from highest to lowest values, then compared among themselves for further behavioral analysis in the “Best” group. Similarly, the runs of lowest-scoring mice within each treatment group were paired sequentially from highest to lowest values, then compared among themselves for further analysis in the “Worst” group. Pairing was evaluated via RM one-way ANOVA.

FIGS. 42A-42C show that individual paw measurements in Catwalk Behavioral task did not yield discernable effects. Individual paw measurements were analyzed by Noldus Gait Analysis system. All comparisons within “Best” and “Worst” groups were made using RM one-way ANOVA. FIG. 42A shows the results of stride length. FIG. 42B shows the results of stand. FIG. 42C shows the results of swing.

FIGS. 43A-43B show accumulation of ferrostatin analogues via drinking water over 45-day course detected via LC/MS. R6/2 mice at ˜6-7 weeks were dosed with ferrostatins via PO and drinking water (H2O) over 45-day course. FIG. 43A shows that ferrostatins accumulated at varying semi-micromolar quantities in R6/2 mouse brains. FIG. 43B shows that TH-4-55-2 preferentially accumulated in the brain over plasma for both routes of administration. Preferential accumulation in the brain over plasma varied for TH-2-31 and TH-4-67.

### DETAILED DESCRIPTION OF THE DISCLOSURE

In the present disclosure, new analogs of Fer-1 are provided. Certain of the analogs have improved microsomal stability and solubility while still maintaining good inhibition potency of ferroptosis. Accordingly, one embodiment of the present disclosure is a compound according to formula (1):

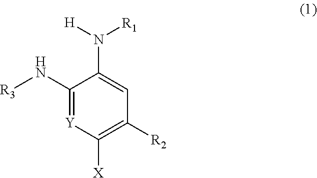

wherein:
R₁ is selected from the group consisting of H, alkyl, aryl, C₁₋₆alkyl-aryl, C₁₋₆alkyl-phenolyl, and C₃₋₁₀carbocycle, wherein each of the alkyl, aryl, C₁₋₆alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
R₃ is a C₃₋₁₂carbocycle, or a polyyne, wherein each of the C₃₋₁₂carbocycle and polyyne are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R₁ and X are both H, Y is —CH and R₃ is

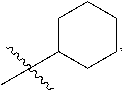 [SMILES: *C1CCCCC1]

R₂ cannot be

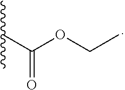 [SMILES: *C(=O)OCC]

In one aspect of this embodiment, the compound has the structure of formula (1a):

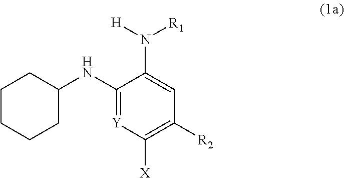

wherein:
R₁ is selected from the group consisting of H, alkyl, aryl, C₁₋₆alkyl-aryl, C₁₋₆alkyl-phenolyl, and C₃₋₁₀carbocycle, wherein each of the alkyl, aryl, C₁₋₆alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R₁ and X are both H and Y is —CH, R₂ cannot be

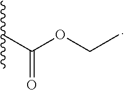 [SMILES: *C(=O)OCC]

In another aspect of this embodiment, the compound has the structure of formula (1b):

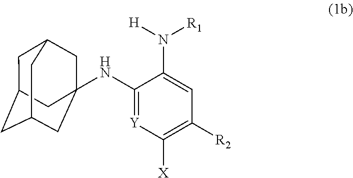

wherein:
R₁ is selected from the group consisting of H, alkyl, aryl, C₁₋₆alkyl-aryl, C₁₋₆alkyl-phenolyl, and C₃₋₁₀carbocycle, wherein each of the alkyl, aryl, C₁₋₆alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups; and
X is selected from the group consisting of H, optionally substituted alkyl, and halo; and
Y is —CH or N;
or an N-oxide, crystalline form, hydrate, or a pharmaceutically acceptable salt thereof.

In another aspect of this embodiment, the compound is selected from the group consisting of:

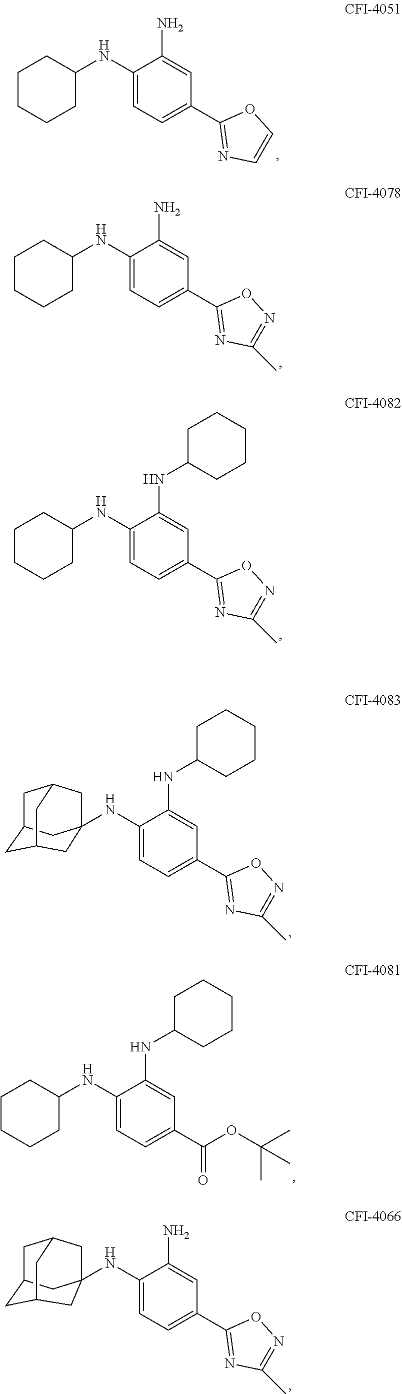 [SMILES: Nc1cc(-c2ncco2)ccc1NC1CCCCC1 ; Cc1noc(-c2ccc(NC3CCCCC3)c(N)c2)n1 ; Cc1noc(-c2ccc(NC3CCCCC3)c(NC3CCCCC3)c2)n1 ; Cc1noc(-c2ccc(NC34CC5CC(CC(C5)C3)C4)c(NC3CCCCC3)c2)n1 ; CC(C)(C)OC(=O)c1ccc(NC2CCCCC2)c(NC2CCCCC2)c1 ; Cc1noc(-c2ccc(NC34CC5CC(CC(C5)C3)C4)c(N)c2)n1]

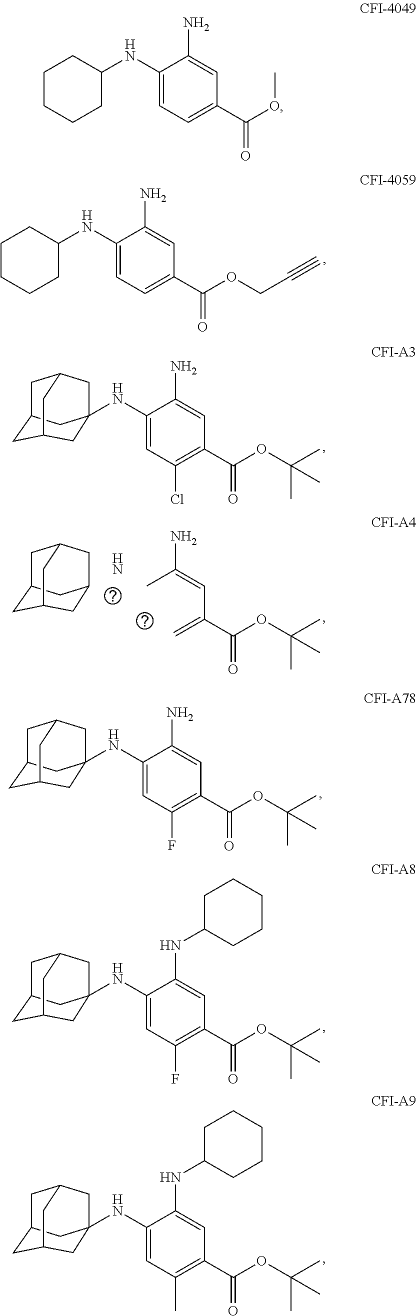

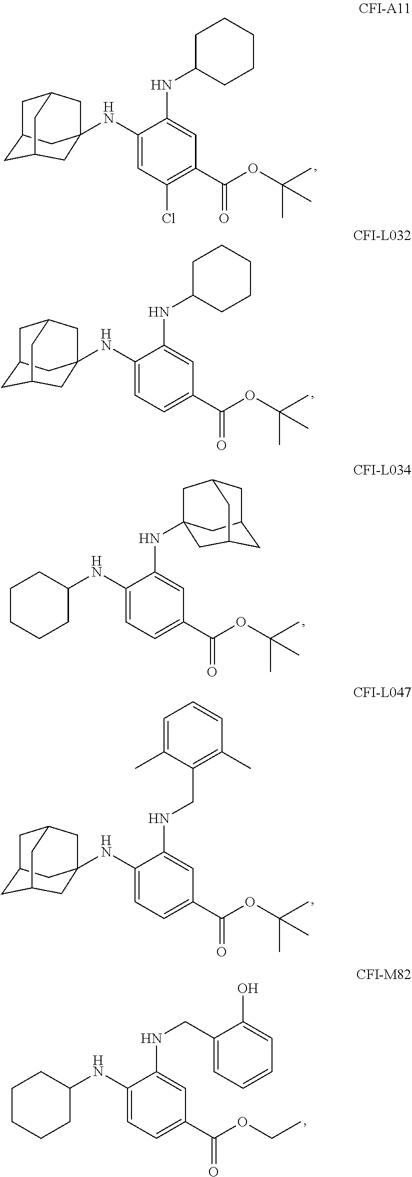

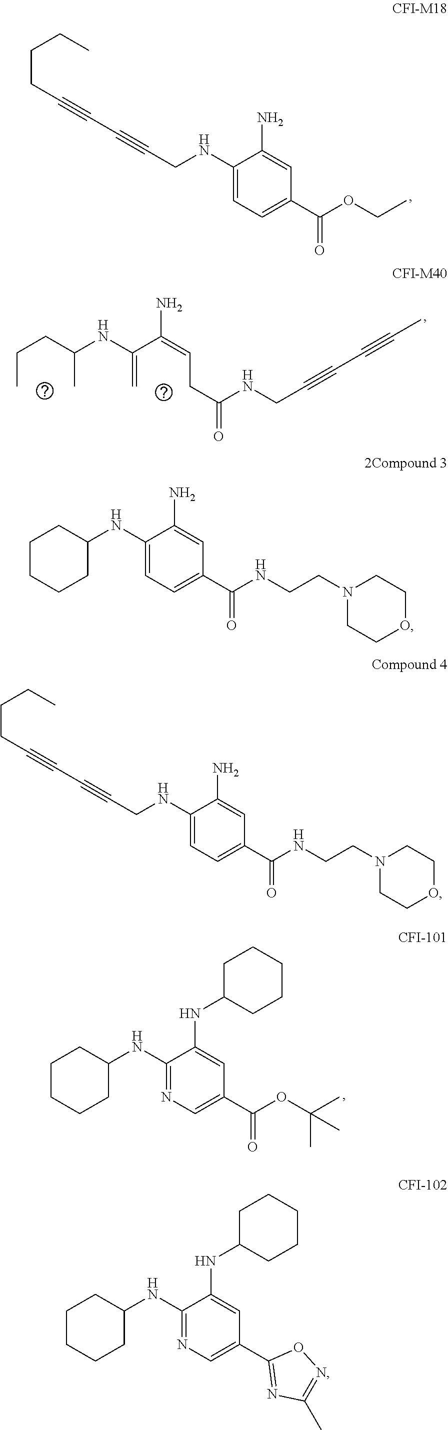 [SMILES: CCCCC#CC#CCNc1ccc(C(=O)OCC)cc1N ; C=C(NC(C)CCC)/C(N)=C\CC(=O)NCC#CC#CC ; Nc1cc(C(=O)NCCN2CCOCC2)ccc1NC1CCCCC1 ; CCCCC#CC#CCNc1ccc(C(=O)NCCN2CCOCC2)cc1N ; CC(C)(C)OC(=O)c1cnc(NC2CCCCC2)c(NC2CCCCC2)c1 ; Cc1noc(-c2cnc(NC3CCCCC3)c(NC3CCCCC3)c2)n1]

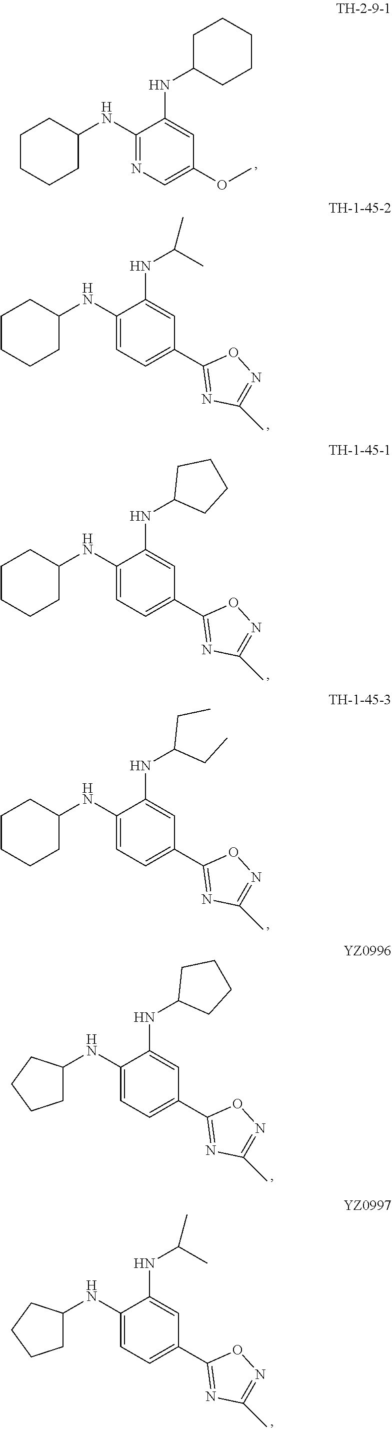

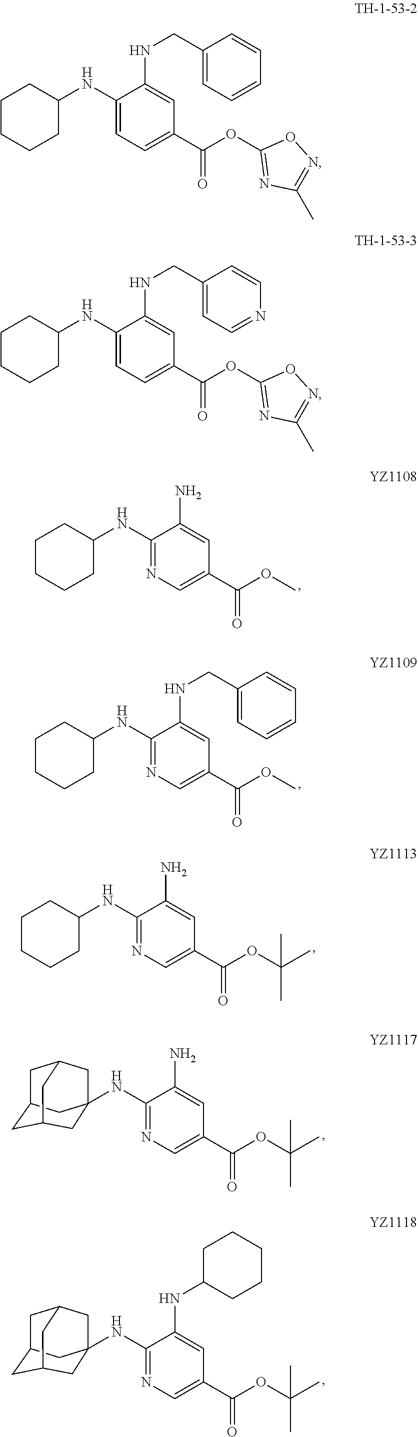

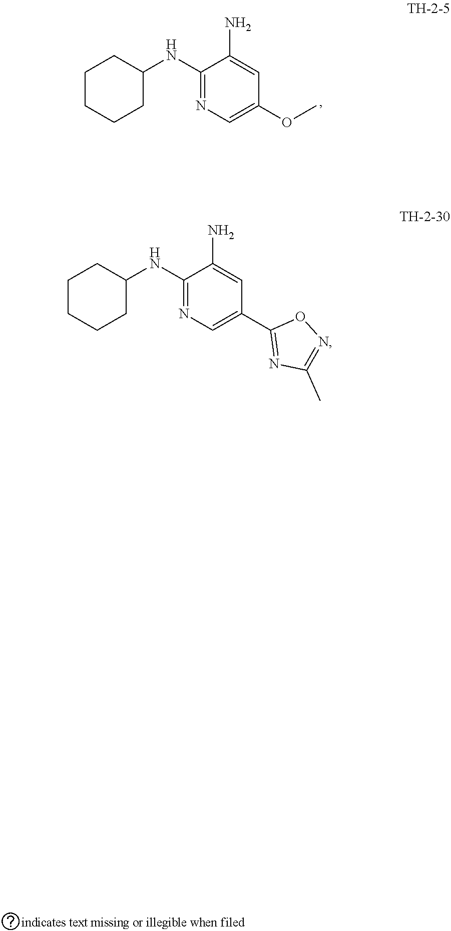 [SMILES: COc1cnc(NC2CCCCC2)c(N)c1 ; Cc1noc(-c2cnc(NC3CCCCC3)c(N)c2)n1]

or an N-oxide, crystalline form, hydrate, or a pharmaceutically acceptable salt thereof.

Preferably, the compound is selected from the group consisting of:

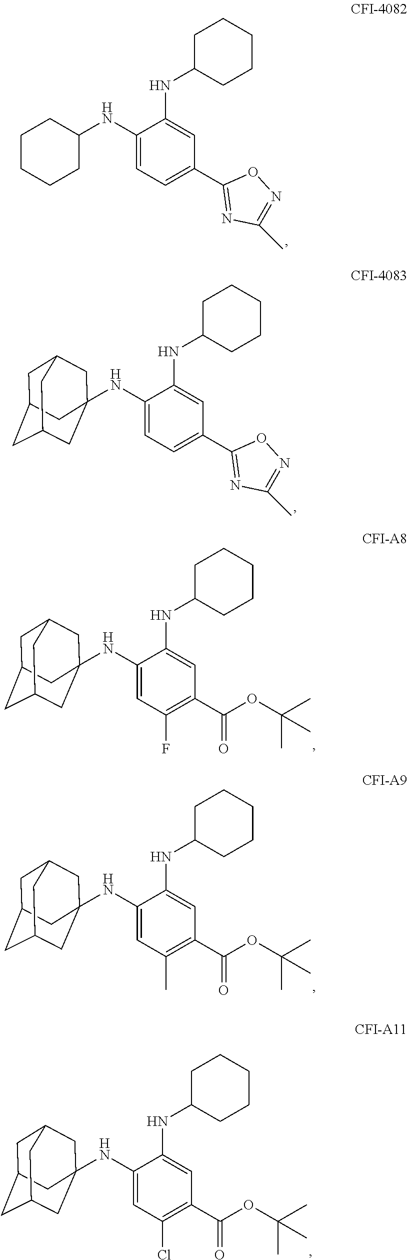

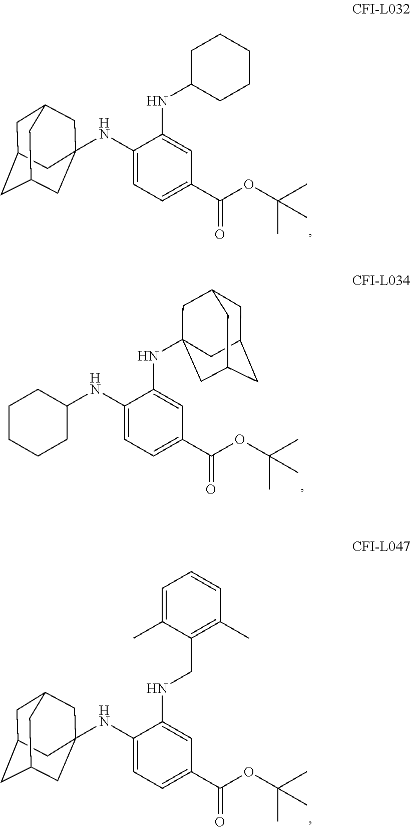 [SMILES: CC(C)(C)OC(=O)c1ccc(NC23CC4CC(CC(C4)C2)C3)c(NC2CCCCC2)c1 ; CC(C)(C)OC(=O)c1ccc(NC2CCCCC2)c(NC23CC4CC(CC(C4)C2)C3)c1 ; Cc1cccc(C)c1CNc1cc(C(=O)OC(C)(C)C)ccc1NC12CC3CC(CC(C3)C1)C2]

and combinations thereof, or an N-oxide, crystalline form, hydrate, or a pharmaceutically acceptable salt thereof.

More preferably, the compound is selected from the group consisting of:

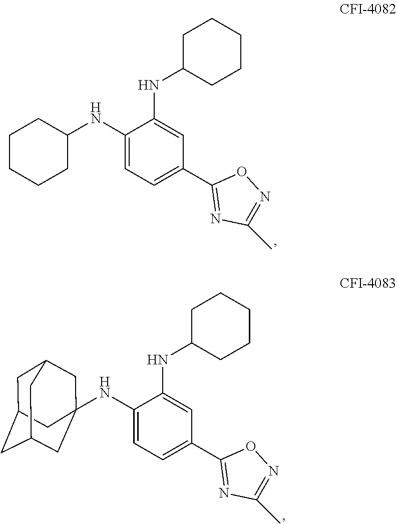 [SMILES: Cc1noc(-c2ccc(NC3CCCCC3)c(NC3CCCCC3)c2)n1 ; Cc1noc(-c2ccc(NC34CC5CC(CC(C5)C3)C4)c(NC3CCCCC3)c2)n1]

or an N-oxide, crystalline form, hydrate, or a pharmaceutically acceptable salt thereof.

Another embodiment of the present disclosure is a compound having the structure selected from the group consisting of:

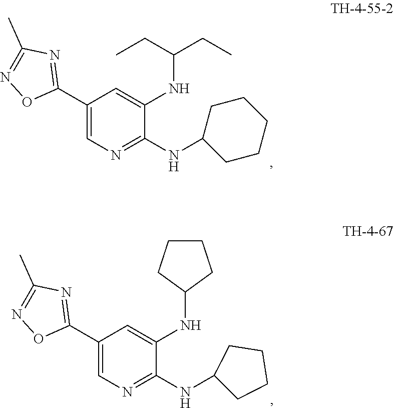 [SMILES: CCC(CC)Nc1cc(-c2nc(C)no2)cnc1NC1CCCCC1 ; Cc1noc(-c2cnc(NC3CCCC3)c(NC3CCCC3)c2)n1]

and combinations thereof, or an N-oxide, crystalline form, hydrate, or a pharmaceutically acceptable salt thereof.

Another embodiment of the present disclosure is a compound according to formula (2):

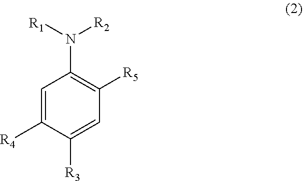

wherein:
R₁ and R₂ are independently selected from the group consisting of H, aryl, C₁₋₆alkyl-aryl, C₁₋₆alkyl-phenolyl, C₁₋₆alkyl-bicycle, and C₃₋₁₀carbocycle, wherein each of the aryl, C₁₋₆alkyl-aryl, C₁₋₆alkyl-phenolyl, C₁₋₆alkyl-bicycle, and C₃₋₁₀carbocycle are optionally substituted with one or more atoms or groups; or together, with the nitrogen attached, form a cyclic or bicyclic structure, wherein the cyclic or bicyclic structure is optionally substituted with one or more atoms or groups;
R₃ is selected from the group consisting of hydroxyl, alkoxy, and alcohol, wherein each of the hydroxyl, alkoxy, and alcohol are optionally substituted with one or more atoms or groups;
R₄ is selected from the group consisting of H, alkyl, and alkoxy; or together with R₃, form a ring structure, wherein the ring structure is optionally substituted with one or more atoms or groups; and
R₅ is selected from the group consisting of H, and alkoxy;
or an N-oxide, crystalline form, hydrate, or a pharmaceutically acceptable salt thereof.

Preferably, the compound is selected from the group consisting of:

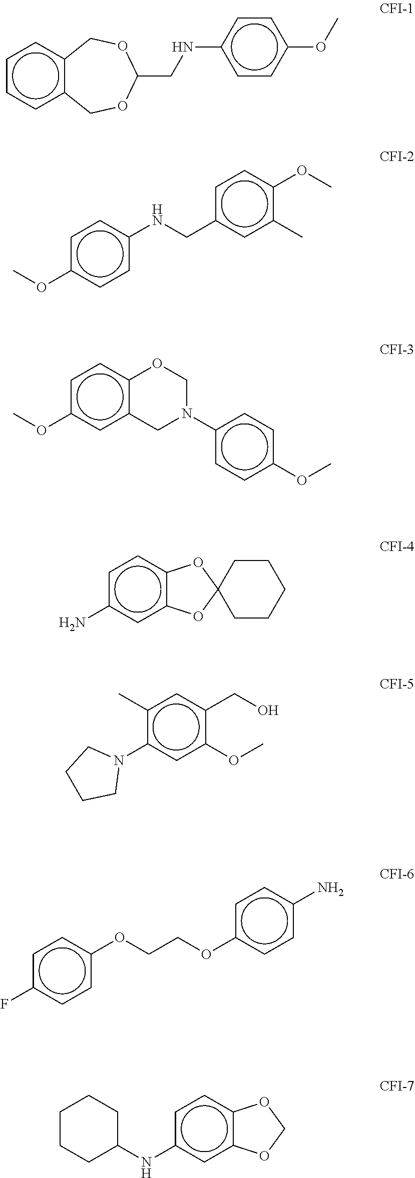

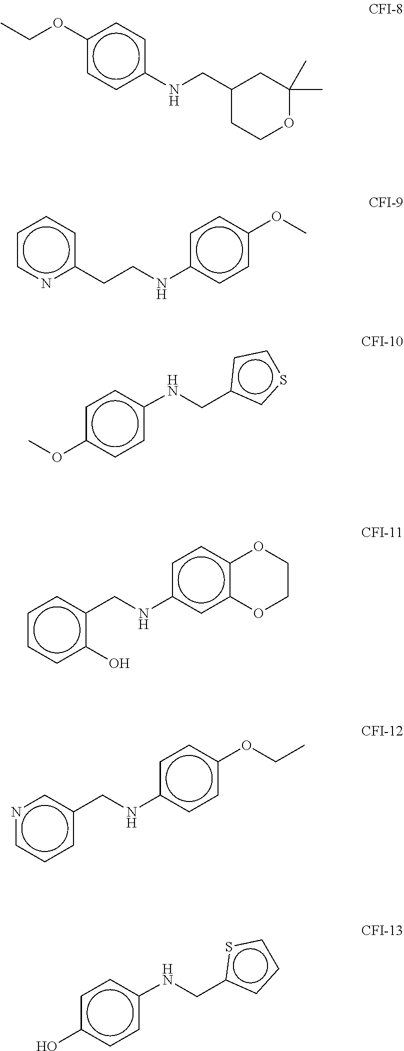

or an N-oxide, crystalline form, hydrate, or a pharmaceutically acceptable salt thereof.

Another embodiment of the present disclosure is a compound according to formula (3):

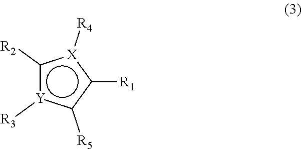

wherein:
X is selected from N, O, and S;
Y is C or N;
R₁ and R₅ are independently selected from the group consisting of H, alkenyl, ester, amino, and aryl, wherein each of the alkenyl, ester, amino, and aryl are optionally substituted with one or more atoms or groups; or together form a ring structure, wherein the ring structure is optionally substituted with one or more atoms or groups;
R₂ and R₃ together form a saturated or unsaturated ring structure, wherein the ring structure is optionally substituted with one or more atoms or groups; and
R₄ is selected from the group consisting of no atom, H, alkyl, alkenyl, and ketone;
or an N-oxide, crystalline form, hydrate, or a pharmaceutically acceptable salt thereof.

In one aspect of this embodiment, the compound has the structure of formula (3a):

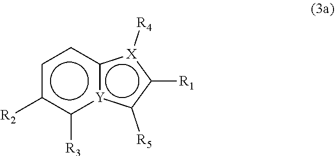

wherein:
X is selected from N, O, and S;
Y is C or N;
R₁ and R₅ are independently selected from the group consisting of H, alkenyl, ester, amino, and aryl, wherein each of the alkenyl, ester, amino, and aryl are optionally substituted with one or more atoms or groups; or together form a ring structure, wherein the ring structure is optionally substituted with one or more atoms or groups;
R₂ and R₃ are independently selected from the group consisting of H, alkyl, amino, and halo; and R₄ is selected from the group consisting of no atom, H, alkyl, alkenyl, and ketone;
or an N-oxide, crystalline form, hydrate, or a pharmaceutically acceptable salt thereof.

In another aspect of this embodiment, the compound is selected from the group consisting of:

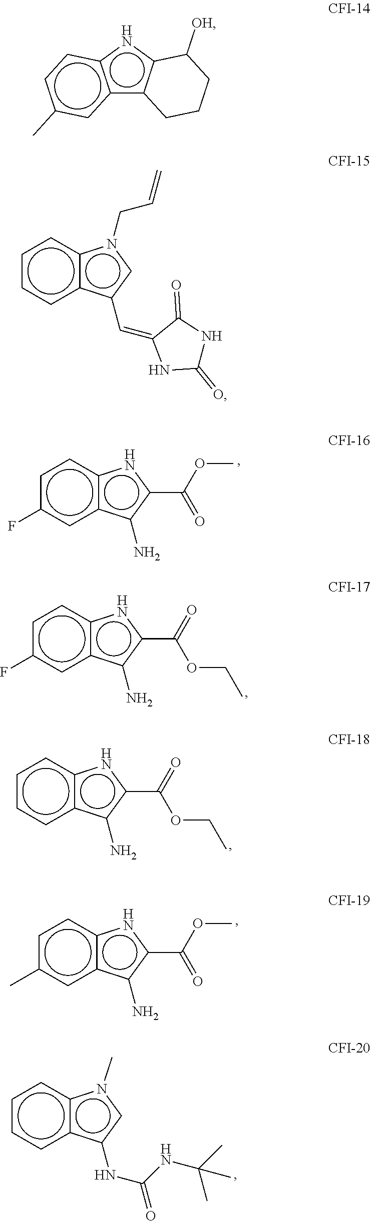

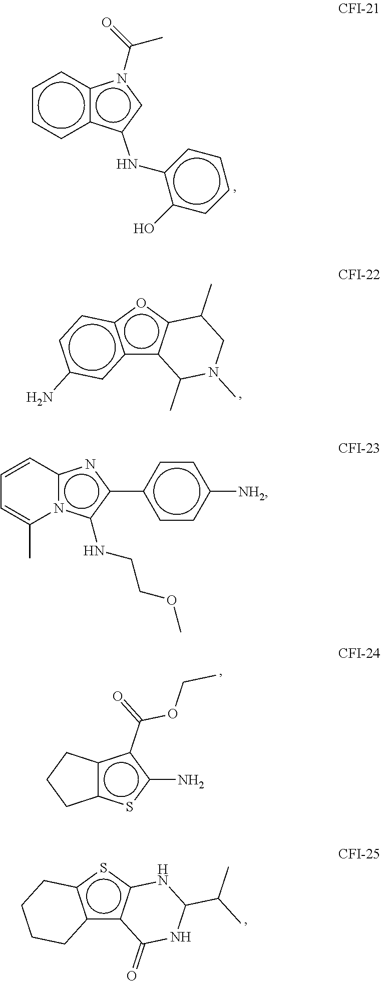

or an N-oxide, crystalline form, hydrate, or a pharmaceutically acceptable salt thereof.

Preferably, the compound is selected from the group consisting of:

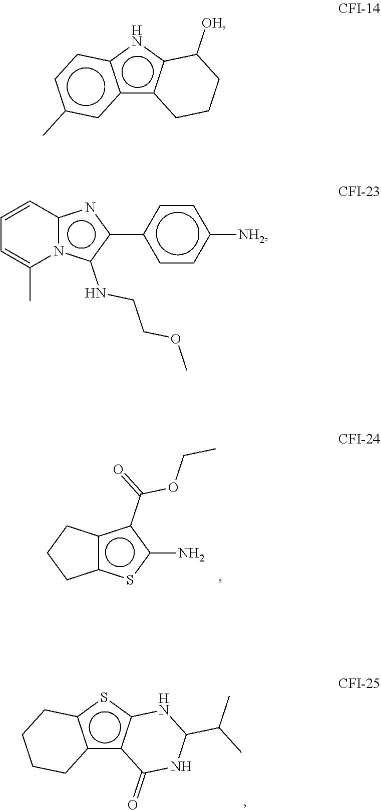

or an N-oxide, crystalline form, hydrate, or a pharmaceutically acceptable salt thereof.

Yet another embodiment of the present disclosure is a compound according to formula (4):

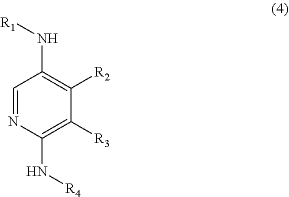

wherein:
R₁ and R₄ are independently selected from the group consisting of H, alkyl, aryl, C₁₋₆alkyl-aryl, C₁₋₆alkyl-phenolyl, C₃₋₁₂carbocycle, and polyyne, wherein each of the alkyl, aryl, C₁₋₆alkyl-aryl, C₁₋₆alkyl-phenolyl, C₃₋₁₂carbocycle, and polyyne are optionally substituted with one or more atoms or groups;
R₂ is selected from the group consisting of H, alkyl, aryl, and ether, wherein each of the alkyl, aryl, and ether are optionally substituted with one or more atoms or groups; and
R₃ is selected from the group consisting fo H, alkyl, aryl, an oxazole, an oxadiazole, an amide, an ether, or an ester, wherein each of the alkyl, aryl, oxazole, oxadiazole, amide, ether, and ester are optionally substituted with one or more atoms or groups;
or an N-oxide, crystalline form, hydrate, or a pharmaceutically acceptable salt thereof.

Preferably, the compound is selected from the group consisting of:

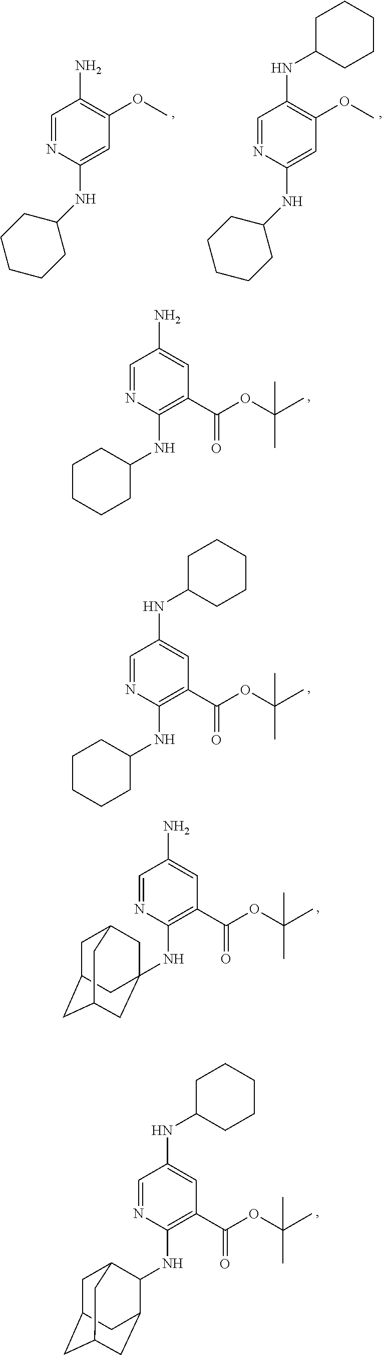 [SMILES: COc1cc(NC2CCCCC2)ncc1N ; COc1cc(NC2CCCCC2)ncc1NC1CCCCC1 ; CC(C)(C)OC(=O)c1cc(N)cnc1NC1CCCCC1 ; CC(C)(C)OC(=O)c1cc(NC2CCCCC2)cnc1NC1CCCCC1 ; CC(C)(C)OC(=O)c1cc(N)cnc1NC12CC3CC(CC(C3)C1)C2 ; CC(C)(C)OC(=O)c1cc(NC2CCCCC2)cnc1NC1C2CC3CC(C2)CC1C3]

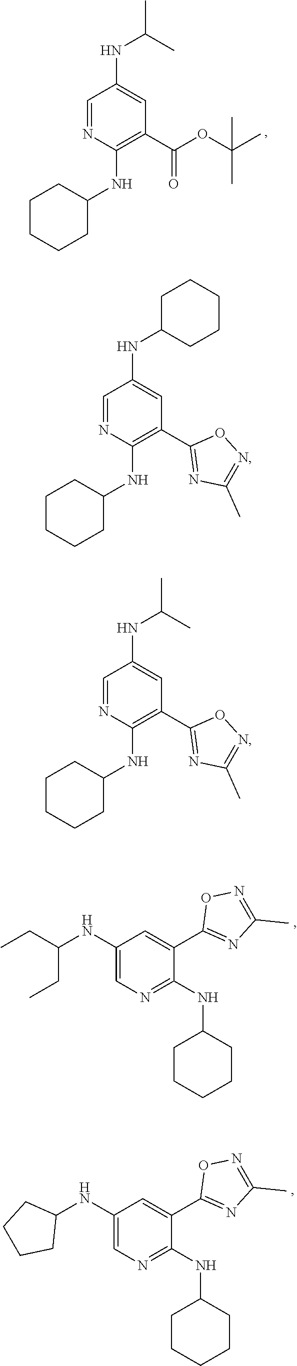

or an N-oxide, crystalline form, hydrate, or a pharmaceutically acceptable salt thereof.

Another embodiment of the present disclosure is a pharmaceutical composition. This pharmaceutical composition comprises a pharmaceutically acceptable carrier or diluent and one or more compounds according to formula (1):

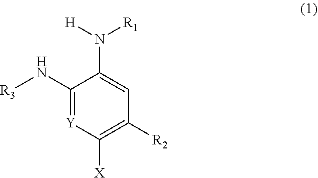

wherein:
R₁ is selected from the group consisting of H, alkyl, aryl, C₁₋₆alkyl-aryl, C₁₋₆alkyl-phenolyl, and C₃₋₁₀carbocycle, wherein each of the alkyl, aryl, C₁₋₆alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
R₃ is a C₃₋₁₂carbocycle, or a polyyne, wherein each of the C₃₋₁₂carbocycle and polyyne are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R₁ and X are both H, Y is —CH and R₃ is

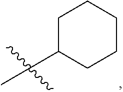 [SMILES: *C1CCCCC1]

R₂ cannot be

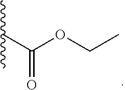 [SMILES: *C(=O)OCC]

Another embodiment of the present disclosure is a pharmaceutical composition. This pharmaceutical composition comprises a pharmaceutically acceptable carrier or diluent and one or more compounds according to formula (2):

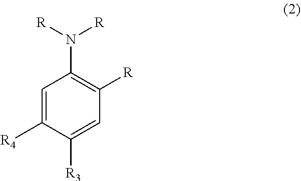

wherein:
R₁ and R₂ are independently selected from the group consisting of H, aryl, C₁₋₆alkyl-aryl, C₁₋₆alkyl-phenolyl, C₁₋₆alkyl-bicycle, and C₃₋₁₀carbocycle, wherein each of the aryl, C₁₋₆alkyl-aryl, C₁₋₆alkyl-phenolyl, C₁₋₆alkyl-bicycle, and C₃₋₁₀carbocycle are optionally substituted with one or more atoms or groups; or together, with the nitrogen attached, form a cyclic or bicyclic structure, wherein the cyclic or bicyclic structure is optionally substituted with one or more atoms or groups;
R₃ is selected from the group consisting of hydroxyl, alkoxy, and alcohol, wherein each of the hydroxyl, alkoxy, and alcohol are optionally substituted with one or more atoms or groups;
R₄ is selected from the group consisting of H, alkyl, and alkoxy; or together with R₃, form a ring structure, wherein the ring structure is optionally substituted with one or more atoms or groups; and
R₅ is selected from the group consisting of H, and alkoxy;
or an N-oxide, crystalline form, hydrate, or a pharmaceutically acceptable salt thereof.

Another embodiment of the present disclosure is a pharmaceutical composition. This pharmaceutical composition comprises a pharmaceutically acceptable carrier or diluent and one or more compounds according to formula (3):

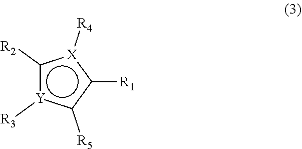

wherein:
X is selected from N, O, and S;
Y is C or N;
R₁ and R₅ are independently selected from the group consisting of H, alkenyl, ester, amino, and aryl, wherein each of the alkenyl, ester, amino, and aryl are optionally substituted with one or more atoms or groups; or together form a ring structure, wherein the ring structure is optionally substituted with one or more atoms or groups;
R₂ and R₃ together form a saturated or unsaturated ring structure, wherein the ring structure is optionally substituted with one or more atoms or groups; and
R₄ is selected from the group consisting of no atom, H, alkyl, alkenyl, and ketone;
or an N-oxide, crystalline form, hydrate, or a pharmaceutically acceptable salt thereof.

Suitable and preferred compounds that are used in the pharmaceutical compositions of the present disclosure are disclosed above in formulas (1), (1a), (1b), (2), (3), (3a) and (4), including the particular compounds also identified above.

A further embodiment of the present disclosure is a kit. This kit comprises a compound or a pharmaceutical composition disclosed herein with instructions for the use of the compound or the pharmaceutical composition, respectively.

The kits may also include suitable storage containers, e.g., ampules, vials, tubes, etc., for each compound of the present disclosure (which, e.g., may be in the form of pharmaceutical compositions) and other reagents, e.g., buffers, balanced salt solutions, etc., for use in administering the active agents to subjects. The compounds and/or pharmaceutical compositions of the disclosure and other reagents may be present in the kits in any convenient form, such as, e.g., in a solution or in a powder form. The kits may further include a packaging container, optionally having one or more partitions for housing the compounds and/or pharmaceutical compositions and other optional reagents.

Another embodiment of the present disclosure is a method for treating or ameliorating the effects of a disorder in a subject in need thereof. This method comprises administering to the subject an effective amount of one or more compounds having the structure of formula (1):

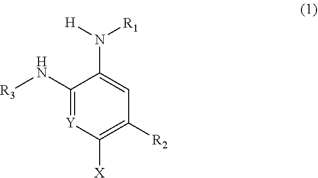

wherein:
R₁ is selected from the group consisting of H, alkyl, aryl, C₁₋₆alkyl-aryl, C₁₋₆alkyl-phenolyl, and C₃₋₁₀carbocycle, wherein each of the alkyl, aryl, C₁₋₆alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
R₃ is a C₃₋₁₂carbocycle, or a polyyne, wherein each of the C₃₋₁₂carbocycle and polyyne are optionally substituted with one or more atoms or groups; and
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R₁ and X are both H, Y is —CH and R₃ is

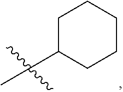 [SMILES: *C1CCCCC1]

R₂ cannot be

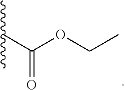 [SMILES: *C(=O)OCC]

As used herein, the terms “treat,” “treating,” “treatment” and grammatical variations thereof mean subjecting an individual subject to a protocol, regimen, process or remedy, in which it is desired to obtain a physiologic response or outcome in that subject, e.g., a patient. In particular, the methods and compositions of the present disclosure may be used to slow the development of disease symptoms or delay the onset of the disease or condition, or halt the progression of disease development. However, because every treated subject may not respond to a particular treatment protocol, regimen, process or remedy, treating does not require that the desired physiologic response or outcome be achieved in each and every subject or subject population, e.g., patient population. Accordingly, a given subject or subject population, e.g., patient population, may fail to respond or respond inadequately to treatment.

As used herein, the terms “ameliorate”, “ameliorating” and grammatical variations thereof mean to decrease the severity of the symptoms of a disease in a subject.

As used herein, a “subject” is a mammal, preferably, a human. In addition to humans, categories of mammals within the scope of the present disclosure include, for example, agricultural animals, veterinary animals, laboratory animals, etc. Some examples of agricultural animals include cows, pigs, horses, goats, etc. Some examples of veterinary animals include dogs, cats, etc. Some examples of laboratory animals include primates, rats, mice, rabbits, guinea pigs, etc.

Suitable and preferred compounds and pharmaceutical compositions for use in this method are as disclosed above in formulas (1), (1a), (1b), (2), (3), (3a) and (4), including the particular compounds identified above.

In one aspect of this embodiment, the disorder is a degenerative disease that involves lipid peroxidation. As used herein, “lipid peroxidation” means the oxidative degradation of fats, oils, waxes, sterols, triglycerides, and the like. Lipid peroxidation has been linked with many degenerative diseases, such as atherosclerosis, ischemia-reperfusion, heart failure, Alzheimer's disease, rheumatic arthritis, cancer, and other immunological disorders. (Ramana et al., 2013).

In another aspect of this embodiment, the disorder is an excitotoxic disease involving oxidative cell death. As used herein, an “excitotoxic disorder” means a disease related to the death of central neurons that are mediated by excitatory amino acids (such as glutamate). Excitotoxic disorders within the scope of the present disclosure include diseases involving oxidative cell death. As used herein, “oxidative” cell death means cell death associated with increased levels of intracellular reactive oxygen species (ROS). In the present disclosure, “reactive oxygen species” means chemically reactive molecules, such as free radicals, containing oxygen. Non-limiting examples of ROS include oxygen ions and peroxides.

Non-limiting examples of disorders according to the present disclosure include epilepsy, kidney disease, stroke, myocardial infarction, type I diabetes, traumatic brain injury (TBI), periventricular leukomalacia (PVL), and neurodegenerative disease. Non-limiting examples of neurodegenerative diseases according to the present disclosure include Alzheimer's, Parkinson's, Amyotrophic lateral sclerosis, Friedreich's ataxia, Multiple sclerosis, Huntington's Disease, Transmissible spongiform encephalopathy, Charcot-Marie-Tooth disease, Dementia with Lewy bodies, Corticobasal degeneration, Progressive supranuclear palsy, Chronic Traumatic Encephalopathy (CTE), and Hereditary spastic paraparesis.

In another aspect of this embodiment, the method further comprises co-administering, together with one or more compounds or pharmaceutical compositions of the present disclosure, to the subject an effective amount of one or more of additional therapeutic agents such as 5-hydroxytryptophan, Activase, AFQ056 (Novartis Corp., New York, NY), Aggrastat, Albendazole, alpha-lipoic acid/L-acetyl carnitine, Alteplase, Amantadine (Symmetrel), amlodipine, Ancrod, Apomorphine (Apokyn), Arimoclomol, Arixtra, Armodafinil, Ascorbic acid, Ascriptin, Aspirin, atenolol, Avonex, baclofen (Lioresal), Banzel, Benztropine (Cogentin), Betaseron, BGG492 (Novartis Corp., New York, NY), Botulinum toxin, Bufferin, Carbatrol®, Carbidopa/levodopa immediate-release (Sinemet), Carbidopa/levodopa oral disintegrating (Parcopa), Carbidopa/levodopa/Entacapone (Stalevo), CERE-110: Adeno-Associated Virus Delivery of NGF (Ceregene, San Diego, CA), cerebrolysin, CinnoVex, citalopram, citicoline, Clobazam, Clonazepam, Clopidogrel, clozapine (Clozaril), Coenzyme Q, Creatine, dabigatran, dalteparin, Dapsone, Davunetide, Deferiprone, Depakene®, Depakote ER®, Depakote®, Desmoteplase, Diastat, Diazepam, Digoxin, Dilantin®, Dimebon, dipyridamole, divalproex (Depakote), Donepezil (Aricept), EGb 761, Eldepryl, ELND002 (Elan Pharmaceuticals, Dublin, Ireland), Enalapril, enoxaparin, Entacapone (Comtan), epoetin alfa, Eptifibatide, Erythropoietin, Escitalopram, Eslicarbazepine acetate, Esmolol, Ethosuximide, Ethyl-EPA (Miraxion™), Exenatide, Extavia, Ezogabine, Felbamate, Felbatol®, Fingolimod (Gilenya), fluoxetine (Prozac), fondaparinux, Fragmin, Frisium, Gabapentin, Gabitril®, Galantamine, Glatiramer (Copaxone), haloperidol (Haldol), Heparin, human chorionic gonadotropin (hCG), Idebenone, Inovelon®, insulin, Interferon beta 1a, Interferon beta 1b, ioflupane 123I (DATSCAN®), IPX066 (Impax Laboratories Inc., Hayward, CA), JNJ-26489112 (Johnson and Johnson, New Brunswick, NJ), Keppra®, Klonopin, Lacosamide, L-Alpha glycerylphosphorylcholine, Lamictal®, Lamotrigine, Levetiracetam, liraglutide, Lisinopril, Lithium carbonate, Lopressor, Lorazepam, losartan, Lovenox, Lu AA24493, Luminal, LY450139 (Eli Lilly, Indianapolis, Indiana), Lyrica, Masitinib, Mecobalamin, Memantine, methylprednisolone, metoprolol tartrate, Minitran, Minocycline, mirtazapine, Mitoxantrone (Novantrone), Mysoline®, Natalizumab (Tysabri), Neurontin®, Niacinamide, Nitro-Bid, Nitro-Dur, nitroglycerin, Nitrolingual, Nitromist, Nitrostat, Nitro-Time, Norepinephrine (NOR), Carbamazepine, octreotide, Onfi®, Oxcarbazepine, Oxybutinin chloride, PF-04360365 (Pfizer, New York, NY), Phenobarbital, Phenytek®, Phenytoin, piclozotan, Pioglitazone, Plavix, Potiga, Pramipexole (Mirapex), pramlintide, Prednisone, Primidone, Prinivil, probenecid, Propranolol, PRX-00023 (EPIX Pharmaceuticals Inc.), PXT3003, Quinacrine, Ramelteon, Rasagiline (Azilect), Rebif, ReciGen, remacemide, Resveratrol, Retavase, reteplase, riluzole (Rilutek), Rivastigmine (Exelon), Ropinirole (Requip), Rotigotine (Neupro), Rufinamide, Sabril, safinamide (EMD Serono, Rockland, MA), Salagen, Sarafem, Selegiline (I-deprenyl, Eldepryl), SEN0014196 (Siena Biotech, Siena, Italy), sertraline (Zoloft), Simvastatin, Sodium Nitroprussiate (NPS), sodium phenylbutyrate, Stanback Headache Powder, Tacrine (Cognex), Tamoxifen, tauroursodeoxycholic acid (TUDCA), Tegretol®, Tenecteplase, Tenormin, Tetrabenazine (Xenazine), THR-18 (Thrombotech Ltd.), Tiagabine, Tideglusib, tirofiban, tissue plasminogen activator (tPA), tizanidine (Zanaflex), TNKase, Tolcapone (Tasmar), Tolterodine, Topamax®, Topiramate, Trihexyphenidyl (formerly Artane), Trileptal®, ursodiol, Valproic Acid, valsartan, Varenicline (Pfizer), Vimpat, Vitamin E, Warfarin, Zarontin®, Zestril, Zonegran®, Zonisamide, Zydis selegiline HCL Oral disintegrating (Zelapar), and combinations thereof.

For example, to treat or ameliorate the effects of epilepsy, a subject may be administered an effective amount of one or more compounds or pharmaceutical compositions of the present disclosure and, e.g., one or more of the following: Albendazole, Banzel, BGG492 (Novartis Corp., New York, NY) Carbamazepine, Carbatrol®, Clobazam, Clonazepam, Depakene®, Depakote®, Depakote ER®, Diastat, Diazepam, Dilantin®, Eslicarbazepine acetate, Ethosuximide, Ezogabine, Felbatol®, Felbamate, Frisium, Gabapentin, Gabitril®, Inovelon®, JNJ-26489112 (Johnson and Johnson, New Brunswick, NJ) Keppra®, Keppra XR™, Klonopin, Lacosamide, Lamictal®, Lamotrigine, Levetiracetam, Lorazepam, Luminal, Lyrica, Mysoline®, Memantine, Neurontin®, Onfi®, Oxcarbazepine, Phenobarbital, Phenytek®, Phenytoin, Potiga, Primidone, probenecid, PRX-00023 (EPIX Pharmaceuticals Inc, Lexington, MA), Rufinamide, Sabril, Tegretol®, Tegretol XR®, Tiagabine, Topamax®, Topiramate, Trileptal®, Valproic Acid, Vimpat, Zarontin®, Zonegran®, and Zonisamide.

To treat or ameliorate the effects of stroke, a subject may be administered an effective amount of one or more compounds or pharmaceutical compositions of the present disclosure and, e.g., one or more of the following: Aspirin, dipyridamole, Clopidogrel, tissue plasminogen activator (tPA), Warfarin, dabigatran, Heparin, Lovenox, citicoline, L-Alpha glycerylphosphorylcholine, cerebrolysin, Eptifibatide, Escitalopram, Tenecteplase, Alteplase, Minocycline, Esmolol, Sodium Nitroprussiate (NPS), Norepinephrine (NOR), Dapsone, valsartan, Simvastatin, piclozotan, Desmoteplase, losartan, amlodipine, Ancrod, human chorionic gonadotropin (hCG), epoetin alfa (EPO), Galantamine, and THR-18 (Thrombotech Ltd., Ness Ziona, Israel).

To treat or ameliorate the effects of myocardial infarction, a subject may be administered an effective amount of one or more compounds or pharmaceutical compositions of the present disclosure and, e.g., one or more of the following: lisinopril, atenolol, Plavix, metoprolol tartrate, Lovenox, Lopressor, Zestril, Tenormin, Prinivil, aspirin, Arixtra, clopidogrel, Salagen, nitroglycerin, metoprolol tartrate, heparin, Nitrostat, Nitro-Bid, Stanback Headache Powder, nitroglycerin, Activase, Nitrolingual, nitroglycerin, fondaparinux, Lopressor, heparin, nitroglycerin TL, Nitro-Time, Nitromist, Ascriptin, alteplase, Retavase, TNKase, Bufferin, Nitro-Dur, Minitran, reteplase, tenecteplase, clopidogrel, Fragmin, enoxaparin, dalteparin, tirofiban, and Aggrastat.

To treat or ameliorate the effects of type I diabetes, a subject may be administered an effective amount of one or more compounds or pharmaceutical compositions of the present disclosure and, e.g., one or more of the following: insulin, such as regular insulin (Humulin R, Novolin R, others), insulin isophane (Humulin N, Novolin N), insulin lispro (Humalog), insulin aspart (NovoLog), insulin glargine (Lantus) and insulin detemir (Levemir), octreotide, pramlintide, and liraglutide.

To treat or ameliorate the effects of Alzheimer's disease, a subject may be administered an effective amount of one or more compounds or pharmaceutical compositions of the present disclosure and, e.g., one or more of the following: Donepezil (Aricept), Rivastigmine (Exelon), Galantamine (Razadyne), Tacrine (Cognex), Memantine (Namenda), Vitamin E, CERE-110: Adeno-Associated Virus Delivery of NGF (Ceregene), LY450139 (Eli Lilly), Exenatide, Varenicline (Pfizer), PF-04360365 (Pfizer), Resveratrol, and Donepezil (Eisai Korea).

To treat or ameliorate the effects of Parkinson's disease, a subject may be administered an effective amount of one or more compounds or pharmaceutical compositions of the present disclosure and, e.g., one or more of the following: Carbidopa/levodopa immediate-release (Sinemet), Carbidopa/levodopa oral disintegrating (Parcopa), Carbidopa/levodopa/Entacapone (Stalevo), Ropinirole (Requip), Pramipexole (Mirapex), Rotigotine (Neupro), Apomorphine (Apokyn), Selegiline (1-deprenyl, Eldepryl), Rasagiline (Azilect), Zydis selegiline HCL Oral disintegrating (Zelapar), Entacapone (Comtan), Tolcapone (Tasmar), Amantadine (Symmetrel), Trihexyphenidyl (formerly Artane), Benztropine (Cogentin), IPX066 (Impax Laboratories Inc.), Rasagiline (Teva Neuroscience, Inc.), ioflupane 123I (DATSCAN®), safinamide (EMD Serono), and Pioglitazone.

To treat or ameliorate the effects of amyotrophic lateral sclerosis, a subject may be administered an effective amount of one or more compounds or pharmaceutical compositions of the present disclosure and, e.g., one or more of the following: riluzole (Rilutek), Lithium carbonate, Arimoclomol, Creatine, Tamoxifen, Mecobalamin, Memantine (Ebixa), and tauroursodeoxycholic acid (TUDCA).

To treat or ameliorate the effects of Friedreich's ataxia, a subject may be administered an effective amount of one or more compounds or pharmaceutical compositions of the present disclosure and, e.g., one or more of the following: Idebenone, Coenzyme Q, 5-hydroxytryptophan, Propranolol, Enalapril, Lisinopril, Digoxin, Erythropoietin, Lu AA24493, Deferiprone, Varenicline, IVIG, Pioglitazone, and EGb 761.

To treat or ameliorate the effects of multiple sclerosis, a subject may be administered an effective amount of one or more compounds or pharmaceutical compositions of the present disclosure and, e.g., one or more of the following: Avonex, Betaseron, Extavia, Rebif, Glatiramer (Copaxone), Fingolimod (Gilenya), Natalizumab (Tysabri), Mitoxantrone (Novantrone), baclofen (Lioresal), tizanidine (Zanaflex), methylprednisolone, CinnoVex, ReciGen, Masitinib, Prednisone, Interferon beta 1a, Interferon beta 1b, and ELND002 (Elan Pharmaceuticals).

To treat or ameliorate the effects of Huntington's disease, a subject may be administered an effective amount of one or more compounds or pharmaceutical compositions of the present disclosure and, e.g., one or more of the following: Tetrabenazine (Xenazine), haloperidol (Haldol), clozapine (Clozaril), clonazepam (Klonopin), diazepam (Valium), escitalopram (Lexapro), fluoxetine (Prozac, Sarafem), sertraline (Zoloft), valproic acid (Depakene), divalproex (Depakote), lamotrigine (Lamictal), Dimebon, AFQ056 (Novartis), Ethyl-EPA (Miraxion™), SEN0014196 (Siena Biotech), sodium phenylbutyrate, citalopram, ursodiol, minocycline, remacemide, and mirtazapine.

To treat or ameliorate the effects of transmissible spongiform encephalopathy, a subject may be administered an effective amount of one or more compounds or pharmaceutical compositions of the present disclosure and e.g., Quinacrine.

To treat or ameliorate the effects of Charcot-Marie-Tooth disease, a subject may be administered an effective amount of one or more compounds or pharmaceutical compositions of the present disclosure and, e.g., one or more of the following: ascorbic acid and PXT3003.

To treat or ameliorate the effects of dementia with Lewy bodies, a subject may be administered an effective amount of one or more compounds or pharmaceutical compositions of the present disclosure and, e.g., one or more of the following: Aricept, Galantamine, Memantine, Armodafinil, Donepezil, and Ramelteon.

To treat or ameliorate the effects of corticobasal degeneration, a subject may be administered an effective amount of one or more compounds or pharmaceutical compositions of the present disclosure and, e.g., one or more of the following: Davunetide and Coenzyme Q10.

To treat or ameliorate the effects of progressive supranuclear palsy, a subject may be administered an effective amount of one or more compounds or pharmaceutical compositions of the present disclosure and, e.g., one or more of the following: Tideglusib, Rasagiline, alpha-lipoic acid/L-acetyl carnitine, Riluzole, Niacinamide, and Rivastigmine.

To treat or ameliorate the effects of hereditary spastic paraparesis, a subject may be administered an effective amount of one or more compounds or pharmaceutical compositions of the present disclosure and, e.g., one or more of the following: Baclofen, Tizanidine, Oxybutinin chloride, Tolterodine, and Botulinum toxin.

In the present disclosure, one or more compounds or pharmaceutical compositions may be co-administered to a subject in need thereof together in the same composition, simultaneously in separate compositions, or as separate compositions administered at different times, as deemed most appropriate by a physician.

An additional embodiment of the present disclosure is a method for treating or ameliorating the effects of a disorder in a subject in need thereof. This method comprises administering to the subject an effective amount of a pharmaceutical composition comprising a pharmaceutically acceptable carrier or diluent and one or more compounds having the structure of formula (1):

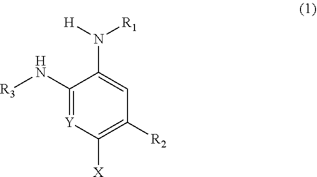

wherein:
R₁ is selected from the group consisting of H, alkyl, aryl, C₁₋₆alkyl-aryl, C₁₋₆alkyl-phenolyl, and C₃₋₁₀carbocycle, wherein each of the alkyl, aryl, C₁₋₆alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
R₃ is a C₃₋₁₂carbocycle, or a polyyne, wherein each of the C₃₋₁₂carbocycle and polyyne are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R₁ and X are both H, Y is —CH and R₃ is

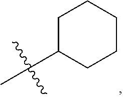 [SMILES: *C1CCCCC1]

R₂ cannot be

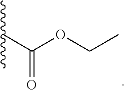 [SMILES: *C(=O)OCC]

Suitable and preferred pharmaceutical compositions for use in this method are as disclosed above in formulas (1), (1a), (1b), (2), (3), (3a) and (4), including pharmaceutical compositions containing the particular compounds identified above. Suitable and preferred subjects who may be treated in accordance with this method are as disclosed above. In this embodiment, the methods may be used to treat disorders set forth above, including degenerative diseases that involve lipid peroxidation and excitotoxic diseases that involve oxidative cell death.

In another aspect of this embodiment, the method further comprises co-administering to the subject an effective amount of one or more additional therapeutic agents disclosed herein.

Another embodiment of the present disclosure is a method of modulating ferroptosis in a subject in need thereof. This method comprises administering to the subject an effective amount of a ferroptosis inhibitor, which comprises one or more compounds having the structure of formula (1):

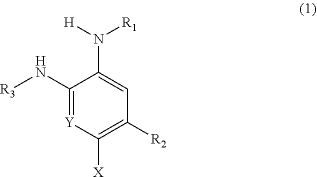

wherein:
R₁ is selected from the group consisting of H, alkyl, aryl, C₁₋₆alkyl-aryl, C₁₋₆alkyl-phenolyl, and C₃₋₁₀carbocycle, wherein each of the alkyl, aryl, C₁₋₆alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
R₃ is a C₃₋₁₂carbocycle, or a polyyne, wherein each of the C₃₋₁₂carbocycle and polyyne are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R₁ and X are both H, Y is —CH and R₃ is

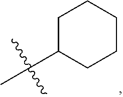 [SMILES: *C1CCCCC1]

R₂ cannot be

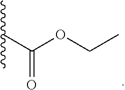 [SMILES: *C(=O)OCC]

As used herein, “ferroptosis” means regulated cell death that is iron-dependent. Ferroptosis is characterized by the overwhelming, iron-dependent accumulation of lethal lipid reactive oxygen species. (Dixon et al., 2012) Ferroptosis is distinct from apoptosis, necrosis, and autophagy. (Id.) Assays for ferroptosis are as disclosed herein, for instance, in the Examples section.

Suitable and preferred compounds for use in this method are as disclosed above in formulas (1), (1a), (1b), (2), (3), (3a) and (4), including the particular compounds identified above. Suitable and preferred subjects who may be treated in accordance with this method are as disclosed above. In this embodiment, the methods may be used to treat the disorders set forth above, including degenerative diseases that involve lipid peroxidation and excitotoxic diseases that involve oxidative cell death.

In another aspect of this embodiment, the method further comprises co-administering to the subject an effective amount of one or more additional therapeutic agents disclosed herein.

A further embodiment of the present disclosure is a method of reducing reactive oxygen species (ROS) in a cell. This method comprises contacting a cell with a ferroptosis modulator, which comprises one or more compounds having the structure of formula (1):

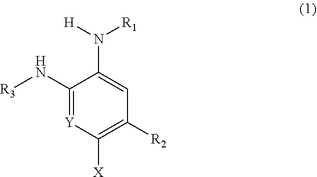

wherein:
R₁ is selected from the group consisting of H, alkyl, aryl, C₁₋₆alkyl-aryl, C₁₋₆alkyl-phenolyl, and C₃₋₁₀carbocycle, wherein each of the alkyl, aryl, C₁₋₆alkyl-aryl, C₁₋₆alkyl-phenolyl, and C₃₋₁₀carbocycle are optionally substituted with one or more atoms or groups;
R₂ is an oxazole, an oxadiazole, an amide, an ether, or an ester, wherein each of the oxazole, oxadiazole, amide, ether, and ester are optionally substituted with one or more atoms or groups;
R₃ is a C₃₋₁₂carbocycle, or a polyyne, wherein each of the C₃₋₁₂carbocycle and polyyne are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R₁ and X are both H, Y is —CH and R₃ is

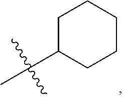 [SMILES: *C1CCCCC1]

R₂ cannot be

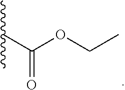 [SMILES: *C(=O)OCC]

As used herein, the terms “modulate”, “modulating”, “modulator” and grammatical variations thereof mean to change, such as decreasing or reducing the occurrence of ferroptosis. In this embodiment, “contacting” means bringing the compound and optionally one or more additional therapeutic agents into close proximity to the cells in need of such modulation. This may be accomplished using conventional techniques of drug delivery to the subject or in the in vitro situation by, e.g., providing the compound and optionally other therapeutic agents to a culture media in which the cells are located.

Suitable and preferred compounds for use in this method are as disclosed above in formulas (1), (1a), (1b), (2), (3), (3a) and (4), including the particular compounds identified above. In this embodiment, reducing ROS may be accomplished in cells obtained from a subject having a disorder as disclosed herein. Suitable and preferred subjects of this embodiment are as disclosed above.

In one aspect of this embodiment, the cell is a mammalian cell. Preferably, the mammalian cell is obtained from a mammal selected from the group consisting of humans, primates, farm animals, and domestic animals. More preferably, the mammalian cell is a human cancer cell.

In another aspect of this embodiment, the method further comprises contacting the cell with at least one additional therapeutic agent as disclosed herein.

An additional embodiment of the present disclosure is a method for treating or ameliorating the effects of a neurodegenerative disease in a subject in need thereof. This method comprises administering to the subject an effective amount of one or more compounds having the structure of formula (1):

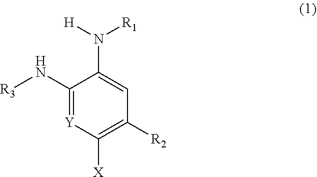

wherein:
R₁ is selected from the group consisting of H, alkyl, aryl, C₁₋₆alkyl-aryl, C₁₋₆alkyl-phenolyl, and C₃₋₁₀carbocycle, wherein each of the alkyl, aryl, C₁₋₆alkyl-aryl, C₁₋₆alkyl-phenolyl, and C₃₋₁₀carbocycle are optionally substituted with one or more atoms or groups,
R₂ is an oxazole, an oxadiazole, an amide, an ether, or an ester, wherein each of the oxazole, oxadiazole, amide, ether, and ester are optionally substituted with one or more atoms or groups;
R₃ is a C₃₋₁₂carbocycle, or a polyyne, wherein each of the C₃₋₁₂carbocycle and polyyne are optionally substituted with one or more atoms or groups;
X is selected from the group consisting of H, optionally substituted alkyl, and halo; and
Y is —CH or N;
or an N-oxide, crystalline form, hydrate, or a pharmaceutically acceptable salt thereof,
with the proviso that:
when R₁ and X are both H, Y is —CH and R₃ is

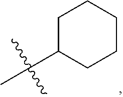 [SMILES: *C1CCCCC1]

R₂ cannot be

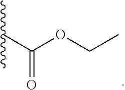 [SMILES: *C(=O)OCC]

Suitable and preferred compounds for use in this method are as disclosed above in formulas (1), (1a), (1b), (2), (3), (3a) and (4), including the particular compounds identified above. In this embodiment, the method may be used to treat the disorders set forth above.

Suitable and preferred subjects are as disclosed herein. In this embodiment, the methods may be used to treat the neurodegenerative disorders set forth above.

In one aspect of this embodiment, the method further comprises co-administering to the subject an effective amount of one or more therapeutic agents disclosed herein.

An additional embodiment of the present disclosure is a compound having the structure selected from the group consisting of:

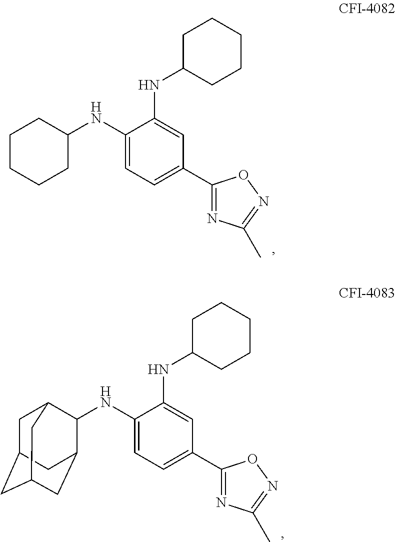 [SMILES: Cc1noc(-c2ccc(NC3CCCCC3)c(NC3CCCCC3)c2)n1 ; Cc1noc(-c2ccc(NC3C4CC5CC(C4)CC3C5)c(NC3CCCCC3)c2)n1]

and combinations thereof, or an N-oxide, crystalline form, hydrate, or a pharmaceutically acceptable salt thereof.

An additional embodiment of the present disclosure is a pharmaceutical composition comprising a pharmaceutically acceptable carrier or diluent and a compound having the structure selected from the group consisting of:

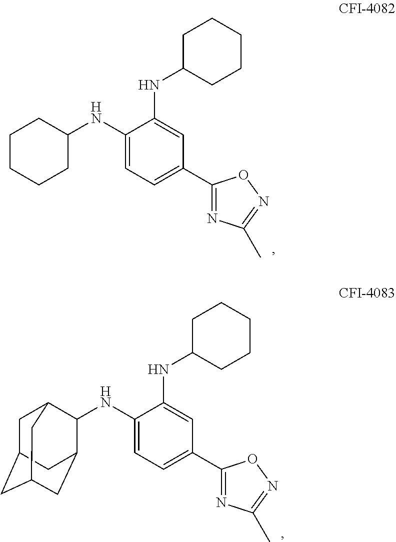 [SMILES: Cc1noc(-c2ccc(NC3CCCCC3)c(NC3CCCCC3)c2)n1 ; Cc1noc(-c2ccc(NC3C4CC5CC(C4)CC3C5)c(NC3CCCCC3)c2)n1]

and combinations thereof, or an N-oxide, crystalline form, hydrate, or a pharmaceutically acceptable salt thereof.

An additional embodiment of the present disclosure is a method for treating or ameliorating the effects of a disorder in a subject in need thereof comprising administering to the subject an effective amount of a compound having the structure selected from the group consisting of:

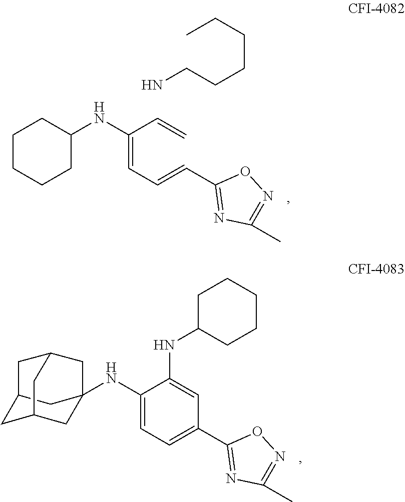

and combinations thereof, or an N-oxide, crystalline form, hydrate, or a pharmaceutically acceptable salt thereof.

An additional embodiment of the present disclosure is a method of modulating ferroptosis in a subject in need thereof comprising administering to the subject an effective amount of a ferroptosis inhibitor, which comprises a compound having the structure selected from the group consisting of:

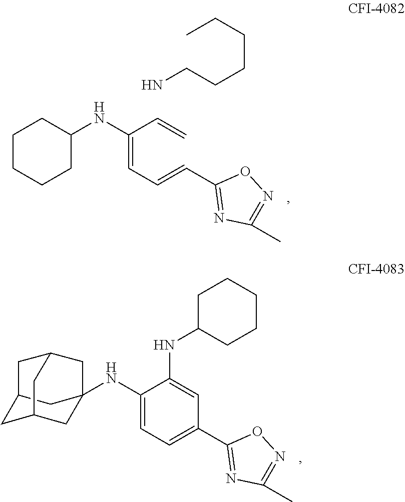

and combinations thereof, or an N-oxide, crystalline form, hydrate, or a pharmaceutically acceptable salt thereof.

An additional embodiment of the present disclosure is a method of reducing reactive oxygen species (ROS) in a cell comprising contacting a cell with a ferroptosis modulator, which comprises a compound having the structure selected from the group consisting of:

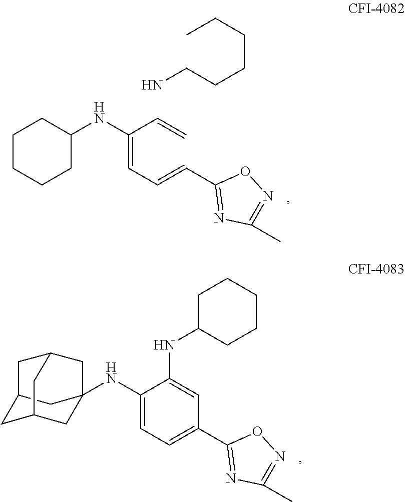

An additional embodiment of the present disclosure is a method for treating or ameliorating the effects of a neurodegenerative disease in a subject in need thereof comprising administering to the subject an effective amount of a compound having the structure selected from the group consisting of:

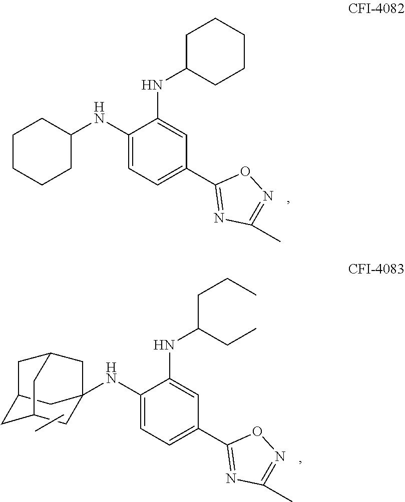

and combinations thereof, or an N-oxide, crystalline form, hydrate, or a pharmaceutically acceptable salt thereof.

Another embodiment of the present disclosure is a pharmaceutical composition comprising a pharmaceutically acceptable carrier or diluent and a compound having the structure selected from the group consisting of:

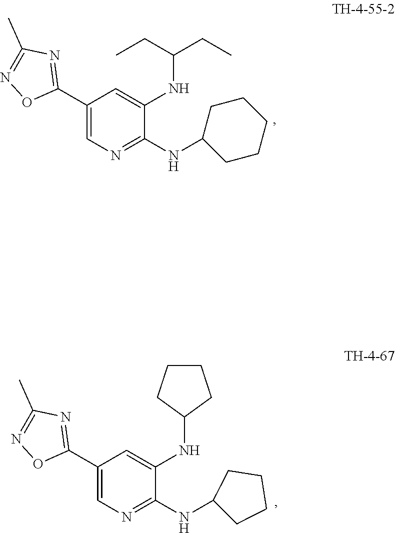 [SMILES: CCC(CC)Nc1cc(-c2nc(C)no2)cnc1NC1CCCCC1 ; Cc1noc(-c2cnc(NC3CCCC3)c(NC3CCCC3)c2)n1]

and combinations thereof, or an N-oxide, crystalline form, hydrate, or a pharmaceutically acceptable salt thereof.

Another embodiment of the present disclosure is a method for treating or ameliorating the effects of a disorder in a subject in need thereof comprising administering to the subject an effective amount of a compound having the structure selected from the group consisting of:

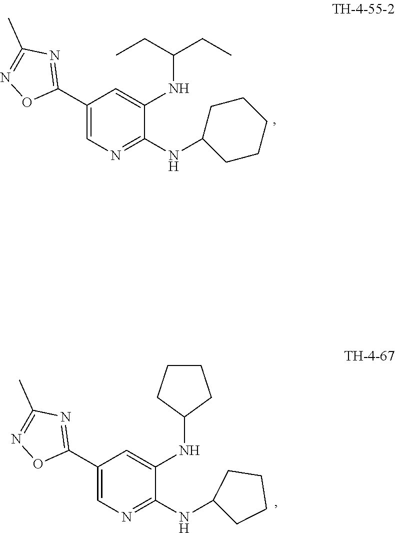 [SMILES: CCC(CC)Nc1cc(-c2nc(C)no2)cnc1NC1CCCCC1 ; Cc1noc(-c2cnc(NC3CCCC3)c(NC3CCCC3)c2)n1]

and combinations thereof, or an N-oxide, crystalline form, hydrate, or a pharmaceutically acceptable salt thereof.

Another embodiment of the present disclosure is a method of modulating ferroptosis in a subject in need thereof comprising administering to the subject an effective amount of a ferroptosis inhibitor, which comprises a compound having the structure selected from the group consisting of:

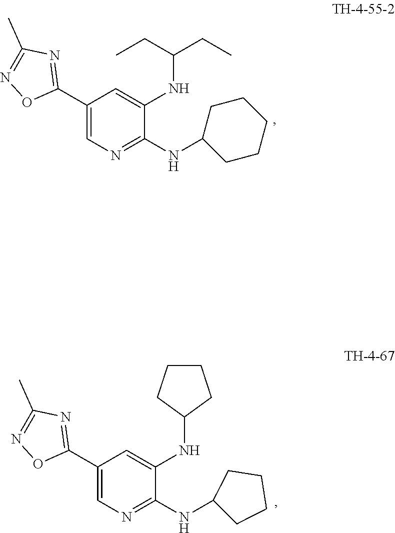 [SMILES: CCC(CC)Nc1cc(-c2nc(C)no2)cnc1NC1CCCCC1 ; Cc1noc(-c2cnc(NC3CCCC3)c(NC3CCCC3)c2)n1]

and combinations thereof, or an N-oxide, crystalline form, hydrate, or a pharmaceutically acceptable salt thereof.

Another embodiment of the present disclosure is a method of reducing reactive oxygen species (ROS) in a cell comprising contacting a cell with a ferroptosis modulator, which comprises a compound having the structure selected from the group consisting of:

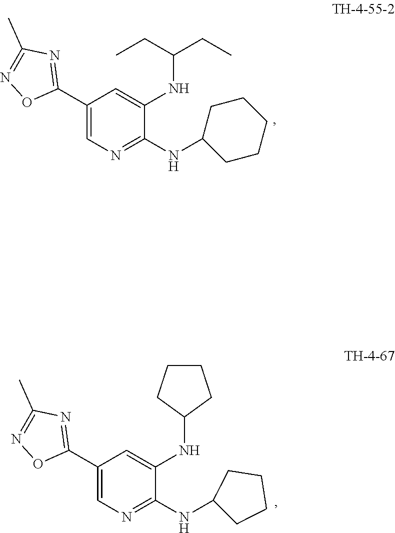 [SMILES: CCC(CC)Nc1cc(-c2nc(C)no2)cnc1NC1CCCCC1 ; Cc1noc(-c2cnc(NC3CCCC3)c(NC3CCCC3)c2)n1]

and combinations thereof, or an N-oxide, crystalline form, hydrate, or a pharmaceutically acceptable salt thereof.

Another embodiment of the present disclosure is a method for treating or ameliorating the effects of a neurodegenerative disease in a subject in need thereof comprising administering to the subject an effective amount of a compound having the structure selected from the group consisting of:

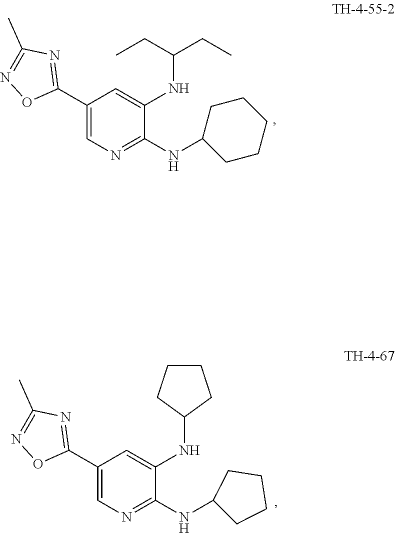 [SMILES: CCC(CC)Nc1cc(-c2nc(C)no2)cnc1NC1CCCCC1 ; Cc1noc(-c2cnc(NC3CCCC3)c(NC3CCCC3)c2)n1]

and combinations thereof, or an N-oxide, crystalline form, hydrate, or a pharmaceutically acceptable salt thereof.

An additional embodiment of the present disclosure is a compound having the structure selected from the group consisting of:

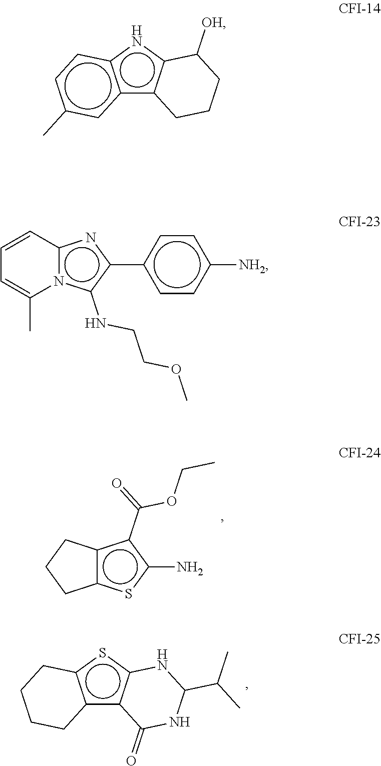

and combinations thereof, or an N-oxide, crystalline form, hydrate, or a pharmaceutically acceptable salt thereof.

An additional embodiment of the present disclosure is a pharmaceutical composition comprising a pharmaceutically acceptable carrier or diluent and a compound having the structure selected from the group consisting of:

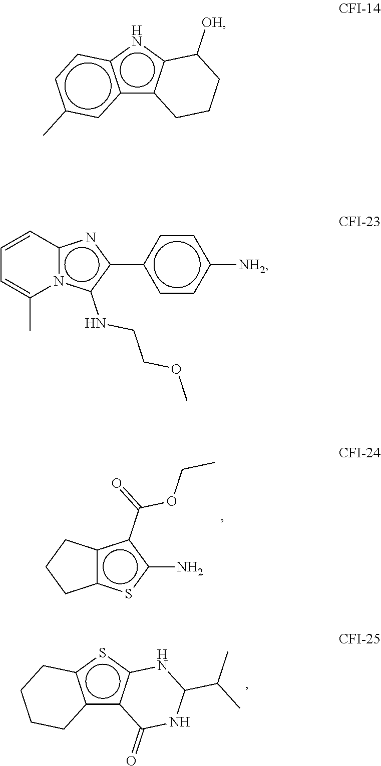

and combinations thereof, or an N-oxide, crystalline form, hydrate, or a pharmaceutically acceptable salt thereof.

An additional embodiment of the present disclosure is a method for treating or ameliorating the effects of a disorder in a subject in need thereof comprising administering to the subject an effective amount of a compound having the structure selected from the group consisting of:

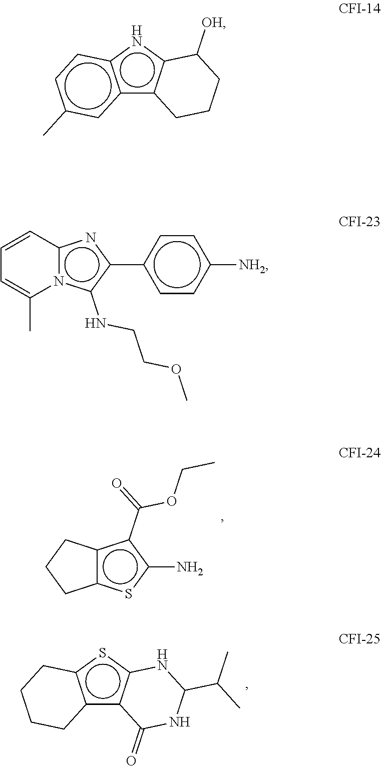

and combinations thereof, or an N-oxide, crystalline form, hydrate, or a pharmaceutically acceptable salt thereof.

An additional embodiment of the present disclosure is a method of modulating ferroptosis in a subject in need thereof comprising administering to the subject an effective amount of a ferroptosis inhibitor, which comprises a compound having the structure selected from the group consisting of:

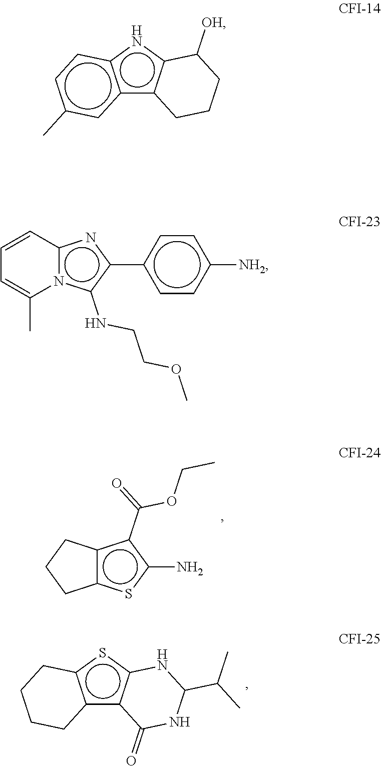

and combinations thereof, or an N-oxide, crystalline form, hydrate, or a pharmaceutically acceptable salt thereof.

An additional embodiment of the present disclosure is a method of reducing reactive oxygen species (ROS) in a cell comprising contacting a cell with a ferroptosis modulator, which comprises a compound having the structure selected from the group consisting of:

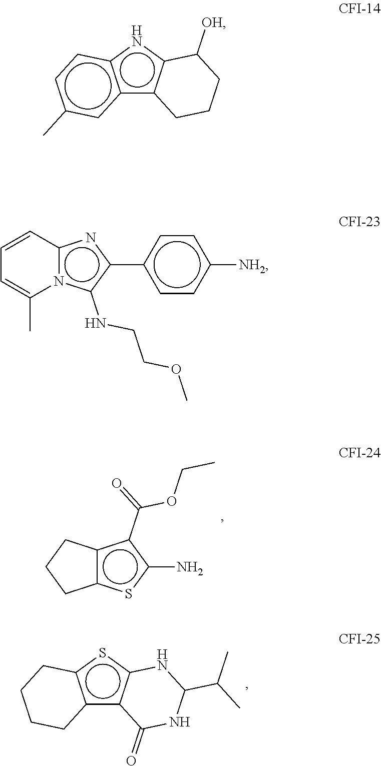

and combinations thereof, or an N-oxide, crystalline form, hydrate, or a pharmaceutically acceptable salt thereof.

An additional embodiment of the present disclosure is a method for treating or ameliorating the effects of a neurodegenerative disease in a subject in need thereof comprising administering to the subject an effective amount of a compound having the structure selected from the group consisting of:

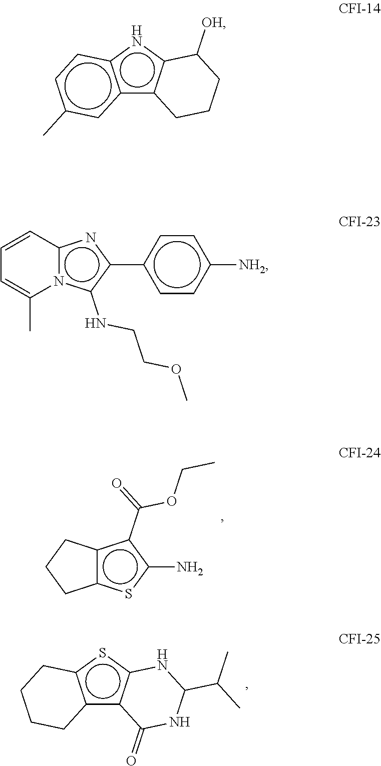

and combinations thereof, or an N-oxide, crystalline form, hydrate, or a pharmaceutically acceptable salt thereof.

Still another embodiment of the present disclosure is a method for alleviating side effects in a subject undergoing radiotherapy and/or immunotherapy, comprising administering to the subject an effective amount of one or more compounds disclosed herein.

As used herein, “radiotherapy” or “radiation therapy” refers to a therapy using ionizing radiation to control or kill malignant cells. Common side effects of radiotherapy include, but are not limited to, acute side effects (such as nausea, vomiting, damage to the epithelial surfaces, mouth, throat and stomach sores, intestinal discomfort, swelling, infertility, etc.), late side effects (such as fibrosis, epilation, dryness, lymphedema, cardiovascular disorder, cognitive decline, radiation enteropathy, radiation-induced polyneuropathy), and cumulative side effects.

As used herein, “immunotherapy” refers to the treatment of disease by activating or suppressing the immune system. It can be classified as an activation immunotherapy that elicits or amplifies an immune response, or a suppression immunotherapy that reduce or suppress an immune response. Common side effects of immunotherapy include, but are not limited to, skin problems (such as pain, swelling, soreness, redness, itchiness, rash, etc.), flu-like symptoms (such as fever, chills, weakness, dizziness, nausea or vomiting, muscle or joint aches, fatigue, headache, trouble breathing, low or high blood pressure, etc.), and other symptoms such as swelling and weight gain from retaining fluid, heart palpitations, sinus congestion, diarrhea, infection, organ inflammation, etc.

A further embodiment of the present disclosure is a method for treating or ameliorating the effects of an infection associated with ferroptosis in a subject, comprising administering to the subject an effective amount of one or more compounds disclosed herein. In some embodiments, the infection is caused by Mycobacterium tuberculosis.

As used herein, a “pharmaceutically acceptable salt” means a salt of the compounds of the present disclosure which are pharmaceutically acceptable, as defined herein, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

In the present disclosure, an “effective amount” or “therapeutically effective amount” of a compound or pharmaceutical composition is an amount of such a compound or composition that is sufficient to effect beneficial or desired results as described herein when administered to a subject. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of the subject, and like factors well known in the arts of, e.g., medicine and veterinary medicine. In general, a suitable dose of a compound or pharmaceutical composition according to the disclosure will be that amount of the compound or composition, which is the lowest dose effective to produce the desired effect with no or minimal side effects. The effective dose of a compound or pharmaceutical composition according to the present disclosure may be administered as two, three, four, five, six or more sub-doses, administered separately at appropriate intervals throughout the day.

A suitable, non-limiting example of a dosage of a compound or pharmaceutical composition according to the present disclosure or a composition comprising such a compound, is from about 1 ng/kg to about 1000 mg/kg, such as from about 1 mg/kg to about 100 mg/kg, including from about 5 mg/kg to about 50 mg/kg. Other representative dosages of a compound or a pharmaceutical composition of the present disclosure include about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, or 1000 mg/kg.

A compound or pharmaceutical composition of the present disclosure may be administered in any desired and effective manner: for oral ingestion, or as an ointment or drop for local administration to the eyes, or for parenteral or other administration in any appropriate manner such as intraperitoneal, subcutaneous, topical, intradermal, inhalation, intrapulmonary, rectal, vaginal, sublingual, intramuscular, intravenous, intraarterial, intrathecal, or intralymphatic. Further, a compound or pharmaceutical composition of the present disclosure may be administered in conjunction with other treatments. A compound or pharmaceutical composition of the present disclosure may be encapsulated or otherwise protected against gastric or other secretions, if desired.

The pharmaceutical compositions of the disclosure are pharmaceutically acceptable and comprise one or more active ingredients in admixture with one or more pharmaceutically-acceptable carriers or diluents and, optionally, one or more other compounds, drugs, ingredients and/or materials. Regardless of the route of administration selected, the compounds/pharmaceutical compositions of the present disclosure are formulated into pharmaceutically-acceptable dosage forms by conventional methods known to those of skill in the art. See, e.g., Remington, The Science and Practice of Pharmacy (21^{st} Edition, Lippincott Williams and Wilkins, Philadelphia, PA). More generally, “pharmaceutically acceptable” means that which is useful in preparing a composition that is generally safe, non-toxic, and neither biologically nor otherwise undesirable and includes that which is acceptable for veterinary use as well as human pharmaceutical use.

Pharmaceutically acceptable carriers and diluents are well known in the art (see, e.g., Remington, The Science and Practice of Pharmacy (21^{st} Edition, Lippincott Williams and Wilkins, Philadelphia, PA) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each pharmaceutically acceptable carrier or diluent used in a composition of the disclosure must be “acceptable” in the sense of being compatible with the other ingredients of the formulation and not injurious to the subject. Carriers or diluents suitable for a selected dosage form and intended route of administration are well known in the art, and acceptable carriers or diluents for a chosen dosage form and method of administration can be determined using ordinary skill in the art.

The pharmaceutical compositions of the disclosure may, optionally, contain additional ingredients and/or materials commonly used in such compositions. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monostearate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monosterate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

Compounds or pharmaceutical compositions suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragees, powders, granules and the like) may be prepared, e.g., by mixing the active ingredient(s) with one or more pharmaceutically acceptable carriers or diluents and, optionally, one or more fillers, extenders, binders, humectants, disintegrating agents, solution retarding agents, absorption accelerators, wetting agents, absorbents, lubricants, and/or coloring agents. Solid compositions of a similar type may be employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Liquid dosage forms for oral administration include pharmaceutically acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

Compositions for rectal or vaginal administration may be presented as a suppository, which may be prepared by mixing one or more active ingredient(s) with one or more suitable nonirritating carriers which are solid at room temperature, but liquid at body temperature and, therefore, will melt in the rectum or vaginal cavity and release the active compound. Compositions which are suitable for vaginal administration also include pessaries, tampons, creams, gels, pastes, foams or spray formulations containing such pharmaceutically acceptable carriers as are known in the art to be appropriate.

Dosage forms for the topical or transdermal administration include powders, sprays, ointments, pastes, creams, lotions, gels, solutions, patches, drops and inhalants. The active agent(s)/compound(s) may be mixed under sterile conditions with a suitable pharmaceutically acceptable carrier or diluent. The ointments, pastes, creams and gels may contain excipients. Powders and sprays may contain excipients and propellants.

Compositions suitable for parenteral administrations comprise one or more agent(s)/compound(s) in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption.

In some cases, in order to prolong the effect of a drug (e.g., pharmaceutical formulation), it is desirable to slow its absorption from subcutaneous or intramuscular injection. This may be accomplished by the use of a liquid suspension of crystalline or amorphous material having poor water solubility.

The rate of absorption of the active agent/drug then depends upon its rate of dissolution which, in turn, may depend upon crystal size and crystalline form. Alternatively, delayed absorption of a parenterally administered agent/drug may be accomplished by dissolving or suspending the active agent/drug in an oil vehicle. Injectable depot forms may be made by forming microencapsule matrices of the active ingredient in biodegradable polymers. Depending on the ratio of the active ingredient to polymer, and the nature of the particular polymer employed, the rate of active ingredient release can be controlled. Depot injectable formulations are also prepared by entrapping the drug in liposomes or microemulsions which are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid carrier or diluent, for example water for injection, immediately prior to use. Extemporaneous injection solutions and suspensions may be prepared from sterile powders, granules and tablets of the type described above.

In the foregoing embodiments, the following definitions apply.

The term “aliphatic”, as used herein, refers to a group composed of carbon and hydrogen that do not contain aromatic rings. Accordingly, aliphatic groups include alkyl, alkenyl, alkynyl, and carbocyclyl groups. Additionally, unless otherwise indicated, the term “aliphatic” is intended to include both “unsubstituted aliphatics” and “substituted aliphatics”, the latter of which refers to aliphatic moieties having substituents replacing a hydrogen on one or more carbons of the aliphatic group. Such substituents can include, for example, a halogen, a deuterium, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, an aromatic, or heteroaromatic moiety.

The term “alkyl” refers to the radical of saturated aliphatic groups that does not have a ring structure, including straight-chain alkyl groups, and branched-chain alkyl groups. In certain embodiments, a straight chain or branched chain alkyl has 6 or fewer carbon atoms in its backbone (e.g., C₁-C₆ for straight chains, C₃-C₆ for branched chains). In other embodiments, the “alkyl” may include up to twelve carbon atoms, e.g., C₁, C₂, C₃, C₄, C₅, C₆, C₇, C₈, C₉, C₁₀, C₁₁ or C₁₂. Such substituents include all those contemplated for aliphatic groups, as discussed below, except where stability is prohibitive.

The term “alkenyl”, as used herein, refers to an aliphatic group containing at least one double bond and unless otherwise indicated, is intended to include both “unsubstituted alkenyls” and “substituted alkenyls”, the latter of which refers to alkenyl moieties having substituents replacing a hydrogen on one or more carbons of the alkenyl group. Such substituents include all those contemplated for aliphatic groups, as discussed below, except where stability is prohibitive. For example, substitution of alkenyl groups by one or more alkyl, carbocyclyl, aryl, heterocyclyl, or heteroaryl groups is contemplated.

Moreover, unless otherwise indicated, the term “alkyl” as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Indeed, unless otherwise indicated, all groups recited herein are intended to include both substituted and unsubstituted options.

The term “C_{x-y}” when used in conjunction with a chemical moiety, such as, alkyl and cycloalkyl, is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-tirfluoroethyl, etc.

The term “aryl” as used herein includes substituted or unsubstituted single-ring aromatic groups in which each atom of the ring is carbon. Preferably the ring is a 3- to 8-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “alkyl-aryl” refers to an alkyl group substituted with at least one aryl group.

The term “alkyl-heteroaryl” refers to an alkyl group substituted with at least one heteroaryl group.

The term “alkenyl-aryl” refers to an alkenyl group substituted with at least one aryl group.

The term “alkenyl-heteroaryl” refers to an alkenyl group substituted with at least one heteroaryl group.

The terms “carbocycle”, “carbocyclyl”, and “carbocyclic”, as used herein, refer to a non-aromatic saturated or unsaturated ring in which each atom of the ring is carbon. Preferably a carbocycle ring contains from 3 to 10 atoms, more preferably from 3 to 8 atoms, including 5 to 7 atoms, such as for example, 6 atoms. The term “cabocycle” also includes bicycles, tricycles and other multicyclic ring systems, including the adamantyl ring system.

The terms “halo” and “halogen” are used interchangeably herein and mean halogen and include chloro, fluoro, bromo, and iodo.

The term “heteroaryl” includes substituted or unsubstituted aromatic single ring structures, preferably 3- to 8-membered rings, more preferably 5- to 7-membered rings, even more preferably 5- to 6-membered rings, whose ring structures include at least one heteroatom, preferably one to four heteroatoms, more preferably one or two heteroatoms. The term “heteroaryl” also includes polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur; more preferably, nitrogen and oxygen.

The term “ketone” means an organic compound with the structure RC(═O)R′, wherein neither R nor R′ can be hydrogen atoms.

The term “ether” means an organic compound with the structure R—O—R′, wherein neither R nor R′ can be hydrogen atoms.

The term “ester” means an organic compound with the structure RC(═O)OR′, wherein neither R nor R′ can be hydrogen atoms.

The term “polyyne” means is an organic compound with alternating single and triple bonds; that is, a series of consecutive alkynes, (—C≡C—) n with n greater than 1.

The term “substituted” refers to moieties having substituents replacing a hydrogen on one or more carbons of the backbone. It will be understood that “substitution” or “substituted with” includes the implicit proviso that such substitution is in accordance with the permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

As set forth previously, unless specifically stated as “unsubstituted,” references to chemical moieties herein are understood to include substituted variants. For example, reference to an “aryl” group or moiety implicitly includes both substituted and unsubstituted variants.

As used herein, the term “oxadiazole” means any compound or chemical group containing the following structure:

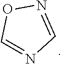

As used herein, the term “oxazole” means any compound or chemical group containing the following structure:

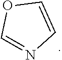

As used herein, the term “triazole” means any compound or chemical group containing the following structure:

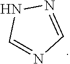

It is understood that the disclosure of a compound herein encompasses all stereoisomers of that compound. As used herein, the term “stereoisomer” refers to a compound made up of the same atoms bonded by the same bonds but having different three-dimensional structures which are not interchangeable. The three-dimensional structures are called configurations. Stereoisomers include enantiomers and diastereomers.

The terms “racemate” or “racemic mixture” refer to a mixture of equal parts of enantiomers. The term “chiral center” refers to a carbon atom to which four different groups are attached. The term “enantiomeric enrichment” as used herein refers to the increase in the amount of one enantiomer as compared to the other.

It is appreciated that to the extent compounds of the present disclosure have a chiral center, they may exist in and be isolated in optically active and racemic forms. Some compounds may exhibit polymorphism. It is to be understood that the present disclosure encompasses any racemic, optically-active, diastereomeric, polymorphic, or stereoisomeric form, or mixtures thereof, of a compound of the disclosure, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase).

Examples of methods to obtain optically active materials are known in the art, and include at least the following:
i) physical separation of crystals—a technique whereby macroscopic crystals of the individual enantiomers are manually separated. This technique can be used if crystals of the separate enantiomers exist, i.e., the material is a conglomerate, and the crystals are visually distinct;
ii) simultaneous crystallization—a technique whereby the individual enantiomers are separately crystallized from a solution of the racemate, possible only if the latter is a conglomerate in the solid state;
iii) enzymatic resolutions—a technique whereby partial or complete separation of a racemate by virtue of differing rates of reaction for the enantiomers with an enzyme;
iv) enzymatic asymmetric synthesis—a synthetic technique whereby at least one step of the synthesis uses an enzymatic reaction to obtain an enantiomerically pure or enriched synthetic precursor of the desired enantiomer;
v) chemical asymmetric synthesis—a synthetic technique whereby the desired enantiomer is synthesized from an achiral precursor under conditions that produce asymmetry (i.e., chirality) in the product, which may be achieved using chiral catalysts as disclosed in more detail herein or chiral auxiliaries;
vi) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
vii) first- and second-order asymmetric transformations—a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;
viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;
ix) enantiospecific synthesis from non-racemic precursors—a synthetic technique whereby the desired enantiomer is obtained from non-chiral starting materials and where the stereochemical integrity is not or is only minimally compromised over the course of the synthesis;
x) chiral liquid chromatography—a technique whereby the enantiomers of a racemate are separated in a liquid mobile phase by virtue of their differing interactions with a stationary phase. The stationary phase can be made of chiral material or the mobile phase can contain an additional chiral material to provoke the differing interactions;
xi) chiral gas chromatography—a technique whereby the racemate is volatilized and enantiomers are separated by virtue of their differing interactions in the gaseous mobile phase with a column containing a fixed non-racemic chiral adsorbent phase;
xii) extraction with chiral solvents—a technique whereby the enantiomers are separated by virtue of preferential dissolution of one enantiomer into a particular chiral solvent;
xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane which allows only one enantiomer of the racemate to pass through.

The stereoisomers may also be separated by usual techniques known to those skilled in the art including fractional crystallization of the bases or their salts or chromatographic techniques such as LC or flash chromatography. The (+) enantiomer can be separated from the (−) enantiomer using techniques and procedures well known in the art, such as that described by J. Jacques, et al., Enantiomers, Racemates, and Resolutions”, John Wiley and Sons, Inc., 1981. For example, chiral chromatography with a suitable organic solvent, such as ethanol/acetonitrile and Chiralpak AD packing, 20 micron can also be utilized to effect separation of the enantiomers.

The following examples are provided to further illustrate the methods of the present disclosure. These examples are illustrative only and are not intended to limit the scope of the disclosure in any way.

### EXAMPLES

The detailed experimental procedures applied to Ferrostatin-1 and its analogs have been described previously in the Internatioanl Application No. PCT/US2014/067977, filed on Dec. 1, 2014, the entirety of which is incorporated herein by reference.

### Example 1

### Synthesis of Ferrostatin-1 Analogs

### Chemicals

Solvents, inorganic salts, and organic reagents were purchased from commercial sources such as Sigma and Fisher and used without further purification unless otherwise noted. Erastin was dissolved in DMSO to a final concentration of 73.1 mM and stored in aliquots at −20° C.

### Chromatography

Merck pre-coated 0.25 mm silica plates containing a 254 nm fluorescence indicator were used for analytical thin-layer chromatography. Flash chromatography was performed on 230-400 mesh silica (SiliaFlash® P60) from Silicycle.

### Spectroscopy

¹H, ¹³C and ¹⁹F NMR spectra were obtained on a Bruker DPX 400 MHz spectrometer. HRMS spectra were taken on double focusing sector type mass spectrometer HX-110A. Maker JEOL Ltd. Tokyo Japan (resolution of 10,000 and 10 KV accel. Volt. Ionization method; FAB (Fast Atom Bombardment) used Xe 3 Kv energy. Used Matrix, NBA (m-Nitro benzyl alcohol)).

### General Procedure A (Esterification)

A representative example is the esterification of the 4-chloro-3-nitrobenzoic acid with tert-butanol. 4-dimethylaminopyridine (DMAP) (2.4607 g, 20.14 mmol, 0.4 equiv) and tert-butanol (24 mL, 250.94 mmol, 5.1 equiv) were added to a solution of 4-chloro-3-nitrobenzoic acid (10.0042 g, 49.63 mmol, 1.0 equiv) dissolved in dichloromethane (350 mL) at room temperature. N,N′-dicyclohexylcarbodiimide (DCC) (13.7853 g, 66.81 mmol, 1.4 equiv) was added to the solution at 0° C. The reaction mixture was allowed to warm to room temperature and stirred overnight under nitrogen atmosphere. The white precipitate was filtered off and the solution was purified by flash-column chromatography on silica gel (hexane, ethyl acetate gradient 40% max).

### General Procedure B (Nucleophilic Aromatic Substitution)

A representative example is the nucleophilic aromatic substitution of tert-butyl 4-chloro-3-nitrobenzoate with 1-admantylamine. Potassium carbonate (2.1570 g, 15.61 mmol, 1.9 equiv) was added to a solution of tert-butyl 4-chloro-3-nitrobenzoate (2.0784 g, 8.07 mmol, 1.0 equiv) dissolved in DMSO (13 mL). A solution of 1-adamantylamine (1.4273 g, 9.44 mmol, 1.2 equiv) dissolved in DMSO (13 mL) was added to the reaction mixture at room temperature. The reaction mixture was heated at 75° C. and stirred overnight under nitrogen atmosphere. After the reaction mixture was cooled to room temperature, water (200 mL) was added and the aqueous layer was extracted three times with ethyl acetate (100 mL). Combined organic layers were extracted with water (30 mL), dried (MgSO₄) and purified by flash-column chromatography on silica gel (hexane, ethyl acetate gradient 40% max).

### General Procedure C (Hydrogenation)

A representative example is the hydrogenation of tert-butyl 4-(1-adamantylamino)-3-nitrobenzoate. Pd(OH)₂ on charcoal (0.5048 g) was added to a solution of tert-butyl 4-(1-adamantylamino)-3-nitrobenzoate (1.0079 g, 2.71 mmol) dissolved in MeOH (100 mL) at room temperature. The reaction mixture was stirred at room temperature overnight under hydrogen atmosphere. The black solid was filtered out and the solution was purified by flash-column chromatography on silica gel (dichloromethane, methanol gradient).

### General Procedure D (Imine Formation)

A representative example is the imine formation reaction between tert-butyl 4-(1-adamantylamino)-3-aminobenzoate and pyrimidine-5-carboxaldehyde. Pyrimidine-5-carboxaldehyde (0.5653 g, 5.23 mmol, 2.9 equiv) and MgSO₄ (0.7850 g) were added to a solution of tert-butyl 4-(1-adamantylamino)-3-aminobenzoate (0.6097 g, 1.78 mmol, 1.0 equiv) dissolved in dichloromethane (122 mL) at room temperature. The reaction mixture was purged once with nitrogen and stirred at room temperature for two overnights under nitrogen atmosphere. The solution was purified by flash-column chromatography on silica gel (hexane, ethyl acetate gradient).

### General Procedure E (Oxidized Imine Formation)

A representative example is the oxidized imine formation reaction between tert-butyl 4-(1-adamantylamino)-3-aminobenzoate and pyrimidine-5-carboxaldehyde. Pyrimidine-5-carboxaldehyde (0.0415 g, 0.38 mmol, 1.3 equiv) was added to a solution of tert-butyl 4-(1-adamantylamino)-3-aminobenzoate (0.1008 g, 0.29 mmol, 1.0 equiv) dissolved in tert-butanol (6 mL). 4M HCl in dioxane (10 μL) was added to the solution at room temperature. The reaction mixture was stirred at 80° C. for 4 hours under nitrogen atmosphere. The solution was purified by flash-column chromatography on silica gel (dichloromethane, methanol gradient).

### General Procedure F (Reductive Amination)

A representative example is the reductive amination reaction between tert-butyl 3-(1-adamantylamino)-4-aminobenzoate and cyclohexanone. Cyclohexanone (0.5 mL, 4.83 mmol, 6.8 equiv) was added dropwise to a solution of tert-butyl 3-(1-adamantylamino)-4-aminobenzoate (0.2416 g, 0.706 mmol, 1 equiv) dissolved in 1,2-dichloroethane (24 mL) at room temperature. Sodium triacetoxyborohydride (0.8913 g, 4.21 mmol, 5.96 mmol) and glacial acetic acid (50 μL, 0.874 mmol, 1.24 equiv) were added to the solution at room temperature. The reaction mixture was stirred at room temperature overnight under nitrogen atmosphere. The solution was purified by flash-column chromatography on silica gel (hexane, ethyl acetate gradient).

### Design and Synthesis of Microsome and Plasma Stable Ferrostatin Analogs

A general route to obtain the compounds of formulas (I) to (Ill) follows a three-step synthesis (see below). An S_{N}Ar reaction between the commercially available ethyl 4-chloro-3-nitrobenzoate and cyclohexylamine, followed by catalytic hydrogenolysis of the nitro group, provided the desired ferrostatin derivatives. The anilines of the latter were reacted through reductive amination with arylaldehydes in the presence of sodium triacetoxyborohydride or through straightforward alkylation with arylalkylhalides in the presence of Hunig's base.

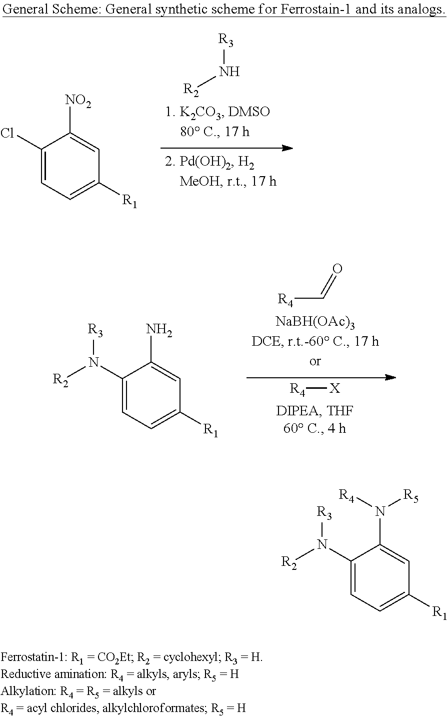

Experimental data pointed to the benzylic position of ferrostatin analogs as the site of metabolic liability in microsomes, and the ester group as the target of plasma esterases. Therefore, analog synthesis focuses on modification of these positions with the goal of improving microsomal and plasma stability in vitro and with the ultimate goal of producing analogs with improved in vivo properties for use in animal models of disease. Because in silico evaluation of Fer-1 analogs' P450 stability using the Schrodinger Suite P450_SOM program showed agreement with the experimental results with liver microsomes, this computer program is used to guide prioritization of compound synthesis and testing of analogs proposed based on modifications known to inhibit metabolism.

One of the most useful methods of blocking metabolism at a specific site is to use a steric shield—a bulky group that hinders oxidation at the position by cytochrome P450. An efficient synthesis of Fer-1 analogs with bulky, blocking groups incorporated at the benzylic site of oxidation is shown in Scheme 1.

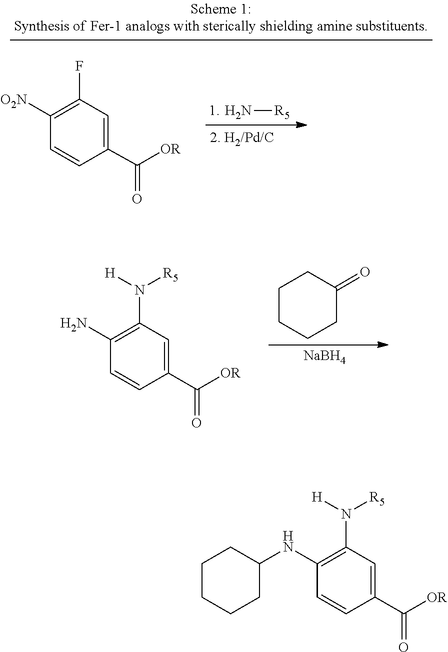

Treatment of commercially available 3-fluoro-4-nitrobenzoic acid with a benzylamine containing the desired bulky substituent at the benzylic position would displace fluoride via an S_{N}Ar reaction to give the corresponding aminonitro compound (Saitoh, et al., 2009). A wide range of benzyl amines are commercially available. Enantiomerically pure amines are important because cytochrome P450s are known to be enantioselective in their oxidations. Benzylically disubstituted amines would increase the amount of steric shielding and have the advantage of being achiral. The 2,6-dimethylbenzyl amine illustrates another mode of shielding the benzylic position.

The synthetic route shown in Scheme 1 also allows ready access to other substituted amine analogs that can be explored, and that may be more resistant to metabolism, as they do not have a benzylic position to react with P450s. Thus, aniline, cyclohexylamine, and adamantly amine may be used as starting materials to give the corresponding analogs.

The t-butyl ester is resistant to plasma esterases; however, this group may be acid labile, and may not be resistant to the acidic conditions in the stomach upon oral dosing. Bioisosteres, functionalities that are biologically equivalent to the functional group they are replacing, are commonly used to produce active analogs with improved properties, such as resistance to metabolism (Hamada, et al., 2012). A number of ester bioisosteres have been reported in the literature and can be incorporated into analogs of Fer-1. As shown in the synthetic route in Scheme 2, the acid or ester group of 3-fluoro-4-nitrobenzoic acid can be readily converted into ester bioisosteres, such as oxazoles (Wu, et al., 2004), oxadiazoles (Pipik, et al., 2004), triazoles (Passaniti, et al., 2002), or ketones (Genna, et al., 2011). These intermediates can then be used in the synthetic route outlined in Scheme 1 to produce the desired Fer-1 analogs with ester bioisosteres that are resistant to esterases.

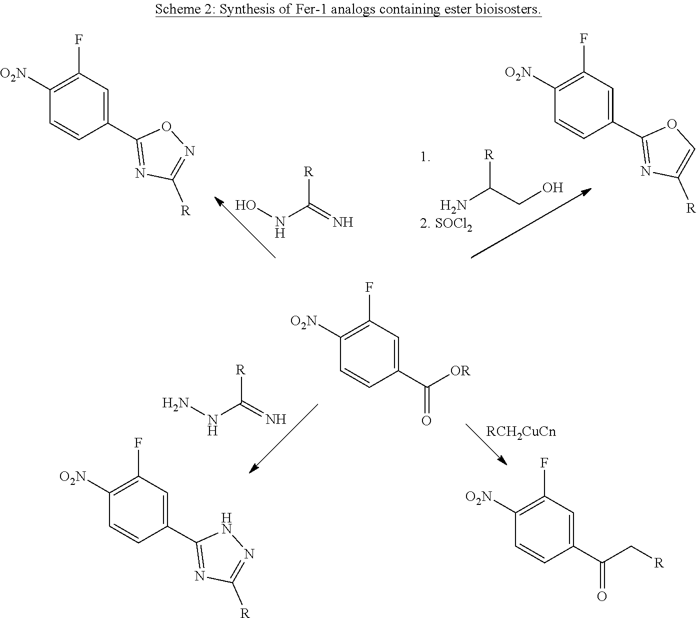

The synthetic routes of representative Fer-1 analogs are illustrated as follow:

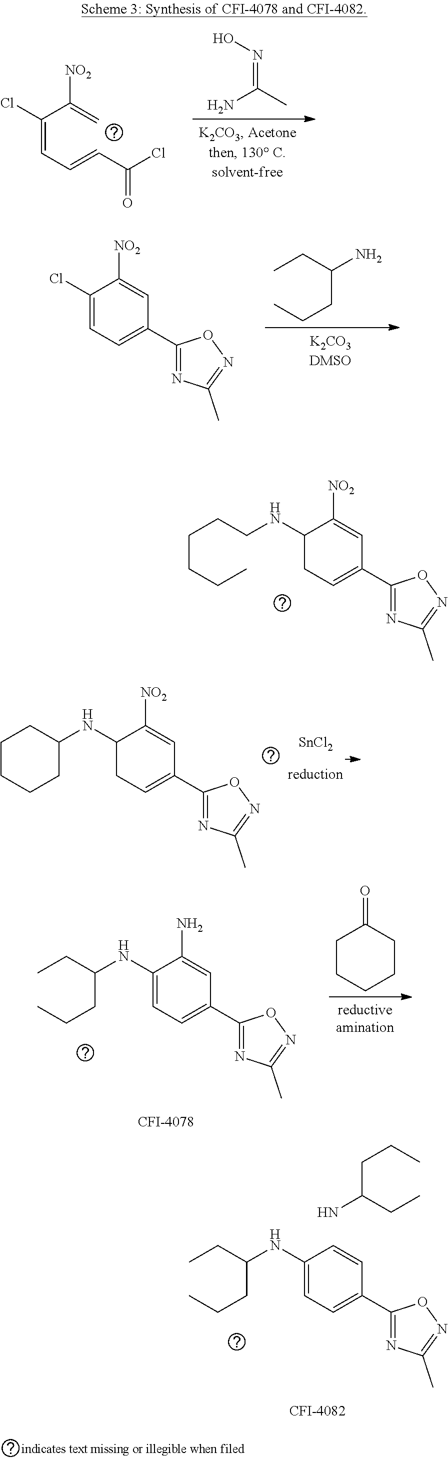

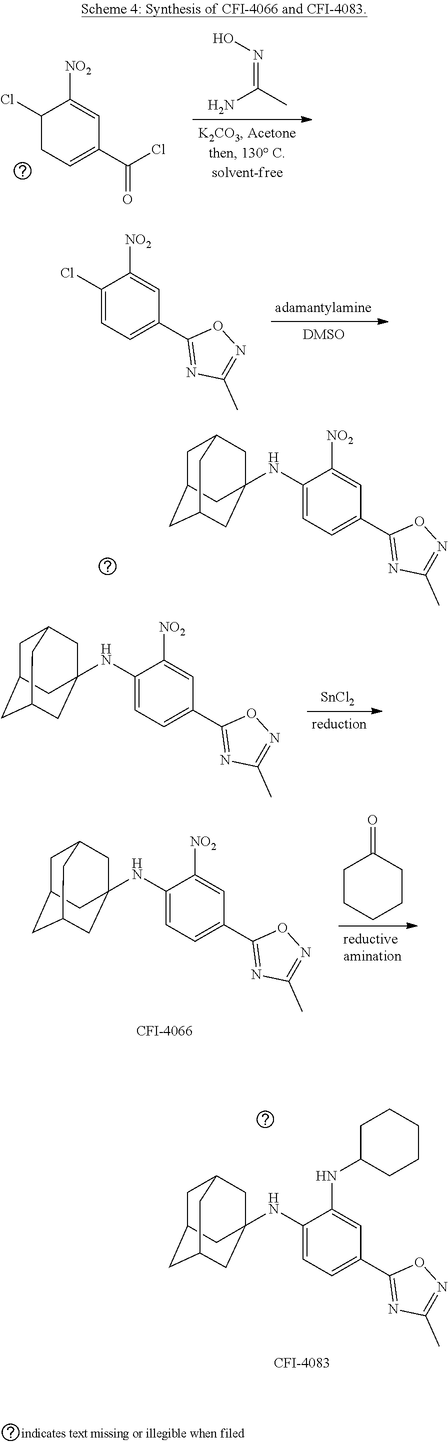

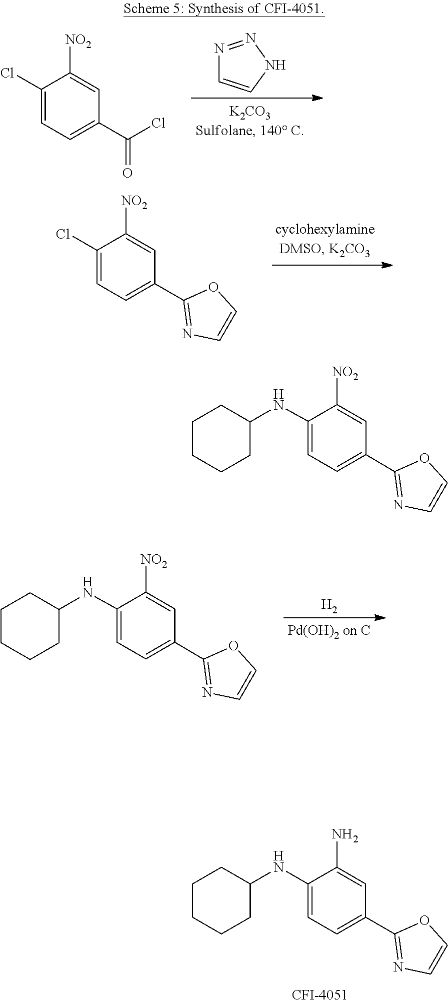

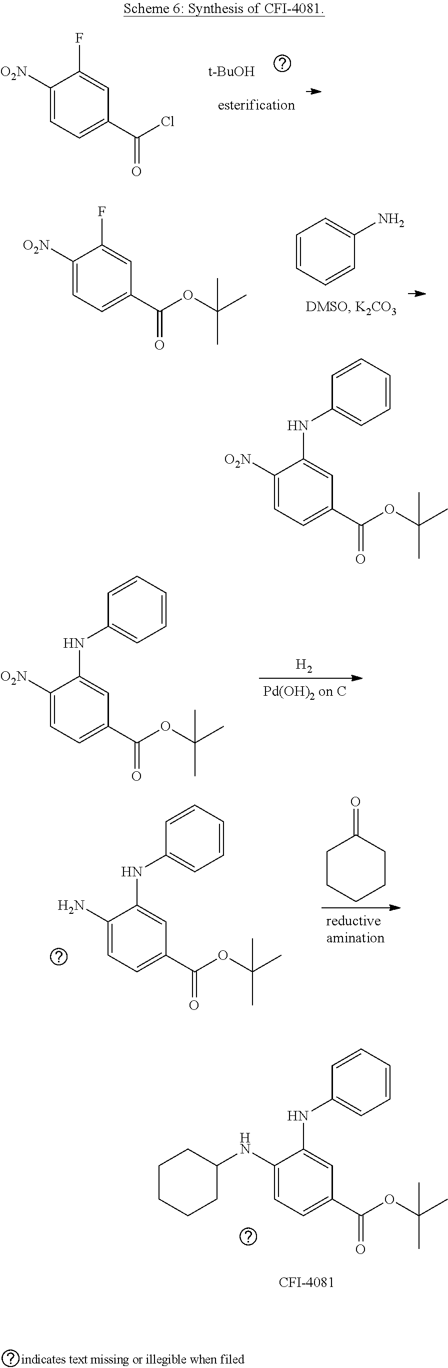

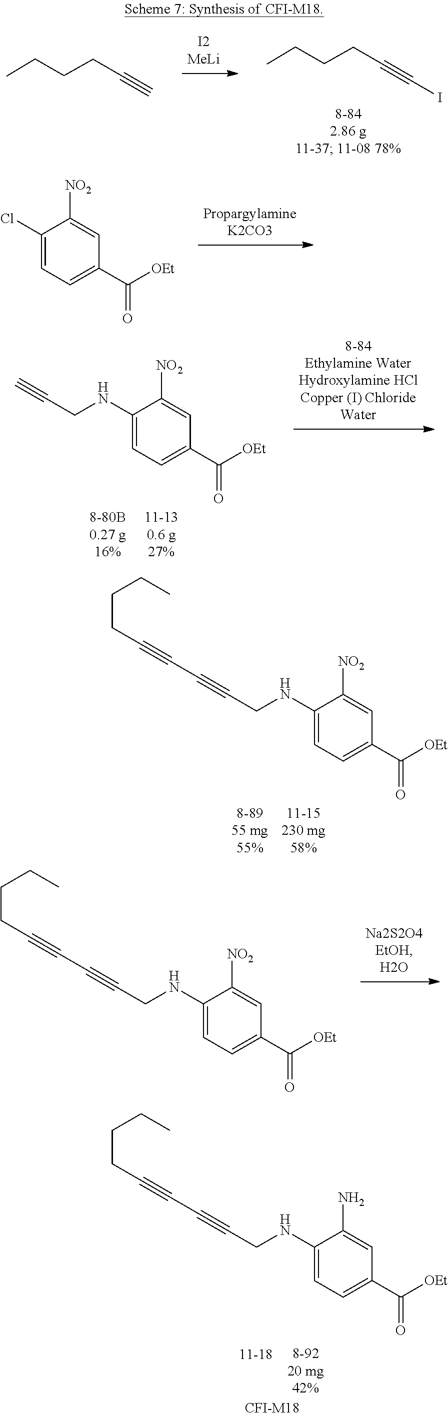

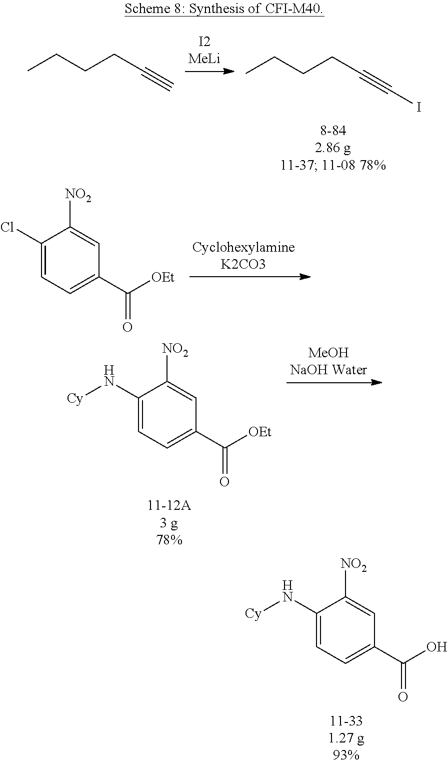

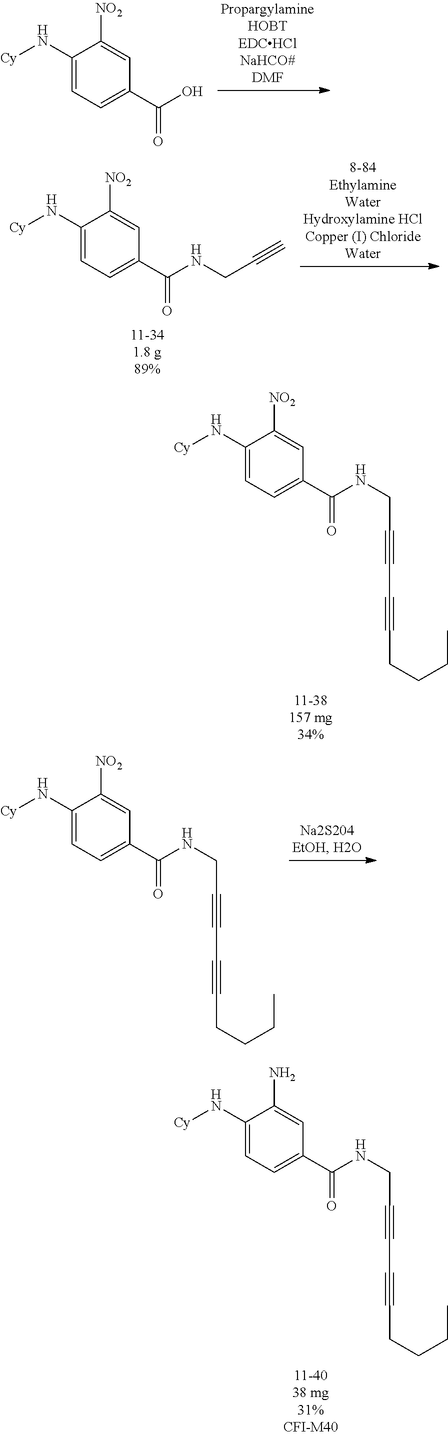

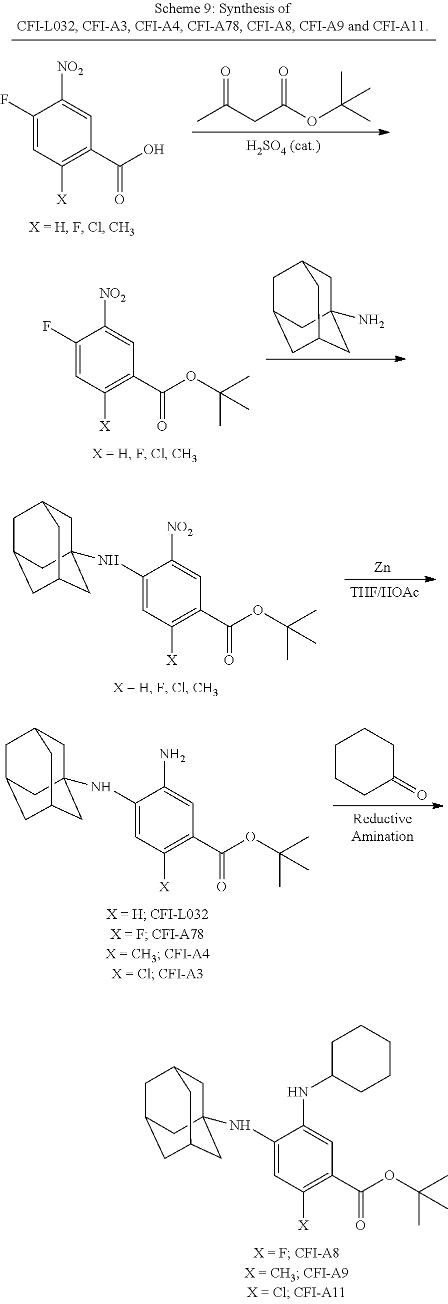

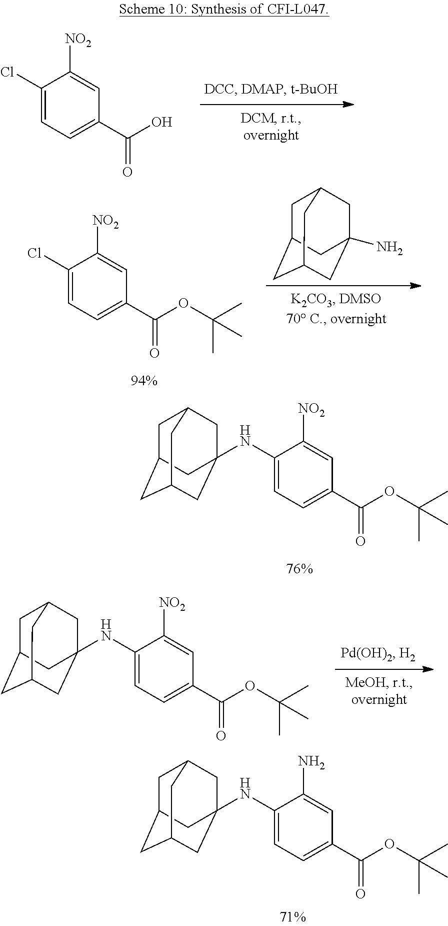

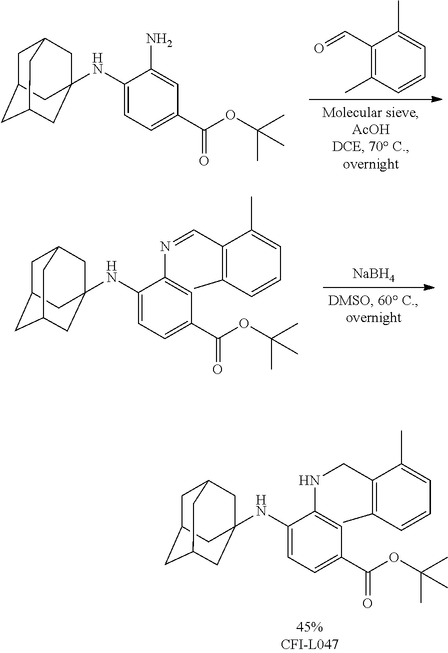

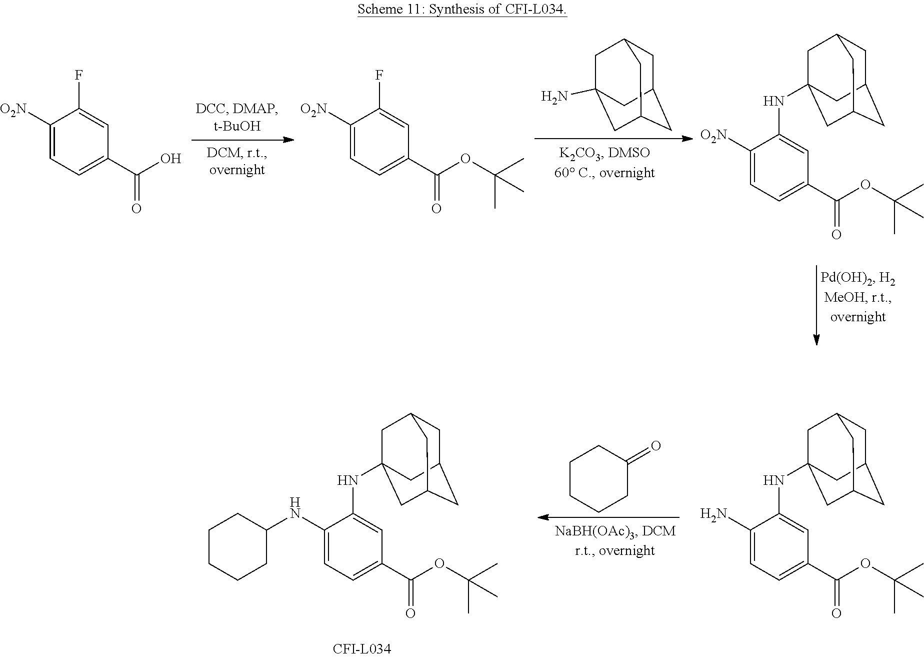

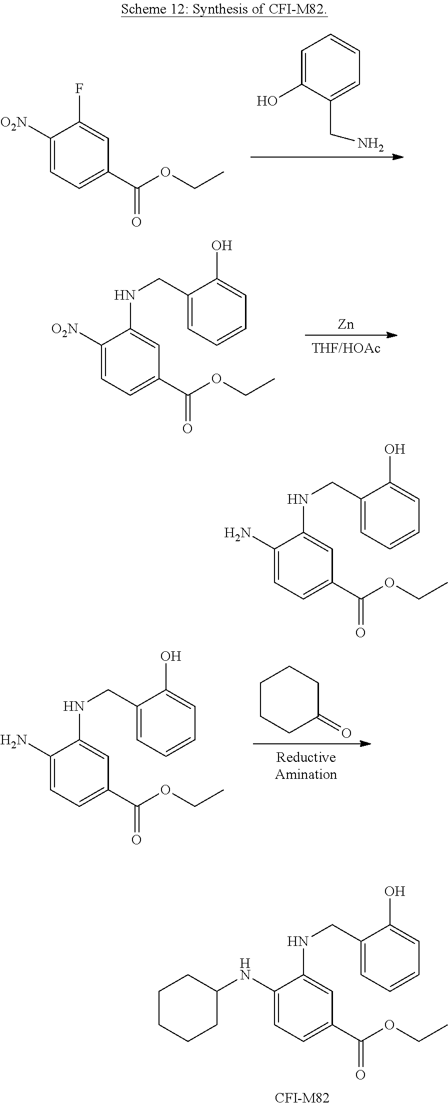

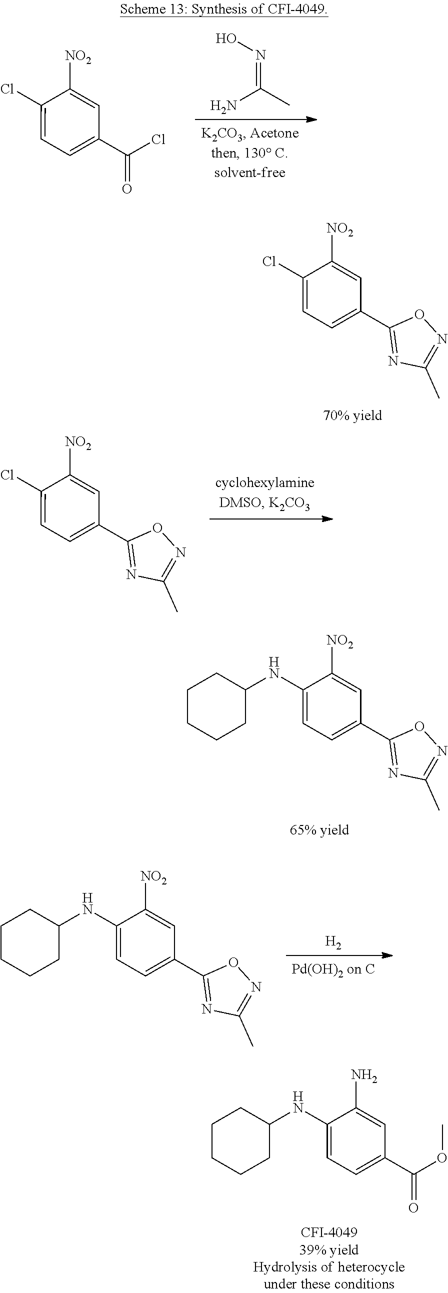

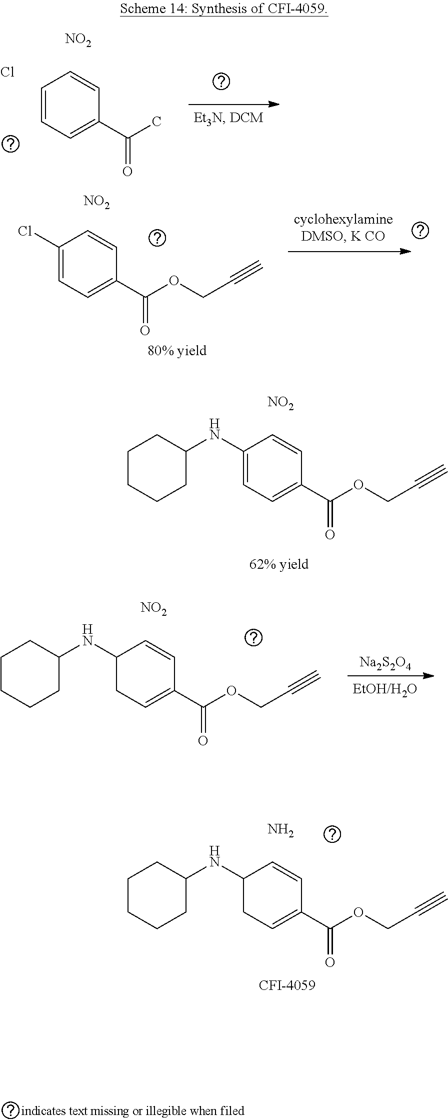

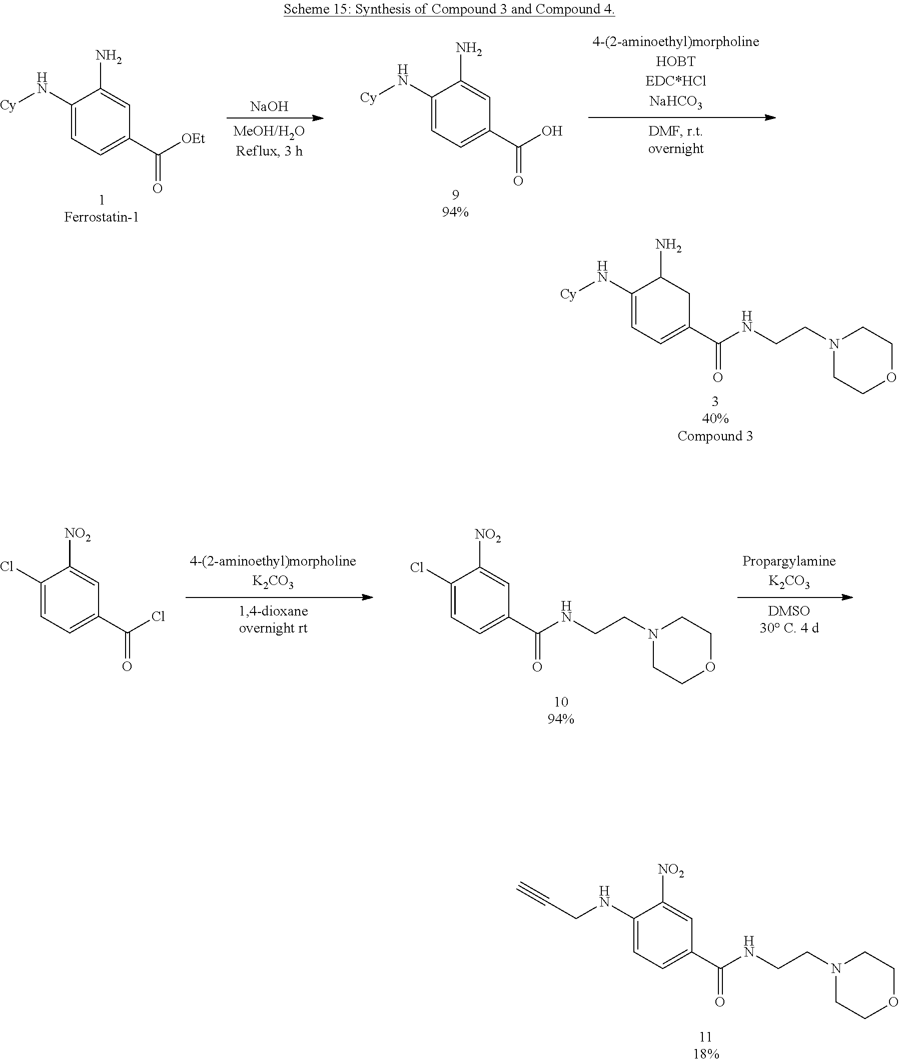

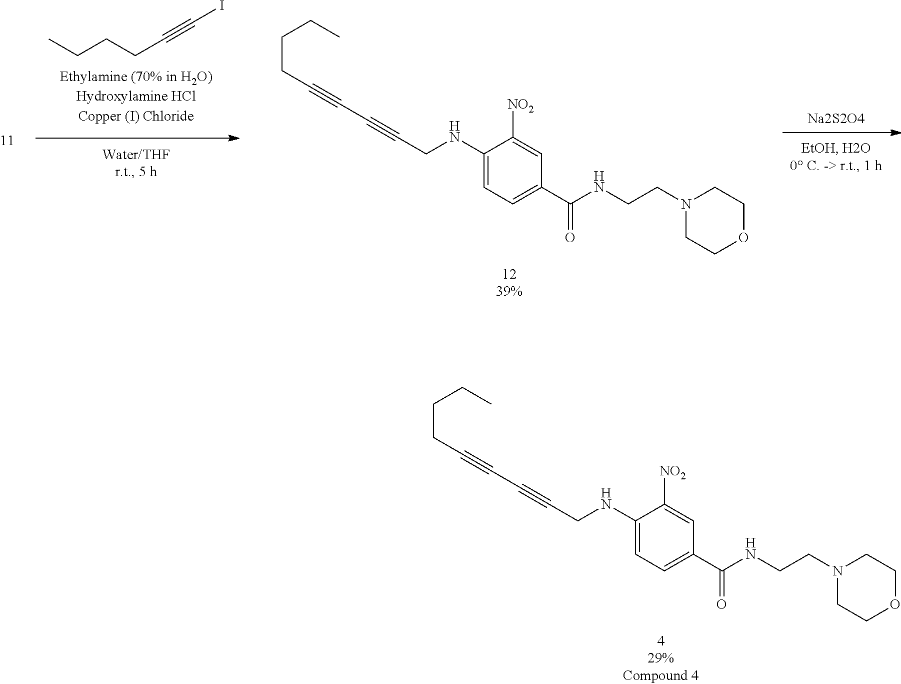

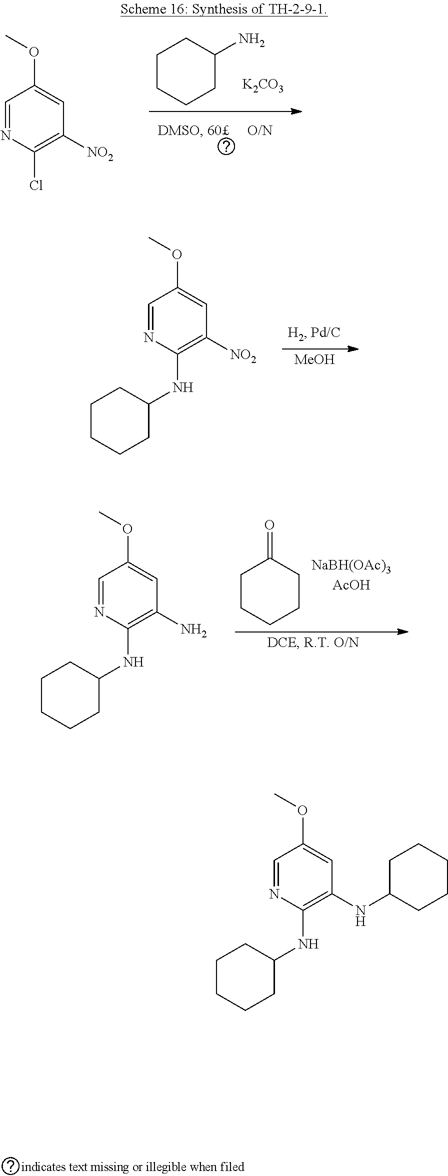

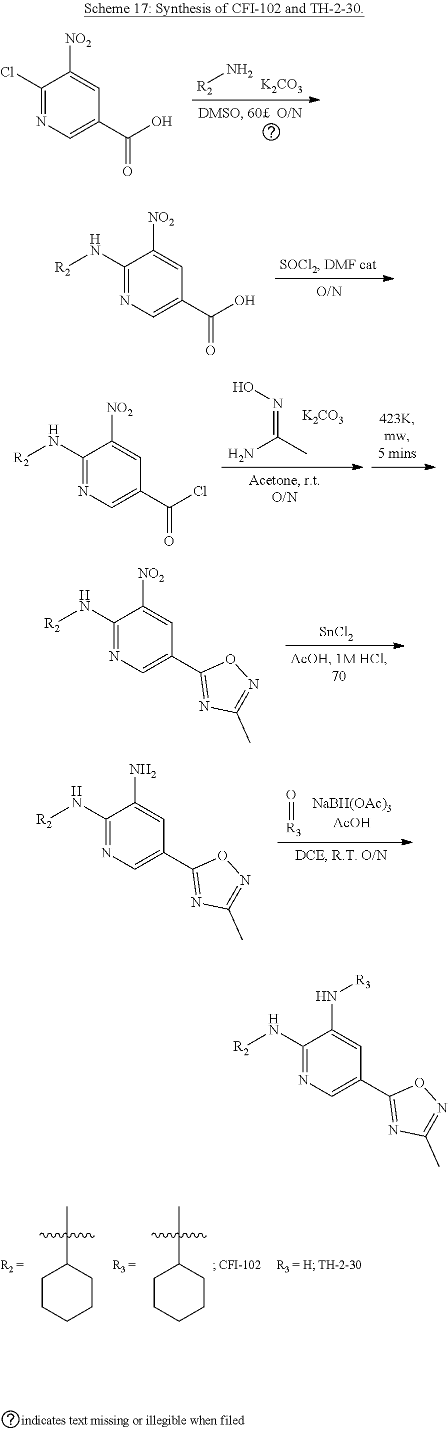

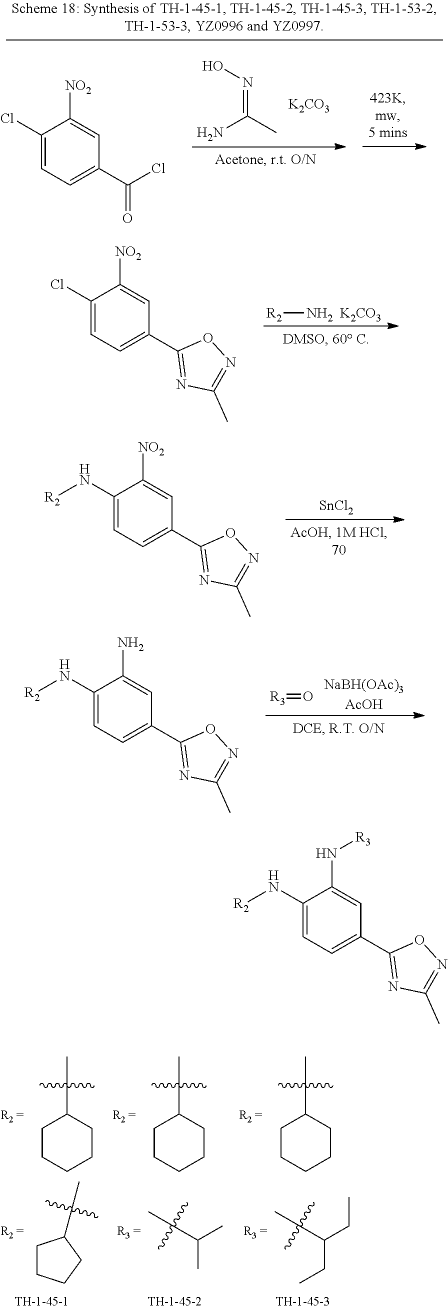

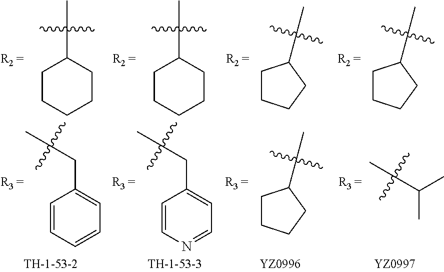

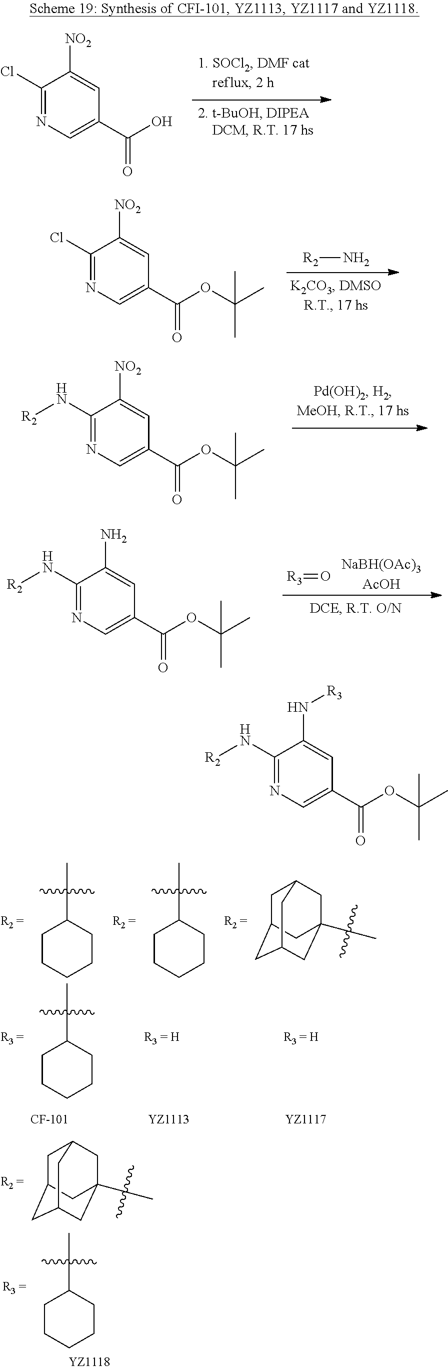

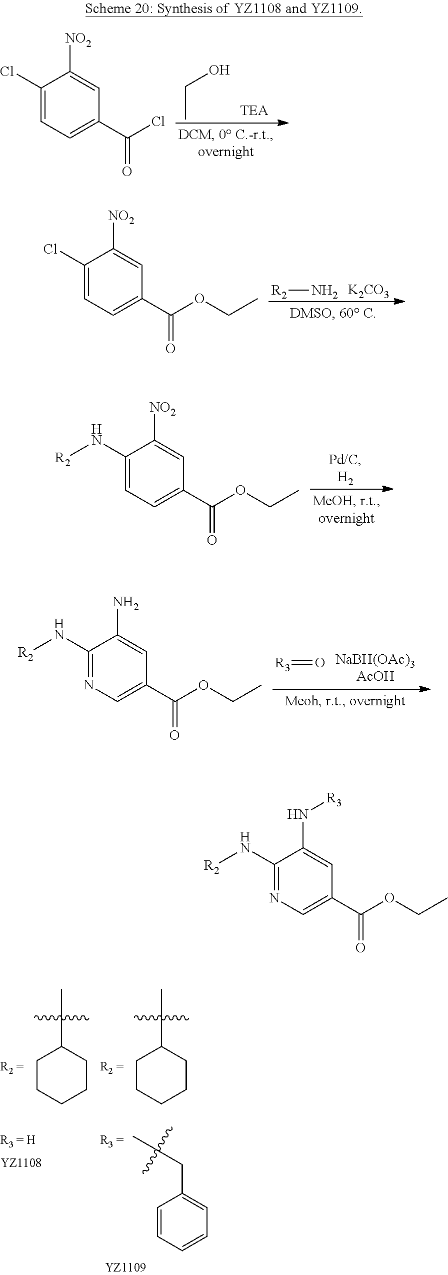

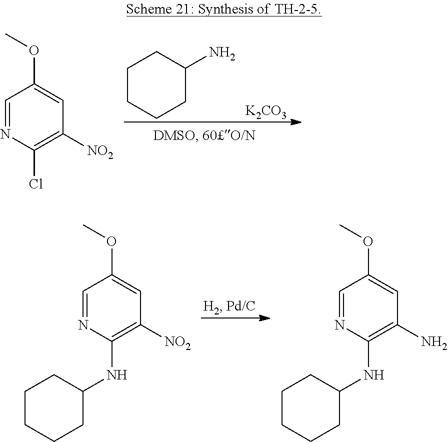

### Example 2

### Biological Activities of Ferrostatin-1 Analogs

All analogs are tested in vitro for their ability to inhibit erastin-induced ferroptosis in cells. Those with an IC₅₀ of <50 nM are tested for metabolic stability in mouse liver microsomes and plasma. Those analogs with T_{1/2}>30 minutes in those assays undergo pharmacokinetic analysis in mice. Those analogs with the best in vivo PK parameters are tested in the HD mouse model (see below).

### Rescue Activity of Fer-1 Analogs (Dixon, et al., 2012)

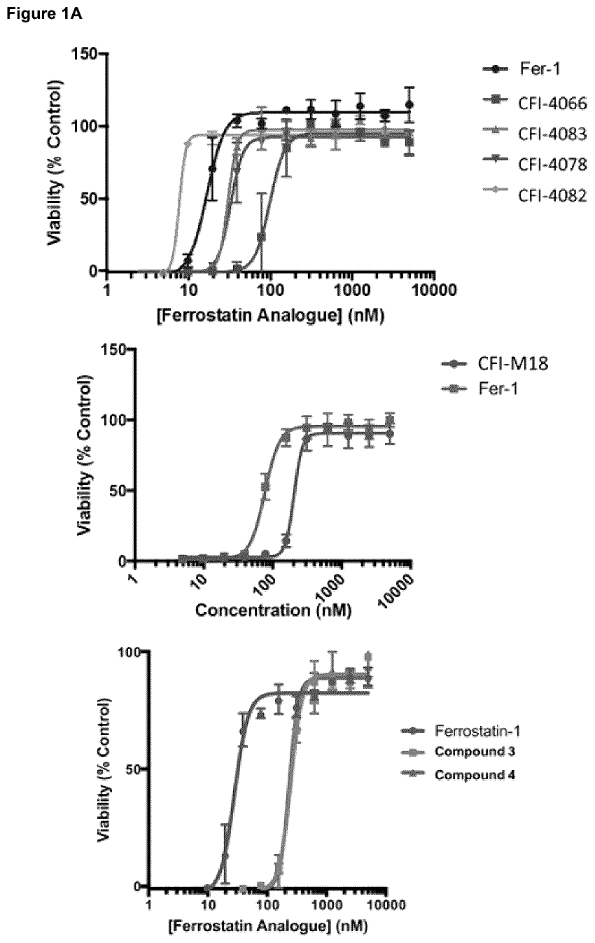

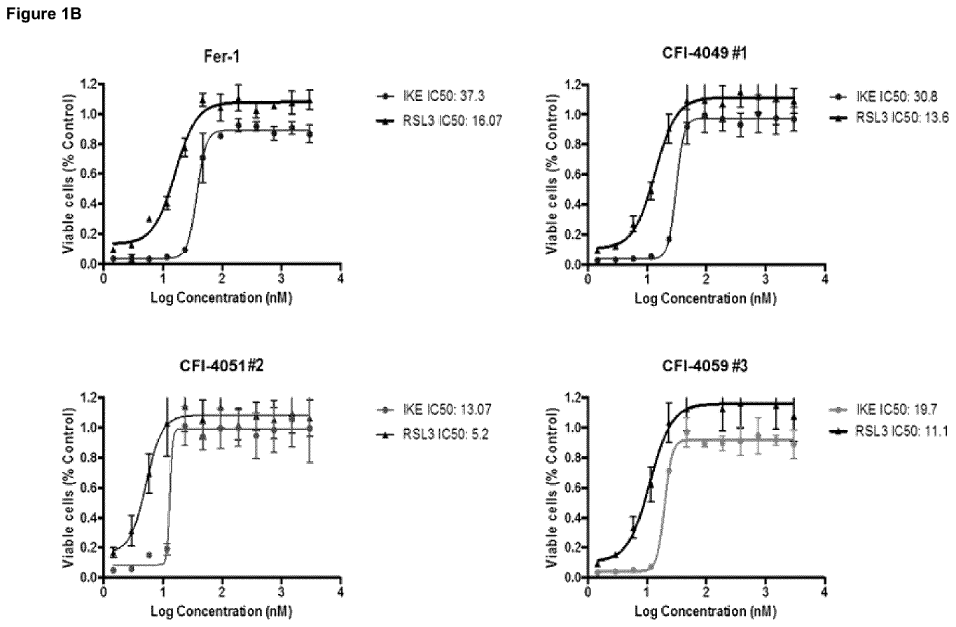

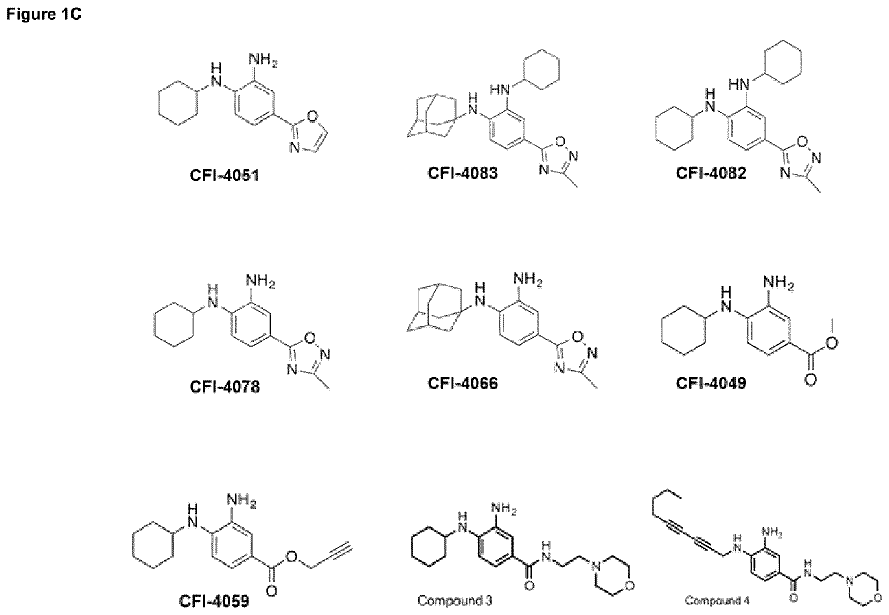

HT-1080 cells are cultured in DMEM containing 10% fetal bovine serum, 1% supplemented non-essential amino acids and 1% pen/strep mixture (Gibco) and maintained in a humidified environment at 37° C. with 5% CO₂ in a tissue culture incubator. 1,000 HT-1080 cells are seeded per well in duplicate 384-well plates (Corning) using a BioMek FX liquid handling robot (Beckman Coulter). The next day, the medium is replaced with 36 μL of medium containing 10 μM erastin with 4 μL of medium containing a dilution series (previously prepared) of DMSO, Fer-1 (positive control) or Fer-1 analogs. 24 hours later, 10 μL Alamar Blue (Invitrogen) cell viability solution is added to the growth media to a final concentration of 10%. Cells are incubated a further 6 hours and then the Alamar Blue fluorescence intensity recorded using a Victor 3 platereader (PerkinElmer)(ex/em 530/590). All experiments are performed at least twice and the background (no cells)-subtracted Alamar Blue values for each combination are averaged between replicates. The same procedure was repeated by replacing erastin (10 μM) with IKE (3 μM) or RSL3 (0.2 μM). From these data, sigmoidal dose-response viability curves (FIG. 1A for erastin, FIG. 1B for IKE and RSL3) and EC₅₀ values (Table 1) are computed using Prism 5.0 (GraphPad).

### Plasma and Metabolic Stability

Each compound (1 μM) is incubated with mouse plasma, for 4 hours at 37° C., with shaking at 100 rpm. The concentration of compound in the buffer and plasma chambers is determined using LC-MS/MS. Metabolism of each compound is predicted using Sites of Metabolism (Schrodinger Suite), which combines intrinsic reactivity analysis (Hammett-Taft) with induced fit docking against 2C9, 2D6 and 3A4. This approach identifies 90% of known metabolism sites and has a false positive rate of 17%. The in vitro metabolic stability of each compound in mouse liver microsomes is determined. Pooled mouse liver microsomes are prepared and stored at −80° C. until needed. Compound stability in liver microsomes is measured at 0, 15, 30, 45 and 60 minutes in duplicate, using LC-MS/MS analysis.

### Pharmacokinetic Evaluation of Compounds in Mice

To evaluate the PK profile of compounds, IV, IP, and PO administration of each compound is used in C57BL/6J wt mice. Mice are dosed IV at 10 mg/kg and sacrificed using Nembutal and CO₂ euthanasia. Six week old mice (Charles River) that have been acclimated to their environment for 2 weeks are used. All animals are observed for morbidity, mortality, injury, availability of food and water twice per day. Animals in poor health are euthanized. Blood samples are collected via cardiac puncture at each time point (0, 30 minutes, 2, 4, 8, 24 h). In addition, brains are collected, and compound concentration determined at each time point using LCO2N MS/MS. Standard PK parameters are calculated for each route of administration, including T_{1/2}, Cₘₐₓ, AUC, clearance, Vd and % F.

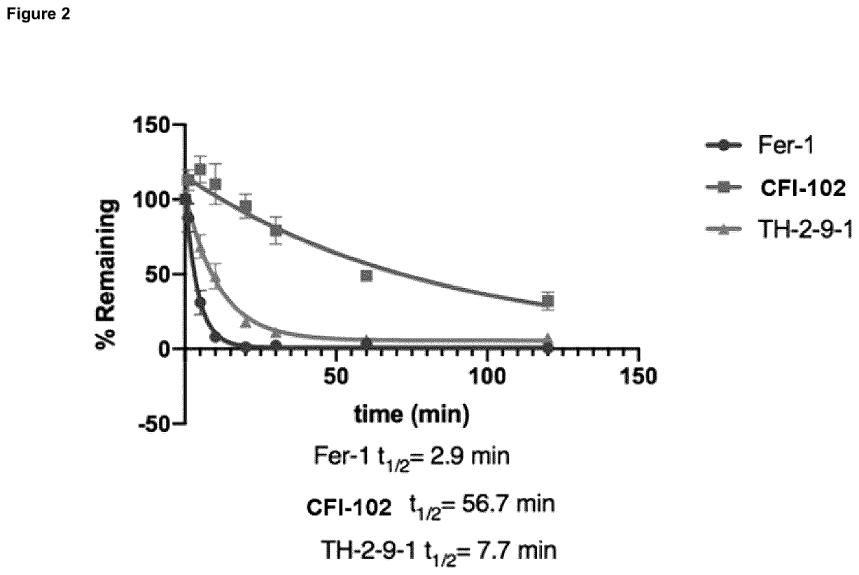

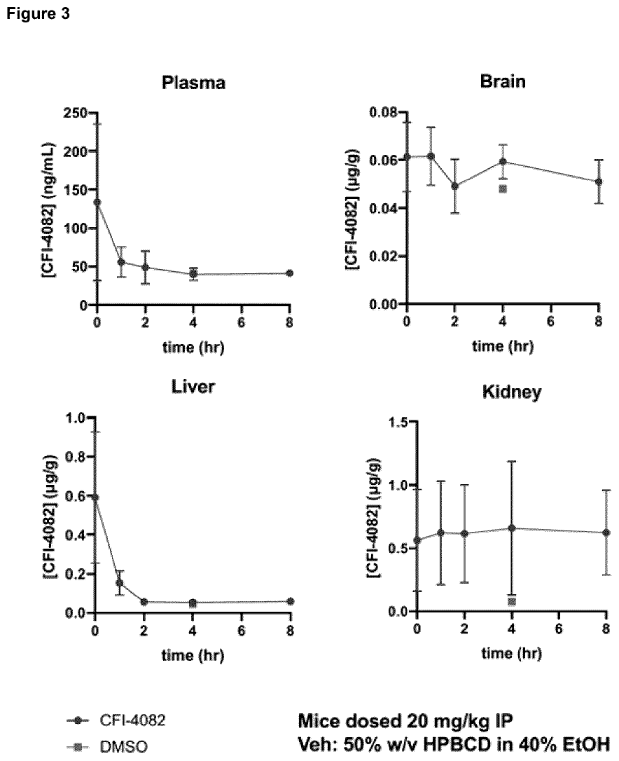

The properties of Ferrostatin-1 and analogs are summarized in Table 1. CFI-A8, CFI-A9, CFI-A11, CFI-L032, CFI-L034, CFI-L047, CFI-4082 and CFI-4083 show T_{1/2}>120 minutes in either mouse or human liver microsomes. Particularly, CFI-4082 and CFI-4083 show T_{1/2}>120 minutes in both mouse and human liver microsomes. The microsomal stability comparison (half-life measured in mouse) of Fer-1, CFI-102 and TH-2-9-1 is also provided in FIG. 2.

TABLE 1
Properties of Ferrostatin-1 and analogs.

| | | Plasma | | | | | | |
|---|---|---|---|---|---|---|---|---|
| | | Stability | | | | | | |
| | Microsomal | at six | | | | | accept | |
| | Stability | hours (% | | | | donor | or | |
| Compound and Structure | t_{1/2} | remaining) | MW | ClogP | PSA | HB | HB | EC₅₀ |
| Fer-1 | Mouse: 2.4 ± | Mouse: | 262.351 | 2.694 | 71.607 | 2.5 | 4 | 50^{a} |
| 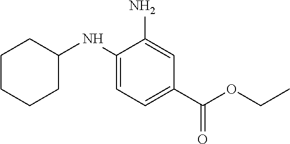 [SMILES: CCOC(=O)c1ccc(NC2CCCCC2)c(N)c1] | 0.6 min 1.9 ± 0.6 min¹ Human: 11.4 ± 0.2 min 6.9 ± 0.2 min¹ Rat: 3.0 ± 11.1 min Dog: 49.5 ± 9.6% | 0-2% Human: 100% Rat: 0-4% Dog: | | | | | | |
| | remaining | | | | | | | |
| | t = 120 | | | | | | | |
| | Pig: 16.2 ± 3.9 | Pig: 100% | | | | | | |
| | min | | | | | | | |
| CFI-4051 | Mouse: 3.2-3.3 | Mouse: — | 257.335 | 2.641 | 57.392 | 2.5 | 4 | 36^{a} |
| 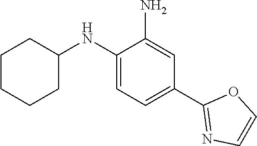 [SMILES: Nc1cc(-c2ncco2)ccc1NC1CCCCC1] | min Human: >120 min Rat: 30 < t_{1/2} < 60 min Dog: >120 min Pig: — | Human: — Rat: — Dog: — Pig: — | | | | | | 13^{b} 5^{c} |
| CFI-4066 | Mouse: 30.2- | Mouse: — | 324.425 | 2.854 | 74.416 | 2.5 | 5 | 123ª |
| 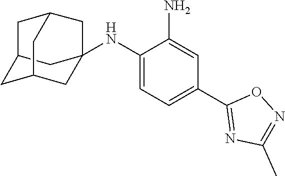 [SMILES: Cc1noc(-c2ccc(NC34CC5CC(CC(C5)C3)C4)c(N)c2)n1] | 32.4 min Human: — Rat: — Dog: — Pig: — | Human: — Rat: — Dog: — Pig: — | | | | | | |
| CFI-A3 | Mouse: 16.2- | Mouse: — | 376.925 | 4.474 | 64.699 | 2.5 | 4 | 26^{b} |
| 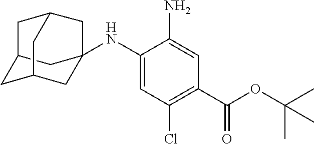 [SMILES: CC(C)(C)OC(=O)c1cc(N)c(NC23CC4CC(CC(C4)C2)C3)cc1Cl] | 16.6 min Human: >120 min Rat: 30 < t_{1/2} < 60 min Dog: >120 min Pig: — | Human: — Rat: — Dog: — Pig: — | | | | | | 6^{c} |
| CFI-A4 | Mouse: 12.5- | Mouse: — | 356.507 | 4.335 | 63.254 | 2.5 | 4 | 16^{b} |
| 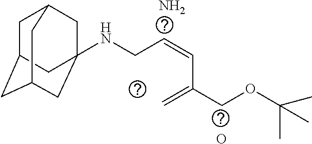 | 13.4 min Human: >120 min Rat: ~20 min Dog: 30 < t_{1/2} < 60 min Pig: — | Human: — Rat: — Dog: — Pig: — | | | | | | 1^{c} |
| CFI-A78 | Mouse: 25.7- | Mouse: — | 360.471 | 4.272 | 65.511 | 2.5 | 4 | 26^{b} |
| 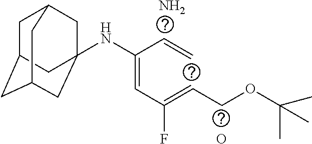 | 27.7 min Human: — Rat: — Dog: — Pig: — | Human: — Rat: — Dog: — Pig: — | | | | | | 18^{c} |
| CFI-A8 | Mouse: >120 | Mouse: | 442.616 | 6.598 | 50.622 | 2 | 4 | 20^{b} |
| 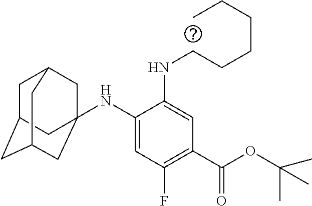 [SMILES: CCCCCCNc1cc(C(=O)OC(C)(C)C)c(F)cc1NC12CC3CC(CC(C3)C1)C2] | min (76.4%) Human: TBD Rat: TBD Dog: Pig: TBD | 100% Human: TBD Rat: TBD Dog Pig: TBD | | | | | | 19^{c} |
| CFI-A9 | Mouse: >120 | Mouse: | 438.652 | 6.692 | 48.468 | 2 | 4 | 65ª |
| 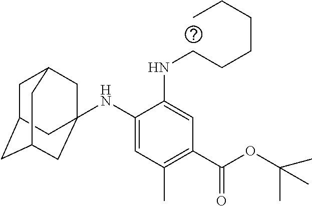 [SMILES: CCCCCCNc1cc(C(=O)OC(C)(C)C)c(C)cc1NC12CC3CC(CC(C3)C1)C2] | min (83.6%) Human: TBD Rat: TBD Dog Pig: TBD | 100% Human: TBD Rat: TBD Dog Pig: TBD | | | | | | |
| CFI-A11 | Mouse: >120 | Mouse: | 459.07 | 6.818 | 49.837 | 2 | 4 | 95 [symbol] |
| 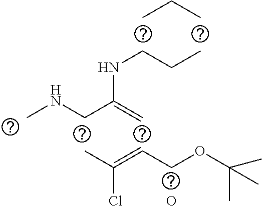 | min (69.1%) Human: TBD Rat: TBD Dog: TBD Pig: TBD | 100% Human: TBD Rat: TBD Dog Pig: TBD | | | | | | |
| CFI-L032 | Mouse: >120 | Mouse: | 424.625 | 6.346 | 50.91 | 2 | 4 | 43 [symbol] |
| 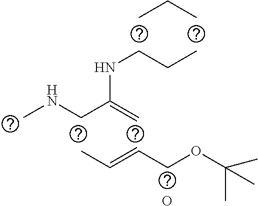 | min (78.7%) Human: TBD Rat: TBD Dog: Pig: TBD | 100% Human: TBD Rat: TBD Dog Pig: TBD | | | | | | 77 |
| CFI-L034 | Mouse: >120 | Mouse: | 424.625 | 6.415 | 50.95 | 2 | 4 | 215 [symbol] |
| 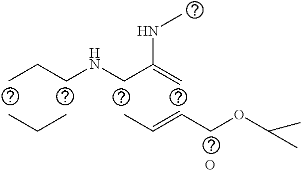 | min (60.2%) Human: TBD Rat: TBD Dog: Pig: TBD | 100% Human: TBD Rat: TBD Dog Pig: TBD | | | | | | 166 |
| CFI-L047 | Mouse: >120 | Mouse: | 460.658 | 7.248 | 50.864 | 2 | 4 | 294 [symbol] |
| 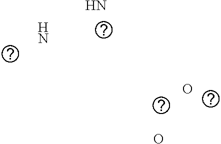 | min (59.3%) Human: TBD Rat: TBD Dog: Pig: TBD | 100% Human: TBD Rat: TBD Dog Pig: TBD | | | | | | |
| CFI-M40 | Mouse: ~60 | Mouse: | 351.491 | 4.564 | 73.747 | 3.5 | 4.5 | 77 [symbol] |
| 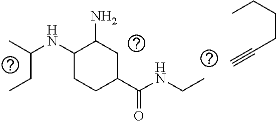 | min Human: TBD Rat: TBD Dog: Pig: TBD | 100% Human: TBD Rat: TBD Dog: Pig: TBD | | | | | | |
| CFI-M18 | Mouse: 20 < | Mouse: — | 298.384 | 3.78 | 73.386 | 2.5 | 4 | 203 [symbol] |
| 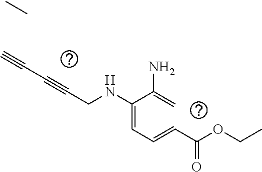 | t_{1/2} < 30 min Human: TBD Rat: TBD Dog: Pig: TBD | Human: — Rat: — Dog Pig: — | | | | | | |
| CFI-M82 | Mouse: 10 min | Mouse: — | 368.475 | 4.557 | 76.996 | 3 | 4.75 | 14 [symbol] |
| 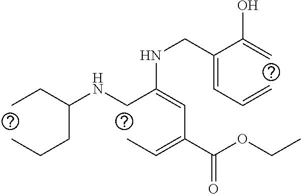 [SMILES: C=C/C=C(/CN/C(=C/C(=C\C)C(=O)OCC)CNC(CC)CCC)C(=C)O] | Human: — Rat: — Dog: Pig: — | Human: — Rat: — Dog Pig: — | | | | | | |
| CFI-4078 | Mouse: ~65 | Mouse: — | 272.349 | 2.202 | 75.449 | 2.5 | 5 | 102 [symbol] |
| 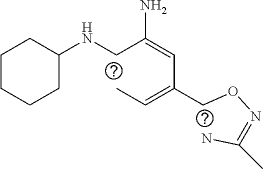 [SMILES: C/C=C(\C=C(\N)CNC1CCCCC1)CO/N=C(/C)N] | min Human: TBD Rat: TBD Deg: TBD Pig: TBD | Human: — Rat: — Dog Pig: — | | | | | | |
| CFI-4082 | Mouse: ~120 | Mouse: | 354.494 | 4.466 | 60.571 | 2 | 5 | 7 [symbol] |
| 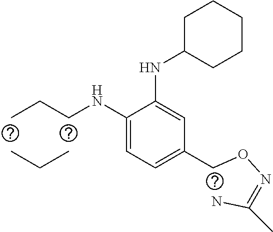 | min Human: >120 min Rat: 30 < t_{1/2} < 60 min Dog: Pig: 60 < t_{1/2} < 120 min | 100% Human: TBD Rat: TBD Dog Pig: TBD | | | | | | |
| CFI-4083 | Mouse: ~120 | Mouse: | 406.570 | 5.122 | 59.507 | 2 | 5 | 22 [symbol] |
| 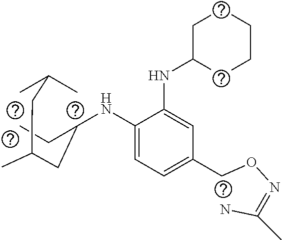 [SMILES: CCC(CC(C)CC(C)C)Nc1ccc(CO/N=C(/C)N)cc1NC1CCCCC1] | min Human: >120 min Rat: TBD Dog: TBD Pig: TBD | TBD Human: TBD Rat: TBD Dog Pig: TBD | | | | | | |
| CFI-4081 | Mouse: 30 < | Mouse: — | 366.51 | 5.764 | 52.203 | 2 | 3.5 | 37 [symbol] |
| 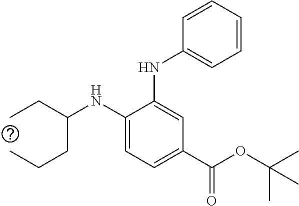 [SMILES: CCCC(CC)Nc1ccc(C(=O)OC(C)(C)C)cc1Nc1ccccc1] | t_{1/2} < 60 min Human: >120 min Rat: 30 < t_{1/2} < 60 min Dog: Pig: 60 < t_{1/2} < 120 min | Human: — Rat: — Dog Pig: — | | | | | | |
| TH-2-9-1 | Mouse: 7.7 min | | 303.23 | 4.519 | 39.778 | 2 | 3.25 | 0.1ª |
| 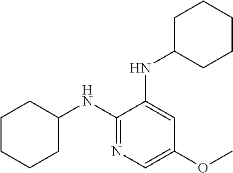 [SMILES: COc1cnc(NC2CCCCC2)c(NC2CCCCC2)c1] | | | | | | | | 0.21^{b} 0.2^{c} |
| CFI-102 | Mouse: 30 < | | 355.24 | 4.132 | 70.599 | 2 | 5.5 | 0.9^{b} |
| 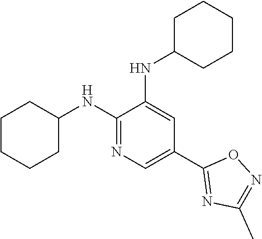 [SMILES: Cc1noc(-c2cnc(NC3CCCCC3)c(NC3CCCCC3)c2)n1] | t_{1/2} < 60 min | | | | | | | 0.9^{c} |
| CFI-101 | | | 373.541 | 5.109 | 59.291 | 2 | 4.5 | 354.2ª |
| 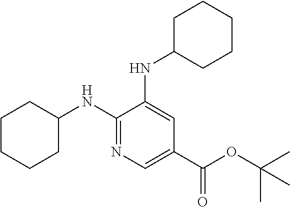 [SMILES: CC(C)(C)OC(=O)c1cnc(NC2CCCCC2)c(NC2CCCCC2)c1] | | | | | | | | 170.6^{b} 162.6^{c} |
| TH-1-45-1 | | | 340.471 | 3.702 | 64.136 | 2 | 5 | 33.8ª |
| 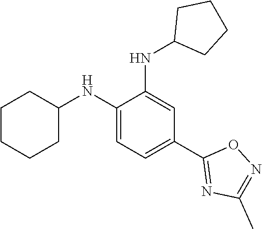 [SMILES: Cc1noc(-c2ccc(NC3CCCCC3)c(NC3CCCC3)c2)n1] | | | | | | | | 17.3^{b} 5.1^{c} |
| TH-1-45-3 | | | 342.487 | 4.081 | 62.834 | 2 | 5 | 37.8ª |
| 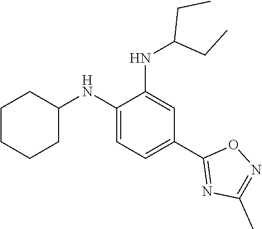 [SMILES: CCC(CC)Nc1cc(-c2nc(C)no2)ccc1NC1CCCCC1] | | | | | | | | 26.9^{b} 6.6^{c} |
| TH-1-45-2 | | | 314.433 | 3.313 | 63.472 | 2 | 5 | 22.3ª |
| 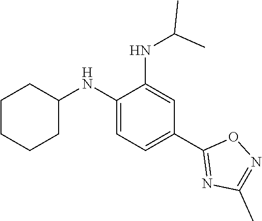 [SMILES: Cc1noc(-c2ccc(NC3CCCCC3)c(NC(C)C)c2)n1] | | | | | | | | 16.2^{b} 4.2^{c} |
| TH-1-53-2 | | | 362.477 | 4.281 | 62.607 | 2 | 5 | 44.5ª |
| 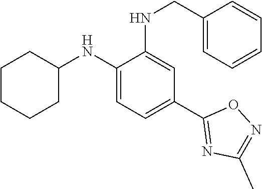 [SMILES: Cc1noc(-c2ccc(NC3CCCCC3)c(NCc3ccccc3)c2)n1] | | | | | | | | 40.2^{b} 9.4^{c} |
| TH-1-53-3 | | | 363.465 | 3.774 | 78.781 | 2 | 6.5 | 63.8ª |
| 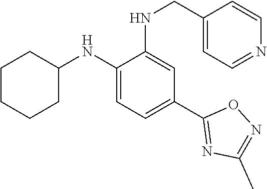 [SMILES: Cc1noc(-c2ccc(NC3CCCCC3)c(NCc3ccncc3)c2)n1] | | | | | | | | 63.0^{b} 31.5^{c} |
| YZ0996 | | | 326.441 | 3.851 | 60.740 | 2 | 5 | 2ª |
| 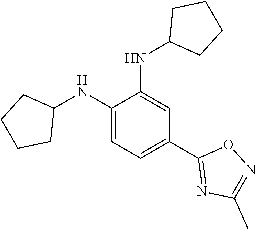 [SMILES: Cc1noc(-c2ccc(NC3CCCC3)c(NC3CCCC3)c2)n1] | | | | | | | | |
| YZ0997 | | | 300.403 | 3.440 | 60.645 | 2 | 5 | 2.5ª |
| 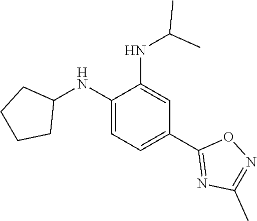 [SMILES: Cc1noc(-c2ccc(NC3CCCC3)c(NC(C)C)c2)n1] | | | | | | | | |
| YZ1108 | | | 263.341 | 2.072 | 78.354 | 2.5 | 4.5 | 314.6ª |
| 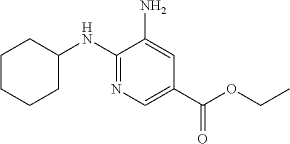 [SMILES: CCOC(=O)c1cnc(NC2CCCCC2)c(N)c1] | | | | | | | | 639^{b} 1038^{c} |
| YZ1109 | | | 353.466 | 4.438 | 65.172 | 2 | 4.5 | 317.2ª |
| 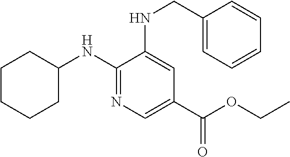 [SMILES: CCOC(=O)c1cnc(NC2CCCCC2)c(NCc2ccccc2)c1] | | | | | | | | 165.5^{b} 115.2^{c} |
| YZ1113 | | | 291.395 | 2.9 | 73.657 | 2.5 | 4.5 | 338.2ª |
| 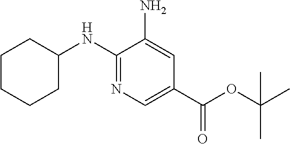 [SMILES: CC(C)(C)OC(=O)c1cnc(NC2CCCCC2)c(N)c1] | | | | | | | | 311.8^{b} 156.2^{c} |
| YZ1117 | | | 343.226 | 3.6 | 71.77 | 2.5 | 4.5 | 85.0ª |
| 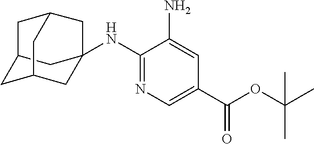 [SMILES: CC(C)(C)OC(=O)c1cnc(NC23CC4CC(CC(C4)C2)C3)c(N)c1] | | | | | | | | 68.4^{b} 39.1^{c} |
| YZ1118 | | | 425.304 | 5.792 | 57.416 | 2 | 4.5 | 40.1^{b} |
| 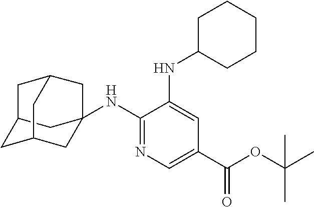 [SMILES: CC(C)(C)OC(=O)c1cnc(NC23CC4CC(CC(C4)C2)C3)c(NC2CCCCC2)c1] | | | | | | | | 37.3^{c} |
| TH-2-5 | | | 221.30 | 2.269 | 54.477 | 2.5 | 3.25 | >350 |
| 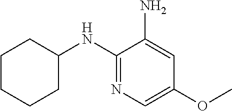 [SMILES: COc1cnc(NC2CCCCC2)c(N)c1] | | | | | | | | |
| TH-2-30 | | | 273.16 | 1.909 | 85.371 | 2.5 | 5.5 | 461.3^{b} |
| 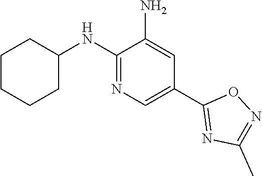 [SMILES: Cc1noc(-c2cnc(NC3CCCCC3)c(N)c2)n1] | | | | | | | | 118.4^{c} |

¹Hofmans et al., 2016, J. Med. Chem, 59, 2041-2053
Mouse: CD1; for compounds with t_{1/2} > 120 min, the average % remaining after 120 minutes is provided in parentheses
Human: Pooled, 50 donors
Rat: Sprague Dawley
Dog: Beagle
Pig: Göttingen Minipig
TBD: to be determined
ClogP: Predicted octanol/water partition coefficient.
PSA: Total Van der Waals surface area of polar nitrogen and oxygen atoms and carbonyl carbon atoms.
donorHB: Estimated number of hydrogen bonds that would be donated by the solute to water molecules in an aqueous solution. Values are averages taken over a number of configurations, so they can be non-integer.
AccptHB: Estimated number of hydrogen bonds that would be accepted by the solute from water molecules in an aqueous solution. Values are averages taken over a number of configurations, so they can be non-integer.
EC₅₀:
^{a}Concentration (nM) of ferrostatin analogue required to achieve 50% viability against HT-1080 cells treated with 10 μM erastin.
^{b}Concentration (nM) of ferrostatin analogue required to achieve 50% viability against HT-1080 treated with 3 μM IKE.
^{c}Concentration (nM) of ferrostatin analogue required to achieve 50% viability against HT-1080 treated with 0.2 μM RSL3. [symbol]
indicates data missing or illegible when filed

### Example 3

### Metabolic Stability of CFI-4082

To determine the suitability of CFI-4082 for further in vivo applications, we administered a single dose of CFI-4082 (20 mg/kg in 50% 2-hydroxypropyl-β-cyclodextrin dissolved in 40% ethanol) to male and female C67Bl/6 mice (Jackson Lab) via intraperitoneal injection over the course of eight hours, with the compound concentration in plasma and tissue determined by LC/MS-MS. CFI-4082 was found to have low in vivo plasma stability, but was found to stably accumulate in kidney over 8 hours (FIG. 3).

### Example 4

### Rescue Activity of Selected Fer-1 Analogs

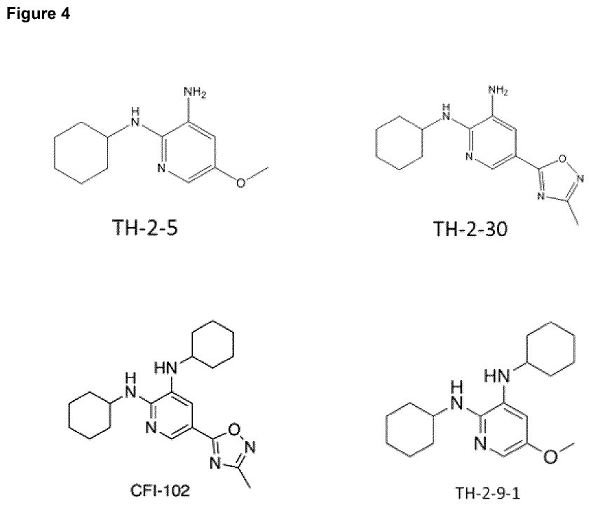

Selected Fer-1 analogs containing a pyridine moiety (FIG. 4) were tested to examine their efficacy and overall potency in inhibiting ferroptosis. For each of these compounds, dose-response curves were generated in HT-1080 cells looking at the effectiveness of the molecules in inhibiting ferroptosis induced by either 3 μM IKE or 0.2 μM RSL3, Fer-1 was used as a positive control. For each dose-response curve, 1,000 cells/well were seeded in a 384 well plate and allowed to adhere overnight prior to treating with compound from a daughter plate. Cells were treated for 48 hours, unless otherwise noted, prior to viability being analyzed using cell titer glo (40 μL per well). All liquid handling was performed using the BioMek. All samples were prepared in triplicate, unless otherwise noted.

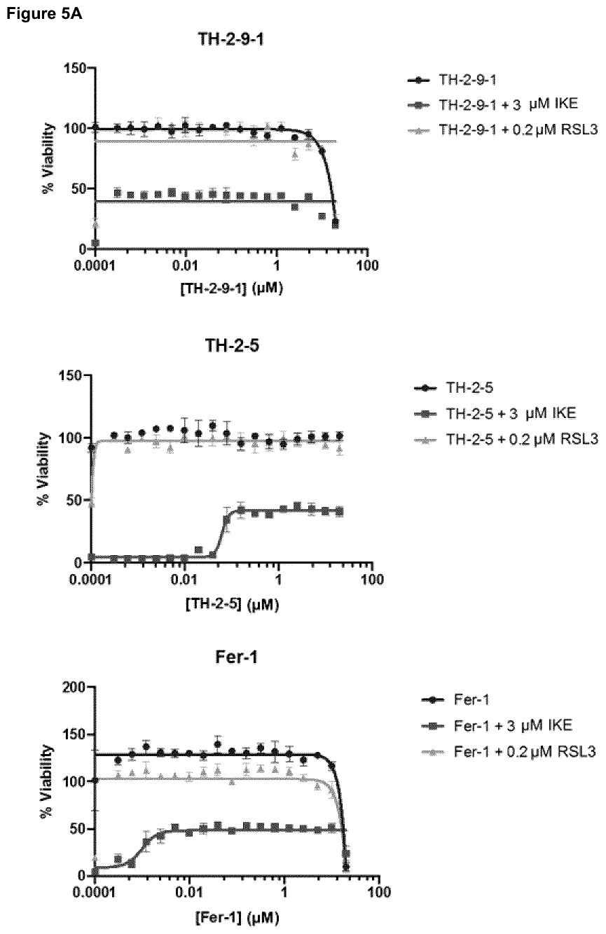

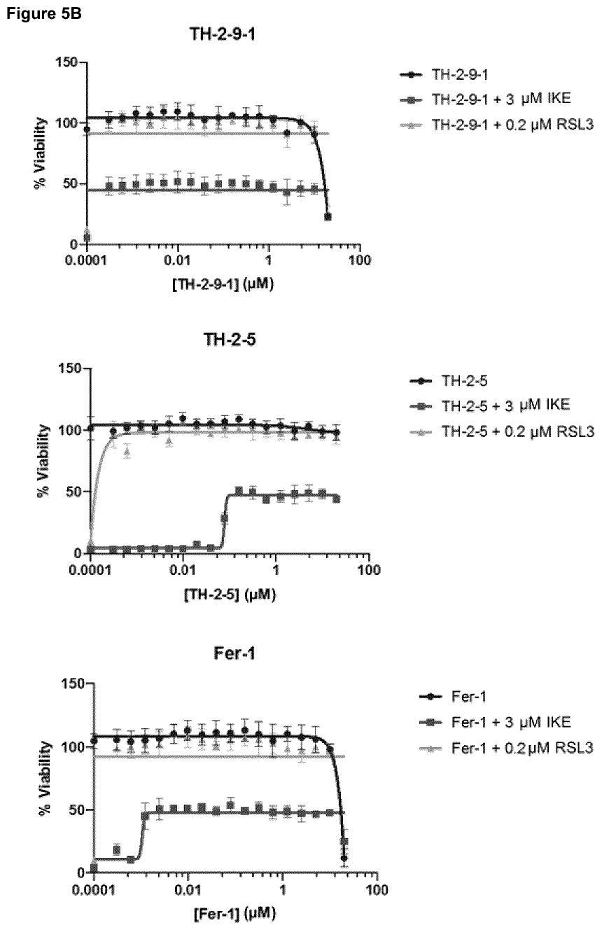

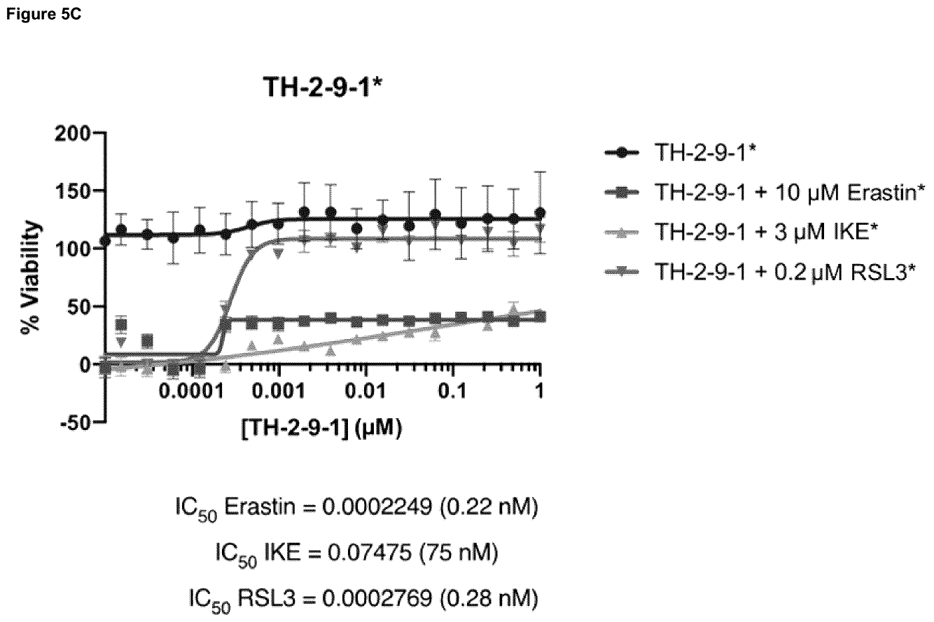

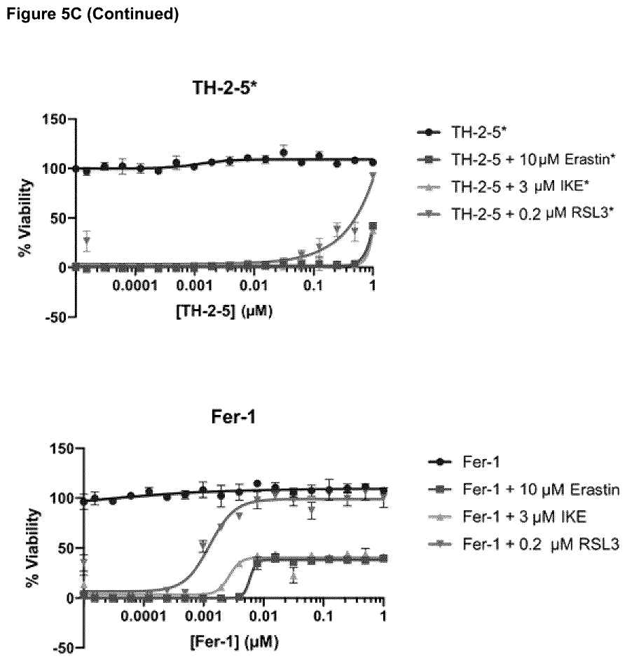

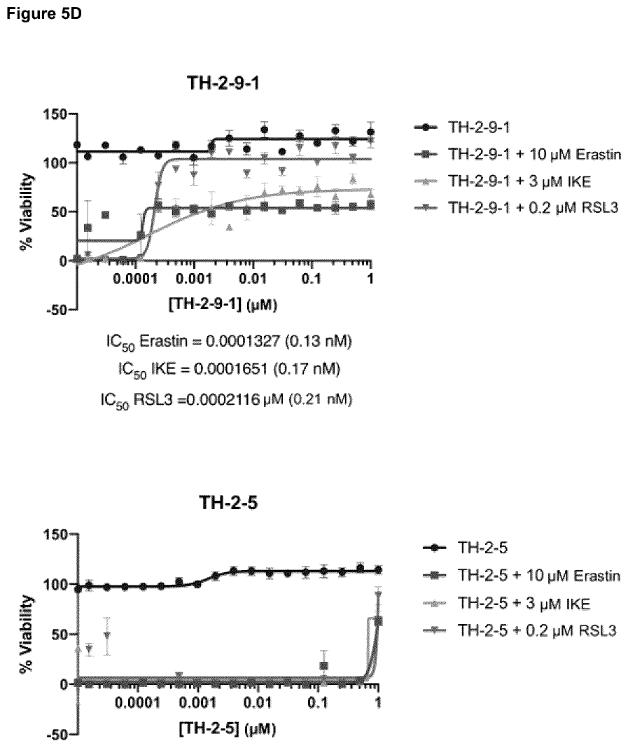

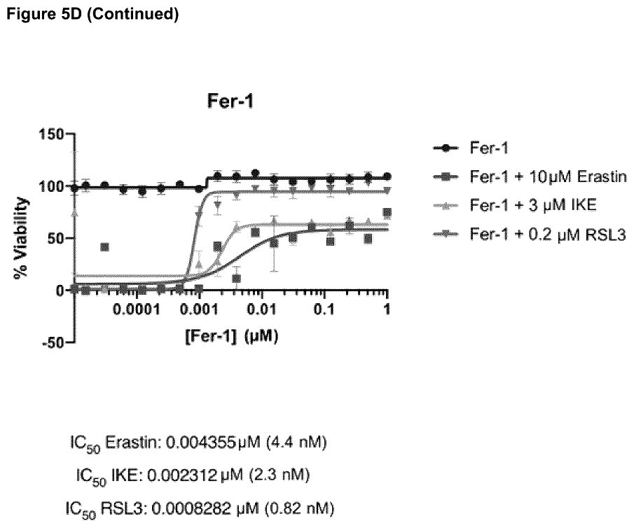

TH-2-9-1 and TH-2-5 compounds were first tested at a concentration range from 20 μM-0 μM. which was too high to capture any death at the lower concentrations, as evidenced by both compounds showing almost full rescue at most concentrations within the range (FIG. 5A).

The tests were repeated at a lower concentration range from 10 μM-0 μM, which was effective in capturing some of the earlier death. No death was observed with RSL3 for Fer-1, TH-2-9-1, and TH-2-5, suggesting that lower inhibitor concentrations were still needed (FIG. 5B).

By further lowering the concentration, compounds were tested at a range from 1 μM-0 μM. Erastin was also used in the test as a ferroptosis inducer. Following the same protocol, cells were treated with 10 μM erastin. As shown in FIG. 5C, TH-2-9-1 was protective against the cell death across the concentration range tested, indicating a higher potency than Fer-1 based on the leftward shift of the curves. Notably, both fer-1 and TH-2-9-1 were only able to produce ˜50% rescue against IKE and erastin. Another set of tests were repeated at the concentration range from 1 μM-0 μM, the results of which were largely consistent with the previous experiments (FIG. 5D). Fer-1 in this repeated experiment was much more potent than previously reported, with the potency nearly an order of magnitude higher than previously observed, while TH-2-9-1 was an order of magnitude more potent than Fer-1 for all inducers beyond RSL3, indicating that TH-2-9-1 can be a potential Fer-1 analog for in vivo applications.

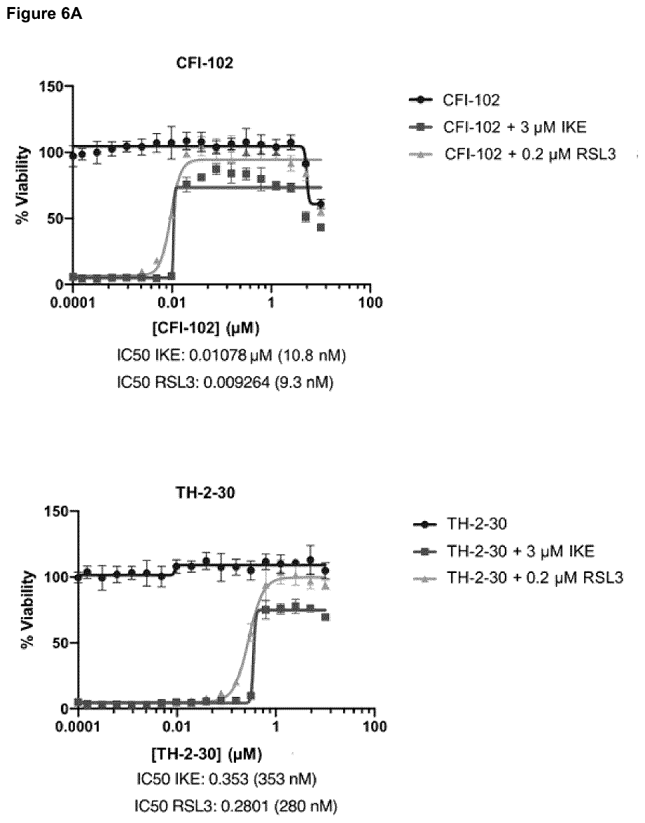

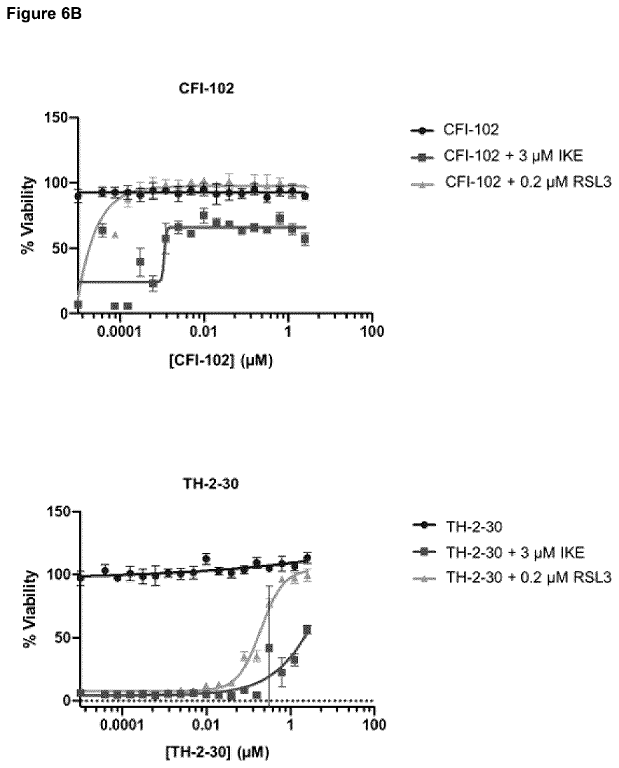

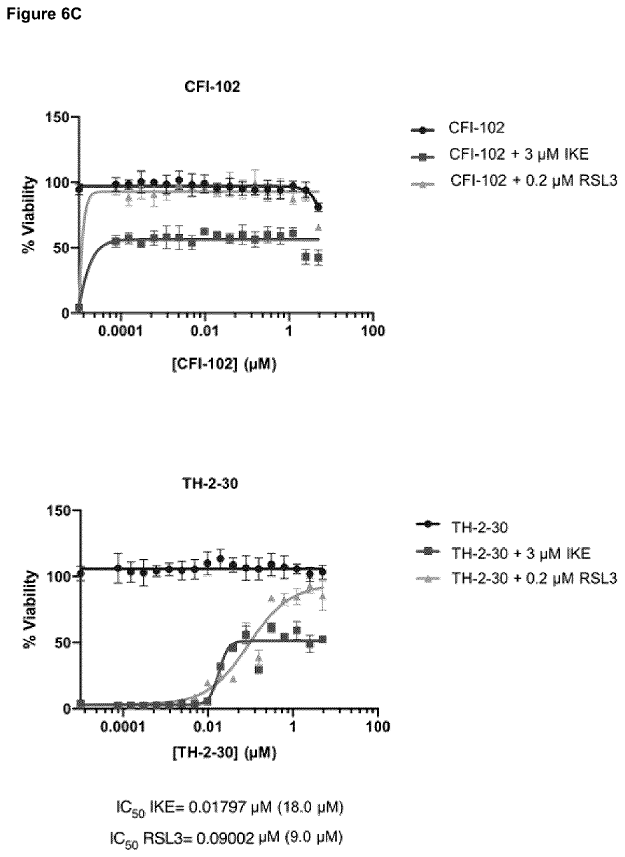

Two more compounds, CFI-102 and TH-2-30 were also tested for their anti-ferroptosis activities, using the same protocol as described above. Starting with a concentration range from 10 μM-0 μM, both compounds demonstrated activity against both IKE and RSL3, with CFI-102 having an IC₅₀ of ˜10-20 nM against both IKE and RSL3. TH-2-30 was relatively less potent. At 10 μM, both compounds appeared to be toxic, as evidenced by the overall drop in viability for all treatment conditions at the concentration (FIG. 6A).

The tests were repeated at a lower concentration range from 2.5 μM-0 μM, While the toxicity issues at 10 μM was not present, it appeared that 2.5 μM was too low of a starting concentration for TH-2-30 to fully establish rescue (FIG. 6B). Therefore, another set of experiments was conducted with the staring concentration of 5 μM. For this set of experiments the samples were treated for 51 hours instead of 48 hours. As shown in FIG. 6C, no compound was able to achieve full resuce against IKE at the highest concentration; this might be due to this batch of IKE being more potent or some other factor. Both compounds showed activity against IKE and RSL3, and CFI-102 was more potent by achieving full rescue at around 0.0001 μM.

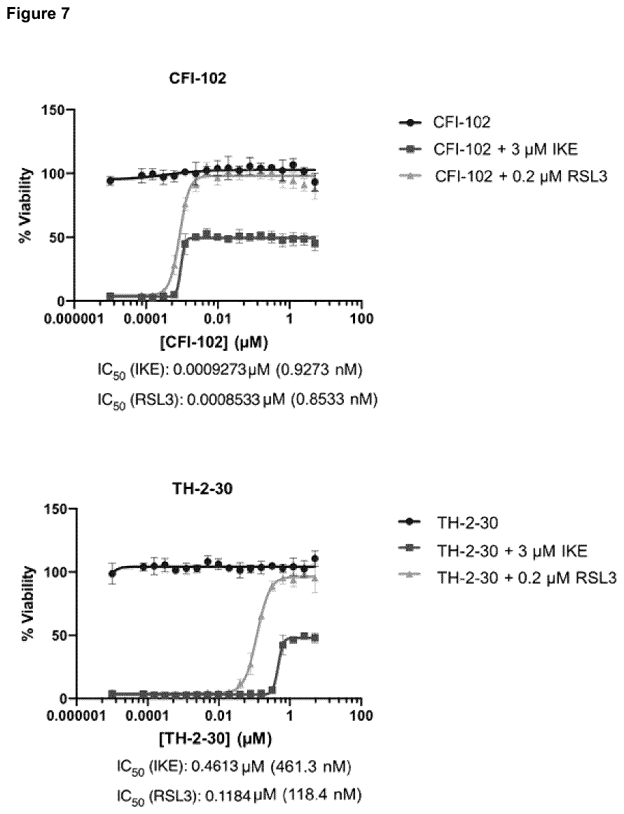

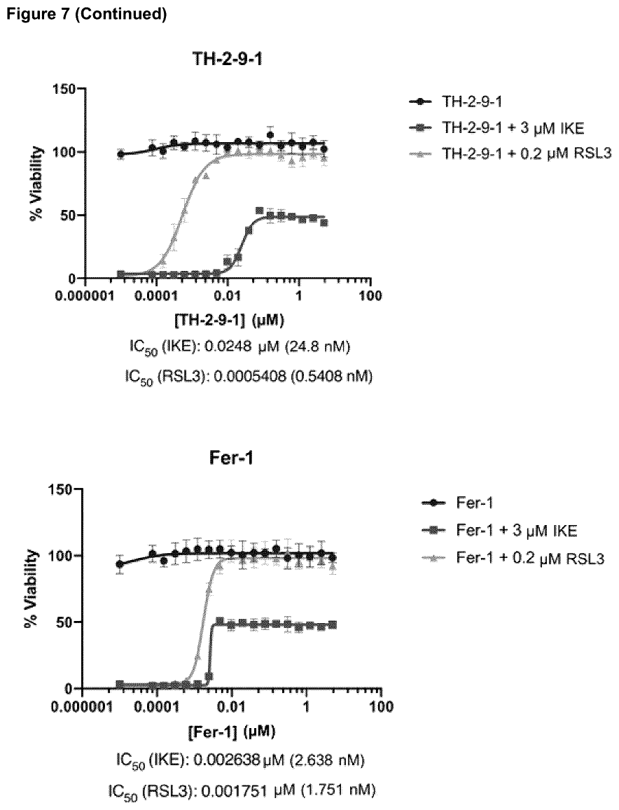

Further experiments were performed with a starting concentration of 5 μM to compare the potency between different compounds. According to the results shown in FIG. 7, CFI-102 was the most potent analog for both IKE an RSL3, TH-2-9-1 was the most potent analog for RSL3 alone, and TH-2-30 had potency comparable to Fer-1 against IKE and RSL3.

### Example 5

### Therapeutical Applications of Fer-1 Analogs

Patients receiving radiotherapy and/or immunotherapy usually suffer from various side effects including, but not limited to, skin reactions (e.g., redness, itching, peeling, blistering, and dryness) and flu-like symptoms (e.g., fatigue, fever, chills, weakness, nausea, vomiting, dizziness, body aches, and high or low blood pressure). There is evidence showing these side effects may be associated with undesired cell death through ferroptosis, which suggests therapeutic potential for molecules that inhibit/reduce ferroptosis.

To explore such applications, we will introduce the Fer-1 analogs disclosed herein into conventional radiotherapy/immunotherapy protocols. We will monitor patients' (animal and then human patient's) reaction to the combined treatment, and determine whether there is any improvement with respect to common side effects, for example, less or even no occurrence, reduced intensity, etc. We anticipate using in vitro models to inform our animal trials.

It is also believed that ferroptosis plays a critical role in bacteria-induced (e.g., Mycobacterium tuberculosis) cell death and tissue necrosis. In light of this, we expect that the Fer-1 analogs disclosed herein would have therapeutic application against various pathogens through inhibiting unwanted ferroptosis.

### Example 6

### Other Optimized Fer-1 Analogs as Ferroptosis Inhibitors

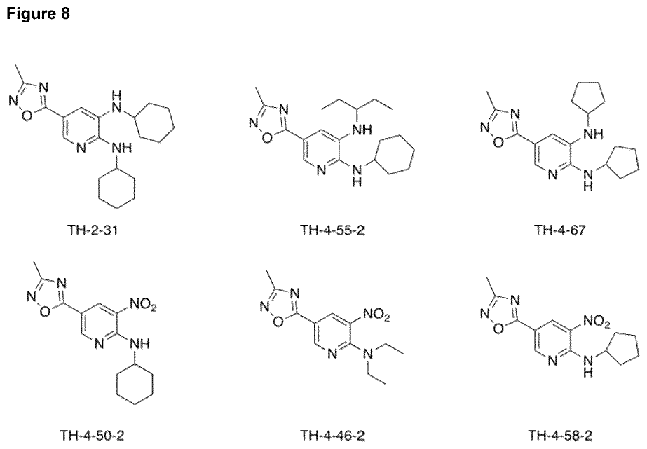

After synthesizing and characterizing a series of ferrostatin-1 analogs (see below for some selected analogs), three active compounds (TH-2-31 (i.e., CFI-102), TH-4-55-2, and TH-4-67) that meet all criteria for success were identified. Three inactive controls derived from the active compounds were also obtained for comparative studies (FIG. 8, compounds TH-4-50-2, TH-4-46-2, and TH-4-58-2). All active analogs can be synthesized on gram scale in high purity, and are suitable for in vivo efficacy studies. The cheminal characteristics, tests performed and detailed test results are shown below.

### Synthesis and Characteristics of Selected Analogs

### TH-2-31

### N²,N³-dicyclohexyl-5-(3-methyl-1,2,4-oxadiazol-5-yl)pyridine-2,3-diamine

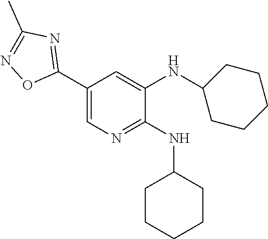 [SMILES: Cc1noc(-c2cnc(NC3CCCCC3)c(NC3CCCCC3)c2)n1]

Following Scheme 17 with N²-cyclohexyl-5-(3-methyl-1,2,4-oxadiazol-5-yl)pyridine-2,3-diamine (43 mg, 0.16 mmol), N²,N³-dicyclohexyl-5-(3-methyl-1,2,4-oxadiazol-5-yl)pyridine-2,3-diamine (38 mg, 67% yield) was obtained as light yellow solid.

¹H NMR (400 MHz, Chloroform-d) δ 7.81 (d, J=1.7 Hz, 1H), 6.86 (d, J=1.9 Hz, 1H), 4.04-3.78 (m, 1H), 3.19 (td, J=10.0, 4.2 Hz, 1H), 2.44 (s, 3H), 2.01 (d, J=10.6 Hz, 4H), 1.89-1.64 (m, 4H), 1.58 (d, J=13.0 Hz, 1H), 1.47-1.01 (m, 9H).

MS (m/z): [MH]⁺ calculated for C₂₀H₂₉N₅O [M+H]⁺: 356.2450, found: 356.2471.

### TH-4-16-1

### N²-cyclohexyl-N³-cyclopentyl-5-(3-methyl-1,2,4-oxadiazol-5-yl)pyridine-2,3-diamine

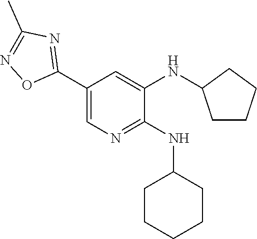 [SMILES: Cc1noc(-c2cnc(NC3CCCCC3)c(NC3CCCC3)c2)n1]

Following Scheme 17 with N²-cyclohexyl-5-(3-methyl-1,2,4-oxadiazol-5-yl)pyridine-2,3-diamine (200 mg, 0.73 mmol), N²-cyclohexyl-N³-cyclopentyl-5-(3-methyl-1,2,4-oxadiazol-5-yl)pyridine-2,3-diamine (35 mg, 14% yield) was obtained as brown solid.

¹H NMR (400 MHz, Chloroform-d) δ 7.78 (d, J=1.8 Hz, 1H), 6.93 (dd, J=1.8, 0.7 Hz, 1H), 3.86-3.71 (m, 1H), 3.64 (t, J=6.0 Hz, 1H), 2.37 (s, 3H), 2.02-1.90 (m, 5H), 1.74-1.60 (m, 4H), 1.60-1.44 (m, 5H), 1.36-1.22 (m, 5H), 1.10-1.00 (m, 1H).

MS (m/z): [MH]⁺ calculated for C₁₉H₂₇N₅O [M+H]⁺: 342.2294, found: 342.2301.

### TH-4-55-1

### N³-cyclobutyl-N²-cyclohexyl-5-(3-methyl-1,2,4-oxadiazol-5-yl)pyridine-2,3-diamine

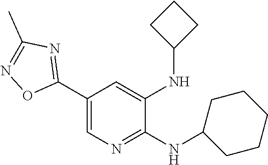 [SMILES: Cc1noc(-c2cnc(NC3CCCCC3)c(NC3CCC3)c2)n1]

Following Scheme 17 with N²-cyclohexyl-5-(3-methyl-1,2,4-oxadiazol-5-yl)pyridine-2,3-diamine (18 mg, 0.066 mmol), N³-cyclobutyl-N²-cyclohexyl-5-(3-methyl-1,2,4-oxadiazol-5-yl)pyridine-2,3-diamine (15 mg, 70% yield) was obtained as brown solid.

¹H NMR (400 MHz, Chloroform-d) δ 8.41 (d, J=2.0 Hz, 1H), 7.15 (d, J=2.0 Hz, 1H), 4.46 (s, 1H), 3.98 (d, J=5.8 Hz, 1H), 3.92-3.78 (m, 1H), 2.51-2.42 (m, 2H), 2.36 (s, 3H), 2.03 (dd, J=12.4, 3.9 Hz, 2H), 1.87-1.74 (m, 4H), 1.70 (dt, J=13.2, 3.6 Hz, 2H), 1.66-1.58 (m, 1H), 1.47-1.34 (m, 2H), 1.18 (td, J=11.7, 11.3, 3.3 Hz, 4H).

MS (m/z): [MH]⁺ calculated for C₁₈H₂₆N₅O, 328.2137; found 328.2148.

### TH-4-55-2

### N²-cyclohexyl-5-(3-methyl-1,2,4-oxadiazol-5-yl)-N³-(pentan-3-yl)pyridine-2,3-diamine

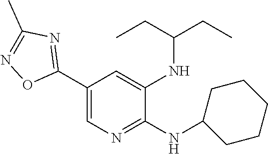 [SMILES: CCC(CC)Nc1cc(-c2nc(C)no2)cnc1NC1CCCCC1]

Following Scheme 17 with N²-cyclohexyl-5-(3-methyl-1,2,4-oxadiazol-5-yl)pyridine-2,3-diamine (23 mg, 0.084 mmol N²-cyclohexyl-5-(3-methyl-1,2,4-oxadiazol-5-yl)-N³-(pentan-3-yl)pyridine-2,3-diamine (16 mg, 56% yield) was obtained as yellow oil.

¹H NMR (400 MHz, Chloroform-d) δ 8.40 (d, J=2.0 Hz, 1H), 7.41-7.24 (m, 1H), 3.97 (tt, J=10.5, 3.9 Hz, 1H), 3.14 (tt, J=5.9 Hz, 1H), 2.36 (s, 3H), 2.07-1.98 (m, 2H), 1.74-1.64 (m, 2H), 1.64-1.33 (m, 7H), 1.24-1.10 (m, 4H), 0.89 (t, J=7.4 Hz, 6H).

MS (m/z): [MH]⁺ calculated for C₁₉H₃₀N₅O, 344.2450; found 344.2467.

### TH-4-46-2

### N,N-diethyl-5-(3-methyl-1,2,4-oxadiazol-5-yl)-3-nitropyridin-2-amine

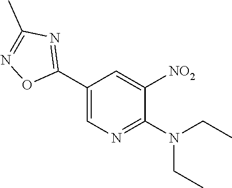 [SMILES: CCN(CC)c1ncc(-c2nc(C)no2)cc1[N+](=O)[O-]]

Following Scheme 17 with 6-(diethylamino)-5-nitronicotinic acid (456 mg, 1.9 mmol), N,N-diethyl-5-(3-methyl-1,2,4-oxadiazol-5-yl)-3-nitropyridin-2-amine (7 mg, 1% yield) was obtained as yellow solid.

¹H NMR (400 MHz, Chloroform-d) δ 8.86 (d, J=2.1 Hz, 1H), 8.59 (d, J=2.1 Hz, 1H), 3.47 (q, J=7.1 Hz, 4H), 2.38 (s, 3H), 1.19 (t, J=7.1 Hz, 6H).

MS (m/z): [MH]⁺ calculated for C₁₂H₁₆N₅O₃, 278.1253; found 278.1276.

### TH-4-50-2

### N-cyclohexyl-5-(3-methyl-1,2,4-oxadiazol-5-yl)-3-nitropyridin-2-amine

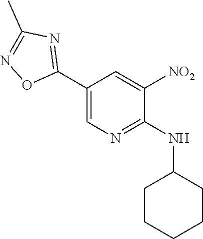 [SMILES: Cc1noc(-c2cnc(NC3CCCCC3)c([N+](=O)[O-])c2)n1]

Following Scheme 17 with 2-chloro-5-nitronicotinic acid (1 g, 4.92 mmol), N-cyclohexyl-5-(3-methyl-1,2,4-oxadiazol-5-yl)-3-nitropyridin-2-amine (25 mg, 2% yield) was obtained as yellow solid.

¹H NMR (400 MHz, Chloroform-d) δ 9.13-9.07 (m, 2H), 8.56 (d, J=7.7 Hz, 1H), 4.49-4.29 (m, 1H), 2.49 (s, 3H), 2.15-2.07 (m, 2H), 1.83 (dt, J=13.1, 4.0 Hz, 2H), 1.75-1.65 (m, 1H), 1.55-1.29 (m, 5H).

MS (m/z): [MH]⁺ calculated for C₁₄H₁₇N₅O₃, 304.1410; found 304.1407.

### TH-4-58-2

### N-cyclopentyl-5-(3-methyl-1,2,4-oxadiazol-5-yl)-3-nitropyridin-2-amine

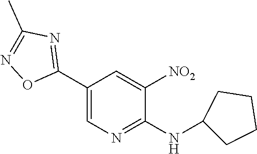 [SMILES: Cc1noc(-c2cnc(NC3CCCC3)c([N+](=O)[O-])c2)n1]

Following Scheme 17 with 6-(cyclopentylamino)-5-nitronicotinic acid (1.51 g, 6 mmol), N-cyclohexyl-5-(3-methyl-1,2,4-oxadiazol-5-yl)-3-nitropyridin-2-amine (220 mg, 13% yield) was obtained as yellow solid.

¹H NMR (400 MHz, Chloroform-d) δ 9.10 (d, J=2.2 Hz, 1H), 9.08 (d, J=2.2 Hz, 1H), 8.59 (d, J=6.8 Hz, 1H), 4.70 (q, J=6.8 Hz, 1H), 2.48 (s, 3H), 2.26-2.11 (m, 2H), 1.90-1.79 (m, 2H), 1.79-1.71 (m, 2H), 1.69-1.57 (m, 3H).

### TH-4-62

### N³-cyclobutyl-N²-cyclopentyl-5-(3-methyl-1,2,4-oxadiazol-5-yl)pyridine-2,3-diamine

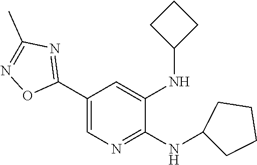 [SMILES: Cc1noc(-c2cnc(NC3CCCC3)c(NC3CCC3)c2)n1]

Following Scheme 17 with N²-cyclohexyl-5-(3-methyl-1,2,4-oxadiazol-5-yl)pyridine-2,3-diamine (15 mg, 0.058 mmol), N³-cyclobutyl-N²-cyclopentyl-5-(3-methyl-1,2,4-oxadiazol-5-yl)pyridine-2,3-diamine (8 mg, 44% yield) was obtained as yellow solid.

¹H NMR (400 MHz, Chloroform-d) δ 8.08 (s, 1H), 6.92 (s, 1H), 4.30 (dd, J=8.0, 4.9 Hz, 1H), 3.79 (t, J=7.5 Hz, 1H), 2.46-2.38 (m, 2H), 2.36 (s, 3H), 2.12-2.00 (m, 2H), 1.95-1.74 (m, 4H), 1.74-1.62 (m, 2H), 1.62-1.48 (m, 4H).

MS (m/z): [MH]⁺ calculated for C₁₇H₂₄N₅O, 314.1981; found 314.1995.

### TH-4-66

### N³-cyclohexyl-N²-cyclopentyl-5-(3-methyl-1,2,4-oxadiazol-5-yl)pyridine-2,3-diamine

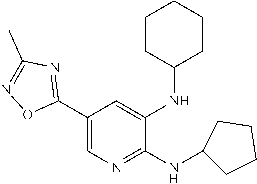 [SMILES: Cc1noc(-c2cnc(NC3CCCC3)c(NC3CCCCC3)c2)n1]

Following Scheme 17 with N²-cyclohexyl-5-(3-methyl-1,2,4-oxadiazol-5-yl)pyridine-2,3-diamine (17 mg, 0.065 mmol), N³-cyclohexyl-N²-cyclopentyl-5-(3-methyl-1,2,4-oxadiazol-5-yl)pyridine-2,3-diamine (13 mg, 58% yield) was obtained as yellow solid.

¹H NMR (400 MHz, Chloroform-d) δ 7.88 (d, J=1.7 Hz, 1H), 7.00 (d, J=1.7 Hz, 1H), 4.29-4.15 (m, 1H), 3.24 (ddt, J=10.1, 7.2, 3.7 Hz, 1H), 2.48 (s, 3H), 2.18-2.00 (m, 4H), 1.89-1.58 (m, 8H), 1.47-1.20 (m, 6H).

MS (m/z): [MH]⁺ calculated for C₁₉H₂₈N₅O, 342.2294; found 342.2304.

### TH-4-67

### N²,N³-dicyclopentyl-5-(3-methyl-1,2,4-oxadiazol-5-yl)pyridine-2,3-diamine

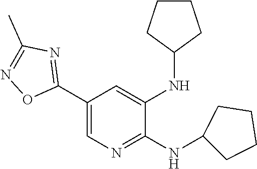 [SMILES: Cc1noc(-c2cnc(NC3CCCC3)c(NC3CCCC3)c2)n1]

Following Scheme 17 with N²-cyclohexyl-5-(3-methyl-1,2,4-oxadiazol-5-yl)pyridine-2,3-diamine (17 mg, 0.065 mmol), N²,N³-dicyclopentyl-5-(3-methyl-1,2,4-oxadiazol-5-yl)pyridine-2,3-diamine (12 mg, 56% yield) was obtained as yellow solid.

¹H NMR (400 MHz, Chloroform-d) δ 7.74 (d, J=1.9 Hz, 1H), 6.84 (d, J=1.7 Hz, 1H), 4.27-4.07 (m, 1H), 3.58 (q, J=6.0 Hz, 1H), 2.49-2.27 (m, 3H), 2.10-1.90 (m, 4H), 1.80-1.45 (m, 13H), 1.20 (d, J=7.1 Hz, 2H), 0.88-0.76 (m, 1H).

MS (m/z): [MH]⁺ calculated for C₁₈H₂₆N₅O, 328.2137; found 328.2147.

### TH-4-68

### N²-cyclopentyl-5-(3-methyl-1,2,4-oxadiazol-5-yl)-N³-(pentan-3-yl)pyridine-2,3-diamine

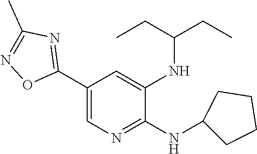 [SMILES: CCC(CC)Nc1cc(-c2nc(C)no2)cnc1NC1CCCC1]

Following Scheme 17 with N²-cyclohexyl-5-(3-methyl-1,2,4-oxadiazol-5-yl)pyridine-2,3-diamine (15 mg, 0.065 mmol), N²,N³-dicyclopentyl-5-(3-methyl-1,2,4-oxadiazol-5-yl)pyridine-2,3-diamine (2 mg, 11% yield) was obtained as yellow solid.

¹H NMR (400 MHz, Chloroform-d) δ 7.91 (d, J=1.7 Hz, 1H), 6.97 (s, 1H), 4.32 (t, J=6.0 Hz, 1H), 3.23 (t, J=6.0 Hz, 1H), 2.47 (s, 3H), 2.14-2.02 (m, 4H), 1.65 (m, 10H), 0.96 (t, J=7.4 Hz, 4H).

MS (m/z): [MH]⁺ calculated for C₁₈H₂₈N₅O, 330.2294; found 330.2304.

### Assays Performed

### Cell Viability Assays

Cell culture assays were incubated at 37° C. with 5% CO₂. HT-1080 cells were grown in DMEM (Corning) supplemented with 10% FBS (Life Technologies), 1% Penicilin-Streptomycin 10,000 U/mL (Gibco), and 1% MEM Non-Essential Amino Acids Solution 100× (Gibco). For cell viability assays, cells were trypsinized, counted, and seeded into 384-well white polypropylene plates at 1,000 cells/well, unless otherwise specified. After allowing cells to adhere overnight, compounds in DMSO stocks were arrayed in a 16-point dilution series prepared in a mother plate, and treated from a daughter plate, [DMSO]=0.28%. After 24 or 48 hr, 50% CellTiter-Glo (Promega) 50% cell culture medium was added to each well and incubated at room temperature with shaking for 15 min. Luminescence was measured using a Victor X5 plate reader (PerkinElmer). All cell viability data were normalized to the DMSO vehicle condition. From these data, dose-response curves and IC50 values were computed using Prism 7.0 (GraphPad). All 384w measurements were performed in triplicate

### Microsomal Stability Assay

To a 96-well polypropylene plate was added phosphate buffer (182.2 μL, pH 7.4, 100 mM) followed by addition of NADPH-regenerating system solution A (10 μL), and NADPH regenerating system solution B (2 μL) (Corning Gentest 3P NADPH regenerating system solution A (#451220) and B (#451200)). A stock solution of analog (0.8 μL. 5 mM) or fer-1 (positive control) was added and the mixture was warmed to 37° C. for 5 min. Mouse microsomes (CD-1, 20 mg/mL, Life Technologies) (5 μL, thawed in 37° C. water bath before use) were added. The resulting reaction mixture was kept at 37° C. with gentle agitation for the duration of the experiment. At selected time points (0, 1, 5, 10, 20, 30, 60 and 120 min) aliquots (15 μL) were withdrawn from the plate and quenched upon addition to cold methanol (60 μL), containing an internal standard (5 μM) in a separate 96-well polypropylene plate. At the completion of the final time-point, the samples were centrifuged at 4,000 rpm for 5 min at 4° C. The supernatant (40 μL) was withdrawn and transferred to a sample vial with insert. The samples were analyzed by LC-MS. LC-MS analysis was performed on a platform comprising a Thermo Scientific Dionex Ultimate 3000 and a Bruker amaZon SL equipped with an electrospray ionization source controlled by Bruker Hystar 3.2. Chromatographic separation was performed by injecting 5 μL of the sample onto an Agilent Eclipse Plus C18 column (2.1×50 mm, 3.5 μm) maintained at 20° C. The flow rate was maintained at 400 μL/min. The initial flow conditions were 80% solvent A (water containing 0.1% acetic acid) and 20% solvent B (methanol containing 0.1% acetic acid). Solvent B was raised to 80% over 0.50 min by 1.50 min. Solvent B was raised to 100% by 5.00 min and held there for 3.25 min. Solvent B was lowered back to initial conditions (20%) over 0.50 min by 8.75 min with a total run time of 12.00 min. All analogs were detected in positive mode as [M+H]*. The percent of compound remaining at each time-point was calculated as the ratio of the integrated compound peak over the internal standard peak and standardized to the t=0 time-point. Values were plotted in GraphPad Prism 9 and fit with a one phase decay.

### Plasma Stability Assay

Mouse plasma (GeneTex) was centrifuged at 3000 rpm for 10 min at 10° C. with the resulting supernatant withdrawn and diluted 1:1 in phosphate buffer pH 7.4. To a 96-well polypropylene plate was added 50% mouse plasma phosphate buffer (195 μL). A stock solution of analog in DMSO (0.8 μL. 5 mM) or fer-1 (positive control) was added to a separate well and the components were warmed to 37° C. for 5 min with gentle agitation. The reaction was initiated with the addition of analog to plasma, and the reaction kept at 37° C. for gentle agitation for the duration of the assay. At selected time points, aliquots (15 μL) were withdrawn from the plate and quenched upon addition to cold methanol (60 μL), containing an internal standard (5 μM) in a separate 96-well polypropylene plate. At the completion of the final time-point, the samples were centrifuged at 4,000 rpm for 5 min at 4° C. The supernatant (40 μL) was withdrawn and transferred to a sample vial with insert. The samples were analyzed by LC-MS. LC-MS analysis was performed on a platform comprising a Thermo Scientific Dionex Ultimate 3000 and a Bruker amaZon SL equipped with an electrospray ionization source controlled by Bruker Hystar 3.2. Chromatographic separation was performed by injecting 5 μL of the sample onto an Agilent Eclipse Plus C18 column (2.1×50 mm, 3.5 μm) maintained at 20° C. The flow rate was maintained at 400 μL/min. The initial flow conditions were 80% solvent A (water containing 0.1% acetic acid) and 20% solvent B (methanol containing 0.1% acetic acid). Solvent B was raised to 80% over 0.50 min by 1.50 min. Solvent B was raised to 100% by 5.00 min and held there for 3.25 min. Solvent B was lowered back to initial conditions (20%) over 0.50 min by 8.75 min with a total run time of 12.00 min. All analogs were detected in positive mode as [M+H]*. The percent compound remaining at each time-point was calculated as the ratio of the integrated compound peak over the internal standard peak and standardized to the t=0 time-point. Values were plotted in GraphPad Prism 9 and fit with a one phase decay.

### Animal Studies

All animal study protocols were approved by the Columbia University Institutional Animal Care and Use Committee (IACUC). C57BL/6 mice (The Jackson Laboratory, stock number 000664) (male and female, 8-weeks of age)) were acclimated after shipping for >3 days before beginning experiments. Mice were maintained on a 12 h light/dark cycle and fed a standard diet (PicoLab 5053)

### TH-2-31 IP PK Study

C57BL/6 mice (8-weeks of age and ˜25 g weight) were weighed before injection and divided into groups of 2 male and female mice per cage. TH-2-31 was dissolved in 5% DMSO/95% of 65% v/v of 25% w/v 2-hydroxypropyl-3-cyclodextrin (Cayman Chemical) dissolved in 20% EtOH, 30% v/v Poly(ethylene glycol)-400 (Sigma Aldrich 202398), 5% v/v Tween 80 (Fluka 59924) to create a 4 mg/mL solution. The same formulation without TH-2-31 was used as a vehicle control. The solution was sterilized using a 0.22 mm Steriflip filter unit (Thomas Scientific 1189Q46). Mice were dosed IP and euthanized by CO₂ asphyxiation for 3 min at 0, 1, 2, 4, and 8 h after administration. To ensure that the vehicle was well-tolerated 4 mice were treated with vehicle and euthanized 4 h after administration. ˜0.5 mL of blood was collected via cardiac puncture and immediately put in K3 EDTA microtubes (SARSTEDT 41.1504.105) and kept on ice. Organs were harvested, placed in Eppendorf tubes, and frozen on dry ice. Blood samples were centrifuged for 10 min at 2,100×g at 4° C., then plasma was transferred to a clean tube and frozen on dry ice. Organ samples were weighed and placed in hard tissue homogenizing tubes (Omni International 19-628) and a volume of DEPC-treated nuclease-free water (IBI Scientific IB42200) was added to make a 500 mg/mL solution and homogenized using the Omni Bead Ruptor 4 at speed 5 for 30 seconds. TH-2-31 was extracted from plasma or organ homogenate by adding 900 μL acetonitrile to 100 μL plasma or organ homogenate. Samples were mixed by vortexing and allowed to extract overnight at 4° C. prior to mixing for at least 5 min by rotating at room temperature, vortexing, and sonicating for at least 30 second prior to centrifugation for 10 min at 4,000×g and 4° C. The supernatant was then transferred to a glass vial and dried under nitrogen. After drying, the samples were resuspended in 100 μL of methanol and analyzed by UPLC-MS described below. The concentration of TH-2-31 was determined against a standard curve with a linear fit and the data plot in GraphPad Prism 9 and fit with a one phase decay.

### TH-2-31, TH-4-55-2, TH-4-67 PK Study

C57BL/6 mice (8-weeks of age and ˜25 g weight) were weighed before injection and divided into groups of two male and female mice per cage. Compound was dissolved in 5% DMSO/95% of 1:1 [(65% v/v of 25% w/v 2-hydroxypropyl-3-cyclodextrin (Cayman Chemical) dissolved in 20% EtOH, 30% v/v Poly(ethylene glycol)-400 (Sigma Aldrich 202398), 5% v/v Tween 80 (Fluka 59924)):MilliQ water] to create a 2 mg/mL solution. The same formulation without compound was used as a vehicle control. The solution was sterilized using a 0.22 mm Steriflip filter unit (Thomas Scientific 1189Q46). Mice were dosed IP, IV, and PO routes of administration and euthanized by CO₂ asphyxiation for 3 min at 0, 1, 2, 4, 8, and 24 h after administration. To ensure that the vehicle was well-tolerated 4 mice were treated with vehicle and euthanized 4 h after administration. ˜0.5 mL of blood was collected via cardiac puncture and immediately put in K3 EDTA microtubes (SARSTEDT 41.1504.105) and kept on ice. Organs were harvested, placed in Eppendorf tubes, and frozen on dry ice. Blood samples were centrifuged for 10 min at 2,100×g at 4° C., then plasma was transferred to a clean tube and frozen on dry ice. Organ samples were weighed and placed in hard tissue homogenizing tubes (Omni International 19-628) and a volume of DEPC-treated nuclease-free water (IBI Scientific IB42200) was added to make a 500 mg/mL solution and homogenized using the Omni Bead Ruptor 4 at speed 5 for 30 seconds. Compound was extracted from plasma or organ homogenate by adding 900 μL acetonitrile to 100 μL plasma or organ homogenate. Samples were mixed by vortexing and allowed to extract overnight at 4° C. prior to mixing for at least 5 min by rotating at room temperature, vortexing, and sonicating for at least 30 second prior to centrifugation for 10 min at 4,000×g and 4° C. The supernatant was then transferred to a glass vial and analyzed by UPLC-MS described below. The concentration of each analog was determined against a standard curve with a nonlinear fit and the data plot in GraphPad Prism 9 and fit with a one phase decay.

### UPLC-MS Analysis

Samples from animal studies were analyzed via UPLC-MS using a Waters Xevo G2-Xs QTof Mass spectrometer equipped with an Acquity UPLC. Chromatographic separation was carried out at 50° C. on a Acquity UPLC BEH C18 column (1.7 μm, 2.1 mm×50 mm, pore size 130 Å) over a 4.5 min gradient elution. The flowrate was held constant at 0.8 mL/min. Mobile phase A consisted of water and mobile phase B consisted of ACN both containing 0.1% formic acid. After injection, the gradient was held at 50% A for 0.25 min. For the next minute, the gradient was ramped in a linear fashion to 100% B and held at this composition for 0.5 min. The eluent composition returned to the initial condition in 0.01 min and the column was re-equilibrated for 2.74 min before the next injection. Injection volumes were 0.5 μL (TH-2-31 40 mg/kg) and 1 μL for all other conditions. The Xevo G2-Xs was operated in positive electrospray ionization (ESI) mode. A capillary voltage and sampling cone voltage of 0.5 kV and 30 V were used. The source and desolvation temperatures were kept at 120° C. and 20° C., respectively. Nitrogen was used as the desolvation gas with a flowrate of 750 L/hr. The protonated molecular ion of leucine encephalin ([M+H]⁺, m/z 556.2771 was used as a lock mass for mass accuracy and reproducibility. Leucine enkephalin was introduced to the lock mass at a concentration of 2 ng/mL (50% ACN containing 0.1% formic acid), and a flow rate of 5 mL/min. To avoid signal saturation, the signal transmission was attenuated to <10%, based off the signal intensity of the highest standard concentration for the duration of the run. The data was collected over the mass range m/z 50 to 1200 Da with an acquisition time of 0.1 seconds per scan. The retention time for each analog is detailed below. All samples were injected twice and the base peak chromatogram was integrated and quantified by standard curve concurrently ran using MassLynx software.

### Results of In Vitro Studies

Potency in Suppressing Ferroptosis Induced by RSL3 in N27 Cells (20 nM, 48 Hours of Incubation) with IC₅₀<10 nM

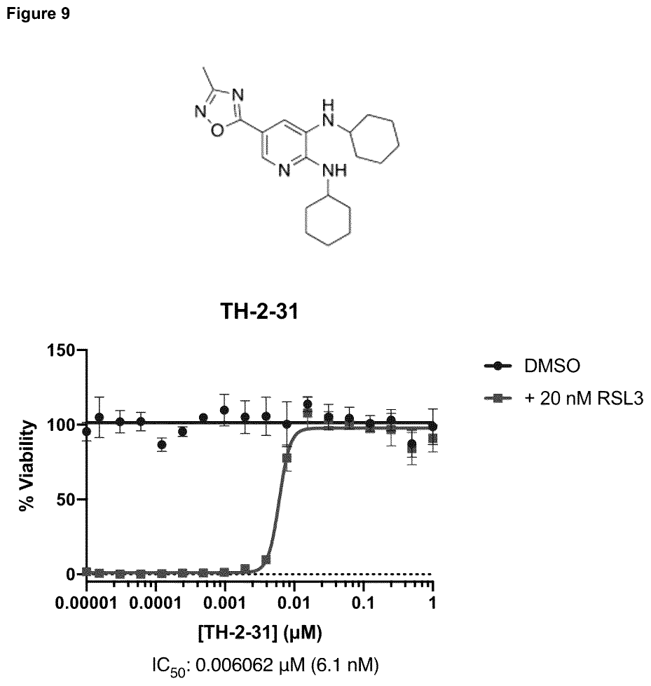

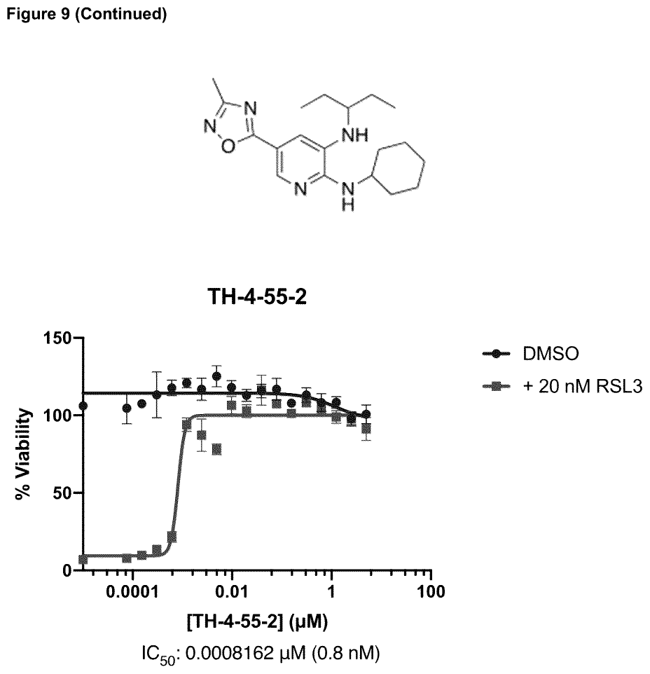

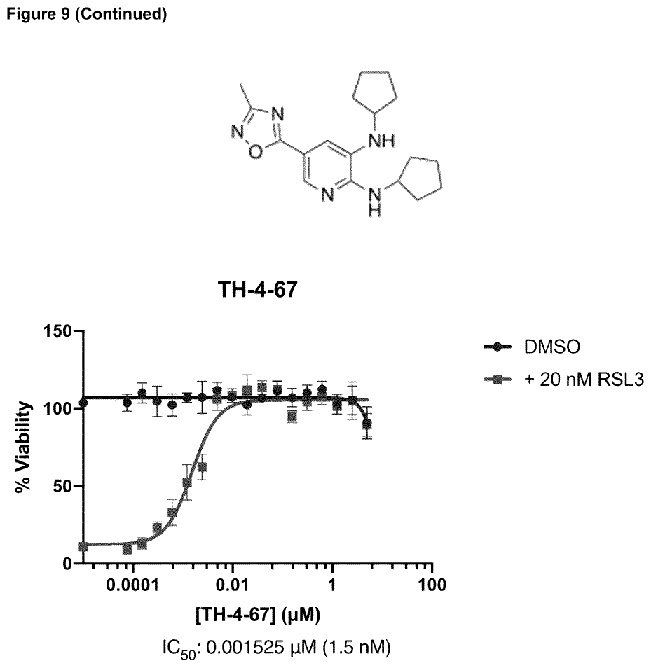

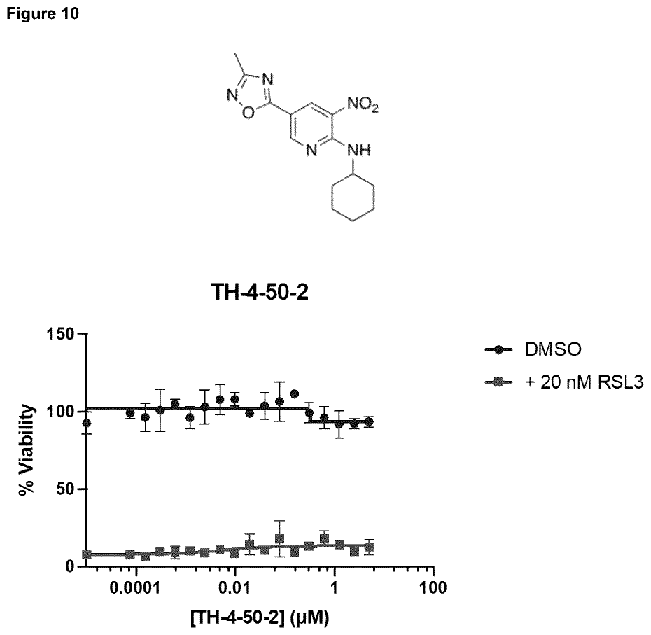

As shown in FIG. 9, there are representative dose-response curves demonstrating that three optimized ferrostatins (TH-2-31, TH-4-55-2, and TH-4-67) suppress ferroptosis induced by RSL3 in N27 cells (20 nM, 24 hours of treatment) with IC₅₀<10 nM. It was found that 20 nM RSL3 can effectively achieve 100% cell death within 24 hours, and potent ferrostatin-1 analogs are able to demonstrate complete ferroptosis rescue in a dose-dependent manner within the same time frame. Additionally, a 24-hour treatment allows for more efficient testing of analogs in a higher throughput fashion than 48-hour treatment.

IC₅₀ values for three separate experiment, n=3 wells/per compound/per condition, are provided in Table 2 below. The average IC₅₀ of TH-2-31, TH-4-55-2, and TH-4-67 as well as other compounds are shown in Table 3 below.

TABLE 2
IC₅₀s of TH-2-31, TH-4-55-2, and TH-4-67 in N27 cells.

| Compound | IC₅₀ I | IC₅₀ II | IC₅₀ III |
|---|---|---|---|
| TH-2-31 | 2.6 nM | 1.4 nM | 6.1 nM |
| TH-4-55-2 | 0.8 nM | 1.0 nM | 4.9 nM |
| TH-4-67 | 1.5 nM | 1.4 nM | 3.4 nM |

TABLE 3
Potency of representative ferrostation analogs
in N27 rat dopaminergic cells.

| Compound ID | IC₅₀ (nM) |
|---|---|
| Fer-1 | 3.2^{d} 12.5^{e} |
| CFI-4082 | 2.6^{d} |
| TH-2-31 | 3.4^{d} |
| TH-4-16-1 | 5.1^{d} 8.2^{e} |
| TH-4-55-1 | 2.2^{d} |
| TH-4-55-2 | 2.2^{d} |
| TH-4-62 | 6.9^{d} |
| TH-4-66 | 3.1^{d} |
| TH-4-67 | 2.1^{d} |
| TH-4-68 | 4.0^{d} |
| TH-4-100-2 | 18.9^{d} |

^{d}24 hr treatment, 1,000 cells/well seeded
^{e}48 hr treatment, 1,000 cells/well seeded

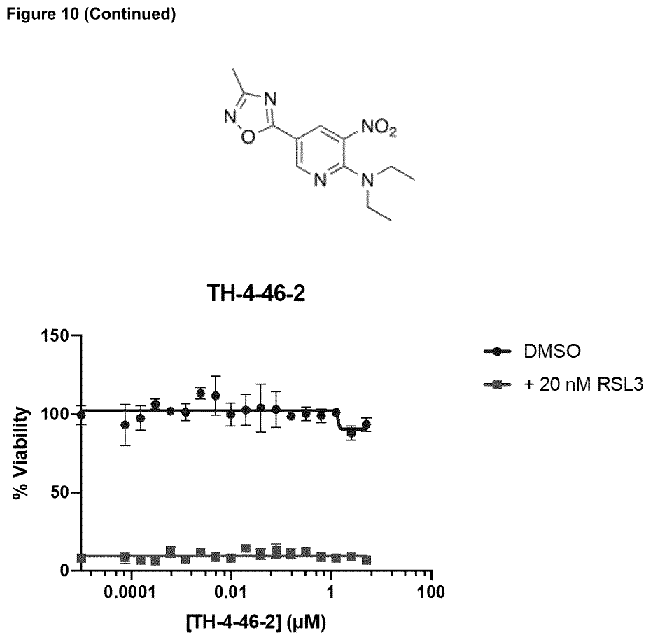

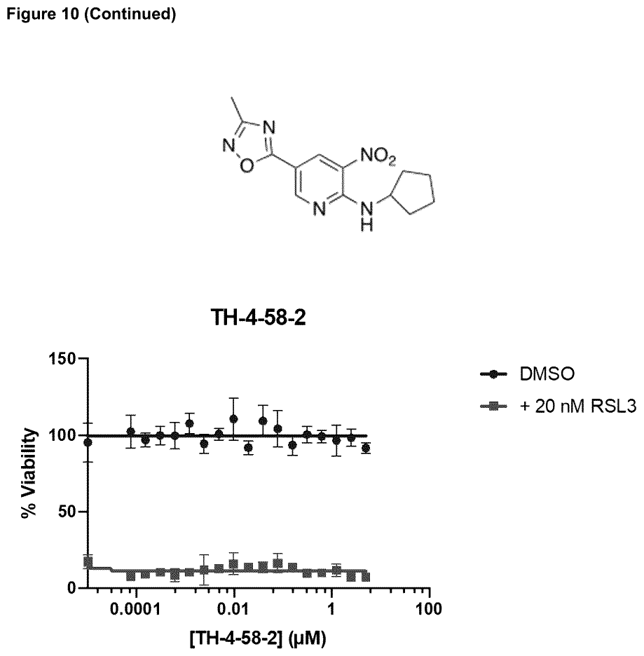

In addition to the optimized ferrostatin compounds described above, three inactive controls (TH-4-50-2, TH-4-58-2, and TH-4-46-2) were developed that are unable to suppress ferroptosis induced by RSL3 in N27 cells (20 nM, 24 hours of incubation). Their structures and representative dose-response curves are shown in FIG. 10.

Metabolic Stability in Mouse Liver Microsomes with Half-Life >60 Min

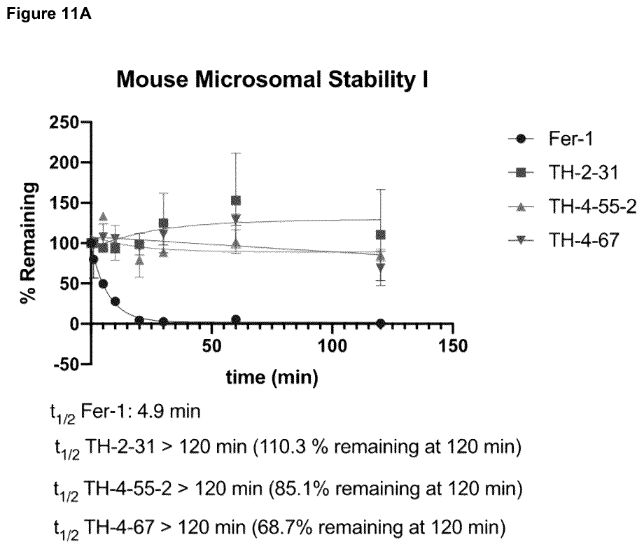

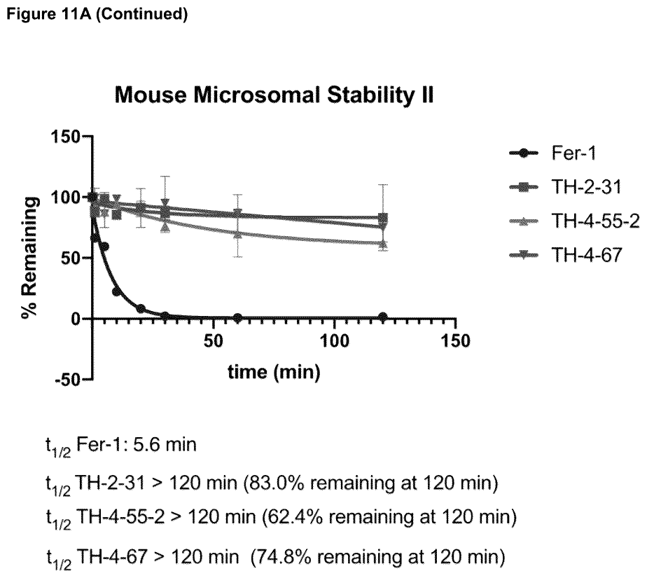

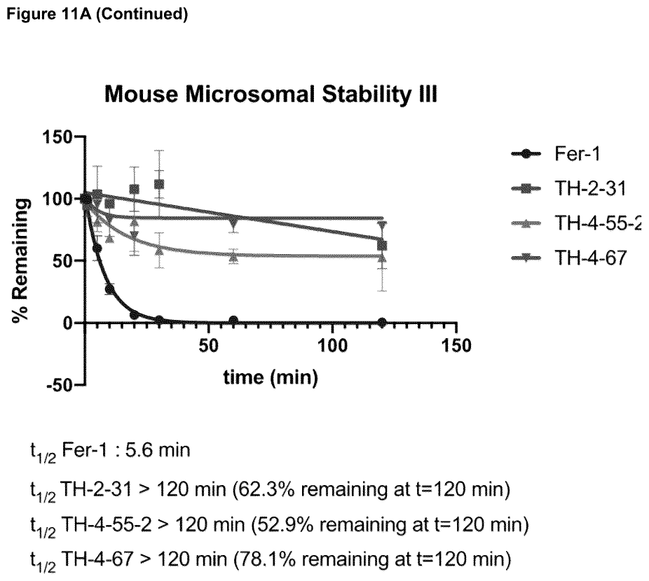

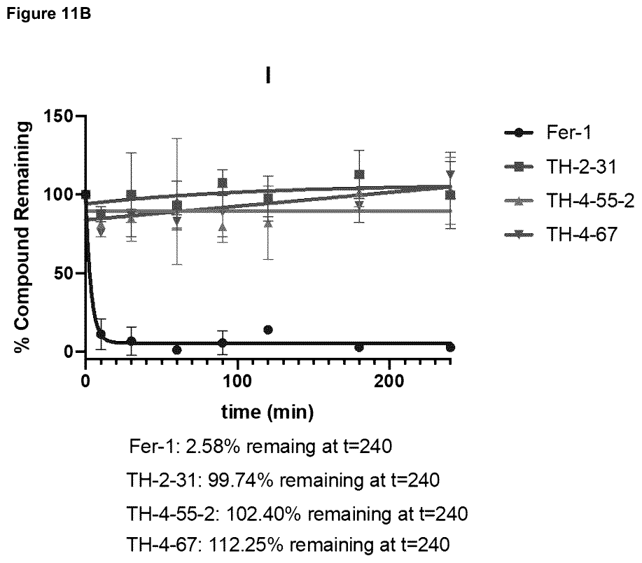

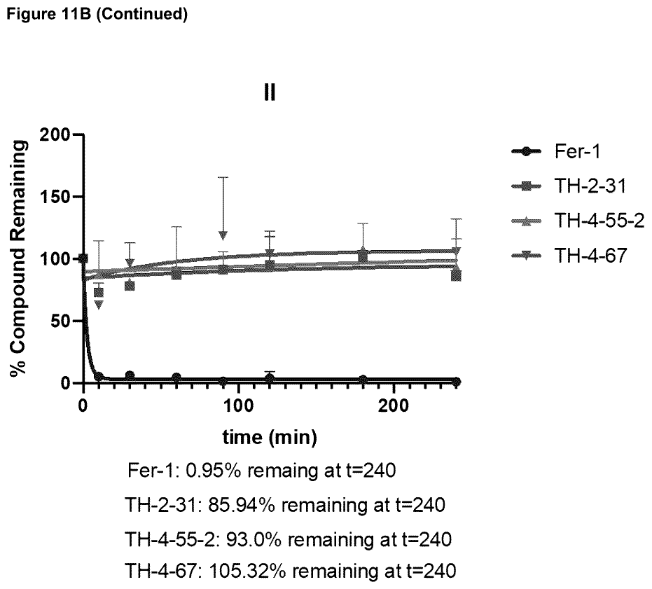

Results from three separate mouse microsomal stability experiments each performed in triplicate demonstrated that the three optimized ferrostatins (TH-2-31, TH-4-55-2, and TH-4-67) were stable in mouse liver microsomes with half-life greater than 60 minutes, with each compound indeed having a half-life greater than two hours (FIG. 11A), and were not metabolized in mouse plasma (FIG. 11B). These findings indicated that these analogs would be suitable to examine in vivo.

A summary of the half-lives from the three independent microsomal stability tests in mouse liver microsomes is provided below in Table 4.

TABLE 4
Results from three independent tests of ferrostatins
TH-2-31, TH-4-55-2 and TH-4-67 for microsomal
stability (n = 2 wells/compound/experiment).

| Compound | t_{1/2} I | t_{1/2} II | t_{1/2} III |
|---|---|---|---|
| TH-2-31 | >120 min | >120 min | >120 min |
| TH-4-55-2 | >120 min | >120 min | >120 min |
| TH-4-67 | >120 min | >120 min | >120 min |

Plasma Stability (Mouse) with Half-Life >120 Min

In two separate experiments, all three compounds were stable in mouse plasma with minimal-to-no degradation of the compounds after a 4 hour incubation (FIG. 11B). Ferrostatin-1 is shown as comparison, which is fully degraded in mouse plasma in less than 15 min. Table 5 summarizes the observed plasma half-lives for all 3 analogs.

TABLE 5
Observed plasma half-lives for all 3 optimized analogs.

| Compound | t_{1/2} I | t_{1/2} II |
|---|---|---|
| TH-2-31 | >240 min | >240 min |
| TH-4-55-2 | >240 min | >240 min |
| TH-4-67 | >240 min | >240 min |

All optimized compounds were synthesized on a gram scale with high purity, ready for in vivo efficacy studies. >1 gram of each compound was synthesized.

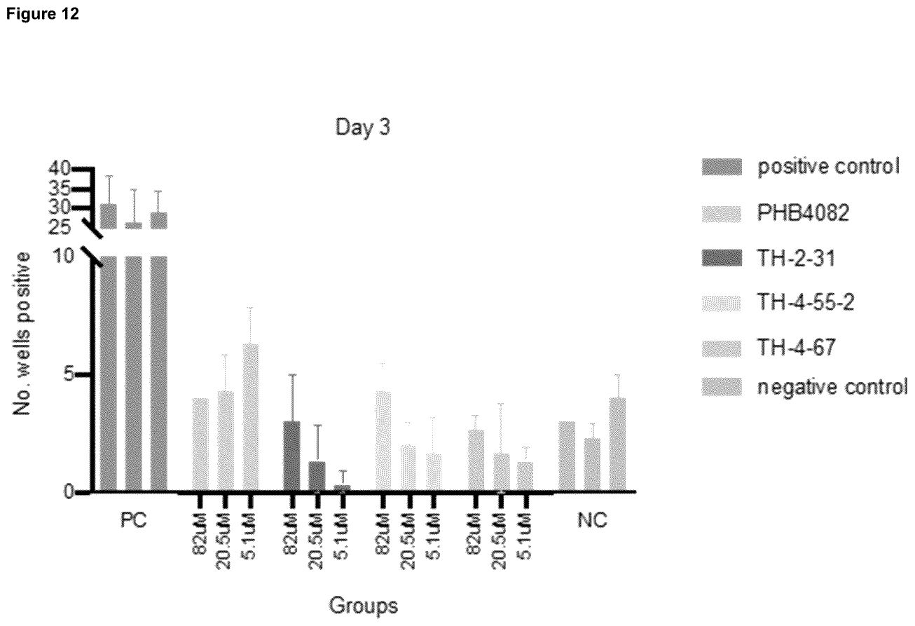

In addition to the Derek Nexus toxicity prediction, we sought to confirm that the new analogs did not have mutagenic potential in the AMES test (Zeiger, 2019). The AMES test uses modified bacteria sensitive to mutagenic agents to assess a compound's ability to cause direct DNA mutations. If the tested drug can induce reverse mutational events, it will cause bacteria to revert back to a prototrophic state and grow on media lacking selected nutrients. We tested TH-2-31, TH-4-55-2, and TH-4-67 using the AMES test. CFI-4082 was also included in the test for comparison. Bacteria strains were incubated under exposure to different concentrations of tested compounds for 3 days and collected 144 data points of mutation status at each concentration. The concentration ranged from 5.1 μM to 82 μM, which is the highest local organ concentration of our compounds in the above mice study (FIG. 12). The result showed that none of the compounds had mutagenic potential, and that the optimized compounds had a lower positive ratio compared to the prior ones.

### Results of In Vivo Studies

For the in vivo studies, the results from which are detailed below. The optimized ferrostatins (TH-2-31, TH-4-55-2, and TH-4-67) were administered to C57BL/6 mice at 8 weeks of age. Compounds were administered at a concentration of 20 mg/kg in a vehicle consisting of 1:1 (65% v/v of 25% w/v 2-hydroxypropyl-β-cyclodextrin dissolved in 20% ethanol, 30% v/v PEG-400, and 5% Tween-80): milliQ H₂O via intravenous injection (IV), intraperitoneal injection (IP), or oral gavage (PO). For each time-point and route of administration, two male and two female mice were used to minimize sex-specific effects. Mice were euthanized, and plasma and brain samples were obtained from each mouse at 0, 1, 2, 4, 8, and 24 hours after compound administration. All three analogs were well-tolerated in the mice with no immediate toxicity issues observed following administration. However, IV administration caused the mice to pass out and they were slow to recover thereafter, usually requiring an average of 15 minutes to become active and mobile again. Once recovered, no other issues were observed with the mice prior to CO₂ euthanasia. Compounds were extracted from plasma and brain homogenate in acetonitrile and analyzed via UHPLC-MS/MS against a standard curve to quantify compound concentrations.

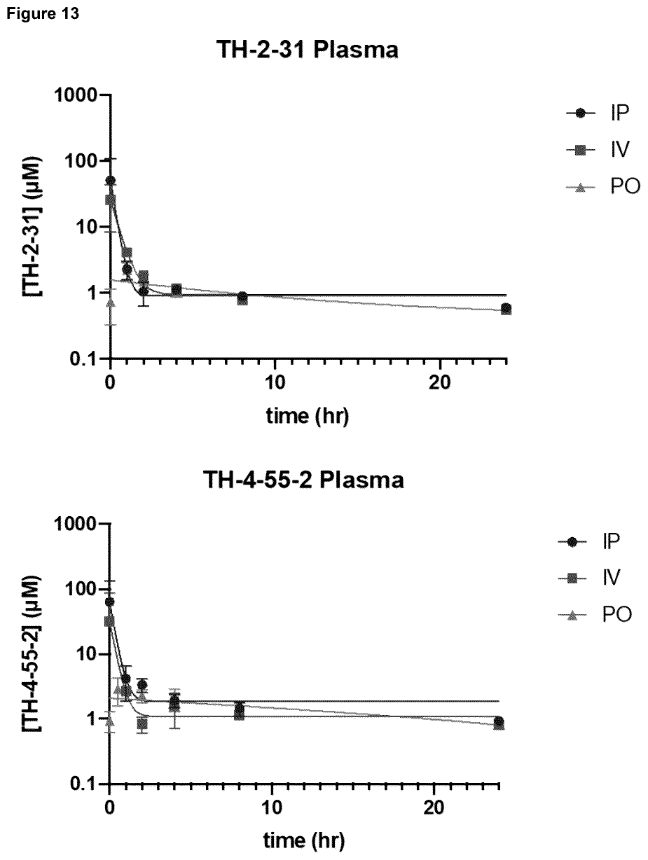

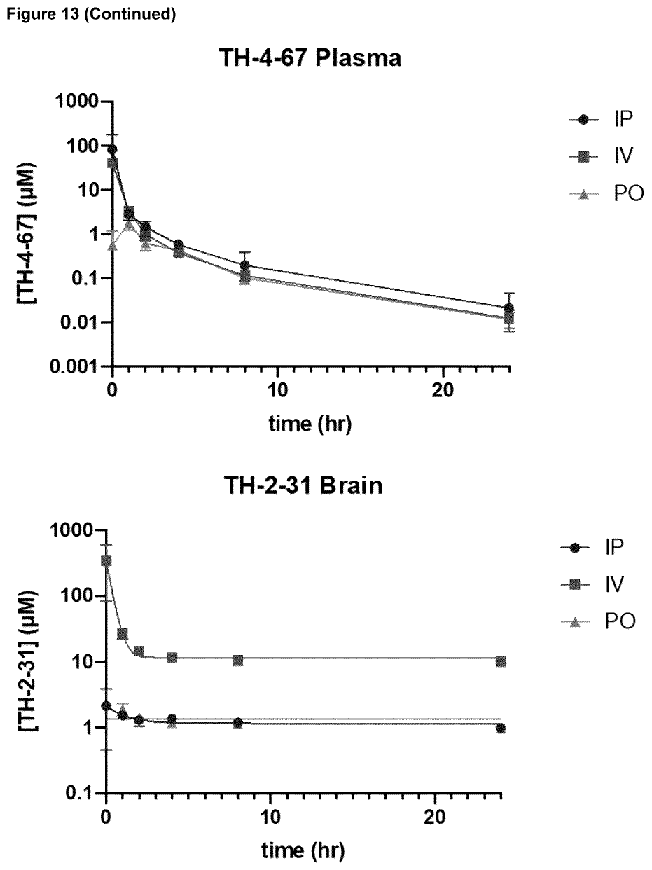

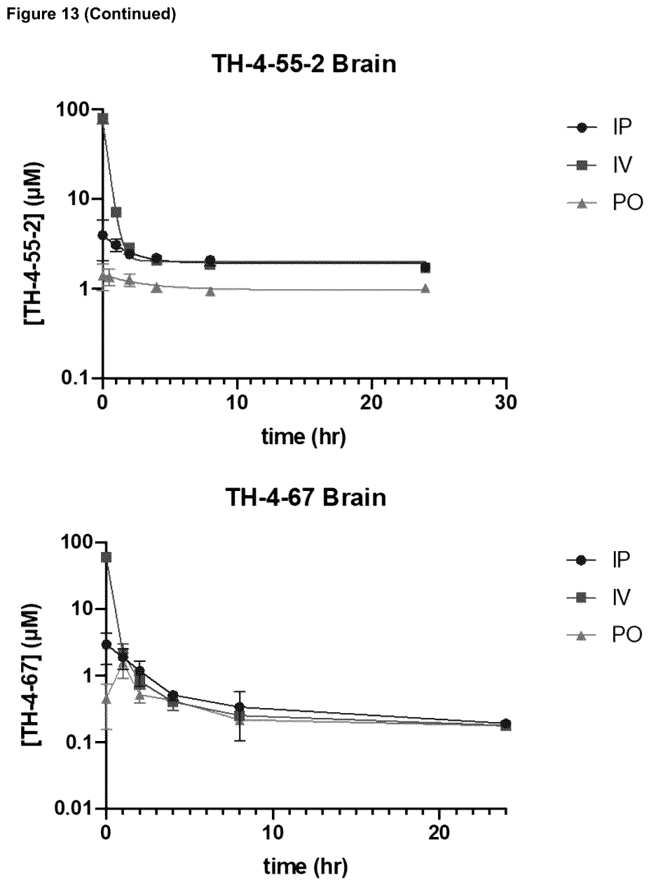

The concentration of each analog in plasma and brain is shown below in FIG. 13. Each analog accumulated at high nM to μM concentrations across the time-points analyzed. The metrics for various tests are described below.

### Stability in Plasma

All three analogs were found to be stable in plasma, independent of the route of administration, for up to 24 hours (FIG. 13). Each analog was found to be orally bioavailable. All three analogs follow expected PK trends; Immediately following IP and IV administration, the concentration of each analog in plasma peaks and exponentially decreases thereafter, while with PO administration the concentration peaks after a delay and slowly decreases thereafter. Comparing the concentration of each analog in plasma at 24 hours, TH-4-55-2 is the most and TH-4-67 is the least stable for all routes of administration. TH-2-31 is present in plasma at μM concentrations for up to 4 hours post administration with a concentration >500 nM in plasma 24 hours after administration for all routes of administration. TH-4-55-2 is present in plasma at μM concentrations for up to 8 hours post administration for all routes of administration with a concentration >800 nM 24 hours after administration for all routes of administration. TH-4-67 has the highest initial concentrations in plasma for both IP and IV administration; however, it is only present in plasma at μM concentrations for up to 1-hour post administration for PO and IV administration, and up to 2 hours post IP administration. At 24 hours post administration, TH-4-67 is present in plasma at a concentration <25 nM, an order of magnitude lower than both TH-2-31 and TH-4-55-2 at the same time-point, for all routes of administration.

### In Vivo Brain Half-Life >3 h

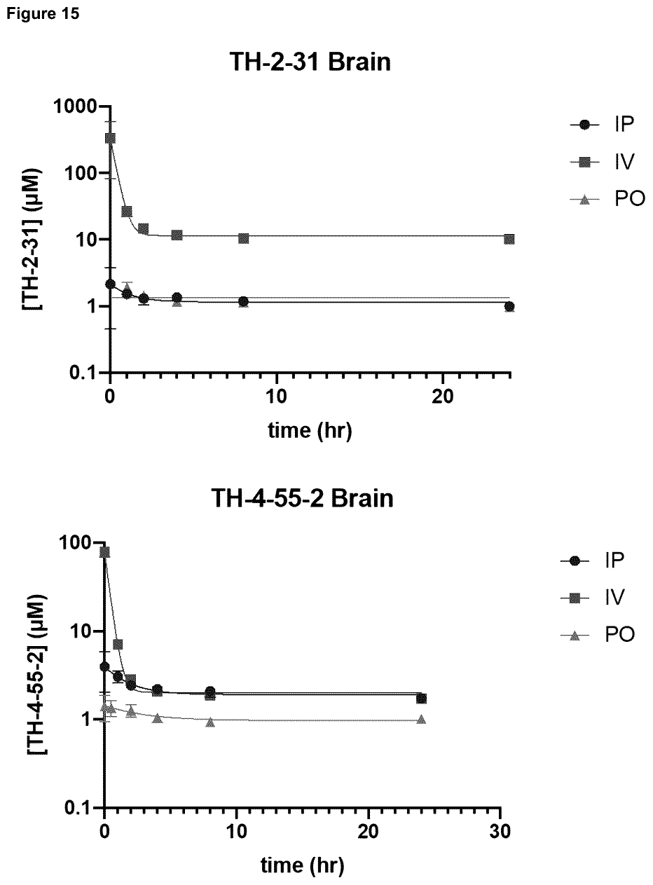

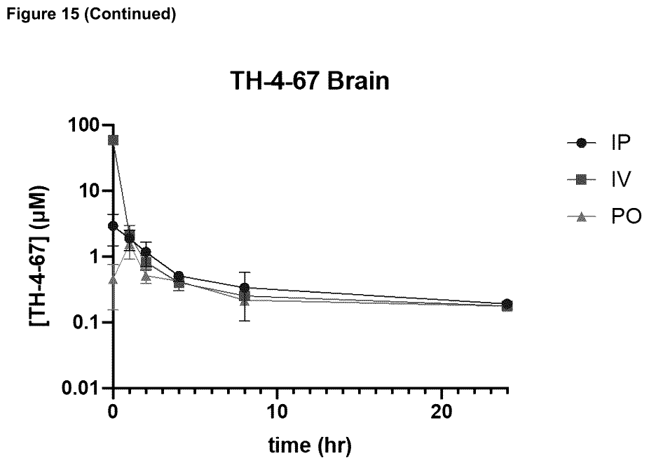

All three analogs were found to be brain penetrant for all routes of administration (FIG. 13). Each analog rapidly accumulated in brain following IV accumulation, at concentrations greater than 300 μM, 75 μM, and 50 μM for TH-2-31, TH-4-55-2, and TH-4-67, respectively, and decreased in concentration thereafter (FIG. 15). We reasoned that this rapid brain accumulation was the cause of the mice passing out described previously and tested this by designing a less brain penetrant analog (TH-4-100-2) that incorporates an adamantyl group at the R₁ position compared to a cyclohexyl group in TH-2-31. Mice dosed with TH-4-100-2 were less impaired immediately following injection and recovered quicker than mice dosed with an equivalent dose of TH-2-31 (data not shown), suggesting that decreasing the brain penetrance of ferrostatins can decrease potential adverse effects observed following IV injection.

A summary with the in vivo brain half-lives is provided in Table 6.

TABLE 6
in vivo brain half-live of each compound.

| Compound | R.O.A | t_{1/2} |
|---|---|---|
| TH-2-31 | IP | >8 h |
| | IV | <1 h |
| | PO | >24 h |
| TH-4-55-2 | IP | >8 h |
| | IV | <1 h |
| | PO | >24 h |
| TH-4-67 | IP | 1 h < t_{1/2} < 2 h |
| | IV | <1 h |
| | PO | 1 h < t_{1/2} < 2 h |

For both TH-2-31 and TH-4-55-2, the IP and PO administrations satisfy the R33 transition criterion. Among the three analogs, TH-2-31 is the most brain penetrant when administered IV, accumulating in brain at a concentration of 10 μM even 24 hours after administration, while TH-4-55-2 is the most stable following IP and PO administration with concentrations >1 μM 24 hours post administration. For TH-4-67, the criterion is not met. It is the least stable of the three analogs in brain with concentrations <200 nM for all routes of administration 24 hours post administration, However, as observed in the data provided below and the corresponding graphs, TH-4-67 accumulated in brain at orders of magnitude higher than the IC₅₀ values at 24 hours post compound administration, and it is expected to be potent irrespective of the half-live in the brain. Indeed, this compounds exceeded its 2 nM EC₅₀ value for the full 24 hours of treatment. Therefore, although the IP and PO half-lives are slightly under 3 hours, the compound is likely to exert a PD and therapeutic effect in mice, due to exceeding its effective concentration in the brain over a 24 hour period.

### Cmax>5× the IC₅₀ in N27 Cells

Table 7 below details the Cₘₐₓ in both plasma and brain and the IC₅₀ values for each analog and route of administration. All three of the optimized ferrostatins easily meet this criterion. In both plasma and brain, all analogs had Cₘₐₓ values in the μM range for all routes of administration. As expected, oral administration resulted in the lowest Cₘₐₓ values for all routes of administration in the single digit μM range, while for IP and IV administration Cₘₐₓ values were in the double-to-triple digit μM range. For each analog, comparing the analog concentration in plasma and brain with the IC₅₀ value revealed that each analog accumulated at concentrations at least 5× greater than the IC₅₀ values for all routes of administration in plasma and at concentrations greater than 50× in brain, even at 24 hours post administration.

TABLE 7
Summary of in vivo Cₘₐₓ values in brain and plasma, and Cₘₐₓ/IC₅₀ for
each route of administration.

| | | Cₘₐₓ | Cₘₐₓ | | Cₘₐₓ | Cₘₐₓ |
|---|---|---|---|---|---|---|
| | | plasma | brain | | plasma/ | brain/ |
| Compound | R.O.A. | (nM) | (nM) | IC₅₀ | IC₅₀ | IC₅₀ |
| TH-2-31 | IP | 50,923 | 2,133 | 3.37 | 15,111 | 633 |
| | IV | 25,791 | 334,759 | nM | 7,653 | 99,335 |
| | PO | 2,238 | 1,190 | | 664 | 353 |
| TH-4-55-2 | IP | 63,834 | 3,936 | 2.23 | 28,625 | 1,765 |
| | IV | 32,109 | 79,314 | nM | 14,399 | 35,567 |
| | PO | 2,929 | 1,410 | | 1,313 | 632 |
| TH-4-67 | IP | 82,120 | 2,916 | 2.10 | 39,105 | 1,389 |
| | IV | 40,640 | 52,121 | nM | 19,352 | 24,820 |
| | PO | 1,768 | 1,554 | | 842 | 740 |

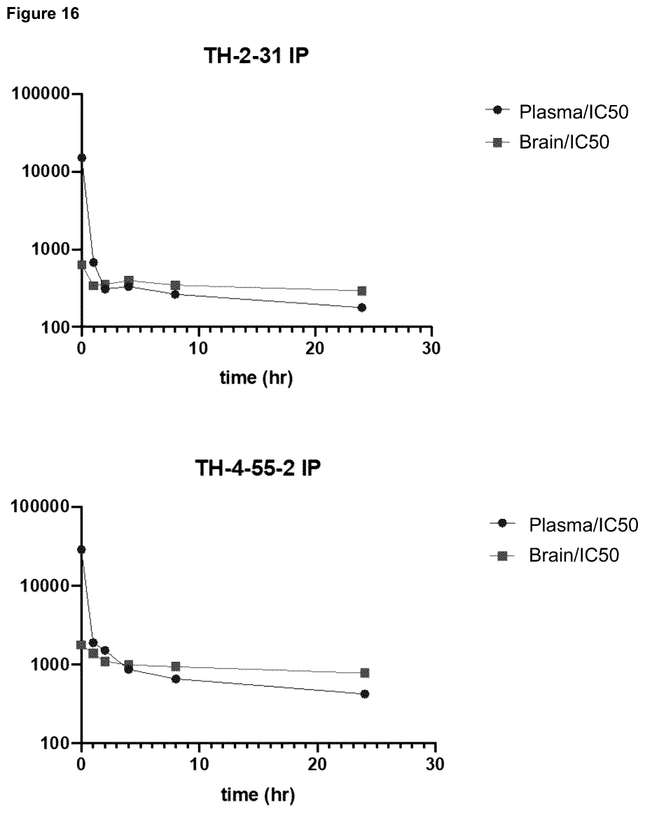

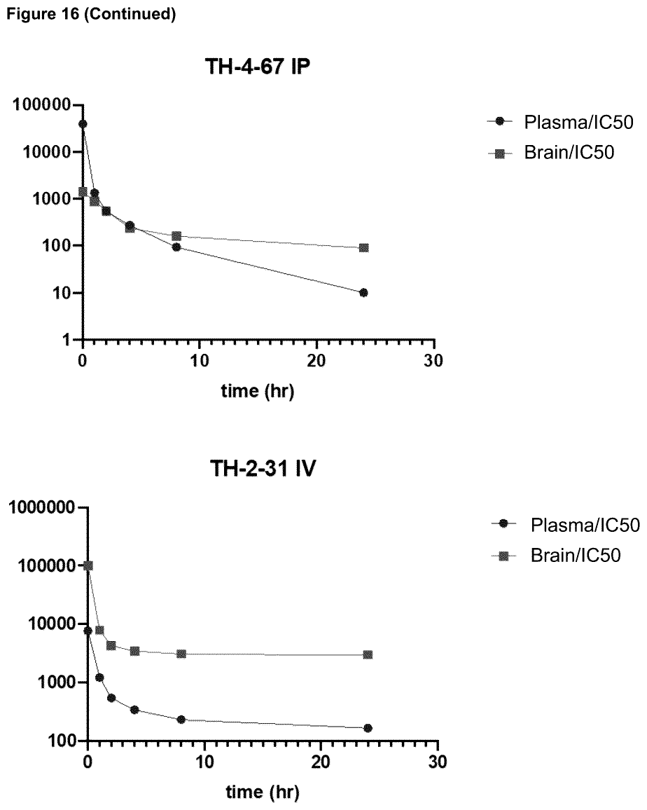

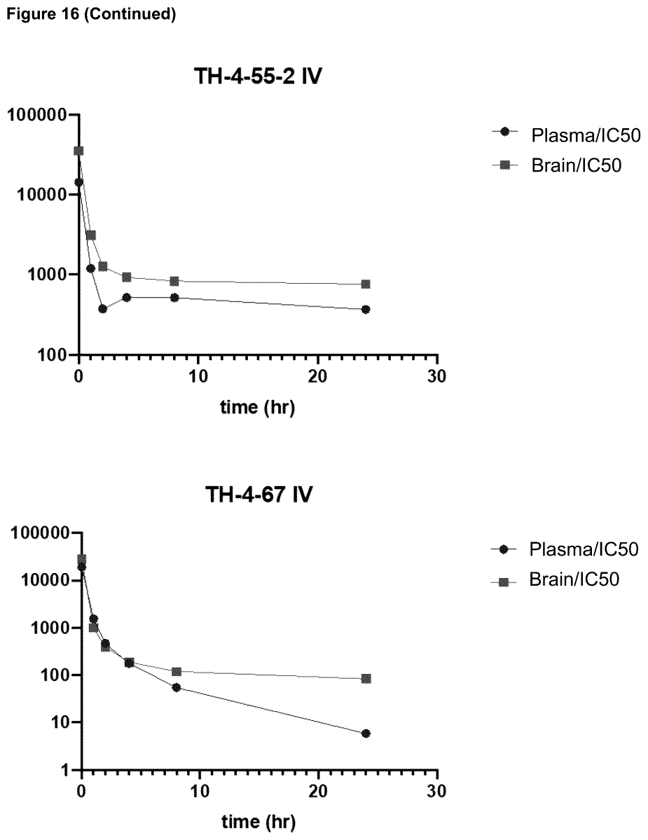

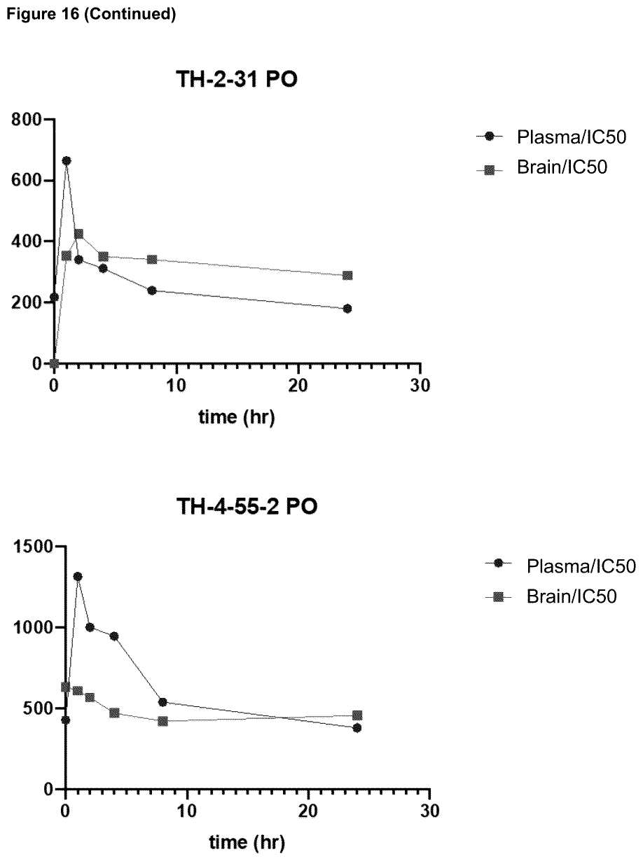

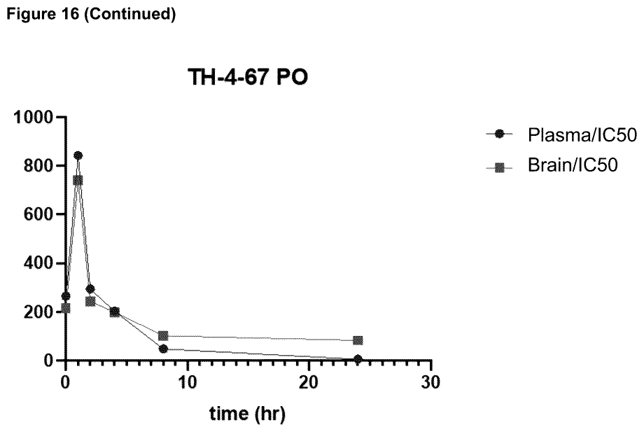

As shown in FIG. 16, the concentration of each optimized analog in brain and plasma was examined and compared to the IC₅₀ value for each time point and route of administration (R.O.A.). As indicated, all 3 compounds accumulate in both plasma and brain at ratios >5 for all time-points and route of administration.

BBB Permeability with Log (Brain/Plasma) Ratio >0

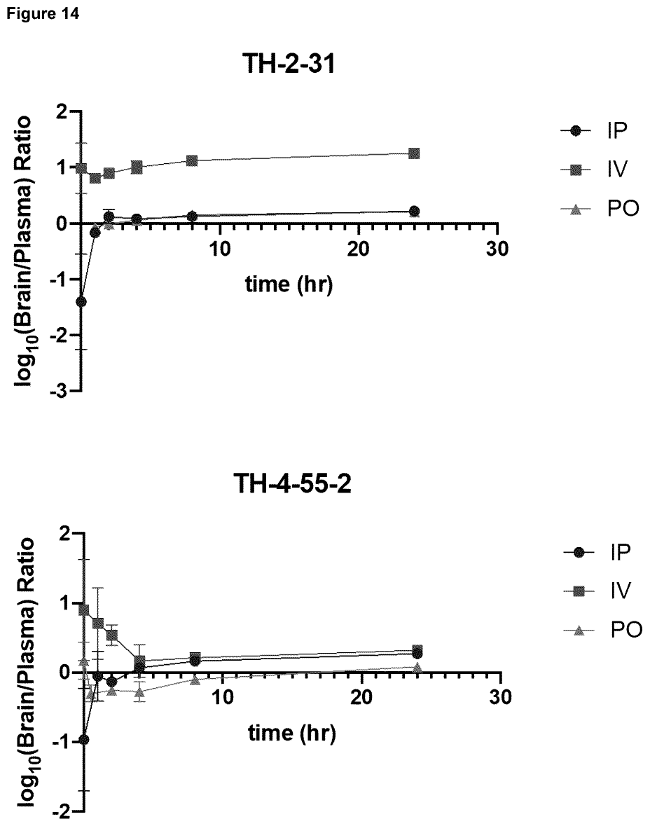

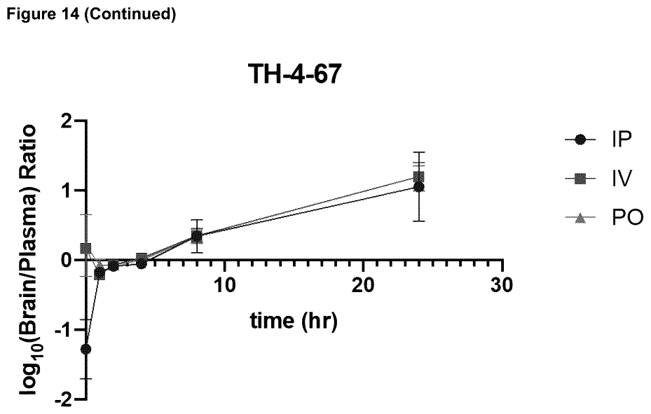

While all three analogs were found to accumulate in brain, to be effective for neurodegenerative disease applications they should preferentially accumulate in brain over plasma to ensure optimal analog distribution. Calculated from the above PK data, BBB permeability was determined using the log ratio of the concentration of analog in brain over plasma, log₁₀(Brain/Plasma) for each time-point and route of administration and plotted for each analog (FIG. 14). As shown in FIG. 14, each optimized analog preferentially accumulated in the brain over time, with all three compounds having a log₁₀(brain/plasma) value >0 at 24 hours for all routes of administration.

TH-2-31 and TH-4-55-2 preferentially accumulate in brain over plasma for all time-points following IV administration. For IP and PO administration for all compounds, the analogs initially accumulate in plasma and over time begin to accumulate in the brain. For all three routes of administration at 24 hours, TH-4-67 has the highest log₁₀(Brain/Plasma) values beyond TH-2-31 IV. This is likely due to the fact that both TH-2-31 and TH-4-55-2 stably accumulate at similar concentrations in both plasma and brain, while TH-4-67 is metabolized in plasma, and to a lesser extent in brain.

### Solubility >1 mM

To achieve the 20 mg/kg dose for each compound, mice were injected with a 2 mg/mL solution in the vehicle described above. For all three optimized compounds, no precipitation was observed in the resulting 2 mg/mL solutions, even several days after preparation. As detailed in Table 8 below, each of the compounds meet this criterion, with solubility greater than concentrations needed for in vivo injections.

TABLE 8
Concentration of each optimized compounds
prepared for in vivo injection.

| | Molecular | Concentration | Concentration |
|---|---|---|---|
| Compound | Weight | (mg/mL) | (mM) |
| TH-2-31 | 355.49 | 2 | 5.6 |
| TH-4-55-2 | 343.48 | 2 | 5.8 |
| TH-4-67 | 327.43 | 2 | 6.1 |

### In Vivo Testing in Disease Models

To determine whether the optimized analogs were suitable to probe whether ferroptosis is involved in the etiology of neurodegenerative diseases, we utilized two mouse models of Huntington's disease: the 3-nitropropionic acid model of striatal degeneration and the N-terminal transgenic R6/2 Huntington's mouse model. (Mangiarini et al., 1996; Tunez et al., 2010).

Male C57BL/6 mice at ˜8 weeks of age were dosed with vehicle or optimized analog at 20 mg/kg IP daily for three days prior to and in addition to daily IP dosing with 3-nitropropionic acid (3-NP) in an escalating dose series over 5 days, with the mice receiving a total of 360 mg/kg of 3-NP (Table 9). The body weight of each mouse was recorded daily and the % weight change from baseline for each treatment group was plotted as a measure of overall health. Any mouse that lost more than 20% of their body weight or had a poor body condition were euthanized prior to the completion of the study.

TABLE 9
Result of 3-nitropropionic acid model of striatal degeneration.

| | | | | | | | | | | | Post Inject |
|---|---|---|---|---|---|---|---|---|---|---|---|
| Day | −5 | −4 | −3 | −2 | −1 | Inject Day 1 | Inject Day 2 | Inject Day 3 | Inject Day 4 | Inject Day 5 | Day 1 |
| 3-NP Administration | | | | | | ▾ | ▾ | [symbol] | [symbol] | [symbol] | |
| Compound administration | | | □ | □ | □ | □ | □ | □ | □ | □ | |
| Behavior | X₁ | | | X₂ | | | | | X₃ | | |
| Euthanasia | | | | | | | | | | | ▪ |

▾ 60 mg/kg IP [symbol]
80 mg/kg IP
□ 20 mg/kg ferrostatin analog or vehicle IP
X₁ Preselection
X₂ Effect of ferrostatin treatment alone
X₃ Characterize 3-NP induced deficits/ Determine if ferrostatin treatment ameliorates 3-NP-induced behavioral deficits
▪ Euthanasia

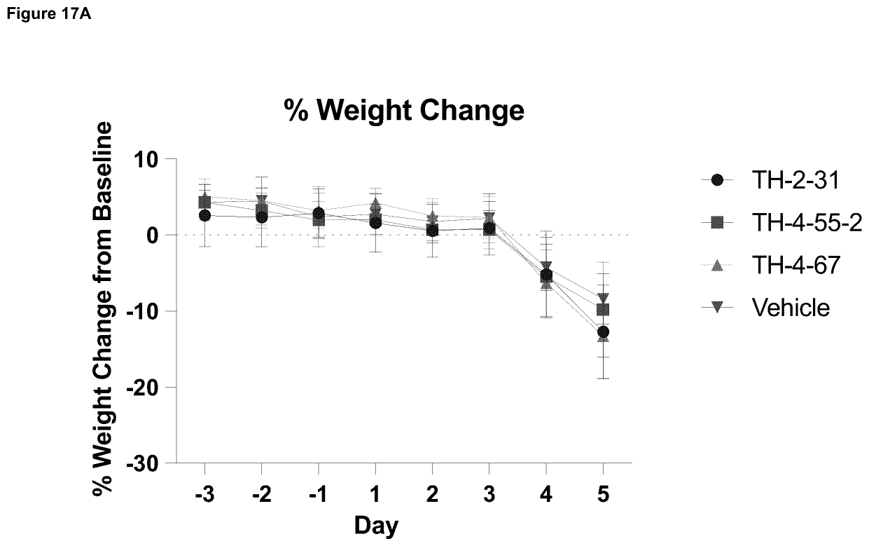

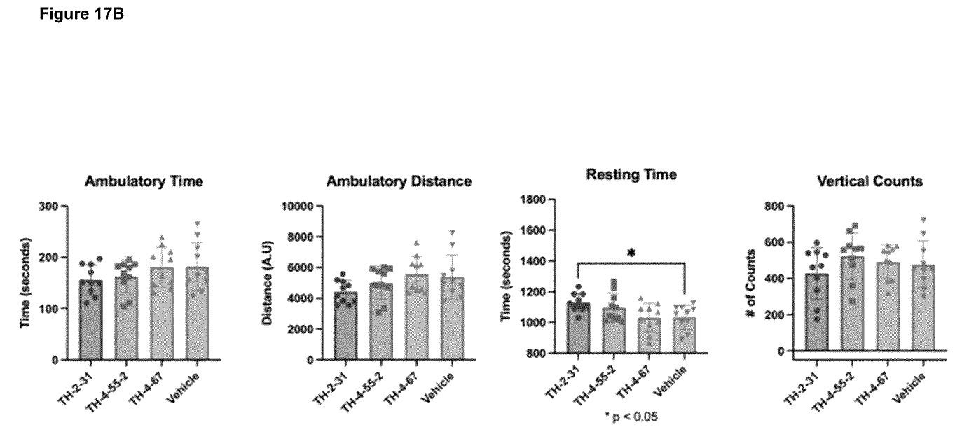

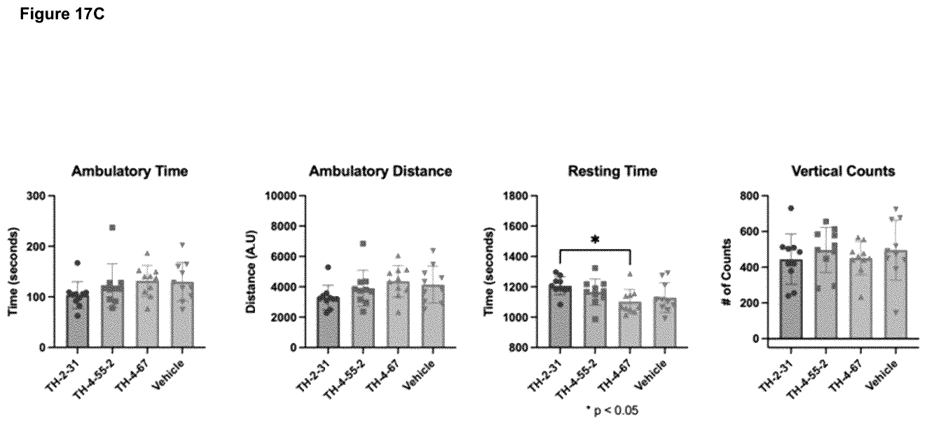

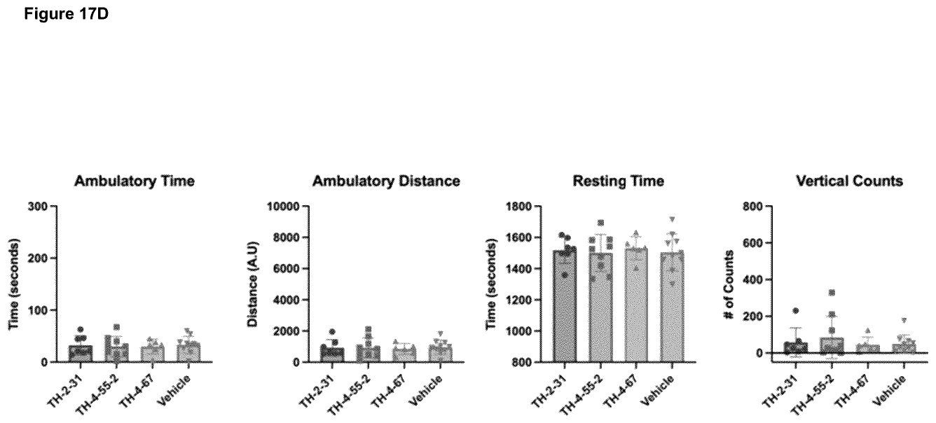

Beginning on Day 3 all mice, independent of treatment group, steadily lost weight and mixed-effects analysis indicated a significant effect of time but not treatment on the change in body weight (FIG. 17A). This indicates that optimized ferrostatins are not able to protect against the loss in body weight observed in the 3-NP model of striatal degeneration. In addition to weighing the mice daily, Open Field behavior in a 30-minute time period was recorded and analyzed at three different points in the study (Table 8): on Day −5 to establish baseline behavior prior to both ferrostatin and 3-NP treatment (FIG. 17B), on Day −2 to assess whether ferrostatin analog treatment had any effect on behavior (FIG. 17C), and on Day 4 to determine whether ferrostatin analog treatment can protect against Open Field deficits induced by 3-NP treatment (FIG. 17D). Open Field performance was assessed across 10 metrics, including time, distance, and vertical counts. There was no difference in ambulatory time, distance, or vertical counts between vehicle and ferrostatin analogs on Days −5 and −2, indicating that ferrostatin treatment alone has no behavioral effects. All mice displayed profound Open Field deficits on Day 4 across all Open Field metrics with no significant difference between the four treatment groups (FIG. 17D). Taken together, these findings suggest that ferrostatin treatment is ineffective in preventing weight loss and behavioral deficits in the 3-NP model, which may allow us to evaluate the specific contribution of ferroptosis to HD etiology and pathology.

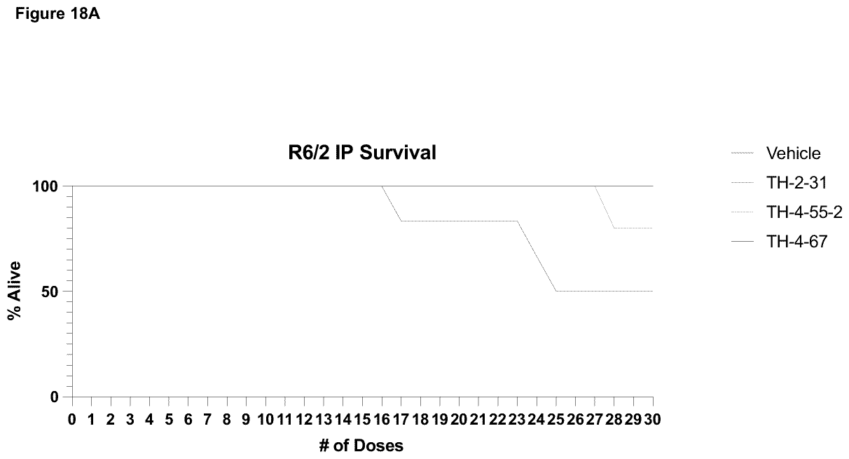

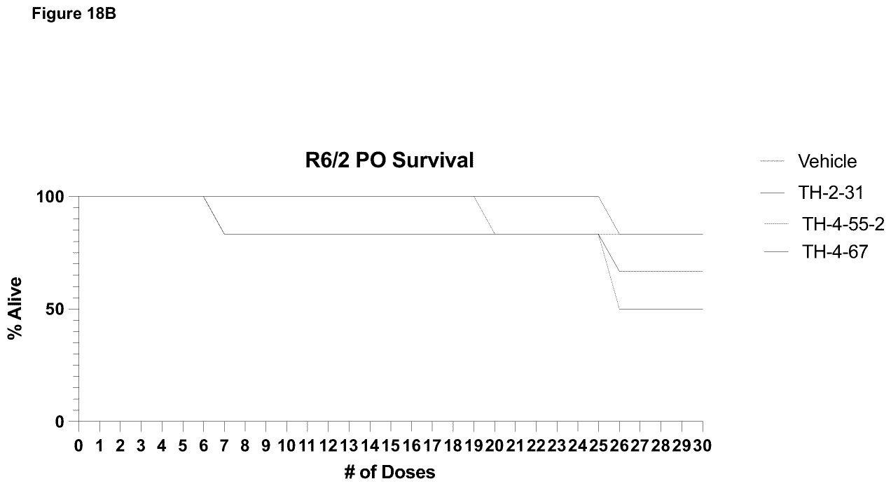

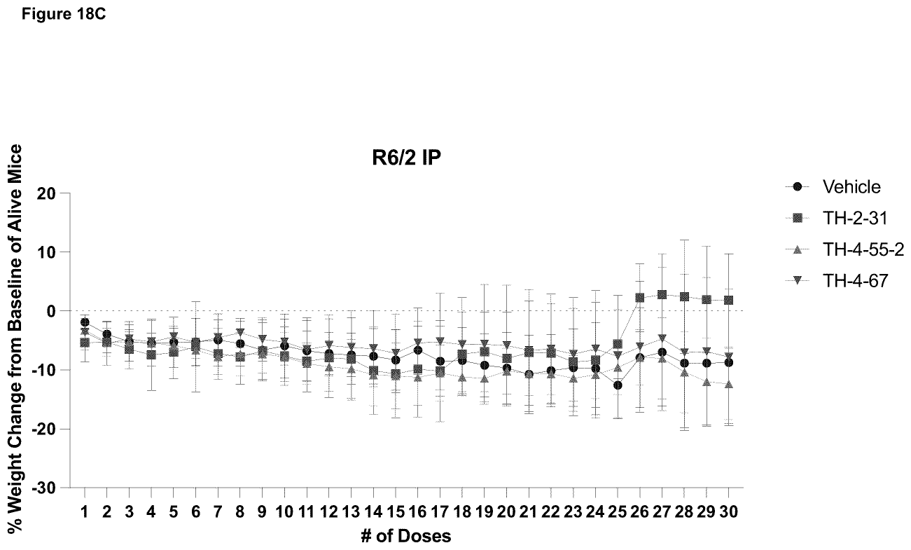

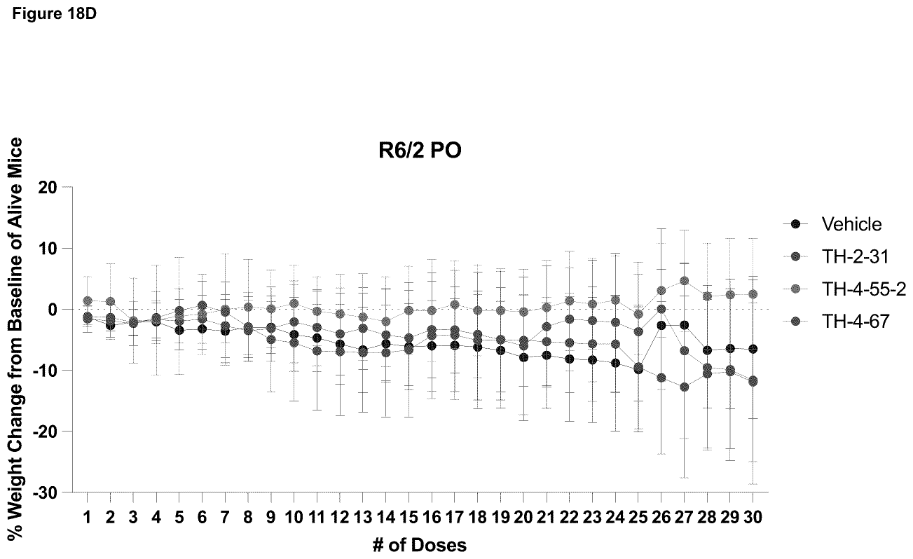

In order to assess whether ferrostatins can be used in a long-term efficacy, we performed a toxicity study with the three analogs to determine whether symptomatic R6/2 mice could tolerate chronic administration of analog. Use TH-4-55-2 as an example: symptomatic R6/2 mice of both sexes at ˜10 weeks of age were dosed daily with 20 mg/kg TH-4-55-2 via both IP and oral gavage for 30 days. Body weight was measured and recorded and the % change in body weight from the baseline calculated. Any mice that lost more than 20% of their body weight for three days were euthanized prior to the completion of the study. After 30 days, with IP administration 0 vehicle and one TH-4-55-2-treated mouse died (FIG. 18A) and with PO administration two vehicle- and one TH-4-55-2-treated mice died (FIG. 18B). Compared to IP administration (FIG. 18C), R6/2 mice treated with TH-4-55-2 via PO administration appeared to have no loss in body weight during the injection series (FIG. 18D). Despite this, mixed-effects analysis revealed no significant effect of time, treatment, or time and treatment with PO treated mice, while there was only a significant effect of time with IP treated mice. As such, this indicates that the optimized ferrostatin analog TH-4-55-2 is non-toxic and well-tolerated by R6/2 mice, allowing for it to be used in future studies requiring chronic administration regimens.

The results from the in vivo PK study indicate that all three analogs are brain penetrant analogs that preferentially accumulate in brain at concentrations greater than 50× the IC₅₀ value for each analog. Additionally, the ferrostatin analogs were demonstrated to be specific for ferroptotic-cell death and TH-4-55-2 was well-tolerated in a 30-day toxicity study in symptomatic R6/2 HD mice. Taken together, these studies indicate that these optimized ferrostatins could have efficacy in HD in vivo, and can be utilized to probe the contribution of ferroptosis to the development of neurodegenerative disease.

### Example 7

### More Fer-1 Analogs

By further modifying the type and postion of functional groups, we synthesized and tested more Fer-1 analogs. Their preparation and characteristics are provided below.

### General Procedure I (3):

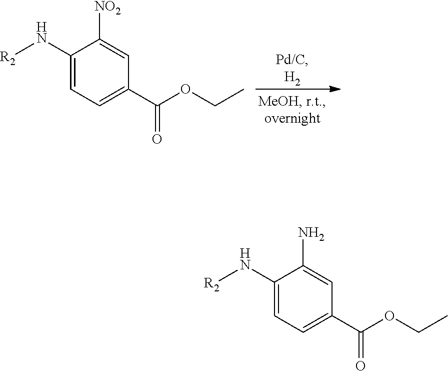

Substituted nitrobenzoate (1.0 eq.) and Pd (10 wt % on charcon, 0.2 eq.) were dissolved in methanol. The reaction was air exchanged to hydrogen gas and stirred under hydrogen gas (1 atm) overnight. The reaction mixture was filtered through celite and concentrated. The product was purified by column chromatography on silica gel (0-50% ethyl acetate in hexanes) to give the product.

### General Procedure I (4

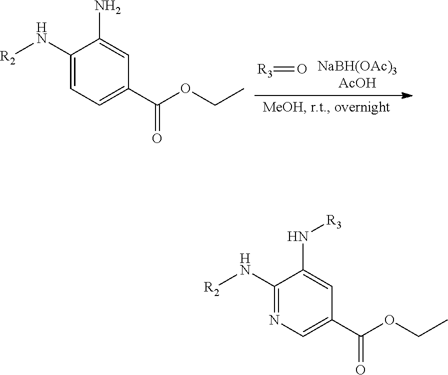

Substituted benzoate (1.0 eq), and ketone (1.0 eq) were dissolved in dichloroethane (0.1M) followed by addition of acetic acid (1.2 eq) and NaBH(OAc)₃ (1.2 eq). The reaction mixture was stirred at room temperature overnight. A saturated aqueous NaHCO₃ solution was added, the layers separated, and the aqueous layer extracted with dichloromethane. The combined organic layers were dried with Na₂SO₄, filtered, and the solvent evaporated. The crude material was purified by column chromatography on silica gel (0-50% ethyl acetate in hexanes) to afford the product.

### General Procedure II (4):

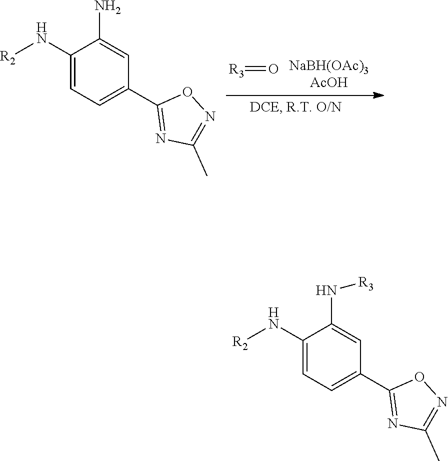

Aniline (1.0 eq), and ketone (1.0 eq) were dissolved in dichloroethane (0.1 M) followed by addition of acetic acid (1.2 eq) and NaBH(OAc)₃ (1.2 eq). The reaction mixture was stirred at room temperature overnight. A saturated aqueous NaHCO₃ solution was added, the layers separated, and the aqueous layer extracted with dichloromethane. The combined organic layers were dried with Na₂SO₄, filtered, and the solvent evaporated. The crude material was purified by column chromatography on silica gel (DCM/MeOH=100/0 to 90/10) to afford the product.

### General Procedure III (1):

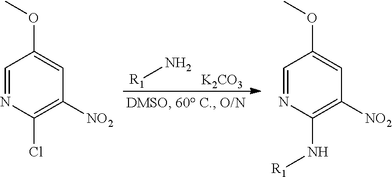

Substituted pyridine (1.0 eq), and substituted amine (1.2 eq.), and potassium carbonate (2.0 eq.) were dissolved in DMSO (0.2 M). the reaction was stirred at 60° C. overnight. After cooling to room temperature, the reaction mixture was partitioned between water and ethyl acetate. The layers were separated and the aqueous layer was extracted with ethyl acetate (3×). The combined organic layers were washed with brine, dried with Na₂SO₄, filtered, and the solvent evaporated. The crude product was purified by column chromatography on silica gel (0-50% ethyl acetate in hexanes) to give the product.

### General Procedure V (1):

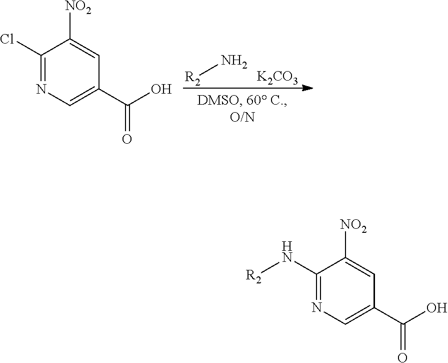

Nitronicotinic acid (1.0 eq), and substituted amine (1.2 eq.), and potassium carbonate (2.0 eq.) were dissolved in DMSO (0.2 M). the reaction was stirred at 60° C. overnight. After cooling to room temperature, the reaction mixture was partitioned between water and ethyl acetate. The layers were separated and the aqueous layer was extracted with ethyl acetate (3×). The combined organic layers were washed with brine, dried with Na₂SO₄, filtered, and the solvent evaporated. The crude product was purified by column chromatography on silica gel (DCM/MeOH=100/0 to 90/10) to give the product.

### General Procedure V (2):

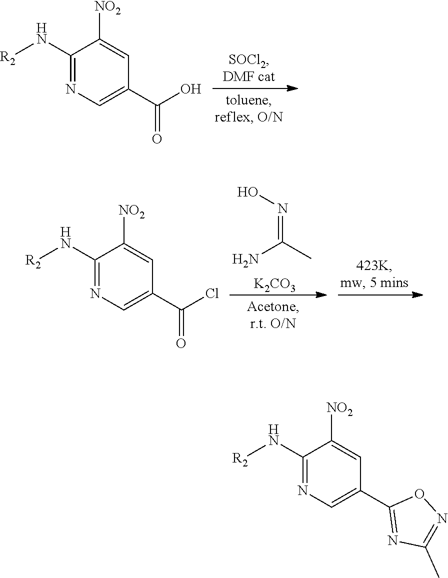

Substituted nitronicotinic acid (1.0 eq), Thionyl chloride (2.0 eq.), and DMF (2 drops) were dissolved in toluene (0.2 M). The reaction was refluxed overnight. After cooling to room temperature, the reaction mixture was evaporated. The resulted solid was added to a solution of N′-hydroxyacetimidamide (1.1 eq) and K₂CO₃ (1.1 eq) in acetone (0.4 M) and stirred at room temperature overnight. The solvent was removed by rotatory evaporation, the residue was treated with water, and the precipitate was filtered off. The solid was heated 150° C. in microwave for 5 minutes. The residue was dissolved in dichloromethane and methanol, dried with MgSO₄, filtered, and the solvent was evaporated. The crude product was purified by column chromatography on silica gel (DCM/MeOH=100/0 to 90/10) to afford the product.

### TH-2-7

### N²-cyclohexyl-4-methoxypyridine-2,5-diamine

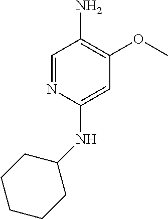 [SMILES: COc1cc(NC2CCCCC2)ncc1N]

Following general procedure III(1) with N-cyclohexyl-4-methoxy-5-nitropyridin-2-amine (13 mg, 0.052 mmol), N²-cyclohexyl-4-methoxypyridine-2,5-diamine (13 mg, 99% yield) was obtained as purple black oil.

¹H NMR (400 MHz, Chloroform-d) δ 7.40 (s, 1H), 5.89 (s, 1H), 3.90 (s, 3H), 3.48 (s, 1H), 3.40 (ddd, J=9.7, 5.9, 3.9 Hz, 2H), 2.04-1.92 (m, 2H), 1.85-1.71 (m, 2H), 1.62 (dt, J=11.9, 4.1 Hz, 1H), 1.44-1.21 (m, 6H).

MS (m/z): [MH]⁺ calculated for C₁₂H₁₉N₃O [M+H]⁺: 222.1606, found: 222.1625.

### TH-2-8

### 6-chloro-N-cyclohexyl-4-methoxypyridin-3-amine

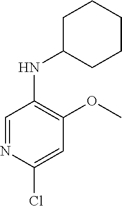 [SMILES: COc1cc(Cl)ncc1NC1CCCCC1]

Following general procedure I(4) with 6-chloro-4-methoxypyridin-3-amine (40 mg, 0.29 mmol), 6-chloro-N-cyclohexyl-4-methoxypyridin-3-amine (51 mg, 73% yield) was obtained as purple black oil.

¹H NMR (400 MHz, Chloroform-d) δ 7.60 (s, 1H), 6.67 (s, 1H), 3.91 (s, 3H), 3.28 (tt, J=10.0, 3.7 Hz, 1H), 2.17-2.00 (m, 2H), 1.83-1.59 (m, 4H), 1.48-1.35 (m, 2H), 1.33-1.16 (m, 3H).

MS (m/z): [MH]⁺ calculated for C₁₂H₁₈ClN₂O, 241.1108; found 241.1108.

### TH-2-9-2

### N²,N⁵-dicyclohexyl-4-methoxypyridine-2,5-diamine

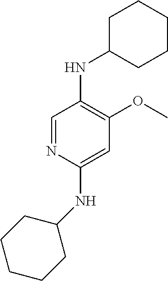 [SMILES: COc1cc(NC2CCCCC2)ncc1NC1CCCCC1]

Following general procedure I(4) with N-cyclohexyl-4-methoxy-5-nitropyridin-2-amine (13 mg, 0.06 mmol), N²,N⁵-dicyclohexyl-4-methoxypyridine-2,5-diamine (4 mg, 22% yield) was obtained as light yellow solid.

¹H NMR (400 MHz, Chloroform-d) δ 6.92 (s, 1H), 5.94 (s, 1H), 3.99 (s, 3H), 3.38-3.25 (m, 1H), 3.00 (tt, J=10.0, 3.7 Hz, 1H), 2.00 (td, J=13.0, 3.6 Hz, 4H), 1.89-1.72 (m, 4H), 1.66 (d, J=5.1 Hz, 2H), 1.54-1.08 (m, 12H).

MS (m/z): [MH]⁺ calculated for C₁₈H₃₀N₃O, 304.2489; found 304.2397.

### TH-3-86-r2

### N²-cyclohexyl-N⁵,N⁵-diisopropyl-3-(3-methyl-1,2,4-oxadiazol-5-yl)pyridine-2,5-diamine

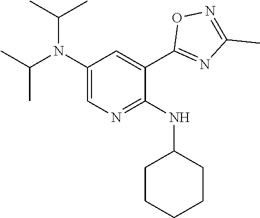 [SMILES: Cc1noc(-c2cc(N(C(C)C)C(C)C)cnc2NC2CCCCC2)n1]

Following general procedure II(4) with N²-cyclohexyl-3-(3-methyl-1,2,4-oxadiazol-5-yl)pyridine-2,5-diamine, N²-isopropyl-6-methoxypyridine-2,3-diamine was obtained as yellow solid as a side product.

¹H NMR (400 MHz, Chloroform-d) δ 8.54 (d, J=2.8 Hz, 1H), 8.32 (d, J=7.7 Hz, 1H), 8.18 (d, J=7.6 Hz, 1H), 2.51 (s, 3H), 2.07 (d, J=12.1 Hz, 2H), 1.85-1.74 (m, 2H), 1.74-1.58 (m, 1H), 1.58-1.31 (m, 6H), 1.36 (d, J=6.5 Hz, 12H).

MS (m/z): [MH]⁺ calculated for C₂₀H₃₂N₅O, 358.2607; found 358.2623.

### TH-1-73

### tert-butyl 5-amino-2-(cyclohexylamino)nicotinate

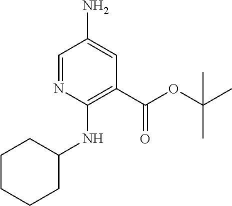 [SMILES: CC(C)(C)OC(=O)c1cc(N)cnc1NC1CCCCC1]

Following general procedure I(3) with tert-butyl 2-(cyclohexylamino)-5-nitronicotinate (60 mg, 0.19 mmol), tert-butyl 5-amino-2-(cyclohexylamino)nicotinate (40 mg, 69% yield) was obtained as yellow solid.

¹H NMR (400 MHz, Chloroform-d) δ 7.92 (d, J=3.0 Hz, 1H), 7.59 (d, J=3.0 Hz, 1H), 4.08-3.92 (m, 1H), 3.87-3.70 (m, 2H), 2.06 (dd, J=12.9, 4.0 Hz, 3H), 1.94-1.82 (m, 2H), 1.76 (dt, J=13.3, 4.1 Hz, 2H), 1.38-1.18 (m, 5H).

### TH-1-75

### tert-butyl 2,5-bis(cyclohexylamino)nicotinate

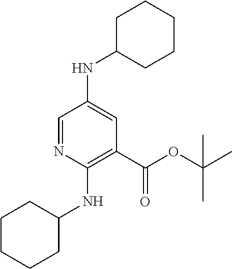 [SMILES: CC(C)(C)OC(=O)c1cc(NC2CCCCC2)cnc1NC1CCCCC1]

Following general procedure I(4) with tert-butyl 5-amino-2-(cyclohexylamino)nicotinate (40 mg, 0.14 mmol), tert-butyl 2,5-bis(cyclohexylamino)nicotinate (40 mg, 76% yield) was obtained as yellow solid.

¹H NMR (400 MHz, DMSO-d₆) δ 7.86 (d, J=3.0 Hz, 1H), 7.44 (d, J=3.0 Hz, 1H), 7.19 (d, J=7.6 Hz, 1H), 4.81 (d, J=8.5 Hz, 1H), 3.95-3.82 (m, 1H), 3.09 (d, J=9.2 Hz, 1H), 1.94 (t, J=15.2 Hz, 4H), 1.73 (t, J=12.2 Hz, 4H), 1.61 (d, J=11.2 Hz, 1H), 1.57 (s, 9H), 1.44-1.09 (m, 12H).

MS (m/z): [MH]⁺ calculated for C₂₂H₃₆N₃O₂, 374.28; found 374.2831.

### TH-1-78

### tert-butyl 2-(((3s,5s,7s)-adamantan-1-yl)amino)-5-aminonicotinate

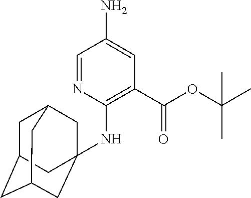 [SMILES: CC(C)(C)OC(=O)c1cc(N)cnc1NC12CC3CC(CC(C3)C1)C2]

Following general procedure I(3) with tert-butyl 2-(((3s,5s,7s)-adamantan-1-yl)amino)-5-nitronicotinate (185 mg, 0.50 mmol), tert-butyl 2-(((3s,5s,7s)-adamantan-1-yl)amino)-5-aminonicotinate (114 mg, 67% yield) was obtained as yellow solid.

¹H NMR (400 MHz, Chloroform-d) δ 7.86 (d, J=3.1 Hz, 1H), 7.52 (d, J=3.1 Hz, 1H), 7.50 (s, 1H), 3.13 (s, 2H), 2.19-2.14 (m, 6H), 2.09 (d, J=4.5 Hz, 3H), 1.77-1.65 (m, 6H), 1.56 (s, 9H).

### TH-1-79

### tert-butyl 2-(((1 r,3r,5r,7r)-adamantan-2-yl)amino)-5-(cyclohexylamino)nicotinate

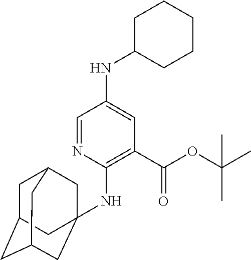 [SMILES: CC(C)(C)OC(=O)c1cc(NC2CCCCC2)cnc1NC12CC3CC(CC(C3)C1)C2]

Following general procedure I(3) with tert-butyl 2-(((3s,5s,7s)-adamantan-1-yl)amino)-5-aminonicotinate (44 mg, 0.13 mmol), tert-butyl 2-(((1r,3r,5r,7r)-adamantan-2-yl)amino)-5-(cyclohexylamino)nicotinate (30 mg, 55% yield) was obtained as yellow solid.

¹H NMR (400 MHz, DMSO-d₆) δ 7.78 (d, J=3.0 Hz, 1H), 7.40 (d, J=3.1 Hz, 1H), 4.74 (s, 1H), 3.06 (s, 1H), 2.11-2.02 (m, 9H), 1.94-1.84 (m, 2H), 1.75-1.69 (m, 2H), 1.66 (s, 6H), 1.53 (s, 9H), 1.38-1.04 (m, 7H).

MS (m/z): [MH]⁺ calculated for C₂₆H₄₀N₃O₂, 426.31; found 426.3120.

### TH-2-64-1

### tert-butyl 2-(cyclohexylamino)-5-(isopropylamino)nicotinate

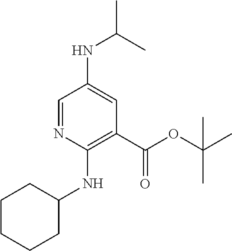 [SMILES: CC(C)Nc1cnc(NC2CCCCC2)c(C(=O)OC(C)(C)C)c1]

Following general procedure I(4) with tert-butyl 5-amino-2-(cyclohexylamino)nicotinate (30 mg, 0.11 mmol), tert-butyl 2-(cyclohexylamino)-5-(isopropylamino)nicotinate (10 mg, 68% yield) was obtained as brown solid.

¹H NMR (400 MHz, Chloroform-d) δ 7.84 (d, J=3.0 Hz, 1H), 7.49 (d, J=2.9 Hz, 1H), 3.99 (d, J=9.8 Hz, 1H), 3.06 (p, J=6.0 Hz, 1H), 2.07 (dd, J=12.3, 3.7 Hz, 2H), 1.76 (dt, J=13.3, 4.0 Hz, 2H), 1.60 (s, 9H), 1.58-1.41 (m, 5H), 1.33-1.26 (m, 2H).

MS (m/z): [MH]⁺ calculated for C₁₉H₃₁N₃O₂ [M+H]⁺: 334.2495, found: 334.2512.

### TH-2-37-1

### N²,N⁵-dicyclohexyl-3-(3-methyl-1,2,4-oxadiazol-5-yl)pyridine-2,5-diamine

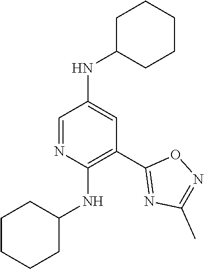 [SMILES: Cc1noc(-c2cc(NC3CCCCC3)cnc2NC2CCCCC2)n1]

Following general procedure II(4) with N²-cyclohexyl-3-(3-methyl-1,2,4-oxadiazol-5-yl)pyridine-2,5-diamine (20 mg, 0.073 mmol), N²,N⁵-dicyclohexyl-3-(3-methyl-1,2,4-oxadiazol-5-yl)pyridine-2,5-diamine (12 mg, 46% yield) was obtained as yellow solid.

¹H NMR (400 MHz, Chloroform-d) δ 8.15 (d, J=2.9 Hz, 1H), 8.09 (d, J=2.9 Hz, 1H), 4.08 (s, 1H), 3.21 (s, 1H), 2.53 (s, 3H), 2.07 (d, J=22.0 Hz, 4H), 1.87-1.66 (m, 6H), 1.62-1.46 (m, 2H), 1.45-1.16 (m, 10H).

MS (m/z): [MH]⁺ calculated for C₂₀H₂₉N₅O [M+H]⁺: 356.2450, found: 356.2469.

### TH-2-37-2

### N²-cyclohexyl-N⁵-isopropyl-3-(3-methyl-1,2,4-oxadiazol-5-yl)pyridine-2,5-diamine

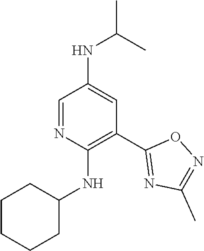 [SMILES: Cc1noc(-c2cc(NC(C)C)cnc2NC2CCCCC2)n1]

Following general procedure II(4) with N²-cyclohexyl-3-(3-methyl-1,2,4-oxadiazol-5-yl)pyridine-2,5-diamine (20 mg, 0.073 mmol), N²-cyclohexyl-N⁵-isopropyl-3-(3-methyl-1,2,4-oxadiazol-5-yl)pyridine-2,5-diamine (18 mg, 78% yield) was obtained as yellow solid.

¹H NMR (400 MHz, Chloroform-d) δ 7.92 (d, J=3.0 Hz, 1H), 7.54 (d, J=3.0 Hz, 1H), 7.50 (d, J=7.8 Hz, 1H), 4.11-3.98 (m, 1H), 2.93 (s, 1H), 2.47 (s, 3H), 2.05 (dd, J=12.3, 4.6 Hz, 2H), 1.76 (dt, J=13.1, 4.2 Hz, 2H), 1.63 (dt, J=12.5, 3.8 Hz, 1H), 1.60-1.40 (m, 4H), 1.40-1.25 (m, 3H), 1.20 (d, J=6.3 Hz, 6H).

MS (m/z): [MH]⁺ calculated for C₁₇H₂₅N₅O [M+H]⁺: 316.2137, found: 316.2162.

### TH-4-45

### N,N-diethyl-3-(3-methyl-1,2,4-oxadiazol-5-yl)-5-nitropyridin-2-amine

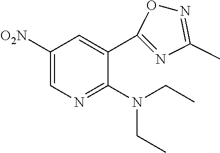 [SMILES: CCN(CC)c1ncc([N+](=O)[O-])cc1-c1nc(C)no1]

Following general procedure V(2) with 2-(diethylamino)-5-nitronicotinic acid (770 mg, 3.2 mmol), N,N-diethyl-3-(3-methyl-1,2,4-oxadiazol-5-yl)-5-nitropyridin-2-amine (153 mg, 19% yield) was obtained as yellow solid.

¹H NMR (400 MHz, Chloroform-d) δ 8.94 (d, J=2.4 Hz, 1H), 8.47 (s, 1H), 3.61 (q, J=7.2 Hz, 4H), 3.48 (s, 3H), 1.31-1.12 (m, 6H).

MS (m/z): [MH]⁺ calculated for C₁₂H₁₆N₅O, 278.1253; found 278.1268.

### TH-4-48-2

### N-cyclohexyl-3-(3-methyl-1,2,4-oxadiazol-5-yl)-5-nitropyridin-2-amine

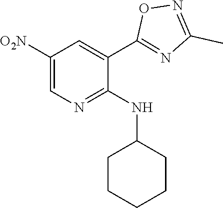 [SMILES: Cc1noc(-c2cc([N+](=O)[O-])cnc2NC2CCCCC2)n1]

Following general procedure V(1) and V(2) with 2-chloro-5-nitronicotinic acid (1 g, 4.92 mmol), N-cyclohexyl-3-(3-methyl-1,2,4-oxadiazol-5-yl)-5-nitropyridin-2-amine (72 mg, 5% yield) was obtained as yellow solid.

¹H NMR (400 MHz, Chloroform-d) δ 9.17 (dd, J=2.7, 0.4 Hz, 1H), 8.96 (d, J=2.7 Hz, 1H), 8.86 (d, J=8.1 Hz, 1H), 4.38-4.18 (m, 1H), 2.51 (s, 3H), 2.12-1.92 (m, 2H), 1.79 (dt, J=13.1, 4.1 Hz, 2H), 1.72-1.63 (m, 1H), 1.55-1.30 (m, 5H).

MS (m/z): [MH]⁺ calculated for C₁₄H₁₇N₅O₃, 304.1410; found 304.1431.

### TH-4-53-1

### N²-cyclohexyl-3-(3-methyl-1,2,4-oxadiazol-5-yl)-N⁵-(pentan-3-yl)pyridine-2,5-diamine

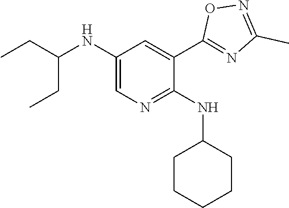 [SMILES: CCC(CC)Nc1cnc(NC2CCCCC2)c(-c2nc(C)no2)c1]

Following general procedure II(4) with N²-cyclohexyl-3-(3-methyl-1,2,4-oxadiazol-5-yl)pyridine-2,5-diamine (18 mg, 0.066 mmol), N²-cyclohexyl-3-(3-methyl-1,2,4-oxadiazol-5-yl)-N⁵-(pentan-3-yl)pyridine-2,5-diamine (8 mg, 36% yield) was obtained as yellow solid.

¹H NMR (400 MHz, Chloroform-d) δ 8.20 (s, 1H), 8.05 (s, 1H), 4.10 (s, 1H), 3.63-3.49 (m, 1H), 2.52 (s, 3H), 2.08 (d, J=12.0 Hz, 2H), 1.78 (d, J=13.4 Hz, 2H), 1.73-1.62 (m, 2H), 1.61-1.47 (m, 3H), 1.45-1.36 (m, 3H), 1.36-1.24 (m, 10H).

MS (m/z): [MH]⁺ calculated for C₁₉H₃₀N₅O, 344.2450; found 344.2440.

### TH-4-53-2

### N²-cyclohexyl-N⁵-cyclopentyl-3-(3-methyl-1,2,4-oxadiazol-5-yl)pyridine-2,5-diamine

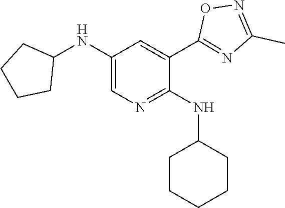 [SMILES: Cc1noc(-c2cc(NC3CCCC3)cnc2NC2CCCCC2)n1]

Following general procedure II(4) with N²-cyclohexyl-3-(3-methyl-1,2,4-oxadiazol-5-yl)pyridine-2,5-diamine (15 mg, 0.164 mmol), N²-cyclohexyl-N⁵-cyclopentyl-3-(3-methyl-1,2,4-oxadiazol-5-yl)pyridine-2,5-diamine (7 mg, 37% yield) was obtained as yellow solid.

¹H NMR (400 MHz, Chloroform-d) δ 8.00 (d, J=2.5 Hz, 1H), 7.92 (d, J=2.8 Hz, 1H), 3.92 (d, J=10.2 Hz, 1H), 3.67 (t, J=6.1 Hz, 1H), 2.46 (s, 3H), 2.08-1.88 (m, 4H), 1.79-1.54 (m, 6H), 1.45 (dd, J=15.4, 9.4 Hz, 4H), 1.39-1.12 (m, 4H), 0.78 (tt, J=13.9, 6.3 Hz, 2H).

MS (m/z): [MH]⁺ calculated for C₁₉H₂₇N₅O, 342.2294; found 342.2303.

### Example 8

### Development of Small Molecule Brain-Penetrant Ferroptosis Inhibitors

Ferroptosis is a non-apoptotic, iron-dependent form of regulated cell death, driven by the loss of activity of lipid repair capacity and subsequent accumulation of lipid-based reactive oxygen species (ROS), particularly lipid hydroperoxides. Accumulating evidence suggests that ferroptosis may be associated with neurodegenerative disease pathology, with ferrostatin-1 (fer-1) inhibiting cell death in cellular models of Huntington disease (HD). However numerous liabilities of fer-1 and its second-generation and third-generation analogs, including low in vitro stability, poor brain penetration, and potential toxicity, limit the applicability of fer-1 and related compounds in disease models and human patients. Herein, we report the development of fourth and fifth generations of ferrostatins. A fourth-generation compound showed low brain penetration, but high kidney retention, which suggested it could be a candidate for evaluating the role of ferroptosis in kidney diseases. Indeed, significant effectiveness was confirmed in a rat ferroptosis model of renal proximal tubules. Fifth generation ferrostatins have high potency, stability, brain penetration, and low toxicity. We found that these fifth generation ferrostatins were well tolerated in wild-type and symptomatic R6/2 Huntington Disease mice following 30 days of intraperitoneal and oral gavage administration; one compound protected against disease-associated weight loss in symptomatic male R6/2 mice. These compounds are suitable for evaluating the contribution of ferroptosis in animal models of degenerative diseases and other ferroptosis-linked brain diseases.

### Introduction

Investigations into the molecular mechanism of cell death are important for understanding disease processes (Galluzzi et al. 2018). Ferroptosis is a non-apoptotic, iron-dependent form of regulated cell death driven by the loss of activity of the lipid repair enzyme GPX4 and other protective mechanisms against lipid oxidation. Ferroptosis, associated with accumulation of lipid-based reactive oxygen species (ROS), particularly lipid hydroperoxides, was first identified through discoveries in the labs of Brent Stockwell and Marcus Conrad (Dixon et al. 2012; Seiler et al. 2008; Dolma et al. 2003). Ferroptosis can be induced by small molecules, including erastin, Imidazole Ketone Erastin (IKE), RSL3, sulfasalazine, and sorafenib, or genetic deletion of GPX4, which results in lipid peroxide accumulation (Wolpaw et al. 2011; Yang and Stockwell 2016; Jiang, Stockwell, and Conrad 2021).

Ferrostatin-1 (Fer-1) is a potent small-molecule ferroptosis inhibitor that was initially discovered in the Stockwell Lab in a high-throughput screen for inhibitors of erastin-induced cell death (Dixon et al. 2012). Fer-1 acts as a radical-trapping agent that prevents propagation of polyunsaturated fatty acyl group peroxidation (PUFAs) (Loscalzo 2008; Conrad and Pratt 2019; Zilka et al. 2017; Dixon et al. 2012). Previous work demonstrated that fer-1 is protective in a variety of disease models, including Huntington Disease, ischemia reperfusion injury in the kidney, Parkinson's disease, and ischemic stroke, among others (Skouta et al. 2014; Li et al. 2017). Inhibiting ferroptosis is protective in models of Huntington disease (HD), with fer-1 treatment preventing cell death in rat corticostriatal brain slices biolistically transfected with pathogenic mutant huntingtin protein in a dose-dependent manner; treatment with ferroptosis inhibitors, including D-PUFAs and the mitochondrially targeted antioxidant XJB-5-131 ameliorates behavioral deficits in rodent models of HD (Skouta et al. 2014; Ji et al. 2012; Xun et al. 2012; Hatami et al. 2018). However, fer-1 is not suitable for in vivo use, due to its low in vitro metabolic and plasma stability, necessitating the development of more drug-like ferrostatins with favorable ADME properties.

It was previously reported the synthesis and characterization of 67 ferrostatin analogs, culminating in SRS11-92 as a second-generation ferrostatin with low nanomolar potency for suppressing erastin-induced death in HT-1080 fibrosarcoma cells (Skouta et al. 2014). While potent, this compound suffered from low stability due to the presence of an ethyl ester moiety that is metabolized by plasma esterases. Efforts to increase plasma stability through the replacement of the ethyl ester moiety with a tert-butyl ester increased in vitro plasma stability; however, the resulting third-generation compound SRS16-86, which also contained an imine group to increase microsomal stability, had a ˜4-fold decrease in potency relative to fer-1 and a >50-fold decrease in potency relative to SRS11-92 (Linkerman et al., 2014 PNAS) (Li et al. 2020).

Additional efforts utilized the SRS11-92 scaffold in analog development efforts, with Augustyn and colleagues reporting fer-1 analogs containing sulfonamide moieties with improved microsomal and plasma stability; however, these analogs suffered from low aqueous solubility, necessitating further derivatization to increase solubility (Hofmans et al. 2016). In addition to the low in vitro stability, the applicability of fer-1 to testing in HD and other neurodegenerative disease models has been limited by the lack of brain penetration, as well as the presence of aniline and phenylenediamine components that have potential toxicities (Devisscher et al. 2018). There is a need to develop potent and brain-penetrant fer-1 analogs without associated toxicities. Thus, a potent, brain-penetrant, stable, and non-toxic ferroptosis inhibitor would be valuable for testing the role of ferroptosis in diverse neurodegenerative disease applications.

This example further reported the development and characterization of a fourth generation ferrostatin (PHB-4082) that accumulates in the kidney, and three fifth generation ferrostatins (TH-2-31, TH-4-55-2, and TH-4-67) that are potent, stable, and brain penetrant in vivo. We evaluated the potential of these latter compounds for neurodegenerative disease applications in a mouse model of Huntington Disease, where we observed that the analogs were specific for protecting against ferroptotic neuronal cell death and that daily administration of one compound to symptomatic male R6/2 mice protected against weight loss (Mangiarini et al. 1996). As such, these fifth generation ferrostatins are suitable for probing the role of ferroptosis in a variety of neurodegenerative disease animal models.

### Methods and Materials

### Cell Viability Assays

Cell culture assays were incubated at 37° C. with 5% CO₂. HT-1080 cells were grown in DMEM (Corning) supplemented with 10% FBS (Life Technologies), 1% Penicillin-Streptomycin 10,000 U/mL (Gibco), and 1% MEM Non-Essential Amino Acids Solution 100× (Gibco). For cell viability assays, cells were trypsinized, counted, and seeded into 384-well white polypropylene plates at 1,000 cells/well, unless otherwise specified. After allowing cells to adhere overnight, compounds in DMSO stocks were arrayed in a 16-point dilution series prepared in a mother plate, and treated from a daughter plate, [DMSO]=0.28%. After 24 or 48 h, 50% CellTiter-Glo (Promega) 50% cell culture medium was added to each well and incubated at room temperature with shaking for 15 min. Luminescence was measured using a Victor X5 plate reader (PerkinElmer). All cell viability data were normalized to the DMSO vehicle condition. From these data, dose-response curves and IC₅₀ values were computed using Prism 7.0 (GraphPad). All 384w measurements were performed in triplicate

### Microsomal Stability Assay

To a 96-well polypropylene plate was added phosphate buffer (182.2 μL, pH 7.4, 100 mM) followed by addition of NADPH-regenerating system solution A (10 μL), and NADPH-regenerating system solution B (2 μL) (Corning Gentest 3P NADPH regenerating system solution A (#451220) and B (#451200)). A stock solution of analog (0.8 μL. 5 mM) or fer-1 (positive control) was added and the mixture was warmed to 37° C. for 5 min. Mouse microsomes (CD-1, 20 mg/mL, Life Technologies) (5 μL, thawed in 37° C. water bath before use) were added. The resulting reaction mixture was kept at 37° C. with gentle agitation for the duration of the experiment. At selected time points (0, 1, 5, 10, 20, 30, 60 and 120 min) aliquots (15 μL) were withdrawn from the plate and quenched upon addition to cold methanol (60 μL), containing an internal standard (5 μM) in a separate 96-well polypropylene plate. At the completion of the final time-point, the samples were centrifuged at 4,000 rpm (1872 g) for 5 min at 4° C. The supernatant (40 μL) was withdrawn and transferred to a sample vial with insert. The samples were analyzed by LC-MS. LC-MS analysis was performed on a platform comprising a Thermo Scientific Dionex Ultimate 3000 and a Bruker amaZon SL equipped with an electrospray ionization source controlled by Bruker Hystar 3.2. Chromatographic separation was performed by injecting 5 μL of the sample onto an Agilent Eclipse Plus C18 column (2.1×50 mm, 3.5 μm) maintained at 20° C. The flow rate was maintained at 400 μL/min. The initial flow conditions were 80% solvent A (water containing 0.1% acetic acid) and 20% solvent B (methanol containing 0.1% acetic acid). Solvent B was raised to 80% over 0.50 min by 1.50 min. Solvent B was raised to 100% by 5.00 min and held there for 3.25 min. Solvent B was lowered back to initial conditions (20%) over 0.50 min by 8.75 min with a total run time of 12.00 min. All analogs were detected in positive mode as [M+H]*. The percent of compound remaining at each time-point was calculated as the ratio of the integrated compound peak over the internal standard peak and standardized to the t=0 time-point. Values were plotted in GraphPad Prism 9 and fit with a one phase decay.

### Plasma Stability Assay

Mouse plasma (GeneTex) was centrifuged at 3000 rpm (1053 g) for 10 min at 10° C. with the resulting supernatant withdrawn and diluted 1:1 in phosphate buffer pH 7.4. To a 96-well polypropylene plate was added 50% mouse plasma phosphate buffer (195 μL). A stock solution of analog in DMSO (0.8 μL. 5 mM) or fer-1 (positive control) was added to a separate well and the components were warmed to 37° C. for 5 min with gentle agitation. The reaction was initiated with the addition of analog to plasma, and the reaction kept at 37° C. for gentle agitation for the duration of the assay. At selected time points, aliquots (15 μL) were withdrawn from the plate and quenched upon addition to cold methanol (60 μL), containing an internal standard (5 μM) in a separate 96-well polypropylene plate. At the completion of the final time-point, the samples were centrifuged at 4,000 rpm (1872 g) for 5 min at 4° C. The supernatant (40 μL) was withdrawn and transferred to a sample vial with insert. The samples were analyzed by LC-MS. LC-MS analysis was performed on a platform comprising a Thermo Scientific Dionex Ultimate 3000 and a Bruker amaZon SL equipped with an electrospray ionization source controlled by Bruker Hystar 3.2. Chromatographic separation was performed by injecting 5 μL of the sample onto an Agilent Eclipse Plus C18 column (2.1×50 mm, 3.5 μm) maintained at 20° C. The flow rate was maintained at 400 μL/min. The initial flow conditions were 80% solvent A (water containing 0.1% acetic acid) and 20% solvent B (methanol containing 0.1% acetic acid). Solvent B was raised to 80% over 0.50 min by 1.50 min. Solvent B was raised to 100% by 5.00 min and held there for 3.25 min. Solvent B was lowered back to initial conditions (20%) over 0.50 min by 8.75 min with a total run time of 12.00 min. All analogs were detected in positive mode as [M+H]*. The percent compound remaining at each time-point was calculated as the ratio of the integrated compound peak over the internal standard peak and standardized to the t=0 time-point. Values were plotted in GraphPad Prism 9 and fit with a one phase decay.

### Animal Studies

All animal study protocols were approved by the Columbia University Institutional Animal Care and Use Committee (IACUC). C57BL/6 mice (The Jackson Laboratory, stock number 000664) (male and female, 8-weeks of age)) were acclimated after shipping for >3 days before beginning experiments. Mice were maintained on a 12 h light/dark cycle and fed a standard diet (PicoLab 5053) PHB4082 IP PK Study

C57BL/6 mice (8-weeks of age and ˜25 g weight) were weighed before injection and divided into groups of two male and female mice per cage. PHB4082 was dissolved in 5% DMSO/95% of 65% v/v of 25% w/v 2-hydroxypropyl-3-cyclodextrin (Cayman Chemical) dissolved in 20% EtOH, 30% v/v Poly(ethylene glycol)-400 (Sigma Aldrich 202398), 5% v/v Tween 80 (Fluka 59924) to create a 2 mg/mL Solution. The same formulation without PHB4082 was used as a vehicle control. The solution was sterilized using a 0.22 mm Steriflip filter unit (Thomas Scientific 1189Q46) and stored at 4° C. Mice were dosed IP and euthanized by CO₂ asphyxiation for 3 min at 0, 1, 2, 4, and 8 h after administration. To ensure that the vehicle was well-tolerated, four mice were treated with vehicle and euthanized 4 h after administration. ˜0.5 mL of blood was collected via cardiac puncture and immediately put in K3 EDTA microtubes (SARSTEDT 41.1504.105) and kept on ice. Organs were harvested, placed in Eppendorf tubes, and frozen on dry ice. Blood samples were centrifuged for 10 min at 2,100×g at 4° C., then plasma was transferred to a clean tube and frozen on dry ice. Organ samples were weighed and placed in hard tissue homogenizing tubes (Omni International 19-628) and a volume of DEPC-treated nuclease-free water (IBI Scientific IB42200) was added to make a 500 mg/mL solution and homogenized using the Omni Bead Ruptor 4 at speed 5 for 30 seconds. PHB4082 was extracted from plasma or organ homogenate by adding 900 μL acetonitrile to 100 μL plasma or organ homogenate. Samples were mixed by vortexing and allowed to extract overnight at 4° C. prior to mixing for at least 5 min by rotating at room temperature, vortexing, and sonicating for at least 30 second prior to centrifugation for 10 min at 4,000×g and 4° C. The supernatant was then transferred to a glass vial and dried under nitrogen. After drying, the samples were resuspended in 100 μL of methanol and analyzed by UPLC-MS described below. The concentration of PHB4082 was determined against a standard curve with a linear fit and the data plot in GraphPad Prism 9 and fit with a one phase decay.

### Renal Ferroptosis Study

Fe(NO₃)₃·12H₂O (Wako, Osaka, Japan) and nitrilotriacetic acid disodium salt (Tokyo Chemical Industry, Tokyo, Japan) were dissolved in deionized water to generate 300 mM and 600 mM solutions, respectively, which were mixed immediately before injections by a ratio of 1:2 (v/v) and then adjusted to pH 7.4 with sodium carbonate to make Fe-NTA solution. PHB4082 was dissolved in 5% DMSO/95% of 65% v/v of 25% w/v 2-hydroxypropyl-β-cyclodextrin (Cayman Chemical) dissolved in 20% EtOH, 30% v/v Poly(ethylene glycol)-400 (Sigma Aldrich 202398), 5% v/v Tween 80 (Fluka 59924) to generate 2 mg/mL solution. The same formulation without PHB4082 was used as vehicle control. For both the solutions ultrasonic disruptor was applied to accelerate the dissolution and they are stored at −20° C. up to one week before injections.

Male wild-type Sprague-Dawley rat (CLEA Japan, Tokyo; n=6 for vehicle control, n=7 for PHB4082 pretreatment) at 4-6 weeks of age were fasted for 12 h prior to intraperitoneal administration of 2 mg/ml PHB4082 solution as pretreatment or with the formulation without PHB4082 as vehicle control. Two hours later all the rats were followed by IP injection of Fe-NTA with a dose of 10 mg iron/kg. The rats were euthanized 24 h after Fe-NTA injection. Fresh kidney tissues were excised and were either fixed in 10% phosphate-buffered formalin or preserved at −80° C. for subsequent analysis. The animal experiment committee of Nagoya University Graduate School of Medicine approved the animal experiments.

### Immunohistochemistry

Immunostainings were performed by BOND MAX/III (Leica, Wetzlar, Germany) with BOND Polymer Refine Detection (ds9800; Leica) as described. Primary antibodies applied were listed as: anti-4-hydroxy-2-nonenal (HNE) modified proteins antibody (anit-HNEJ-1, in house), anti-transferrin receptor antibody (13-6800, Invitrogen). Quantitation of renal tubular ferroptosis was performed by measuring the necrotized cortical area after H&E staining by identifying the area of necrotized tubules with loss of nuclei. ImageJ was used for quantification of % necrosis area with randomly selected microscopic areas (×20, objective lens; n=9).

### TH-2-31 IP PK Study

C57BL/6 mice (8-weeks of age and ˜25 g weight) were weighed before injection and divided into groups of 2 male and female mice per cage. TH-2-31 was dissolved in 5% DMSO/95% of 65% v/v of 25% w/v 2-hydroxypropyl-β-cyclodextrin (Cayman Chemical) dissolved in 20% EtOH, 30% v/v Poly(ethylene glycol)-400 (Sigma Aldrich 202398), 5% v/v Tween 80 (Fluka 59924) to create a 4 mg/mL solution. The same formulation without TH-2-31 was used as a vehicle control. The solution was sterilized using a 0.22 mm Steriflip filter unit (Thomas Scientific 1189Q46). Mice were dosed IP and euthanized by CO₂ asphyxiation for 3 min at 0, 1, 2, 4, and 8 h after administration. To ensure that the vehicle was well-tolerated 4 mice were treated with vehicle and euthanized 4 h after administration. ˜0.5 mL of blood was collected via cardiac puncture and immediately put in K3 EDTA microtubes (SARSTEDT 41.1504.105) and kept on ice. Organs were harvested, placed in Eppendorf tubes, and frozen on dry ice. Blood samples were centrifuged for 10 min at 2,100×g at 4° C., then plasma was transferred to a clean tube and frozen on dry ice. Organ samples were weighed and placed in hard tissue homogenizing tubes (Omni International 19-628) and a volume of DEPC-treated nuclease-free water (IBI Scientific IB42200) was added to make a 500 mg/mL solution and homogenized using the Omni Bead Ruptor 4 at speed 5 for 30 seconds. TH-2-31 was extracted from plasma or organ homogenate by adding 900 μL acetonitrile to 100 μL plasma or organ homogenate. Samples were mixed by vortexing and allowed to extract overnight at 4° C. prior to mixing for at least 5 min by rotating at room temperature, vortexing, and sonicating for at least 30 second prior to centrifugation for 10 min at 4,000×g and 4° C. The supernatant was then transferred to a glass vial and dried under nitrogen. After drying, the samples were resuspended in 100 μL of methanol and analyzed by UPLC-MS described below. The concentration of TH-2-31 was determined against a standard curve with a linear fit and the data plot in GraphPad Prism 9 and fit with a one phase decay.

### TH-2-31, TH-4-55-2, TH-4-67 PK Study

C57BL/6 mice (8-weeks of age and ˜25 g weight) were weighed before injection and divided into groups of two male and female mice per cage. Compound was dissolved in 5% DMSO/95% of 1:1 [(65% v/v of 25% w/v 2-hydroxypropyl-3-cyclodextrin (Cayman Chemical) dissolved in 20% EtOH, 30% v/v Poly(ethylene glycol)-400 (Sigma Aldrich 202398), 5% v/v Tween 80 (Fluka 59924)):MilliQ water] to create a 2 mg/mL solution. The same formulation without compound was used as a vehicle control. The solution was sterilized using a 0.22 mm Steriflip filter unit (Thomas Scientific 1189Q46) and stored at 4° C. Mice were dosed IP, IV, and PO routes of administration and euthanized by CO₂ asphyxiation for 3 min at 0, 1, 2, 4, 8, and 24 h after administration. To ensure that the vehicle was well-tolerated 4 mice were treated with vehicle and euthanized 4 h after administration. ˜0.5 mL of blood was collected via cardiac puncture and immediately put in K3 EDTA microtubes (SARSTEDT 41.1504.105) and kept on ice. Organs were harvested, placed in Eppendorf tubes, and frozen on dry ice. Blood samples were centrifuged for 10 min at 2,100×g at 4° C., then plasma was transferred to a clean tube and frozen on dry ice. Organ samples were weighed and placed in hard tissue homogenizing tubes (Omni International 19-628) and a volume of DEPC-treated nuclease-free water (IBI Scientific IB42200) was added to make a 500 mg/mL solution and homogenized using the Omni Bead Ruptor 4 at speed 5 for 30 seconds. Compound was extracted from plasma or organ homogenate by adding 900 μL acetonitrile to 100 μL plasma or organ homogenate. Samples were mixed by vortexing and allowed to extract overnight at 4° C. prior to mixing for at least 5 min by rotating at room temperature, vortexing, and sonicating for at least 30 second prior to centrifugation for 10 min at 4,000×g and 4° C. The supernatant was then transferred to a glass vial and analyzed by UPLC-MS described below. The concentration of each analog was determined against a standard curve with a nonlinear fit and the data plot in GraphPad Prism 9 and fit with a one phase decay.

### PHB4082 IP, IV Study

C57BL/6 mice (8-weeks of age and ˜25 g weight) were weighed before injection and divided into groups of two male and female mice per cage. PHB4082 was dissolved in 5% DMSO/95% of 1:1 [(65% v/v of 25% w/v 2-hydroxypropyl-3-cyclodextrin (Cayman Chemical) dissolved in 20% EtOH, 30% v/v Poly(ethylene glycol)-400 (Sigma Aldrich 202398), 5% v/v Tween 80 (Fluka 59924)):MilliQ water] to create a 2 mg/mL solution. The same formulation without PHB4082 was used as a vehicle control. The solution was sterilized using a 0.22 mm Steriflip filter unit (Thomas Scientific 1189Q46). Mice were dosed IP, and IV routes of administration and euthanized by CO₂ asphyxiation for 3 min at 2 and 4 h after administration. To ensure that the vehicle was well-tolerated, four mice for each timepoint were treated with vehicle and euthanized 4 h and 24 h after administration. ˜0.5 mL of blood was collected via cardiac puncture and immediately put in K3 EDTA microtubes (SARSTEDT 41.1504.105) and kept on ice. Organs were harvested, placed in Eppendorf tubes, and frozen on dry ice. Blood samples were centrifuged for 10 min at 2,100×g at 4° C., then plasma was transferred to a clean tube and frozen on dry ice. Organ samples were weighed and placed in hard tissue homogenizing tubes (Omni International 19-628) and a volume of DEPC-treated nuclease-free water (IBI Scientific IB42200) was added to make a 500 mg/mL solution and homogenized using the Omni Bead Ruptor 4 at speed 5 for 30 seconds. Compound was extracted from plasma or organ homogenate by adding 900 μL acetonitrile to 100 μL plasma or organ homogenate. Samples were mixed by vortexing and allowed to extract overnight at 4° C. prior to mixing for at least 5 min by rotating at room temperature, vortexing, and sonicating for at least 30 second prior to centrifugation for 10 min at 4,000×g and 4° C. The supernatant was then transferred to a glass vial and analyzed by UPLC-MS described below. The base peak chromatogram was integrated and quantified by standard curve concurrently ran.

### UPLC-MS Analysis

Samples from animal studies were analyzed via UPLC-MS using a Waters Xevo G2-Xs QTof Mass spectrometer equipped with an Acquity UPLC. Chromatographic separation was carried out at 50° C. on a Acquity UPLC BEH C18 column (1.7 μm, 2.1 mm×50 mm, pore size 130 Å) over a 4.5 min gradient elution. The flowrate was held constant at 0.8 mL/min. Mobile phase A consisted of water and mobile phase B consisted of ACN both containing 0.1% formic acid. After injection, the gradient was held at 50% A for 0.25 min. For the next minute, the gradient was ramped in a linear fashion to 100% B and held at this composition for 0.5 min. The eluent composition returned to the initial condition in 0.01 min and the column was re-equilibrated for 2.74 min before the next injection. Injection volumes were 0.5 μL (TH-2-31 40 mg/kg) and 1 μL for all other conditions. The Xevo G2-Xs was operated in positive electrospray ionization (ESI) mode. A capillary voltage and sampling cone voltage of 0.5 kV and 30 V were used. The source and desolvation temperatures were kept at 120° C. and 20° C., respectively. Nitrogen was used as the desolvation gas with a flowrate of 750 L/hr. The protonated molecular ion of leucine encephalin ([M+H]⁺, m/z 556.2771 was used as a lock mass for mass accuracy and reproducibility. Leucine enkephalin was introduced to the lock mass at a concentration of 2 ng/mL (50% ACN containing 0.1% formic acid), and a flow rate of 5 mL/min. To avoid signal saturation, the signal transmission was attenuated to <10%, based off the signal intensity of the highest standard concentration for the duration of the run. The data was collected over the mass range m/z 50 to 1200 Da with an acquisition time of 0.1 seconds per scan. The retention time for each analog is detailed below. All samples were injected twice and the base peak chromatogram was integrated and quantified by standard curve concurrently ran using MassLynx software.

Retention Times:

| Compound | RT |
|---|---|
| PHB4082 | 1.61 |
| TH-2-31 | 0.83 |
| TH-4-55-2 | 0.64 |
| TH-4-67 | 0.44 |

### R6/2 PK Study

C57BL/6 and R6/2 mice at ˜8 weeks of age were weighed before injection and divided into groups of 2 male and female mice per cage. Ferrostatin analog was dissolved in 5% DMSO/95% of 1:1 [(65% v/v of 25% w/v 2-hydroxypropyl-3-cyclodextrin dissolved in 20% EtOH, 30% v/v Poly(ethylene glycol)-400, 5% v/v Tween 80):MilliQ water] to create a 2 mg/mL solution. The same formulation without analog was used as a vehicle control. The solution was sterilized using a 0.22 mm Steriflip filter unit (Thomas Scientific 1189Q46). Mice were dosed IP and PO routes of administration and euthanized by CO₂ asphyxiation for 3 min at 2, 8, and 24 hours after administration. Approximately 0.5 mL of blood was collected via cardiac puncture and immediately put in K3 EDTA microtubes and kept on ice. Brains were harvested, placed in Eppendorf tubes, and frozen on dry ice. Blood samples were centrifuged for 10 min at 2,100×g at 4° C., then plasma was transferred to a clean tube and frozen on dry ice. Organ samples were weighed and placed in hard tissue homogenizing tubes. A volume of DEPC-treated nuclease-free water was added to make a 500 mg/mL solution and homogenized using the Omni Bead Ruptor 4 at speed 5 for two 30 second runs. The product was aliquoted and diluted to 100 mg/mL.

Prior to UHPLC-MS analysis, plasma was diluted 1:5 in MilliQ water and 500 μL of 100 mg/mL brain homogenate were collected. The plasma and brain samples were acidified by adding 500 μL of 4% H₃PO₄ prior to extracting with the Waters Oasis MCX 1 cc Vacuum cartridge with 30 mg sorbent per cartridge, 30 μm to remove PEG from the samples. (Brain samples were filtered through a 70 uM filter prior to acidification). The columns were conditioned with 1000 μL methanol and 1000 μL MilliQ water and the flow through discarded. The acidified samples were then loaded onto the column, the column washed with 1000 μL of 2% formic acid followed by two washes with 1000 μL of methanol and the flow through discarded. Samples were eluted from the column with 2, 250 μL washes with methanol containing 5% NH₄OH.

### UHPLC-MS Analysis

Samples from animal studies were analyzed via UPLC-MS using a Waters Xevo G2-Xs QTof Mass spectrometer equipped with an Acquity UPLC. Chromatographic separation was carried out at 50° C. on an Acquity UPLC BEH C₁₈ column (1.7 μm, 2.1 mm×50 mm, pore size 130 Å) over a 4.5 min gradient elution. The flowrate was held constant at 0.8 mL/min. Mobile phase A consisted of water and mobile phase B consisted of ACN both containing 0.1% formic acid. After injection, the gradient was held at 50% A for 0.25 min. For the next minute, the gradient was ramped in a linear fashion to 100% B and held at this composition for 0.5 min. The eluent composition returned to the initial condition in 0.01 min and the column was re-equilibrated for 2.74 min before the next injection. Injection volumes were 0.5 μL (TH-2-31 40 mg/kg) and 1 μL for all other conditions. The Xevo G2-Xs was operated in positive electrospray ionization (ESI) mode. A capillary voltage and sampling cone voltage of 0.5 kV and 30 V were used. The source and desolvation temperatures were kept at 120° C. and 20° C., respectively. Nitrogen was used as the desolvation gas with a flowrate of 750 L/hr. The protonated molecular ion of leucine encephalin ([M+H]⁺, m/z 556.2771 was used as a lock mass for mass accuracy and reproducibility. Leucine enkephalin was introduced to the lock mass at a concentration of 2 ng/mL (50% ACN containing 0.1% formic acid), and a flow rate of 5 mL/min. To avoid signal saturation, the signal transmission was attenuated to <10%, based off the signal intensity of the highest standard concentration for the duration of the run. The data was collected over the mass range m/z 50 to 1200 Da with an acquisition time of 0.1 seconds per scan. The retention time for each analog is detailed below. All samples were injected twice, and the base peak chromatogram was integrated and quantified by standard curve concurrently ran using MassLynx software.

### 3-NP Pilot and Efficacy Studies

### 3-NP Administration

3-NP (Sigma Aldrich) was dissolved in PBS at a concentration of 10 mg/mL, pH adjusted to pH 7.4 and sterilized by filtering through a 0.22 mm Steriflip filter unit (Thomas Scientific 1189Q46). Aliquots were stored at −80° C., prior to use. Male C57BL/6 Mice were weighed and injected with 3-NP IP according to the prescribed dosing regimen.

### Ferrostatin Analog Administration

Ferrostatin analog was dissolved in 5% DMSO/95% of 1:1 [(65% v/v of 25% w/v 2-hydroxypropyl-β-cyclodextrin dissolved in 20% EtOH, 30% v/v Poly(ethylene glycol)-400, 5% v/v Tween 80):MilliQ water] to create a 2 mg/mL solution. A vehicle control consisting of 5% dissolved in the cyclodextrin-PEG-Tween formulation was also prepared. Solutions were sterilized using a 0.22 mm Steriflip filter unit. Male C57BL/6 mice were weighed and dosed at 20 mg/kg ferrostatin or vehicle IP. On days where mice received both ferrostatin or vehicle and 3-NP, mice were first dosed with ferrostatin or vehicle, followed by 3-NP˜30 minutes later with all injections were performed at the same time of day. The weight of each mouse was compared to the baseline weight, and the change in weight loss calculated and plotted.

### Open Field Behavioral Analysis

Open Field behavior was assessed during a 30 min session with Activity Monitor Version 7 tracking software (Med Associates Inc.). Briefly, each mouse was gently placed in the center of a clear Plexiglas arena (27.31×27.31×20.32 cm, Med Associates ENV-510) lit with dim light (˜5 lux), and was allowed to ambulate freely. Infrared (IR) beams embedded along the x, y, z axes of the arena automatically track distance moved, horizontal movement, vertical movement, stereotypies, and time spent in the center zone.

### Blinding and Statistics

Experimental blinding was achieved by separating the syringe preparation and the injection and data analysis components, with one researcher preparing the syringes and the other injecting the mice and analyzing the data. Unblinding occurred at the termination of the study after the completion of data analysis. Data were plotted and analyzed in Graph Pad Prism 9.0. Significance was determined via Unpaired t test in the pilot study, or via One-Way ANOVA with multiple comparisons in the efficacy study.

### Toxicity Study

Ferrostatin analogs were dissolved in 5% DMSO/95% of 1:1 [(65% v/v of 25% w/v 2-hydroxypropyl-β-cyclodextrin dissolved in 20% EtOH, 30% v/v Poly(ethylene glycol)-400, 5% v/v Tween 80):MilliQ water] to create a 2 mg/mL solution. The same formulation without analog was used as a vehicle control. The solution was sterilized using a 0.22 mm Steriflip filter unit. C57BL/6 and R6/2 mice at ˜10 weeks of age, after onset of symptoms in R6/2 mice, were injected with ferrostatin analog or vehicle at a dose of 20 mg/kg daily for 30 days via IP and PO routes of administration. A total of 12 C57BL/6 mice (6 male and 6 female) were utilized for each treatment condition and route of administration and a total of 6 R6/2 (3 male and 3 female) were utilized for each treatment and route of administration; TH-4-55-2 IP had 2 male and 3 female mice due to premature death prior to onset of the study. Mice were randomly assigned to each condition, weighed daily, the weight recorded, and the change in body weight compared to the baseline, pre-injection weight was calculated. Any mouse that lost more than 20% of its baseline body weight for three consecutive days was euthanized prior to the completion of the study. Injections occurred daily at the same time in the day to ensure consistency. Experimental blinding was achieved by separating the syringe preparation and the injection and data analysis components, with one researcher preparing the syringes and the other injecting the mice and analyzing the data. Unblinding occurred at the termination of the study after the completion of data analysis. Data were plotted and analyzed in Graph Pad Prism 9.0 with significance determined via Two Way ANOVA or Mixed-effects model with Tukey's test for multiple comparisons.

### Formulation Test

The method used in FIG. 10A: 1.4 mM TH-2-31 was dissolved in 5% DMSO/95% of 1:1 [(65% v/v of 25% w/v 2-hydroxypropyl-β-cyclodextrin dissolved in 20% EtOH, 35% w/v of co-solvent: MilliQ water]. 100 μL sample was placed in 96 well plates by Greiner and analyzed via Nephelometry (ID: 09082203) using software NEPHELOgalaxy, Units measured in Nephelometric Turbidity unit (NTU). NTU values of 24985 is the max value of the machine, indicating a max level of turbidity. Each co-solvent was used in terms of its SDS solubility capabilities in water with temperature adjustment as indicated.

The method used in FIG. 10B: The following formulations were used: 1.4 mM TH-2-31 in 5% DMSO, 30% v/v co-solvent, 20% Ethanol, 45% MilliQ water; 1.4 mM TH-2-31 in 5% DMSO, 30% v/v co-solvent, 65% MilliQ water. 100 μL of the respective samples were placed in a 96-well plate (Greiner) at RT and analyzed via Nephelometry (ID: 09082203) using the software NEPHELOgalaxy. Units measured in Nephelometric Turbidity unit (NTU).

The method used in FIG. 10C: 1.4 mM ferrostatins and 42 uM Citric acid were dissolved in 5% DMSO, 5% ethanol, and 90% PBS. 100 μL sample was placed in a 96 well plate (Greiner) at RT and analyzed via Nephelometry (ID: 09082203) using the software NEPHELOgalaxy. Units measured in Nephelometric Turbidity unit (NTU).

### In Vivo Pharmacokinetic Study of TH-4-55-2 in Drinking Water

TH-4-55-2 and 42 uM Citric acid were dissolved in 5% DMSO, 5% ethanol, and 90% PBS rendering a final concentration of 0.29 mg/mL Th-4-55-2 in drinking water. The mice drink ˜2 mL water per day, and the resulting dosage was around 20-30 mg/kg/day. C57BL/6 mice at ˜8 weeks of age were treated using drinking water daily for four days. 6 mice were randomly picked, weighed daily, the weight recorded, and the change in body weight compared to the baseline, pre-injection weight was calculated. Any mouse that lost more than 20% of its baseline body weight for three consecutive days was euthanized prior to the completion of the study. Mice were euthanized by CO₂ asphyxiation for 3 min after administration. Approximately 0.5 mL of blood was collected via cardiac puncture and immediately put in K3 EDTA microtubes and kept on ice. Brains were harvested, placed in Eppendorf tubes, and frozen on dry ice. Blood samples were centrifuged for 10 min at 2,100×g at 4° C., then plasma was transferred to a clean tube and frozen on dry ice.

Brain samples were weighed and placed in hard tissue homogenizing tubes. Two volume of HPLC grade methanol was added to make a solution and homogenized using the Omni Bead Ruptor 4 at speed 5 for two 30-second runs. The product was aliquoted and diluted by adding 900 μL HPLC grade ice-cold methanol. After centrifugation for 20 mins at 14,000×g at 4° C., supernatant was collected in a clean vial and dried by vacuum centrifugation (GeneVac).

Prior to LC-MS analysis, the samples were resuspended in 100 μL methanol and centrifuged for 5 minutes at 14,000×g at 4° C. The supernatant was analyzed via SYNAPT High Resolution Mass Spectrometer.

### UPLC-MS Analysis

Samples from animal studies were analyzed via UPLC-MS using a Waters SYNAPT XS Mass spectrometer equipped with an Acquity UPLC. Chromatographic separation was carried out at 55° C. on an Acquity UPLC BEH C¹⁸ column (ACQUITY™ 1.7 μm, 2.1 mm×50 mm, pore size 130 Å) over a 10 min gradient elution. The flow rate was held constant at 0.4 mL/min. Mobile phase A consisted of water and mobile phase B consisted of Methanol. Both phases contained 0.1% formic acid. After injection, the gradient was held at 100% A for 0.25 min. For the next minute, the gradient was ramped in a linear fashion to 100% B and held at this composition for 0.5 min. The eluent composition returned to the initial condition in 0.01 min and the column was re-equilibrated for 2.74 min before the next injection. The injection volume was 1 μL for TH-4-55-2. The SYNAPT XS was operated in positive electrospray ionization (ESI) mode. A capillary voltage and sampling cone voltage of 2.2 kV and 40 V were used. The source and desolvation temperatures were kept at 120° C. and 500° C. respectively. Nitrogen was used as the desolvation gas with a flow rate of 800 L/hr. The protonated molecular ion of leucine encephalin ([M+H]⁺, m/z 556.2771 was used as a lock mass for mass accuracy and reproducibility. Leucine enkephalin was introduced to the lock mass at a concentration of 2 ng/mL (50% ACN containing 0.1% formic acid), and a flow rate of 5 mL/min. To avoid signal saturation, the signal transmission was attenuated to <10%, based off the signal intensity of the highest standard concentration for the duration of the run. The data was collected over the mass range m/z 50 to 1200 Da with an acquisition time of 0.1 seconds per scan. The retention time for each analog is detailed below. All samples were injected twice and the base peak chromatogram was integrated and quantified by a standard curve concurrently ran using MassLynx software and exported.

### In Vivo Pharmacokinetic Study of Ferrostatins in Drinking Water

Ferrostatins and 42 μM Citric acid were dissolved in 5% DMSO, 5% ethanol, and 90% PBS rendering a final concentration of 0.29 mg/mL of each ferrostatin in drinking water. The mice drink ˜2 mL water per day, and the resulting dosage was around 20-30 mg/kg/day. R6/2 mice at ˜6-7 weeks of age were treated via drinking water and PO at 20 mg/kg. Mice were weighed daily, the weight recorded, and the change in body weight compared to the baseline, pre-injection weight was calculated. Any mouse that lost more than 20% of its baseline body weight for three consecutive days was euthanized prior to the completion of the study. Mice were euthanized by CO₂ asphyxiation for 3 min after administration. Approximately 0.5 mL of blood was collected via cardiac puncture and immediately put in K3 EDTA microtubes and kept on ice. Brains were harvested, placed in Eppendorf tubes, and frozen on dry ice. Blood samples were centrifuged for 10 min at 2,100×g at 4° C., then plasma was transferred to a clean tube and frozen on dry ice.

Brain samples were weighed and placed in hard tissue homogenizing tubes. Two volume of HPLC grade methanol was added to make a solution and homogenized using the Omni Bead Ruptor 4 at speed 5 for two 30-second runs. The product was aliquoted and diluted by adding 900 μL HPLC grade ice-cold methanol. After centrifugation for 20 mins at 14,000×g at 4° C., supernatant was collected in a clean vial and dried by vacuum centrifugation (GeneVac).

Prior to LC-MS analysis, the samples were resuspended in 100 μL methanol and centrifuged for 5 minutes at 14,000×g at 4° C. The supernatant was analyzed via SYNAPT High Resolution Mass Spectrometer.

### Rotarod Behavioral Task

Rotarod “Linear Incline” experiment was performed with 11- to 12-week-old R6/2 mice at Day 25 with a rotations per minute (RPM) ramp over a 300s duration, start speed of 5 RPM, and max speed of 40 RPM. Mice were acclimated to testing room for 30 minutes prior to experimentation. Latency to fall was recorded in seconds for each mouse, and lanes were cleaned with 70% ethanol between mice.

### Catwalk Behavioral Task

The Catwalk apparatus (Noldus Information Technology, Leesburg, VA) consisted of an illuminated walled glass walkway (130 cm×10 cm) and a high-speed camera underneath. Mice were allowed 30 minutes to acclimate to the testing room, then behavioral task was performed with 11- to 12-week-old R6/2 mice on Day 25. Mice were placed on the glass walkway and allowed to ambulate freely across the runway towards the goal box at the end of the walkway. A goal box blocked the other end of the walkway, forcing the mouse to return to the entryway. Each mouse completed Catwalk runs until three compliant runs (fluent crossings without stopping/hesitation), were detected and verified, running for as long as needed to do so. Behavioral measures were calculated using Noldus Gait Analysis Software, and pairing and further comparisons were done using RM one-way ANOVAs.

### UHPLC-MS Analysis

Samples from animal studies were analyzed via UPLC-MS using a Waters Xevo G2-Xs QTof Mass spectrometer equipped with an Acquity UPLC. Chromatographic separation was carried out at 50° C. on an Acquity UPLC BEH C₁₈ column (1.7 μm, 2.1 mm×50 mm, pore size 130 Å) over a 4.5 min gradient elution. The flowrate was held constant at 0.8 mL/min. Mobile phase A consisted of water and mobile phase B consisted of ACN both containing 0.1% formic acid. After injection, the gradient was held at 50% A for 0.25 min. For the next minute, the gradient was ramped in a linear fashion to 100% B and held at this composition for 0.5 min. The eluent composition returned to the initial condition in 0.01 min and the column was re-equilibrated for 2.74 min before the next injection. Injection volumes were 0.5 μL (TH-2-31 40 mg/kg) and 1 μL for all other conditions. The Xevo G2-Xs was operated in positive electrospray ionization (ESI) mode. A capillary voltage and sampling cone voltage of 0.5 kV and 30 V were used. The source and desolvation temperatures were kept at 120° C. and 20° C., respectively. Nitrogen was used as the desolvation gas with a flowrate of 750 L/hr. The protonated molecular ion of leucine encephalin ([M+H]⁺, m/z 556.2771 was used as a lock mass for mass accuracy and reproducibility. Leucine enkephalin was introduced to the lock mass at a concentration of 2 ng/mL (50% ACN containing 0.1% formic acid), and a flow rate of 5 mL/min. To avoid signal saturation, the signal transmission was attenuated to <10%, based off the signal intensity of the highest standard concentration for the duration of the run. The data was collected over the mass range m/z 50 to 1200 Da with an acquisition time of 0.1 seconds per scan. The retention time for each analog is detailed below. All samples were injected twice, and the base peak chromatogram was integrated and quantified by standard curve concurrently ran using MassLynx software.

### Organic Synthesis

### General Synthetic Routes

Synthetic Route I

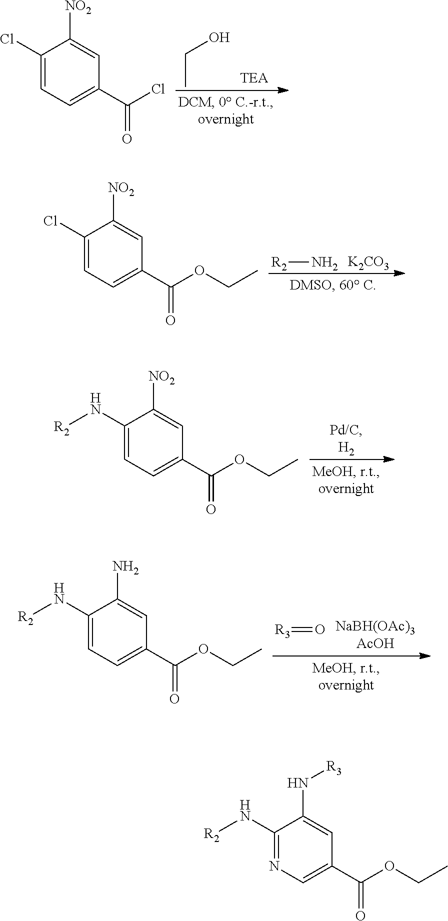

Synthetic Route II

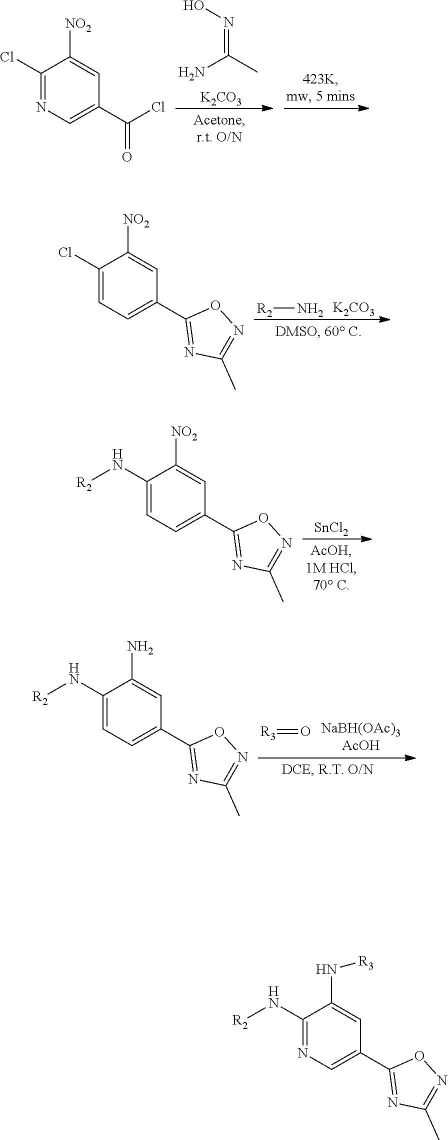

Synthetic Route III

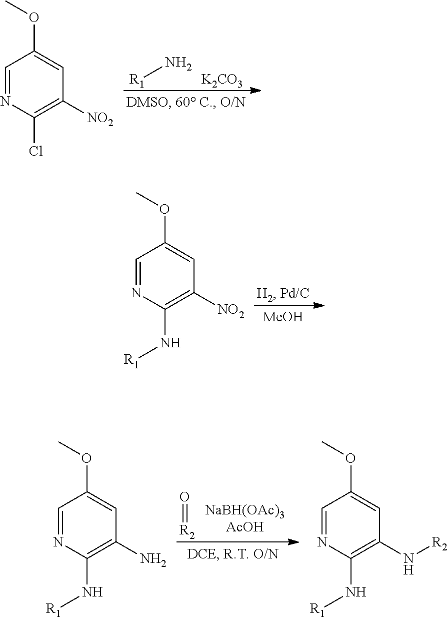

Synthetic Route IV

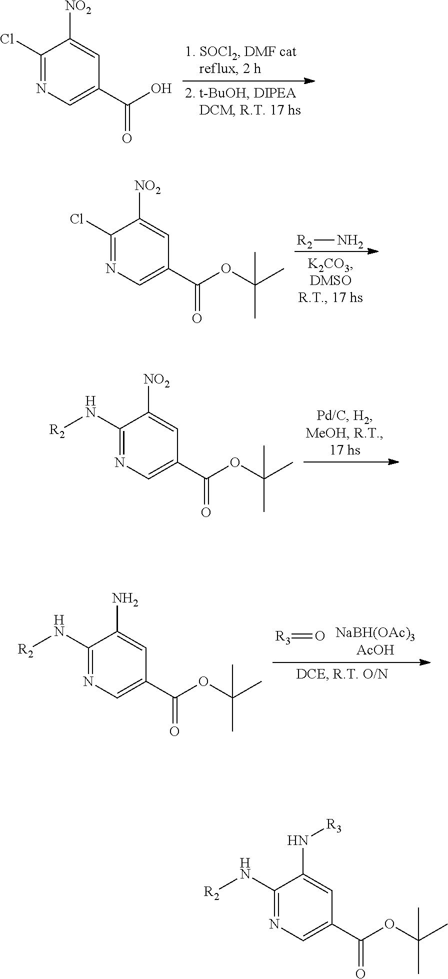

Synthetic Route V

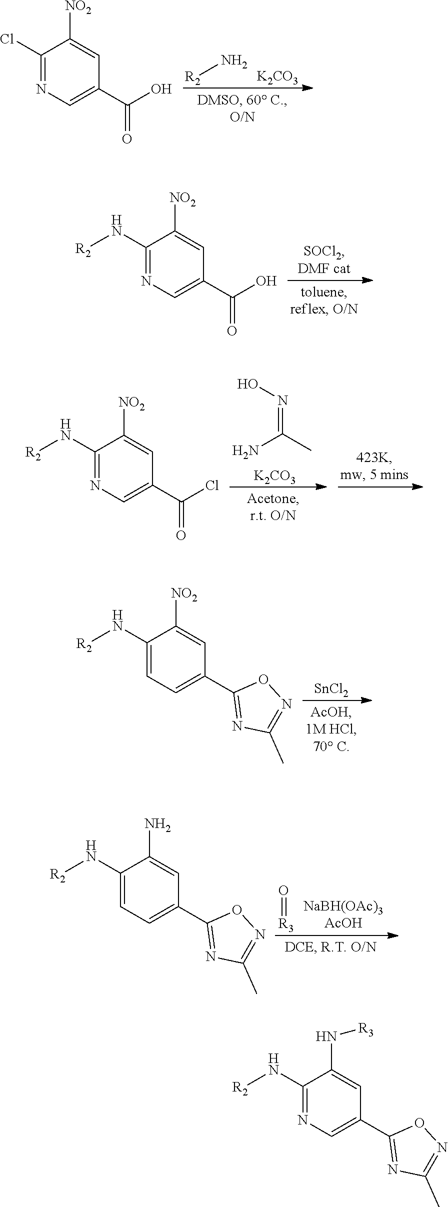

Synthetic Procedures

General Information

Starting materials were purchased form Sigma-Aldrich, Fisher Scientific, Ark Pharm, Oakwood Chemical, Cambridge Isotope Laboratory, or AK Scientific and were used as received unless stated otherwise. All solvents were reagent grade. Column chromatography was performed on a Teledyne ISCO CombiFlash® Rf+ using RediSep® Normal-phase silica flash columns. Thin layer chromatography (TLC) was performed on Silicycle SiliaPlate™ Glass TLC Plates (250 μm, 20×20 cm). 1H NMR spectra were recorded at ambient temperature using 400 MHz, or 500 MHz spectrometers as indicated. Chemical shifts are reported in ppm relative to the residual solvent peaks (1H NMR: DMSO-d6, δ 2.50; chloroform-d, δ 7.26; methanol-d4, δ 3.31). The following abbreviations are used to indicate multiplicity: s (singlet), d (doublet), t (triplet), q (quartet), hept (heptuplet), m (multiplet), br (broad). Mass spectra (MS) were acquired on a time-of-flight spectrometer with atmospheric pressure chemical ionization (APCI) or electron spray ionization (ESI), as indicated, and were obtained by peak matching. All reactions were run under an atmosphere of nitrogen or argon in glassware that was flame-dried under argon unless otherwise stated. Aqueous solutions were prepared from nanopure water with a resistivity over 18 MΩ·cm. Unless otherwise noted, all reagents were commercially available.

Abbreviations. AcOH=acetic acid, DCM=dichloromethane, DIPEA=diisopropylethyl amine, DMAP=4-dimethylaminopyridine, EtOAc=ethyl acetate, EtOH=ethanol, Et₂O=diethyl ether, MeCN=acetonitrile, MeOH=methanol, Na₂SO₄=sodium sulfate, NaHCO₃=sodium bicarbonate, NEt₃=triethylamine, NaBH(OAc)₃=Sodium triacetoxyborohydride, r.t.=room temperature, THF=terahydrofuran.

### General Procedure I (1)

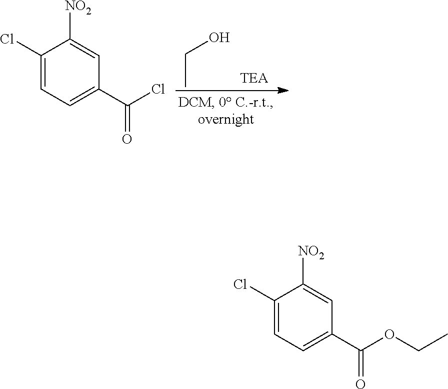

4-chloro-3-nitrobenzoyl chloride (220 mg, 1 mmol, 1.0 eq) and triethyl amine (209 uL, 1.5 mmol, 1.5 eq) were dissolved in dichloromethane (5 mL) and cooled to 0° C. in ice bath. Ethanol (70 uL, 1.2 mmol, 1.2 eq) was added dropwise. The reaction was slowly warmed to room temperature and stirred overnight. A saturated aqueous NaHCO₃ solution was added, the layers separated, and the aqueous layer extracted with dichloromethane. The combined organic layers were dried with Na₂SO₄, filtered, and the solvent evaporated. The crude material was purified by column chromatography on silica gel (0-40% ethyl acetate in hexanes) to afford the product.

### General Procedure I (2)

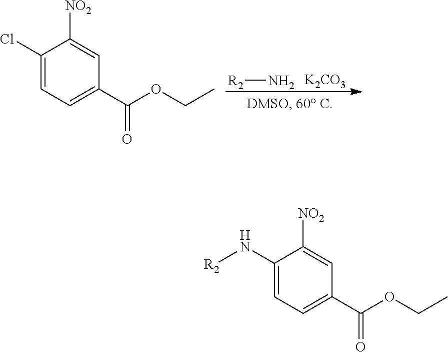

Ethyl 4-chloro-3-nitrobenzoate (1.0 eq.), substituted amine (1.2 eq.), and potassium carbonate (2.0 eq.) were dissolved in DMSO (0.25 M). the reaction was stirred at 60° C. overnight. After cooling to room temperature, the reaction mixture was partitioned between water and ethyl acetate. The layers were separated, and the aqueous layer was extracted with ethyl acetate (3×). The combined organic layers were washed with brine, dried with Na₂SO₄, filtered, and the solvent evaporated. The crude product was purified by column chromatography on silica gel (0-50% ethyl acetate in hexanes) to give the product.

### General Procedure I (3)

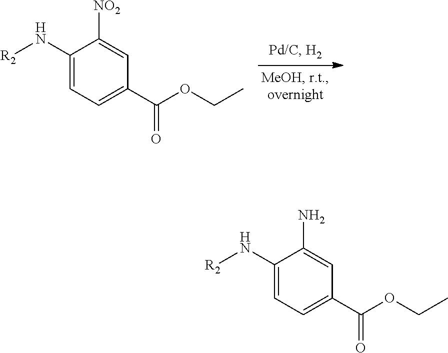

Substituted nitrobenzoate (1.0 eq.) and Pd (10 wt % on carbon, 0.2 eq.) were dissolved in methanol. The reaction was air exchanged to hydrogen gas and stirred under hydrogen gas (1 atm) overnight. The reaction mixture was filtered through celite and concentrated. The product was purified by column chromatography on silica gel (0-50% ethyl acetate in hexanes) to give the product.

### General Procedure I (4)

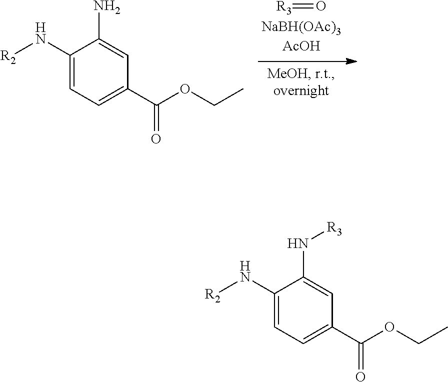

Substituted benzoate (1.0 eq), and ketone (1.0 eq) were dissolved in dichloroethane (0.1M) followed by addition of acetic acid (1.2 eq) and NaBH(OAc)₃ (1.2 eq). The reaction mixture was stirred at room temperature overnight. A saturated aqueous NaHCO₃ solution was added, the layers separated, and the aqueous layer extracted with dichloromethane. The combined organic layers were dried with Na₂SO₄, filtered, and the solvent evaporated. The crude material was purified by column chromatography on silica gel (0-50% ethyl acetate in hexanes) to afford the product.

### General Procedure II (1)

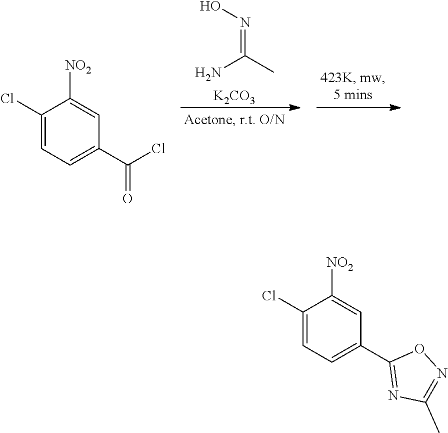

4-Chloro-3-nitrobenzoyl chloride (1.1 eq) was added to a solution of N′-hydroxyacetimidamide (1.0 eq) and K₂CO₃ (1.1 eq) in acetone (0.4 M) and stirred at room temperature overnight. The solvent was removed by rotatory evaporation, the residue was treated with water, and the precipitate was filtered off. The solid was heated 150° C. in microwave for 5 minutes. The residue was dissolved in dichloromethane and methanol, dried with MgSO₄, filtered, and the solvent was evaporated. The crude product was purified by column chromatography on silica gel (0-50% ethyl acetate in hexanes) to afford the product

### General Procedure II (2)

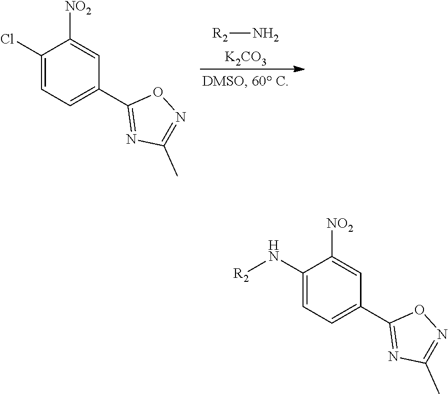

Substituted oxadiazole (1.0 eq.), substituted amine (1.2 eq.), and potassium carbonate (2.0 eq.) were dissolved in DMSO (0.2 M). the reaction was stirred at 60° C. overnight. After cooling to room temperature, the reaction mixture was partitioned between water and ethyl acetate. The layers were separated, and the aqueous layer was extracted with ethyl acetate (3×). The combined organic layers were washed with brine, dried with Na₂SO₄, filtered, and the solvent evaporated. The crude product was purified by column chromatography on silica gel (0-50% ethyl acetate in hexanes) to give the product.

### General Procedure II (3)

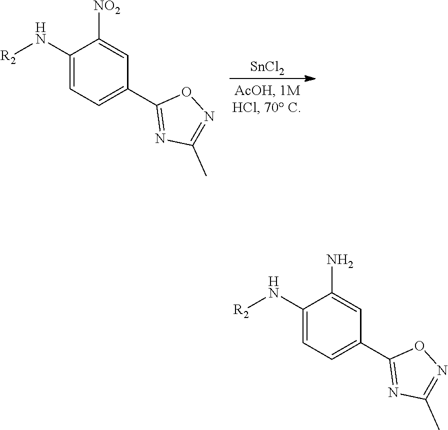

To a solution of substituted oxadiazole (1 eq.) in AcOH (0.2 M), HCl (1 N aqueous solution, 4 equiv.), and stannous chloride (5 equiv.) were added, and the reaction mixture was heated to 70° C. for 2 h. Upon completion the mixture was quenched with saturated aqueous sodium bicarbonate or sodium hydroxide (6N), filtered and the crude product was extracted twice with ethyl acetate. The combined organic layers were dried over sodium sulfate and concentrated under reduced pressure. Silica gel column chromatography (DCM/MeOH=100/0 to 90/10) afforded the corresponding aniline.

### General Procedure II (4)

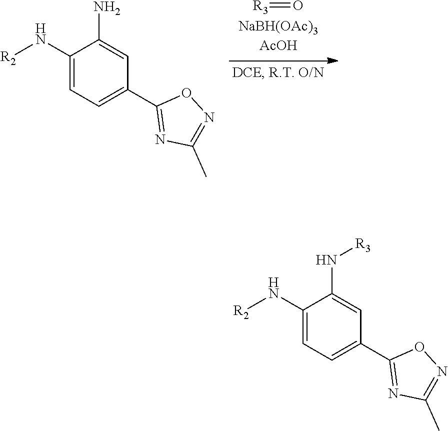

Aniline (1.0 eq), and ketone (1.0 eq) were dissolved in dichloroethane (0.1M) followed by addition of acetic acid (1.2 eq) and NaBH(OAc)₃ (1.2 eq). The reaction mixture was stirred at room temperature overnight. A saturated aqueous NaHCO₃ solution was added, the layers separated, and the aqueous layer extracted with dichloromethane. The combined organic layers were dried with Na₂SO₄, filtered, and the solvent evaporated. The crude material was purified by column chromatography on silica gel (DCM/MeOH=100/0 to 90/10) to afford the product.

### General Procedure III (1)

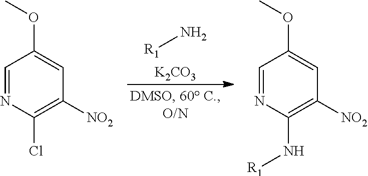

Substituted pyridine (1.0 eq), and substituted amine (1.2 eq.), and potassium carbonate (2.0 eq.) were dissolved in DMSO (0.2 M). the reaction was stirred at 60° C. overnight. After cooling to room temperature, the reaction mixture was partitioned between water and ethyl acetate. The layers were separated, and the aqueous layer was extracted with ethyl acetate (3×). The combined organic layers were washed with brine, dried with Na₂SO₄, filtered, and the solvent evaporated. The crude product was purified by column chromatography on silica gel (0-50% ethyl acetate in hexanes) to give the product.

### General Procedure III (2)

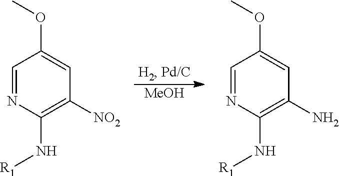

Substituted nitropyridine (1.0 eq.) and Pd (10 wt % on carbon, 0.2 eq.) were dissolved in methanol. The reaction was air exchanged to hydrogen gas and stirred under hydrogen gas (1 atm) overnight. The reaction mixture was filtered through celite and concentrated. The product was purified by column chromatography on silica gel (0-50% ethyl acetate in hexanes) to give the product.

### General Procedure III (3)

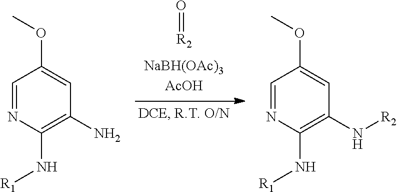

Aniline (1.0 eq), and ketone (1.0 eq) were dissolved in dichloroethane (0.1M) followed by the addition of acetic acid (1.2 eq) and NaBH(OAc)₃ (1.2 eq). The reaction mixture was stirred at room temperature overnight. A saturated aqueous NaHCO₃ solution was added, the layers separated, and the aqueous layer extracted with dichloromethane. The combined organic layers were dried with Na₂SO₄, filtered, and the solvent evaporated. The crude material was purified by column chromatography on silica gel (0-50% ethyl acetate in hexanes) to afford the product.

### General Procedure IV (1)

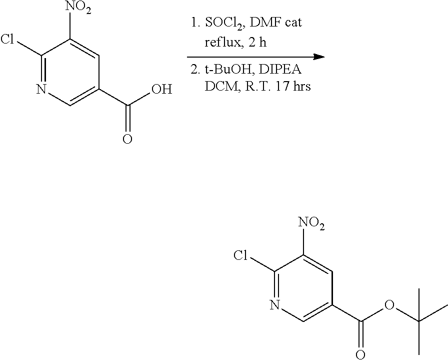 [SMILES: O=C(O)c1cnc(Cl)c([N+](=O)[O-])c1 ; CC(C)(C)OC(=O)c1cnc(Cl)c([N+](=O)[O-])c1]

Nitronicotinic acid (1.0 eq), thionyl chloride (2.0 eq.), and DMF (2 drops) were dissolved in toluene (0.2 M). The reaction was refluxed for 2 h. After cooling to room temperature, the reaction mixture was evaporated. The resulted solid was added to a solution of tert-Butyl alcohol (2.0 eq), DIPEA (2.0 eq) in DCM. The reaction mixture was stirred at room temperature overnight. A saturated aqueous NaHCO₃ solution was added, the layers separated, and the aqueous layer extracted with dichloromethane. The combined organic layers were dried with Na₂SO₄, filtered, and the solvent evaporated. The crude material was purified by column chromatography on silica gel (DCM/MeOH=100/0 to 90/10) to afford the product.

### General Procedure V (1)

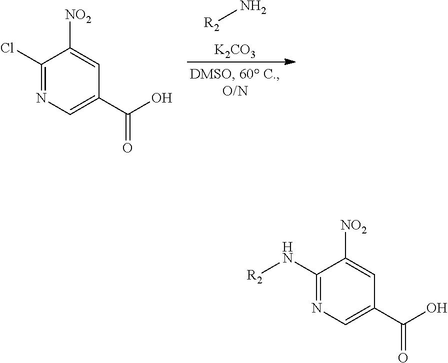

Nitronicotinic acid (1.0 eq), and substituted amine (1.2 eq.), and potassium carbonate (2.0 eq.) were dissolved in DMSO (0.2 M). the reaction was stirred at 60° C. overnight. After cooling to room temperature, the reaction mixture was partitioned between water and ethyl acetate. The layers were separated, and the aqueous layer was extracted with ethyl acetate (3×). The combined organic layers were washed with brine, dried with Na₂SO₄, filtered, and the solvent evaporated. The crude product was purified by column chromatography on silica gel (DCM/MeOH=100/0 to 90/10) to give the product.

### General Procedure V (2)

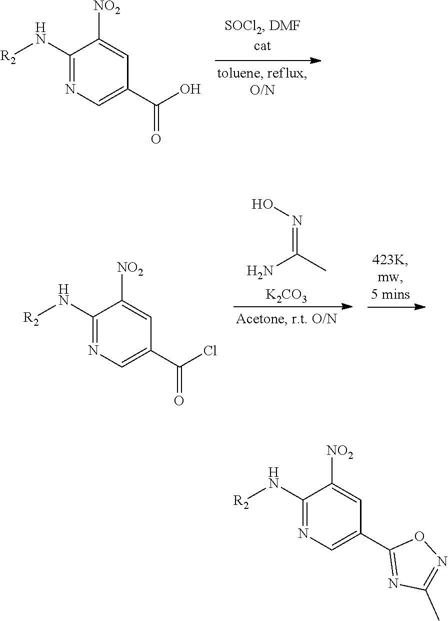

Substituted nitronicotinic acid (1.0 eq), Thionyl chloride (2.0 eq.), and DMF (2 drops) were dissolved in toluene (0.2 M). The reaction was refluxed overnight. After cooling to room temperature, the reaction mixture was evaporated. The resulted solid was added to a solution of N′-hydroxyacetimidamide (1.1 eq) and K₂CO₃ (1.1 eq) in acetone (0.4 M) and stirred at room temperature overnight. The solvent was removed by rotatory evaporation, the residue was treated with water, and the precipitate was filtered off. The solid was heated 150° C. in microwave for 5 minutes. The residue was dissolved in dichloromethane and methanol, dried with MgSO₄, filtered, and the solvent was evaporated. The crude product was purified by column chromatography on silica gel (DCM/MeOH=100/0 to 90/10) to afford the product.

### TH-1-45-1

### N1-cyclohexyl-N2-cyclopentyl-4-(3-methyl-1,2,4-oxadiazol-5-yl)benzene-1,2-diamine

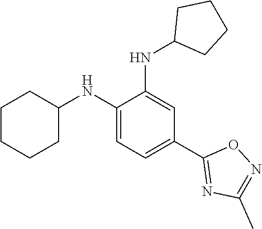 [SMILES: Cc1noc(-c2ccc(NC3CCCCC3)c(NC3CCCC3)c2)n1]

Following general procedure II(4) with N1-cyclohexyl-4-(3-methyl-1,2,4-oxadiazol-5-yl)benzene-1,2-diamine (80 mg, 0.29 mmol), N1-cyclohexyl-N2-cyclopentyl-4-(3-methyl-1,2,4-oxadiazol-5-yl)benzene-1,2-diamine (28.8 mg, 29% yield) was obtained as white solid.

¹H NMR (400 MHz, Chloroform-d) δ 7.60 (s, 1H), 7.42 (s, 1H), 6.67 (s, 1H), 3.85 (s, 1H), 3.33 (s, 1H), 2.42 (s, 3H), 2.08 (d, J=6.5 Hz, 4H), 1.89-1.56 (m, 13H), 1.48-1.34 (m, 3H).

MS (m/z): [MH]⁺ calculated for C₂₀H₂₉N₄O [M+H]⁺: 341.2341, found: 341.2351

### TH-1-45-2

### N1-cyclohexyl-N2-isopropyl-4-(3-methyl-1,2,4-oxadiazol-5-yl)benzene-1,2-diamine

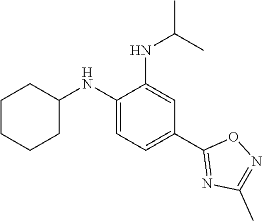 [SMILES: Cc1noc(-c2ccc(NC3CCCCC3)c(NC(C)C)c2)n1]

Following general procedure II(4) with N1-cyclohexyl-4-(3-methyl-1,2,4-oxadiazol-5-yl)benzene-1,2-diamine (80 mg, 0.29 mmol), N1-cyclohexyl-N2-isopropyl-4-(3-methyl-1,2,4-oxadiazol-5-yl)benzene-1,2-diamine (43 mg, 43% yield) was obtained as yellow solid.

¹H NMR (400 MHz, Chloroform-d) δ 7.60 (s, 1H), 7.41 (s, 1H), 6.66 (s, 1H), 3.85 (s, 1H), 3.33 (s, 1H), 2.42 (s, 3H), 2.16-2.07 (m, 3H), 1.89-1.48 (m, 12H), 1.48-1.34 (m, 3H).

MS (m/z): [MH]⁺ calculated for C₁₈H₂₇N₄O [M+H]⁺: 315.2185, found: 315.2199

### TH-1-45-3

### N1-cyclohexyl-4-(3-methyl-1,2,4-oxadiazol-5-yl)-N2-(pentan-3-yl)benzene-1,2-diamine

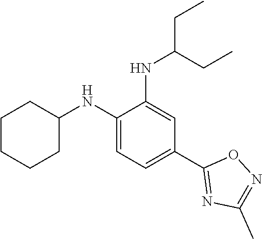 [SMILES: CCC(CC)Nc1cc(-c2nc(C)no2)ccc1NC1CCCCC1]

Following general procedure II(4) with N1-cyclohexyl-4-(3-methyl-1,2,4-oxadiazol-5-yl)benzene-1,2-diamine (80 mg, 0.29 mmol), N1-cyclohexyl-4-(3-methyl-1,2,4-oxadiazol-5-yl)-N2-(pentan-3-yl)benzene-1,2-diamine (21.8 mg, 22% yield) was obtained as white solid.

¹H NMR (400 MHz, Chloroform-d) δ 7.61 (s, 1H), 7.41 (s, 1H), 6.69 (s, 1H), 3.30 (d, J=41.8 Hz, 2H), 2.44 (s, 3H), 2.19-2.08 (m, 2H), 1.81 (s, 2H), 1.75-1.51 (m, 8H), 1.51-1.38 (m, 2H), 0.98 (m, 8H).

MS (m/z): [MH]⁺ calculated for C₂₀H₃₁N₄O [M+H]⁺: 343.2498, found: 343.2505

### TH-1-53-3

### N1-cyclohexyl-4-(3-methyl-1,2,4-oxadiazol-5-yl)-N2-(pyridin-4-ylmethyl)benzene-1,2-diamine

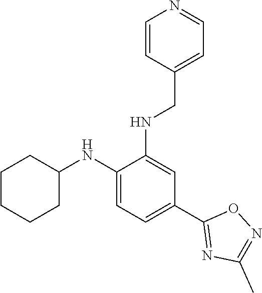 [SMILES: Cc1noc(-c2ccc(NC3CCCCC3)c(NCc3ccncc3)c2)n1]

Following general procedure II(4) with N1-cyclohexyl-4-(3-methyl-1,2,4-oxadiazol-5-yl)benzene-1,2-diamine (80 mg, 0.29 mmol), N1-cyclohexyl-4-(3-methyl-1,2,4-oxadiazol-5-yl)-N2-(pyridin-4-ylmethyl)benzene-1,2-diamine (25.2 mg, 24% yield) was obtained as yellow solid.

¹H NMR (400 MHz, Chloroform-d) δ 8.89-8.75 (m, 2H), 8.69 (s, 1H), 7.93 (dd, J=8.7, 2.0 Hz, 1H), 7.84 (d, J=1.9 Hz, 1H), 7.79-7.61 (m, 2H), 6.76 (d, J=8.7 Hz, 1H), 5.54 (s, 1H), 3.47 (s, 1H), 2.46 (s, 3H), 2.12 (q, J=5.3, 4.6 Hz, 2H), 1.84 (dt, J=12.9, 4.0 Hz, 2H), 1.71 (dd, J=12.5, 4.3 Hz, 2H), 1.53-1.24 (m, 6H).

MS (m/z): [M]⁺ calculated for C₂₂H₂₇N₄O [M]⁺: 362.2107, found: 362.1995

### YZ0996

### methyl 3,4-bis(cyclopentylamino)benzoate

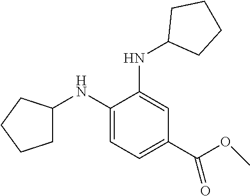 [SMILES: COC(=O)c1ccc(NC2CCCC2)c(NC2CCCC2)c1]

Following general procedure I(4) with methyl 3-amino-4-(cyclopentylamino)benzoate.

¹H NMR (400 MHz, Chloroform-d) δ 7.60-7.53 (m, 1H), 7.39 (s, 1H), 6.62 (d, J=8.3 Hz, 1H), 3.85 (s, 5H), 2.14-1.99 (m, 4H), 1.83-1.56 (m, 8H), 1.59-1.43 (m, 4H).

MS (m/z): [MH]⁺ calculated for C₁₈H₂₆N₂O₂ [M+H]⁺: 303.2072, found: 303.2069.

### YZ0997

### methyl 4-(cyclopentylamino)-3-(isopropylamino)benzoate

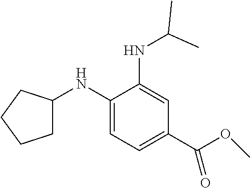 [SMILES: COC(=O)c1ccc(NC2CCCC2)c(NC(C)C)c1]

Following general procedure I(4) with methyl 3-amino-4-(cyclopentylamino)benzoate.

¹H NMR (400 MHz, Chloroform-d) δ 7.58 (s, 1H), 7.39 (s, 1H), 6.62 (d, J=8.3 Hz, 1H), 3.85 (s, 4H), 3.56 (s, 1H), 2.14-1.99 (m, 2H), 1.83-1.57 (m, 3H), 1.56-1.46 (m, 2H), 1.46-1.33 (m, 1H), 1.22 (d, J=6.2 Hz, 7H).

MS (m/z): [MH]⁺ calculated for C₁₆H₂₄N₂O₂ [M+H]⁺: 277.1916, found: 277.1914.

### YZ1108

### ethyl 5-amino-6-(cyclohexylamino)nicotinate

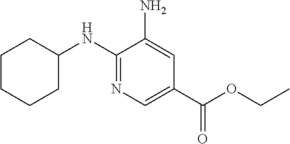 [SMILES: CCOC(=O)c1cnc(NC2CCCCC2)c(N)c1]

Following general procedure I(4) with ethyl 3-amino-4-(cyclohexylamino)benzoate.

¹H NMR (400 MHz, Chloroform-d) δ 8.46 (d, J=2.0 Hz, 1H), 7.40 (d, J=2.0 Hz, 1H), 4.72 (s, 1H), 4.31 (q, J=7.1 Hz, 2H), 3.19 (s, 2H), 2.08 (dq, J=14.1, 3.9 Hz, 2H), 1.81-1.60 (m, 3H), 1.53-1.45 (m, 1H), 1.45-1.29 (m, 4H), 1.29-1.14 (m, 3H).

MS (m/z): [MH]⁺ calculated for C₁₄H₂₁N₃O₂ [M+H]⁺: 264.1712, found: 264.1728

### YZ1109

### ethyl 5-(benzylamino)-6-(cyclohexylamino)nicotinate

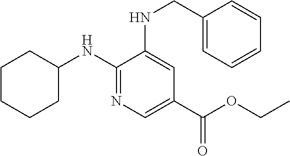 [SMILES: CCOC(=O)c1cnc(NC2CCCCC2)c(NCc2ccccc2)c1]

Following general procedure I(4) with ethyl 3-amino-4-(cyclohexylamino)benzoate.

¹H NMR (400 MHz, Chloroform-d) δ 8.71 (d, J=2.0 Hz, 1H), 8.62 (s, 1H), 7.95-7.87 (m, 2H), 7.73 (d, J=2.0 Hz, 1H), 7.57-7.45 (m, 3H), 7.44-7.24 (m, 2H), 5.91-5.84 (m, 1H), 4.35 (q, J=7.1 Hz, 2H), 4.10 (pd, J=7.4, 6.6, 4.1 Hz, 1H), 2.09 (dd, J=12.6, 4.1 Hz, 2H), 1.72 (ddt, J=41.7, 12.8, 4.0 Hz, 3H), 1.55-1.46 (m, 1H), 1.46-1.32 (m, 4H), 1.32-1.30 (m, 1H), 1.30-1.17 (m, 2H).

MS (m/z): [MH]⁺ calculated for C₂₁H₂₇N₃O₂ [M+H]⁺: 354.2181, found: 354.2157

### TH-2-5

### N²-cyclohexyl-5-methoxypyridine-2,3-diamine

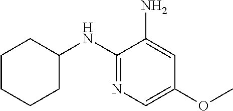 [SMILES: COc1cnc(NC2CCCCC2)c(N)c1]

Following general procedure III(2) with N-cyclohexyl-5-methoxy-3-nitropyridin-2-amine (15 mg, 0.06 mmol), N²-cyclohexyl-5-methoxypyridine-2,3-diamine (17 mg, 99% yield) was obtained as brown oil.

¹H NMR (400 MHz, Chloroform-d) δ 7.43 (d, J=2.6 Hz, 1H), 6.56 (d, J=2.6 Hz, 1H), 3.80 (ddd, J=14.4, 7.1, 4.1 Hz, 1H), 3.75 (s, 3H), 3.43-3.13 (m, 2H), 2.11-2.00 (m, 2H), 1.73 (dq, J=11.7, 3.8 Hz, 2H), 1.69-1.56 (m, 1H), 1.49-1.36 (m, 2H), 1.27-1.12 (m, 4H).

MS (m/z): [MH]⁺ calculated for C₁₂H₁₉N₃O [M+H]⁺: 222.1606, found: 222.1629

### TH-2-7

### N²-cyclohexyl-4-methoxypyridine-2,5-diamine

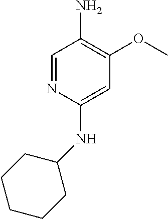 [SMILES: COc1cc(NC2CCCCC2)ncc1N]

Following general procedure III(1) with N-cyclohexyl-4-methoxy-5-nitropyridin-2-amine (13 mg, 0.052 mmol), N²-cyclohexyl-4-methoxypyridine-2,5-diamine (13 mg, 99% yield) was obtained as purple black oil.

¹H NMR (400 MHz, Chloroform-d) δ 7.40 (s, 1H), 5.89 (s, 1H), 3.90 (s, 3H), 3.48 (s, 1H), 3.40 (ddd, J=9.7, 5.9, 3.9 Hz, 2H), 2.04-1.92 (m, 2H), 1.85-1.71 (m, 2H), 1.62 (dt, J=11.9, 4.1 Hz, 1H), 1.44-1.21 (m, 6H).

MS (m/z): [MH]⁺ calculated for C₁₂H₁₉N₃O [M+H]⁺: 222.1606, found: 222.1625

### TH-2-8

### 6-chloro-N-cyclohexyl-4-methoxypyridin-3-amine

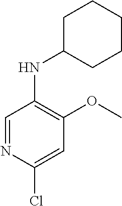 [SMILES: COc1cc(Cl)ncc1NC1CCCCC1]

Following general procedure I(4) with 6-chloro-4-methoxypyridin-3-amine (40 mg, 0.29 mmol), 6-chloro-N-cyclohexyl-4-methoxypyridin-3-amine (51 mg, 73% yield) was obtained as purple black oil.

¹H NMR (400 MHz, Chloroform-d) δ 7.60 (s, 1H), 6.67 (s, 1H), 3.91 (s, 3H), 3.28 (tt, J=10.0, 3.7 Hz, 1H), 2.17-2.00 (m, 2H), 1.83-1.59 (m, 4H), 1.48-1.35 (m, 2H), 1.33-1.16 (m, 3H).

MS (m/z): [MH]⁺ calculated for C₁₂H₁₈ClN₂O, 241.1108; found 241.1108.

### TH-2-9-1

### N²,N³-dicyclohexyl-5-methoxypyridine-2,3-diamine

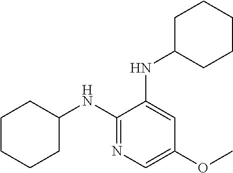 [SMILES: COc1cnc(NC2CCCCC2)c(NC2CCCCC2)c1]

Following general procedure I(4) with N²-cyclohexyl-5-methoxypyridine-2,3-diamine (17 mg, 0.08 mmol), N²,N³-dicyclohexyl-5-methoxypyridine-2,3-diamine (11 mg, 45% yield) was obtained as light yellow solid.

¹H NMR (400 MHz, Chloroform-d) δ 7.24 (d, J=2.5 Hz, 1H), 6.41 (dd, J=2.6, 0.6 Hz, 1H), 3.72 (s, 3H), 3.62 (tt, J=10.4, 3.8 Hz, 1H), 3.08 (tt, J=10.0, 3.7 Hz, 1H), 1.98 (dt, J=12.8, 3.8 Hz, 4H), 1.90-1.77 (m, 2H), 1.70 (tt, J=13.2, 3.7 Hz, 4H), 1.64-1.54 (m, 2H), 1.39-1.10 (m, 12H).

MS (m/z): [MH]⁺ calculated for C₁₈H₃₀N₃O, 304.2489; found 304.2396.

### TH-2-9-2

### N²,N⁵-dicyclohexyl-4-methoxypyridine-2,5-diamine

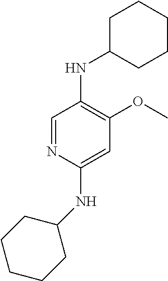 [SMILES: COc1cc(NC2CCCCC2)ncc1NC1CCCCC1]

Following general procedure I(4) with N-cyclohexyl-4-methoxy-5-nitropyridin-2-amine (13 mg, 0.06 mmol), N²,N⁵-dicyclohexyl-4-methoxypyridine-2,5-diamine (4 mg, 22% yield) was obtained as light yellow solid.

¹H NMR (400 MHz, Chloroform-d) δ 6.92 (s, 1H), 5.94 (s, 1H), 3.99 (s, 3H), 3.38-3.25 (m, 1H), 3.00 (tt, J=10.0, 3.7 Hz, 1H), 2.00 (td, J=13.0, 3.6 Hz, 4H), 1.89-1.72 (m, 4H), 1.66 (d, J=5.1 Hz, 2H), 1.54-1.08 (m, 12H).

MS (m/z): [MH]⁺ calculated for C₁₈H₃₀N₃O, 304.2489; found 304.2397.

### TH-3-83-2

### N2-isopropyl-6-methoxypyridine-2,3-diamine

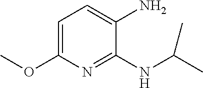

Following general procedure I(3) with N-isopropyl-6-methoxy-3-nitropyridin-2-amine (302 mg, 1.42 mmol), N2-isopropyl-6-methoxypyridine-2,3-diamine (214 mg, 47% yield) was obtained as purple solid.

¹H NMR (400 MHz, Chloroform-d) δ 6.80 (d, J=7.9 Hz, 1H), 5.81 (d, J=7.9 Hz, 1H), 4.14 (p, J=6.4 Hz, 2H), 3.76 (s, 3H), 2.70 (s, 1H), 1.50 (s, 1H), 1.18 (d, J=6.3 Hz, 6H).

MS (m/z): [M]⁺ calculated for C₉H₁₅N₃O, 181.1215; found 181.1237.

### TH-3-86-r2

### N2-cyclohexyl-N5,N5-diisopropyl-3-(3-methyl-1,2,4-oxadiazol-5-yl)pyridine-2,5-diamine

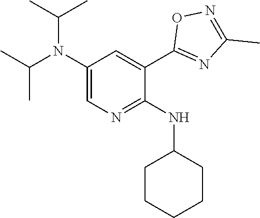 [SMILES: Cc1noc(-c2cc(N(C(C)C)C(C)C)cnc2NC2CCCCC2)n1]

Following general procedure II(4) with N2-cyclohexyl-3-(3-methyl-1,2,4-oxadiazol-5-yl)pyridine-2,5-diamine, N2-isopropyl-6-methoxypyridine-2,3-diamine was obtained as yellow solid as a side product.

¹H NMR (400 MHz, Chloroform-d) δ 8.54 (d, J=2.8 Hz, 1H), 8.32 (d, J=7.7 Hz, 1H), 8.18 (d, J=7.6 Hz, 1H), 2.51 (s, 3H), 2.07 (d, J=12.1 Hz, 2H), 1.85-1.74 (m, 2H), 1.74-1.58 (m, 1H), 1.58-1.31 (m, 6H), 1.36 (d, J=6.5 Hz, 12H).

MS (m/z): [MH]⁺ calculated for C₂₀H₃₂N₅O, 358.2607; found 358.2623.

### TH-3-95-1-r1

### N3-cyclohexyl-N2-isopropyl-6-methoxypyridine-2,3-diamine

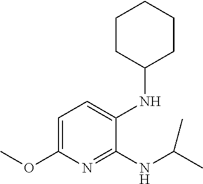 [SMILES: COc1ccc(NC2CCCCC2)c(NC(C)C)n1]

Following general procedure II(4) with N2-isopropyl-6-methoxypyridine-2,3-diamine (130 mg, 0.71 mmol), N3-cyclohexyl-N2-isopropyl-6-methoxypyridine-2,3-diamine (12 mg, 7% yield) was obtained as purple solid.

¹H NMR (400 MHz, Chloroform-d) δ 7.19 (d, J=8.4 Hz, 1H), 5.79 (d, J=8.4 Hz, 1H), 3.77 (s, 3H), 3.16 (tt, J=11.7, 3.8 Hz, 1H), 1.90 (d, J=12.4 Hz, 2H), 1.72 (d, J=10.1 Hz, 2H), 1.57 (s, 1H), 1.45-1.23 (m, 3H), 1.19 (t, J=7.1 Hz, 6H), 1.14 (d, J=6.5 Hz, 7H), 1.10 (d, J=9.4 Hz, 2H).

MS (m/z): [MH]⁺ calculated for C₁₅H₂₆N₃O, 264.2076; found 264.2101.

### TH-3-95-2-r1

### N2,N3-diisopropyl-6-methoxypyridine-2,3-diamine

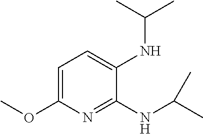 [SMILES: COc1ccc(NC(C)C)c(NC(C)C)n1]

Following general procedure II(4) with N2-isopropyl-6-methoxypyridine-2,3-diamine (86 mg, 0.47 mmol), N2,N3-diisopropyl-6-methoxypyridine-2,3-diamine (8 mg, 8% yield) was obtained as purple solid.

¹H NMR (400 MHz, Chloroform-d) δ 7.10 (d, J=8.3 Hz, 1H), 6.25 (d, J=8.2 Hz, 1H), 4.52 (p, J=6.6 Hz, 1H), 4.00 (p, J=6.5 Hz, 1H), 3.85 (s, 3H), 1.35 (t, J=6.5 Hz, 12H).

MS (m/z): [MH]⁺ calculated for C₁₂H₂₂N₃O, 224.1763; found 224.1785.

### TH-1-73

### tert-butyl 5-amino-2-(cyclohexylamino)nicotinate

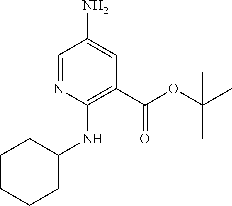 [SMILES: CC(C)(C)OC(=O)c1cc(N)cnc1NC1CCCCC1]

Following general procedure I(3) with tert-butyl 2-(cyclohexylamino)-5-nitronicotinate (60 mg, 0.19 mmol), tert-butyl 5-amino-2-(cyclohexylamino)nicotinate (40 mg, 69% yield) was obtained as yellow solid.

¹H NMR (400 MHz, Chloroform-d) δ 7.92 (d, J=3.0 Hz, 1H), 7.59 (d, J=3.0 Hz, 1H), 4.08-3.92 (m, 1H), 3.87-3.70 (m, 2H), 2.06 (dd, J=12.9, 4.0 Hz, 3H), 1.94-1.82 (m, 2H), 1.76 (dt, J=13.3, 4.1 Hz, 2H), 1.38-1.18 (m, 5H).

### TH-1-75

### tert-butyl 2,5-bis(cyclohexylamino)nicotinate

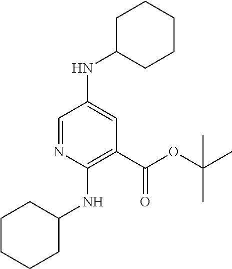 [SMILES: CC(C)(C)OC(=O)c1cc(NC2CCCCC2)cnc1NC1CCCCC1]

Following general procedure I(4) with tert-butyl 5-amino-2-(cyclohexylamino)nicotinate (40 mg, 0.14 mmol), tert-butyl 2,5-bis(cyclohexylamino)nicotinate (40 mg, 76% yield) was obtained as yellow solid.

¹H NMR (400 MHz, DMSO-d6) δ 7.86 (d, J=3.0 Hz, 1H), 7.44 (d, J=3.0 Hz, 1H), 7.19 (d, J=7.6 Hz, 1H), 4.81 (d, J=8.5 Hz, 1H), 3.95-3.82 (m, 1H), 3.09 (d, J=9.2 Hz, 1H), 1.94 (t, J=15.2 Hz, 4H), 1.73 (t, J=12.2 Hz, 4H), 1.61 (d, J=11.2 Hz, 1H), 1.57 (s, 9H), 1.44-1.09 (m, 12H).

MS (m/z): [MH]⁺ calculated for C22H36N3O2, 374.28; found 374.2831.

### TH-1-78

### tert-butyl 2-(((3s,5s,7s)-adamantan-1-yl)amino)-5-aminonicotinate

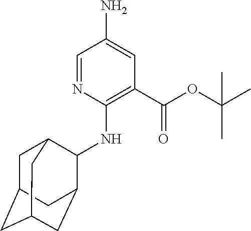 [SMILES: CC(C)(C)OC(=O)c1cc(N)cnc1NC1C2CC3CC(C2)CC1C3]

Following general procedure I(3) with tert-butyl 2-(((3s,5s,7s)-adamantan-1-yl)amino)-5-nitronicotinate (185 mg, 0.50 mmol), tert-butyl 2-(((3s,5s,7s)-adamantan-1-yl)amino)-5-aminonicotinate (114 mg, 67% yield) was obtained as yellow solid.

¹H NMR (400 MHz, Chloroform-d) δ 7.86 (d, J=3.1 Hz, 1H), 7.52 (d, J=3.1 Hz, 1H), 7.50 (s, 1H), 3.13 (s, 2H), 2.19-2.14 (m, 6H), 2.09 (d, J=4.5 Hz, 3H), 1.77-1.65 (m, 6H), 1.56 (s, 9H).

### TH-1-79

### tert-butyl 2-(((1r,3r,5r,7r)-adamantan-2-yl)amino)-5-(cyclohexylamino)nicotinate

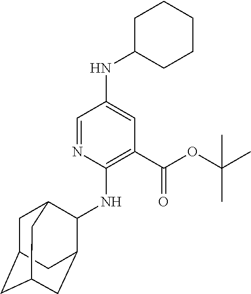 [SMILES: CC(C)(C)OC(=O)c1cc(NC2CCCCC2)cnc1NC1C2CC3CC(C2)CC1C3]

Following general procedure I(3) with tert-butyl 2-(((3s,5s,7s)-adamantan-1-yl)amino)-5-aminonicotinate (44 mg, 0.13 mmol), tert-butyl 2-(((1r,3r,5r,7r)-adamantan-2-yl)amino)-5-(cyclohexylamino)nicotinate (30 mg, 55% yield) was obtained as yellow solid.

¹H NMR (400 MHz, DMSO-d6) δ 7.78 (d, J=3.0 Hz, 1H), 7.40 (d, J=3.1 Hz, 1H), 4.74 (s, 1H), 3.06 (s, 1H), 2.11-2.02 (m, 9H), 1.94-1.84 (m, 2H), 1.75-1.69 (m, 2H), 1.66 (s, 6H), 1.53 (s, 9H), 1.38-1.04 (m, 7H).

MS (m/z): [MH]⁺ calculated for C26H40N3O2, 426.31; found 426.3120.

### TH-2-64-1

### tert-butyl 2-(cyclohexylamino)-5-(isopropylamino)nicotinate

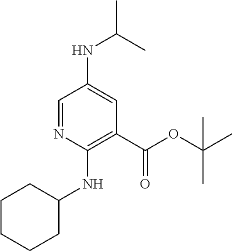 [SMILES: CC(C)Nc1cnc(NC2CCCCC2)c(C(=O)OC(C)(C)C)c1]

Following general procedure I(4) with tert-butyl 5-amino-2-(cyclohexylamino)nicotinate (30 mg, 0.11 mmol), tert-butyl 2-(cyclohexylamino)-5-(isopropylamino)nicotinate (10 mg, 68% yield) was obtained as brown solid.

¹H NMR (400 MHz, Chloroform-d) δ 7.84 (d, J=3.0 Hz, 1H), 7.49 (d, J=2.9 Hz, 1H), 3.99 (d, J=9.8 Hz, 1H), 3.06 (p, J=6.0 Hz, 1H), 2.07 (dd, J=12.3, 3.7 Hz, 2H), 1.76 (dt, J=13.3, 4.0 Hz, 2H), 1.60 (s, 9H), 1.58-1.41 (m, 5H), 1.33-1.26 (m, 2H).

MS (m/z): [MH]⁺ calculated for C19H31N3O2 [M+H]⁺: 334.2495, found: 334.2512.

### YZ1113

### tert-butyl 5-amino-6-(cyclohexylamino)nicotinate

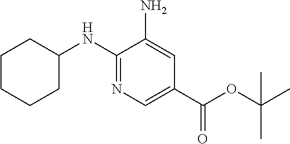 [SMILES: CC(C)(C)OC(=O)c1cnc(NC2CCCCC2)c(N)c1]

Following general procedure I(3) with tert-butyl 6-(cyclohexylamino)-5-nitronicotinate.

¹H NMR (400 MHz, Chloroform-d) δ 8.41 (d, J=2.0 Hz, 1H), 7.35 (d, J=2.0 Hz, 1H), 4.65 (s, 1H), 4.03 (d, J=6.9 Hz, 1H), 3.15 (s, 2H), 2.08 (dt, J=12.3, 4.0 Hz, 3H), 1.80-1.70 (m, 2H), 1.70-1.61 (m, 1H), 1.57 (s, 9H), 1.45 (dddd, J=18.1, 16.7, 7.7, 4.2 Hz, 2H), 1.29-1.14 (m, 3H).

MS (m/z): [MH]⁺ calculated for C16H25N3O2 [M+H]⁺: 292.2025, found: 292.2037

### YZ1114

### tert-butyl 5,6-bis(cyclohexylamino)nicotinate

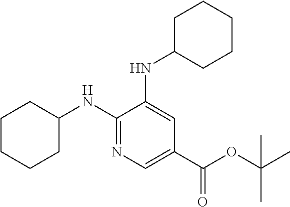 [SMILES: CC(C)(C)OC(=O)c1cnc(NC2CCCCC2)c(NC2CCCCC2)c1]

Following general procedure I(4) with tert-butyl 5-amino-6-(cyclohexylamino)nicotinate

¹H NMR (400 MHz, Chloroform-d) δ 8.37 (d, J=1.9 Hz, 1H), 7.31 (d, J=2.0 Hz, 1H), 4.70 (s, 1H), 4.00 (td, J=11.2, 10.6, 5.3 Hz, 1H), 3.14 (tt, J=10.2, 3.7 Hz, 1H), 2.12-1.96 (m, 2H), 1.78-1.60 (m, 3H), 1.55 (s, 9H), 1.51-1.12 (m, 10H).

MS (m/z): [MH]⁺ calculated for C22H35N3O2 [M+H]⁺: 374.2808, found: 374.2816

### YZ1117

### tert-butyl 6-(((3s,5s,7s)-adamantan-1-yl)amino)-5-aminonicotinate

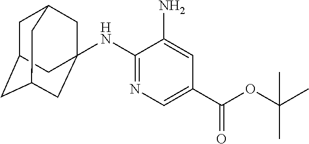 [SMILES: CC(C)(C)OC(=O)c1cnc(NC23CC4CC(CC(C4)C2)C3)c(N)c1]

Following general procedure I(3) with tert-butyl 6-(((3s,5s,7s)-adamantan-1-yl)amino)-5-nitronicotinate

¹H NMR (400 MHz, Chloroform-d) δ 8.41 (s, 1H), 7.36 (s, 1H), 7.25 (d, J=5.9 Hz, 8H), 3.07 (s, 2H), 2.19 (d, J=6.3 Hz, 7H), 2.13 (s, 4H), 1.77 (s, 3H), 1.72 (s, 5H), 1.30 (d, J=8.4 Hz, 1H), 1.25 (d, J=6.9 Hz, 3H), 0.85 (s, 1H).

MS (m/z): [MH]⁺ calculated for C20H29N3O2 [M+H]⁺: 344.2338, found: 344.2332.

### YZ1118

### tert-butyl 6-(((3s,5s,7s)-adamantan-1-yl)amino)-5-(cyclohexylamino)nicotinate

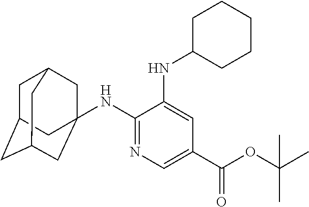 [SMILES: CC(C)(C)OC(=O)c1cnc(NC23CC4CC(CC(C4)C2)C3)c(NC2CCCCC2)c1]

Following general procedure I(4) with tert-butyl 6-(((3s,5s,7s)-adamantan-1-yl)amino)-5-aminonicotinate.

¹H NMR (400 MHz, Chloroform-d) δ 8.39 (s, 1H), 7.26 (t, J=5.0 Hz, 1H), 2.34 (s, 2H), 2.19 (s, 7H), 2.00 (s, 2H), 1.87 (s, 3H), 1.73 (s, 10H), 1.30 (d, J=36.1 Hz, 11H), 0.85 (s, 2H).

MS (m/z): [MH]⁺ calculated for C26H39N3O2 [M+H]⁺: 426.3120, found: 426.3112.

### TH-2-31

### N²,N³-dicyclohexyl-5-(3-methyl-1,2,4-oxadiazol-5-yl)pyridine-2,3-diamine

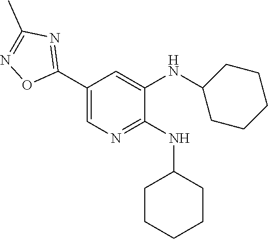 [SMILES: Cc1noc(-c2cnc(NC3CCCCC3)c(NC3CCCCC3)c2)n1]

Following general procedure II(4) with N²-cyclohexyl-5-(3-methyl-1,2,4-oxadiazol-5-yl)pyridine-2,3-diamine (43 mg, 0.16 mmol), N²,N³-dicyclohexyl-5-(3-methyl-1,2,4-oxadiazol-5-yl)pyridine-2,3-diamine (38 mg, 67% yield) was obtained as light yellow solid.

¹H NMR (400 MHz, Chloroform-d) δ 7.81 (d, J=1.7 Hz, 1H), 6.86 (d, J=1.9 Hz, 1H), 4.04-3.78 (m, 1H), 3.19 (td, J=10.0, 4.2 Hz, 1H), 2.44 (s, 3H), 2.01 (d, J=10.6 Hz, 4H), 1.89-1.64 (m, 4H), 1.58 (d, J=13.0 Hz, 1H), 1.47-1.01 (m, 9H).

MS (m/z): [MH]⁺ calculated for C20H29N5O [M+H]⁺: 356.2450, found: 356.2471.

### TH-2-37-1

### N²,N⁵-dicyclohexyl-3-(3-methyl-1,2,4-oxadiazol-5-yl)pyridine-2,5-diamine

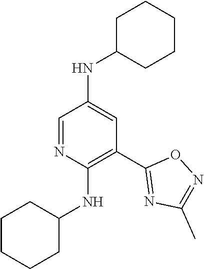 [SMILES: Cc1noc(-c2cc(NC3CCCCC3)cnc2NC2CCCCC2)n1]

Following general procedure II(4) with N²-cyclohexyl-3-(3-methyl-1,2,4-oxadiazol-5-yl)pyridine-2,5-diamine (20 mg, 0.073 mmol), N²,N⁵-dicyclohexyl-3-(3-methyl-1,2,4-oxadiazol-5-yl)pyridine-2,5-diamine (12 mg, 46% yield) was obtained as yellow solid.

¹H NMR (400 MHz, Chloroform-d) δ 8.15 (d, J=2.9 Hz, 1H), 8.09 (d, J=2.9 Hz, 1H), 4.08 (s, 1H), 3.21 (s, 1H), 2.53 (s, 3H), 2.07 (d, J=22.0 Hz, 4H), 1.87-1.66 (m, 6H), 1.62-1.46 (m, 2H), 1.45-1.16 (m, 10H).

MS (m/z): [MH]⁺ calculated for C20H29N5O [M+H]⁺: 356.2450, found: 356.2469.

### TH-2-37-2

### N²-cyclohexyl-N⁵-isopropyl-3-(3-methyl-1,2,4-oxadiazol-5-yl)pyridine-2,5-diamine

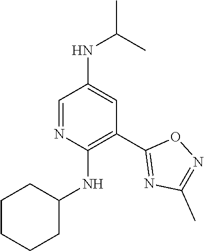 [SMILES: Cc1noc(-c2cc(NC(C)C)cnc2NC2CCCCC2)n1]

Following general procedure II(4) with N²-cyclohexyl-3-(3-methyl-1,2,4-oxadiazol-5-yl)pyridine-2,5-diamine (20 mg, 0.073 mmol), N²-cyclohexyl-N⁵-isopropyl-3-(3-methyl-1,2,4-oxadiazol-5-yl)pyridine-2,5-diamine (18 mg, 78% yield) was obtained as yellow solid.

¹H NMR (400 MHz, Chloroform-d) δ 7.92 (d, J=3.0 Hz, 1H), 7.54 (d, J=3.0 Hz, 1H), 7.50 (d, J=7.8 Hz, 1H), 4.11-3.98 (m, 1H), 2.93 (s, 1H), 2.47 (s, 3H), 2.05 (dd, J=12.3, 4.6 Hz, 2H), 1.76 (dt, J=13.1, 4.2 Hz, 2H), 1.63 (dt, J=12.5, 3.8 Hz, 1H), 1.60-1.40 (m, 4H), 1.40-1.25 (m, 3H), 1.20 (d, J=6.3 Hz, 6H).

MS (m/z): [MH]⁺ calculated for C17H25N5O [M+H]⁺: 316.2137, found: 316.2162.

### TH-4-16-1

### N²-cyclohexyl-N³-cyclopentyl-5-(3-methyl-1,2,4-oxadiazol-5-yl)pyridine-2,3-diamine

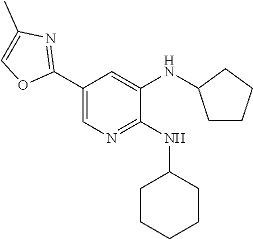 [SMILES: Cc1coc(-c2cnc(NC3CCCCC3)c(NC3CCCC3)c2)n1]

Following general procedure II(4) with N2-cyclohexyl-5-(3-methyl-1,2,4-oxadiazol-5-yl)pyridine-2,3-diamine (200 mg, 0.73 mmol), N²-cyclohexyl-N³-cyclopentyl-5-(3-methyl-1,2,4-oxadiazol-5-yl)pyridine-2,3-diamine (35 mg, 14% yield) was obtained as brown solid.

¹H NMR (400 MHz, Chloroform-d) δ 7.78 (d, J=1.8 Hz, 1H), 6.93 (dd, J=1.8, 0.7 Hz, 1H), 3.86-3.71 (m, 1H), 3.64 (t, J=6.0 Hz, 1H), 2.37 (s, 3H), 2.02-1.90 (m, 5H), 1.74-1.60 (m, 4H), 1.60-1.44 (m, 5H), 1.36-1.22 (m, 5H), 1.10-1.00 (m, 1H).

MS (m/z): [MH]⁺ calculated for C19H27N5O [M+H]⁺: 342.2294, found: 342.2301.

### TH-4-45

### N,N-diethyl-3-(3-methyl-1,2,4-oxadiazol-5-yl)-5-nitropyridin-2-amine

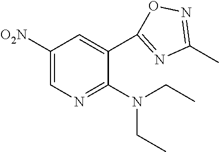 [SMILES: CCN(CC)c1ncc([N+](=O)[O-])cc1-c1nc(C)no1]

Following general procedure V(2) with 2-(diethylamino)-5-nitronicotinic acid (770 mg, 3.2 mmol), N,N-diethyl-3-(3-methyl-1,2,4-oxadiazol-5-yl)-5-nitropyridin-2-amine (153 mg, 19% yield) was obtained as yellow solid.

¹H NMR (400 MHz, Chloroform-d) δ 8.94 (d, J=2.4 Hz, 1H), 8.47 (s, 1H), 3.61 (q, J=7.2 Hz, 4H), 3.48 (s, 3H), 1.31-1.12 (m, 6H).

MS (m/z): [MH]⁺ calculated for C12H16N5O, 278.1253; found 278.1268.

### TH-4-46-2

### N,N-diethyl-5-(3-methyl-1,2,4-oxadiazol-5-yl)-3-nitropyridin-2-amine

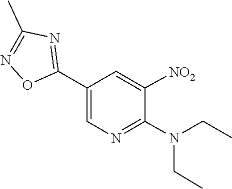 [SMILES: CCN(CC)c1ncc(-c2nc(C)no2)cc1[N+](=O)[O-]]

Following general procedure V(2) with 6-(diethylamino)-5-nitronicotinic acid (456 mg, 1.9 mmol), N,N-diethyl-5-(3-methyl-1,2,4-oxadiazol-5-yl)-3-nitropyridin-2-amine (7 mg, 1% yield) was obtained as yellow solid.

¹H NMR (400 MHz, Chloroform-d) δ 8.86 (d, J=2.1 Hz, 1H), 8.59 (d, J=2.1 Hz, 1H), 3.47 (q, J=7.1 Hz, 4H), 2.38 (s, 3H), 1.19 (t, J=7.1 Hz, 6H).

MS (m/z): [MH]⁺ calculated for C12H16N5O, 278.1253; found 278.1276.

### TH-4-48-2

### N-cyclohexyl-3-(3-methyl-1,2,4-oxadiazol-5-yl)-5-nitropyridin-2-amine

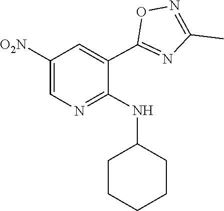 [SMILES: Cc1noc(-c2cc([N+](=O)[O-])cnc2NC2CCCCC2)n1]

Following general procedure V(1) and V(2) with 2-chloro-5-nitronicotinic acid (1 g, 4.92 mmol), N-cyclohexyl-3-(3-methyl-1,2,4-oxadiazol-5-yl)-5-nitropyridin-2-amine (72 mg, 5% yield) was obtained as yellow solid.

¹H NMR (400 MHz, Chloroform-d) δ 9.17 (dd, J=2.7, 0.4 Hz, 1H), 8.96 (d, J=2.7 Hz, 1H), 8.86 (d, J=8.1 Hz, 1H), 4.38-4.18 (m, 1H), 2.51 (s, 3H), 2.12-1.92 (m, 2H), 1.79 (dt, J=13.1, 4.1 Hz, 2H), 1.72-1.63 (m, 1H), 1.55-1.30 (m, 5H).

MS (m/z): [MH]⁺ calculated for C14H17N5O3, 304.1410; found 304.1431.

### TH-4-50-2

### N-cyclohexyl-5-(3-methyl-1,2,4-oxadiazol-5-yl)-3-nitropyridin-2-amine

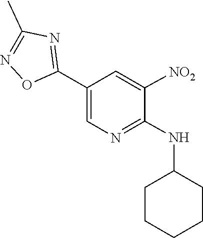 [SMILES: Cc1noc(-c2cnc(NC3CCCCC3)c([N+](=O)[O-])c2)n1]

Following general procedure V(1) and V(2) with 2-chloro-5-nitronicotinic acid (1 g, 4.92 mmol), N-cyclohexyl-5-(3-methyl-1,2,4-oxadiazol-5-yl)-3-nitropyridin-2-amine (25 mg, 2% yield) was obtained as yellow solid.

¹H NMR (400 MHz, Chloroform-d) δ 9.13-9.07 (m, 2H), 8.56 (d, J=7.7 Hz, 1H), 4.49-4.29 (m, 1H), 2.49 (s, 3H), 2.15-2.07 (m, 2H), 1.83 (dt, J=13.1, 4.0 Hz, 2H), 1.75-1.65 (m, 1H), 1.55-1.29 (m, 5H).

MS (m/z): [MH]⁺ calculated for C14H17N5O3, 304.1410; found 304.1407.

### TH-4-52-2

### N,N-diethyl-3-(3-methyl-1,2,4-oxadiazol-5-yl)-5-nitropyridin-2-amine

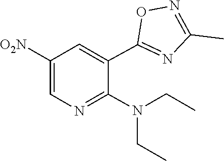 [SMILES: CCN(CC)c1ncc([N+](=O)[O-])cc1-c1nc(C)no1]

Following general procedure V(1) and V(2) with 2-amino-5-nitronicotinic acid (500 mg, 2.5 mmol), N,N-diethyl-3-(3-methyl-1,2,4-oxadiazol-5-yl)-5-nitropyridin-2-amine (30 mg, 4% yield) was obtained as yellow solid.

¹H NMR (400 MHz, Chloroform-d) δ 9.13 (d, J=2.7 Hz, 1H), 8.65 (d, J=2.7 Hz, 1H), 3.49 (q, J=7.1 Hz, 4H), 2.51 (s, 3H), 1.23 (t, J=7.1 Hz, 6H).

MS (m/z): [MH]⁺ calculated for C12H16N5O3, 278.1253; found 278.1269.

### TH-4-53-1

### N²-cyclohexyl-3-(3-methyl-1,2,4-oxadiazol-5-yl)-N⁵-(pentan-3-yl)pyridine-2,5-diamine

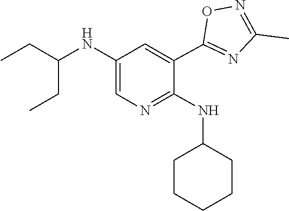 [SMILES: CCC(CC)Nc1cnc(NC2CCCCC2)c(-c2nc(C)no2)c1]

Following general procedure II(4) with N2-cyclohexyl-3-(3-methyl-1,2,4-oxadiazol-5-yl)pyridine-2,5-diamine (18 mg, 0.066 mmol), N²-cyclohexyl-3-(3-methyl-1,2,4-oxadiazol-5-yl)-N⁵-(pentan-3-yl)pyridine-2,5-diamine (8 mg, 36% yield) was obtained as yellow solid.

¹H NMR (400 MHz, Chloroform-d) δ 8.20 (s, 1H), 8.05 (s, 1H), 4.10 (s, 1H), 3.63-3.49 (m, 1H), 2.52 (s, 3H), 2.08 (d, J=12.0 Hz, 2H), 1.78 (d, J=13.4 Hz, 2H), 1.73-1.62 (m, 2H), 1.61-1.47 (m, 3H), 1.45-1.36 (m, 3H), 1.36-1.24 (m, 10H).

MS (m/z): [MH]⁺ calculated for C19H30N5O, 344.2450; found 344.2440.

### TH-4-53-2

### N²-cyclohexyl-N⁵-cyclopentyl-3-(3-methyl-1,2,4-oxadiazol-5-yl)pyridine-2,5-diamine

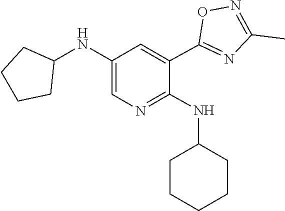 [SMILES: Cc1noc(-c2cc(NC3CCCC3)cnc2NC2CCCCC2)n1]

Following general procedure II(4) with N2-cyclohexyl-3-(3-methyl-1,2,4-oxadiazol-5-yl)pyridine-2,5-diamine (15 mg, 0.164 mmol), N²-cyclohexyl-N⁵-cyclopentyl-3-(3-methyl-1,2,4-oxadiazol-5-yl)pyridine-2,5-diamine (7 mg, 37% yield) was obtained as yellow solid.

¹H NMR (400 MHz, Chloroform-d) δ 8.00 (d, J=2.5 Hz, 1H), 7.92 (d, J=2.8 Hz, 1H), 3.92 (d, J=10.2 Hz, 1H), 3.67 (t, J=6.1 Hz, 1H), 2.46 (s, 3H), 2.08-1.88 (m, 4H), 1.79-1.54 (m, 6H), 1.45 (dd, J=15.4, 9.4 Hz, 4H), 1.39-1.12 (m, 4H), 0.78 (tt, J=13.9, 6.3 Hz, 2H).

MS (m/z): [MH]⁺ calculated for C19H27N5O, 342.2294; found 342.2303.

### TH-4-55-1

### N³-cyclobutyl-N²-cyclohexyl-5-(3-methyl-1,2,4-oxadiazol-5-yl)pyridine-2,3-diamine

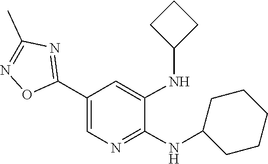 [SMILES: Cc1noc(-c2cnc(NC3CCCCC3)c(NC3CCC3)c2)n1]

Following general procedure II(4) with N2-cyclohexyl-5-(3-methyl-1,2,4-oxadiazol-5-yl)pyridine-2,3-diamine (18 mg, 0.066 mmol), N³-cyclobutyl-N²-cyclohexyl-5-(3-methyl-1,2,4-oxadiazol-5-yl)pyridine-2,3-diamine (15 mg, 70% yield) was obtained as brown solid.

¹H NMR (400 MHz, Chloroform-d) δ 8.41 (d, J=2.0 Hz, 1H), 7.15 (d, J=2.0 Hz, 1H), 4.46 (s, 1H), 3.98 (d, J=5.8 Hz, 1H), 3.92-3.78 (m, 1H), 2.51-2.42 (m, 2H), 2.36 (s, 3H), 2.03 (dd, J=12.4, 3.9 Hz, 2H), 1.87-1.74 (m, 4H), 1.70 (dt, J=13.2, 3.6 Hz, 2H), 1.66-1.58 (m, 1H), 1.47-1.34 (m, 2H), 1.18 (td, J=11.7, 11.3, 3.3 Hz, 4H).

MS (m/z): [MH]⁺ calculated for C18H26N5O, 328.2137; found 328.2148.

### TH-4-55-2

### N²-cyclohexyl-5-(3-methyl-1,2,4-oxadiazol-5-yl)-N³-(pentan-3-yl)pyridine-2,3-diamine

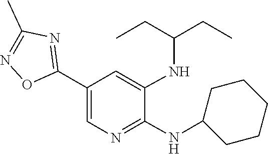 [SMILES: CCC(CC)Nc1cc(-c2nc(C)no2)cnc1NC1CCCCC1]

Following general procedure II(4) with N2-cyclohexyl-5-(3-methyl-1,2,4-oxadiazol-5-yl)pyridine-2,3-diamine (23 mg, 0.084 mmol N²-cyclohexyl-5-(3-methyl-1,2,4-oxadiazol-5-yl)-N³-(pentan-3-yl)pyridine-2,3-diamine (16 mg, 56% yield) was obtained as yellow oil.

¹H NMR (400 MHz, Chloroform-d) δ 8.40 (d, J=2.0 Hz, 1H), 7.41-7.24 (m, 1H), 3.97 (tt, J=10.5, 3.9 Hz, 1H), 3.14 (tt, J=5.9 Hz, 1H), 2.36 (s, 3H), 2.07-1.98 (m, 2H), 1.74-1.64 (m, 2H), 1.64-1.33 (m, 7H), 1.24-1.10 (m, 4H), 0.89 (t, J=7.4 Hz, 6H).

MS (m/z): [MH]⁺ calculated for C19H30N5O, 344.2450; found 344.2467.

### TH-4-58-2

### N-cyclopentyl-5-(3-methyl-1,2,4-oxadiazol-5-yl)-3-nitropyridin-2-amine

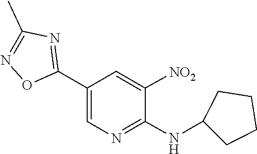 [SMILES: Cc1noc(-c2cnc(NC3CCCC3)c([N+](=O)[O-])c2)n1]

Following general procedure V(2) with 6-(cyclopentylamino)-5-nitronicotinic acid (1.51 g, 6 mmol), N-cyclohexyl-5-(3-methyl-1,2,4-oxadiazol-5-yl)-3-nitropyridin-2-amine (220 mg, 13% yield) was obtained as yellow solid.

¹H NMR (400 MHz, Chloroform-d) δ 9.10 (d, J=2.2 Hz, 1H), 9.08 (d, J=2.2 Hz, 1H), 8.59 (d, J=6.8 Hz, 1H), 4.70 (q, J=6.8 Hz, 1H), 2.48 (s, 3H), 2.26-2.11 (m, 2H), 1.90-1.79 (m, 2H), 1.79-1.71 (m, 2H), 1.69-1.57 (m, 3H).

### TH-4-62

### N³-cyclobutyl-N²-cyclopentyl-5-(3-methyl-1,2,4-oxadiazol-5-yl)pyridine-2,3-diamine

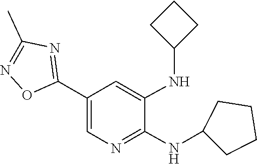 [SMILES: Cc1noc(-c2cnc(NC3CCCC3)c(NC3CCC3)c2)n1]

Following general procedure II(4) with N2-cyclohexyl-5-(3-methyl-1,2,4-oxadiazol-5-yl)pyridine-2,3-diamine (15 mg, 0.058 mmol), N³-cyclobutyl-N²-cyclopentyl-5-(3-methyl-1,2,4-oxadiazol-5-yl)pyridine-2,3-diamine (8 mg, 44% yield) was obtained as yellow solid.

¹H NMR (400 MHz, Chloroform-d) δ 8.08 (s, 1H), 6.92 (s, 1H), 4.30 (dd, J=8.0, 4.9 Hz, 1H), 3.79 (t, J=7.5 Hz, 1H), 2.46-2.38 (m, 2H), 2.36 (s, 3H), 2.12-2.00 (m, 2H), 1.95-1.74 (m, 4H), 1.74-1.62 (m, 2H), 1.62-1.48 (m, 4H).

MS (m/z): [MH]⁺ calculated for C17H24N5O, 314.1981; found 314.1995.

### TH-4-66

### N³-cyclohexyl-N²-cyclopentyl-5-(3-methyl-1,2,4-oxadiazol-5-yl)pyridine-2,3-diamine

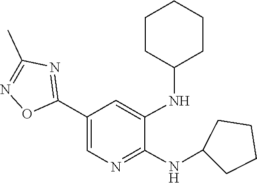 [SMILES: Cc1noc(-c2cnc(NC3CCCC3)c(NC3CCCCC3)c2)n1]

Following general procedure II(4) with N2-cyclohexyl-5-(3-methyl-1,2,4-oxadiazol-5-yl)pyridine-2,3-diamine (17 mg, 0.065 mmol), N³-cyclohexyl-N²-cyclopentyl-5-(3-methyl-1,2,4-oxadiazol-5-yl)pyridine-2,3-diamine (13 mg, 58% yield) was obtained as yellow solid.

¹H NMR (400 MHz, Chloroform-d) δ 7.88 (d, J=1.7 Hz, 1H), 7.00 (d, J=1.7 Hz, 1H), 4.29-4.15 (m, 1H), 3.24 (ddt, J=10.1, 7.2, 3.7 Hz, 1H), 2.48 (s, 3H), 2.18-2.00 (m, 4H), 1.89-1.58 (m, 8H), 1.47-1.20 (m, 6H).

MS (m/z): [MH]⁺ calculated for C19H28N5O, 342.2294; found 342.2304.

### TH-4-67

### N²²,N³-dicyclopentyl-5-(3-methyl-1,2,4-oxadiazol-5-yl)pyridine-2,3-diamine

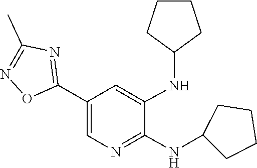 [SMILES: Cc1noc(-c2cnc(NC3CCCC3)c(NC3CCCC3)c2)n1]

Following general procedure II(4) with N2-cyclohexyl-5-(3-methyl-1,2,4-oxadiazol-5-yl)pyridine-2,3-diamine (17 mg, 0.065 mmol), N²,N³-dicyclopentyl-5-(3-methyl-1,2,4-oxadiazol-5-yl)pyridine-2,3-diamine (12 mg, 56% yield) was obtained as yellow solid.

¹H NMR (400 MHz, Chloroform-d) δ 7.74 (d, J=1.9 Hz, 1H), 6.84 (d, J=1.7 Hz, 1H), 4.27-4.07 (m, 1H), 3.58 (q, J=6.0 Hz, 1H), 2.49-2.27 (m, 3H), 2.10-1.90 (m, 4H), 1.80-1.45 (m, 13H), 1.20 (d, J=7.1 Hz, 2H), 0.88-0.76 (m, 1H).

MS (m/z): [MH]⁺ calculated for C18H26N5O, 328.2137; found 328.2147.

### TH-4-68

### N²-cyclopentyl-5-(3-methyl-1,2,4-oxadiazol-5-yl)-N³-(pentan-3-yl)pyridine-2,3-diamine

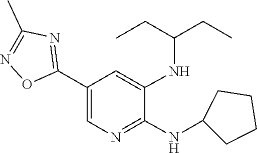 [SMILES: CCC(CC)Nc1cc(-c2nc(C)no2)cnc1NC1CCCC1]

Following general procedure II(4) with N2-cyclohexyl-5-(3-methyl-1,2,4-oxadiazol-5-yl)pyridine-2,3-diamine (15 mg, 0.065 mmol), N²,N³-dicyclopentyl-5-(3-methyl-1,2,4-oxadiazol-5-yl)pyridine-2,3-diamine (2 mg, 11% yield) was obtained as yellow solid.

¹H NMR (400 MHz, Chloroform-d) δ 7.91 (d, J=1.7 Hz, 1H), 6.97 (s, 1H), 4.32 (t, J=6.0 Hz, 1H), 3.23 (t, J=6.0 Hz, 1H), 2.47 (s, 3H), 2.14-2.02 (m, 4H), 1.65 (m, 10H), 0.96 (t, J=7.4 Hz, 4H).

MS (m/z): [MH]⁺ calculated for C18H28N5O, 330.2294; found 330.2304.

### TH-4-99-2-mw-r2

### N-((3s,5s,7s)-adamantan-1-yl)-5-(3-methyl-1,2,4-oxadiazol-5-yl)-3-nitropyridin-2-amine

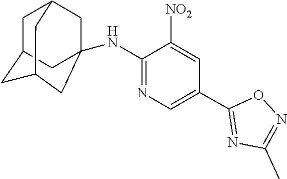 [SMILES: Cc1noc(-c2cnc(NC34CC5CC(CC(C5)C3)C4)c([N+](=O)[O-])c2)n1]

Following general procedure V(2) with 6-(((3s,5s,7s)-adamantan-1-yl)amino)-5-nitronicotinic acid (480 mg, 1.51 mmol), N-cyclohexyl-5-(3-methyl-1,2,4-oxadiazol-5-yl)-3-nitropyridin-2-amine (150 mg, 28% yield) was obtained as white solid.

¹H NMR (400 MHz, Chloroform-d) δ 9.08 (d, J=2.3 Hz, 1H), 9.06 (d, J=2.3 Hz, 1H), 8.62 (s, 1H), 2.48 (s, 3H), 2.28 (d, J=3.1 Hz, 6H), 2.23-2.17 (m, 3H), 1.79 (p, J=2.3 Hz, 6H).

MS (m/z): [MH]⁺ calculated for C18H21N5O3, 357.1751; found 357.1756.

### TH-4-100-2

### N²-((3s,5s,7s)-adamantan-1-yl)-N³-cyclohexyl-5-(3-methyl-1,2,4-oxadiazol-5-yl) pyridine-2,3-diamine

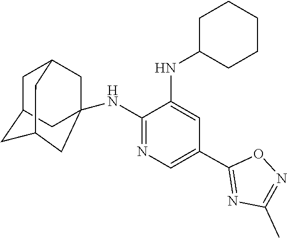 [SMILES: Cc1noc(-c2cnc(NC34CC5CC(CC(C5)C3)C4)c(NC3CCCCC3)c2)n1]

Following general procedure II(4) with N2-((3s,5s,7s)-adamantan-1-yl)-5-(3-methyl-1,2,4-oxadiazl-5-yl)pyridine-2,3-diamine (100 mg, 0.31 mmol), N²-((3s,5s,7s)-adamantan-1-yl)-N³-cyclohexyl-5-(3-methyl-1,2,4-oxadiazol-5-yl)pyridine-2,3-diamine (83 mg, 66% yield) was obtained as yellow solid.

¹H NMR (400 MHz, Chloroform-d) δ 8.48 (d, J=2.1 Hz, 1H), 7.36 (d, J=2.1 Hz, 1H), 4.75 (s, 1H), 3.19 (ddd, J=10.2, 6.3, 3.8 Hz, 1H), 2.65 (s, 1H), 2.44 (s, 3H), 2.21 (d, J=2.9 Hz, 6H), 2.19-2.11 (m, 3H), 2.04 (dd, J=13.4, 4.0 Hz, 2H), 1.84-1.66 (m, 10H), 1.49-1.35 (m, 2H), 1.32-1.20 (m, 4H).

### AZIII4

### tert-butyl 4-(((3s,5s,7s)-adamantan-1-yl)amino)-5-amino-2-methylbenzoate

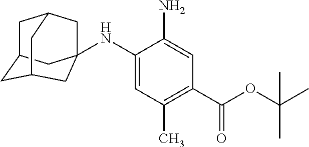 [SMILES: Cc1cc(NC23CC4CC(CC(C4)C2)C3)c(N)cc1C(=O)OC(C)(C)C]

¹H NMR (400 MHz, Chloroform-d) δ 7.32 (s, 1H), 6.80 (s, 1H), 2.51 (s, 3H), 2.15 (s, 3H), 2.00 (d, J=2.9 Hz, 6H), 1.71 (d, J=3.3 Hz, 6H), 1.59 (s, 9H).

MS (m/z): [MH]⁺ calculated for C22H33N2O2, 357.2542; found 357.2557.

### AZIII78

### tert-butyl 4-(((3s,5s,7s)-adamantan-1-yl)amino)-5-amino-2-fluorobenzoate

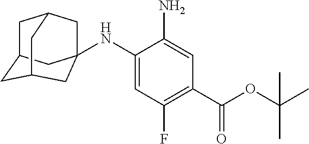 [SMILES: CC(C)(C)OC(=O)c1cc(N)c(NC23CC4CC(CC(C4)C2)C3)cc1F]

¹H NMR (400 MHz,) δ 7.22 (d, J=7.6 Hz, 1H), 6.58 (d, J=13.9 Hz, 1H), 2.96 (s, 2H), 2.15 (s, 3H), 1.98 (d, J=2.9 Hz, 6H), 1.72 (d, J=3.2 Hz, 6H), 1.56 (s, 9H).

### AZIII8

### tert-butyl 4-(((3s,5s,7s)-adamantan-1-yl)amino)-5-(cyclohexylamino)-2-fluorobenzoate

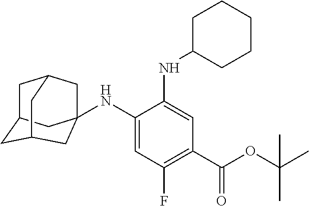 [SMILES: CC(C)(C)OC(=O)c1cc(NC2CCCCC2)c(NC23CC4CC(CC(C4)C2)C3)cc1F]

¹H NMR (500 MHz,) δ 7.26 (d, 1H), 6.53 (d, J=14.0 Hz, 1H), 2.94-2.80 (m, 1H), 2.14 (s, 3H), 1.96 (d, J=2.9 Hz, 8H), 1.79-1.65 (m, 8H), 1.63 (dt, J=12.3, 3.6 Hz, 1H), 1.56 (s, 9H), 1.35-1.10 (m, 6H).

MS (m/z): [MH]⁺ calculated for C27H40FN₂O2, 443.3074; found 443.3099.

### AZIII9

### tert-butyl 4-(((3s,5s,7s)-adamantan-1-yl)amino)-5-(cyclohexylamino)-2-methylbenzoate

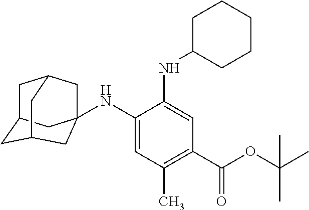 [SMILES: Cc1cc(NC23CC4CC(CC(C4)C2)C3)c(NC2CCCCC2)cc1C(=O)OC(C)(C)C]

¹H NMR (500 MHz,) δ 7.32 (s, 1H), 6.69 (s, 1H), 3.08-2.94 (m, 1H), 2.48 (s, 3H), 2.12 (s, 3H), 2.03-1.90 (m, 8H), 1.82-1.67 (m, 8H), 1.67-1.60 (m, 1H), 1.57 (s, 9H), 1.38-1.12 (m, 6H).

MS (m/z): [MH]⁺ calculated for C28H43N2O2, 439.3325; found 439.3341.

### AZII11

### tert-butyl 4-(((3s,5s,7s)-adamantan-1-yl)amino)-2-chloro-5-(cyclohexylamino)benzoate

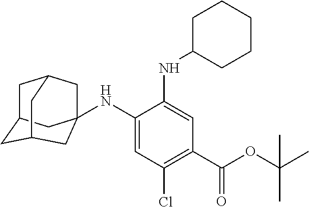 [SMILES: CC(C)(C)OC(=O)c1cc(NC2CCCCC2)c(NC23CC4CC(CC(C4)C2)C3)cc1Cl]

¹H NMR (500 MHz,) δ 7.19 (s, 1H), 6.87 (s, 1H), 3.09-2.98 (m, 1H), 2.34 (t, J=6.8 Hz, 1H), 2.13 (s, 3H), 1.97 (d, J=12.7 Hz, 2H), 1.91 (d, J=2.9 Hz, 6H), 1.87 (t, J=6.4 Hz, 1H), 1.81-1.61 (m, 8H), 1.58 (s, 9H), 1.41-1.07 (m, 6H).

MS (m/z): [MH]⁺ calculated for C27H40N2O2Cl, 459.2778; found 459.2766.

### DL032

### tert-butyl 4-(((3s,5s,7s)-adamantan-1-yl)amino)-3-(cyclohexylamino)benzoate

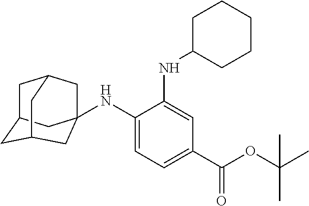 [SMILES: CC(C)(C)OC(=O)c1ccc(NC23CC4CC(CC(C4)C2)C3)c(NC2CCCCC2)c1]

Following general procedure I(4) with tert-butyl 4-(((3s,5s,7s)-adamantan-1-yl)amino)-3-aminobenzoate, tert-butyl 4-(((3s,5s,7s)-adamantan-1-yl)amino)-3-(cyclohexylamino)benzoate (0.1269 g, 40% yield) was obtained as a brown solid.

¹H NMR (400 MHz, CDCl₃) δ ppm: 7.39 (dd, 1H), 7.34 (d, 1H), 6.88 (d, 1H), 3.15-3.08 (m, 1H) 2.12 (br s, 3H), 2.03-1.98 (br m, 2H), 1.93 (br d, 6H), 1.78-1.62 (br m, 9H), 1.56 (s, 9H) 1.40-1.15 (br m, 5H)

MS (m/z): [MH]⁺ calculated for C27H41N2O2, 425.3168; found 425.3160.

### DL034

### tert-butyl 3-(((3s,5s,7s)-adamantan-1-yl)amino)-4-(cyclohexylamino)benzoate

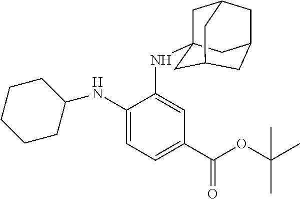 [SMILES: CC(C)(C)OC(=O)c1ccc(NC2CCCCC2)c(NC23CC4CC(CC(C4)C2)C3)c1]

Following general procedure I(4) with tert-butyl 3-(1-adamantylamino)-4-aminobenzoate (0.2416 g, 0.71 mmol), tert-butyl 3-(((3s,5s,7s)-adamantan-1-yl)amino)-4-(cyclohexylamino)benzoate (0.1561 g, 52% yield) was obtained as a white solid.

¹H NMR (400 MHz, CDCl₃) δ ppm: 7.66 (dd, 1H), 7.52 (d, 1H), 6.49 (d, 1H), 5.39 (br d, 1H), 3.34-3.25 (m, 1H), 2.25 (br s, 1H), 2.06-2.00 (br m, 5H), 1.80-1.73 (br m, 8H), 1.65-1.60 (br m, 7H), 1.56 (s, 9H), 1.40-1.19 (br m, 5H)

MS (m/z): [MH]⁺ calculated for C27H41N2O2, 425.3168; found 425.3160.

### DL047

### tert-butyl 4-(((3s,5s,7s)-adamantan-1-yl)amino)-3-((2,6-dimethylbenzyl)amino)benzoate

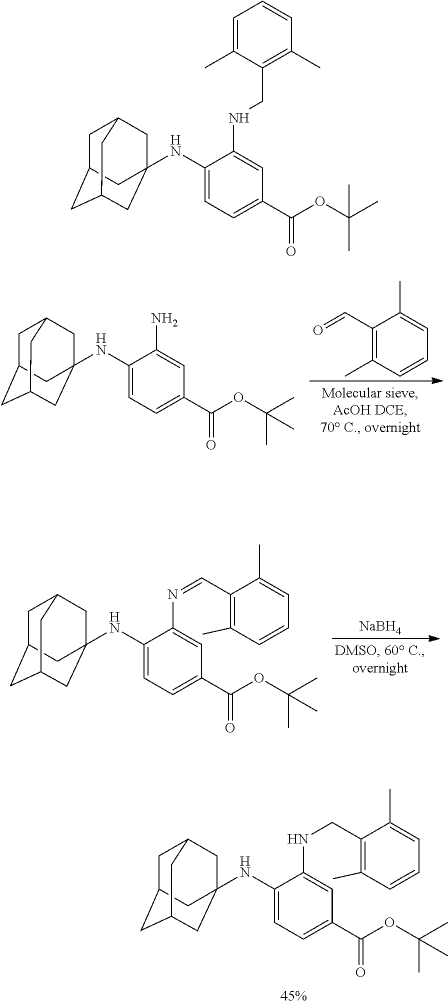

4-(1-adamantylamino)-3-aminobenoate (0.0506 g, 0.15 mmol, 1.0 equiv) was dissolved in 1,2-dichloroethane (2 mL). A solution of 2,6-dimethylbenzaldehyde (0.0460 g, 0.34 mmol, 2.3 equiv) dissolved in 1,2-dichloroethane (2 mL) was added to the reaction flask at room temperature. Molecular sieve (4 Å) and glacial acetic acid (15 μL, 0.26 mmol, 1.7 equiv) were added to the solution at room temperature. The reaction mixture was heated at 70° C. and stirred overnight under nitrogen atmosphere.

After completion of the reaction, 1,2-dichloroethane was evaporated and DMSO (19 mL) was added to dissolve the product. Sodium borohydride (0.4184 g, 11.06 mmol, 73.7 equiv) was added to reaction flask at room temperature. The reaction mixture was heated at 60° C. and stirred overnight under nitrogen atmosphere. Then, the reaction mixture was quenched with saturated NaHCO₃ aqueous solution, and the product was extracted with ethyl acetate. Combined organic layers were washed with water, dried (MgSO₄) and purified by flash-column chromatography on silica gel (hexane, ethyl acetate gradient 18% max). The yellow solid (0.3067 g, 0.067 mmol) with 45% yield.

¹H NMR (400 MHz, CDCl₃) δ ppm: 7.51 (d, 1H), 7.47 (dd, 1H), 7.15 (dd, 1H), 7.08 (d, 2H), 6.94 (d, 1H), 4.23 (s, 2H), 2.40 (s, 6H), 2.09 (br s, 3H), 1.89 (br d, 6H), 1.71-1.63 (br m, 6H), 1.60 (s, 9H)

MS (m/z): [MH]⁺ calculated for C30H41N2O2, 461.3168; found 461.3163.

### PHB4051

### N1-cyclohexyl-4-(oxazol-2-yl)benzene-1,2-diamine

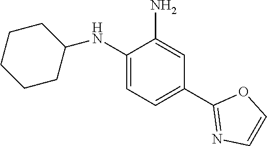 [SMILES: Nc1cc(-c2ncco2)ccc1NC1CCCCC1]

A mixture of 4-chloro-3-nitrobenzoyl chloride (726 mg, 3.3 mmol, 1.1 eq), 1H-1,2,3-triazole (207 mg, 174 μL, 3.0 mmol, 1.0 eq), and K₂CO₃ (912 mg, 6.6 mmol, 2.2 eq) in sulfolane (40 mL) was heated at 140° C. for 15 h. After cooling to room temperature, the mixture was partitioned between 100 mL of water and 100 mL of ethyl acetate. The aqueous layer was extracted with ethyl acetate and the combined organic layer was washed with water, dried with MgSO₄, filtered, and concentrated. The crude product was purified by column chromatography on silica gel (0-50% ethyl acetate in hexanes) to afford 2-(4-chloro-3-nitrophenyl)oxazole (108 mg, 16% yield). Following general procedure II(2) and 1(3) with the above intermediate (100 mg, 0.445 mmol), N1-cyclohexyl-4-(oxazol-2-yl)benzene-1,2-diamine (21.3 mg, 3% yield) was obtained as a colorless solid.

¹H NMR (400 MHz, Chloroform-d) δ 7.60 (d, J=0.8 Hz, 1H), 7.51 (dd, J=8.3, 2.0 Hz, 1H), 7.42 (d, J=2.0 Hz, 1H), 7.14 (d, J=0.8 Hz, 1H), 6.66 (d, J=8.4 Hz, 1H), 3.41 (s, 3H), 3.31 (tt, J=10.2, 3.7 Hz, 1H), 2.15-2.01 (m, 2H), 1.85-1.73 (m, 2H), 1.73-1.60 (m, 1H), 1.49-1.32 (m, 2H), 1.32-1.12 (m, 3H) ppm.

MS (ESI⁺, m/z): calcd. for C₁₅H₂₀N₃O [M+H]⁺: 258.1618, found: 258.1606.

### PHB4066

### N1-((3s,5s,7s)-adamantan-1-yl)-4-(3-methyl-1,2,4-oxadiazol-5-yl)benzene-1,2-diamine

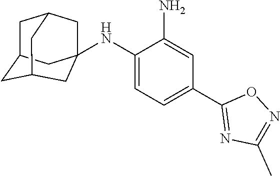 [SMILES: Cc1noc(-c2ccc(NC34CC5CC(CC(C5)C3)C4)c(N)c2)n1]

Following general procedure II(3) with N-(4-(3-methyl-1,2,4-oxadiazol-5-yl)-2-nitrophenyl)adamantan-1-amine (150 mg, 0.421 mmol), N1-((3s,5s,7s)-adamantan-1-yl)-4-(3-methyl-1,2,4-oxadiazol-5-yl)benzene-1,2-diamine (38 mg, 28% yield) was obtained as a colorless solid.

¹H NMR (400 MHz, Chloroform-d) δ 7.54 (dd, J=8.4, 2.0 Hz, 1H), 7.45 (d, J=2.0 Hz, 1H), 6.99 (d, J=8.4 Hz, 1H), 3.84 (s, 1H), 3.33 (s, 2H), 2.42 (s, 3H), 2.16 (s, 3H), 2.01 (d, J=2.9 Hz, 6H), 1.73 (d, J=3.1 Hz, 6H).

MS (ESI⁺, m/z): calcd. for C₁₉H₂₅N₄O [M+H]⁺: 325.2028, found: 325.2023.

### PHB4078

### N1-cyclohexyl-4-(3-methyl-1,2,4-oxadiazol-5-yl)benzene-1,2-diamine

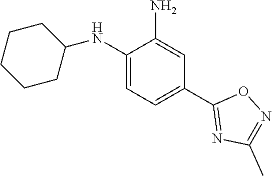 [SMILES: Cc1noc(-c2ccc(NC3CCCCC3)c(N)c2)n1]

Following general procedure II(3) with N-cyclohexyl-4-(3-methyl-1,2,4-oxadiazol-5-yl)-2-nitroaniline (90.7 mg, 0.3 mmol), N1-cyclohexyl-4-(3-methyl-1,2,4-oxadiazol-5-yl)benzene-1,2-diamine (57.3 mg, 70% yield) was obtained as a colorless solid.

¹H NMR (400 MHz, Chloroform-d) δ 7.61 (dd, J=8.4, 2.0 Hz, 1H), 7.45 (d, J=2.0 Hz, 1H), 6.67 (dd, J=8.4, 0.6 Hz, 1H), 3.95 (s, 1H), 3.36 (ddd, J=13.9, 9.9, 3.6 Hz, 1H), 3.26 (s, 2H), 2.42 (s, 3H), 2.17-2.02 (m, 2H), 1.80 (dt, J=12.8, 3.8 Hz, 2H), 1.69 (dt, J=12.8, 3.8 Hz, 1H), 1.49-1.34 (m, 2H), 1.33-1.15 (m, 3H) ppm.

MS (ESI⁺, m/z): calcd. for C₁₅H₂₀N₄O [M+H]⁺: 273.1724, found: 273.1715.

### PHB4081

### tert-butyl 4-(cyclohexylamino)-3-(phenylamino)benzoate

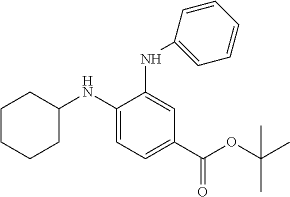 [SMILES: CC(C)(C)OC(=O)c1ccc(NC2CCCCC2)c(Nc2ccccc2)c1]

Following general procedure I(4) with tert-butyl 4-amino-3-(phenylamino)benzoate (0.1424 g, 0.50 mmol), tert-butyl 4-(cyclohexylamino)-3-(phenylamino)benzoate (0.0548 g, 30% yield) was obtained as a white solid.

¹H NMR (400 MHz, DMSO) δ ppm: 7.48-7.44 (m, 2H), 7.34-7.30 (m, 3H), 7.19 (s, 1H), 7.16 (dd, 1H), 6.46 (d, 1H), 6.34 (d, 1H), 1.87 (d, 2H), 1.61-1.23 (m, 17H), 0.98-0.91 (m, 1H); ¹³C NMR (400 MHz, DMSO)

MS (ESI⁺, m/z): calcd. for C₂₃H₃₁N₂O₂ [M+H]⁺: 367.2386, found: 367.2386.

### PHB4082

### N1,N2-dicyclohexyl-4-(3-methyl-1,2,4-oxadiazol-5-yl)benzene-1,2-diamine

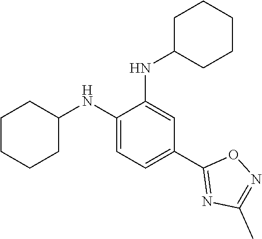 [SMILES: Cc1noc(-c2ccc(NC3CCCCC3)c(NC3CCCCC3)c2)n1]

Following general procedure II(4) with N¹-Cyclohexyl-4-(3-methyl-1,2,4-oxadiazol-5-yl)benzene-1,2-diamine (372 mg, 1.37 mmol), N1,N2-dicyclohexyl-4-(3-methyl-1,2,4-oxadiazol-5-yl)benzene-1,2-diamine (341 mg, 70% yield) was obtained as a colorless solid.

¹H NMR (400 MHz, Chloroform-d) δ 7.60 (d, J=8.3 Hz, 1H), 7.41 (s, 1H), 6.66 (d, J=8.4 Hz, 1H), 4.12 (s, 1H), 3.33 (s, 1H), 3.21 (s, 1H), 2.76 (s, 1H), 2.42 (s, 3H), 2.07 (d, J=12.4 Hz, 4H), 1.77 (s, 4H), 1.68 (dq, J=11.9, 3.7 Hz, 2H), 1.50-1.33 (m, 4H), 1.33-1.14 (m, 6H) ppm.

MS (ESI⁺, m/z): calcd. for C₂₁H₃₁N₄O [M+H]⁺: 355.2500, found: 355.2498.

### PHB4083

### N1-((3s,5s,7s)-adamantan-1-yl)-N2-cyclohexyl-4-(3-methyl-1,2,4-oxadiazol-5-yl)benzene-1,2-diamine

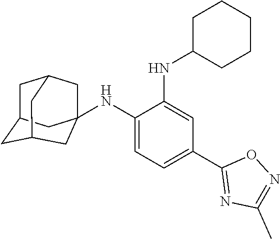 [SMILES: Cc1noc(-c2ccc(NC34CC5CC(CC(C5)C3)C4)c(NC3CCCCC3)c2)n1]

Following general procedure II(4) with N¹-(Adamantan-1-yl)-4-(3-methyl-1,2,4-oxadiazol-5-yl)benzene-1,2-diamine (32 mg, 0.1 mmol), N1-((3s,5s,7s)-adamantan-1-yl)-N2-cyclohexyl-4-(3-methyl-1,2,4-oxadiazol-5-yl)benzene-1,2-diamine (16.5 mg, 41% yield) was obtained as a colorless solid.

¹H NMR (400 MHz, Chloroform-d) δ 7.50 (dd, J=8.3, 2.0 Hz, 1H), 7.39 (d, J=2.0 Hz, 1H), 6.96 (d, J=8.4 Hz, 1H), 3.55 (bs, 2H), 3.16 (tt, J=10.0, 3.8 Hz, 1H), 2.43 (s, 3H), 2.14 (s, 3H), 2.07-1.98 (m, 2H), 1.95 (d, J=2.9 Hz, 6H), 1.82-1.59 (m, 9H), 1.46-1.31 (m, 2H), 1.31-1.14 (m, 3H) ppm.

MS (ESI⁺, m/z): calcd. for C₂₅H₃₅N₄O [M+H]⁺: 407.2808, found: 407.2811.

### Results

### Development and Characterization of a 4^{th} Ferrostatin

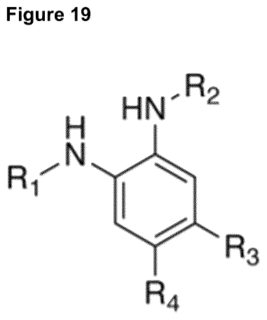

It was previously reported on the structure-activity-relationship (SAR) for ferrostatin analogs, indicating that compound potency is dependent on compound lipophilicity, with the incorporation of substitutions in the aniline ring being tolerated (Hofmans et al. 2016; Devisscher et al. 2018; Galluzzi et al. 2018). This SAR resulted in a general ferrostatin structure with four substitution sites for further analog development (FIG. 19). Our initial efforts were focused on increasing lipophilicity at the R₁ and R₂ positions. Large cycloalkyl groups were adopted on both amines, as they can confer favorable ADME properties and potency (Stimac et al. 2017; Lin, Kwong, and Perni 2006). The incorporation of adamantyl, cyclohexyl, and cyclopentyl rings at these positions resulted in analogs with improved in vitro potency relative to fer-1.

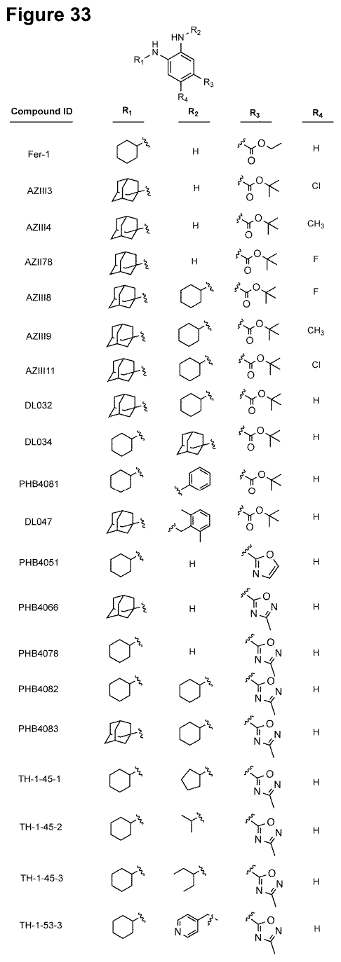

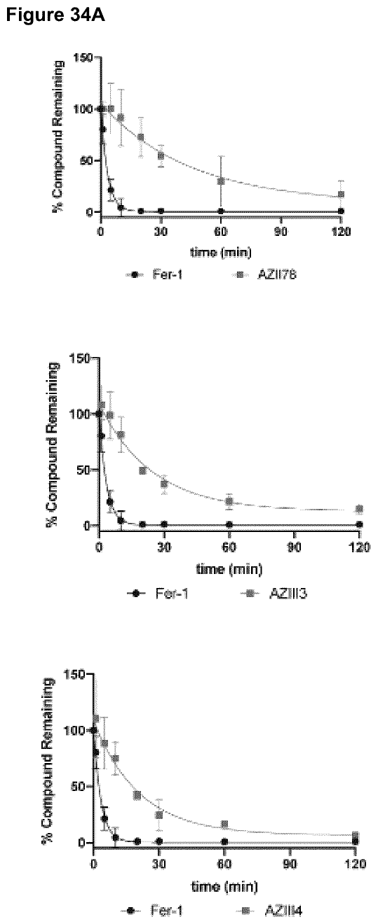

Given the susceptibility of the benzylic position to oxidation, we designed fer-1 analogs with a steric blocking tert-butyl ester on the R₃ position to hinder accessibility of this position to cytochrome P450 enzymes. Additionally, analogs with ester bioisosteres were designed and synthesized to minimize metabolism by plasma esterases (Kumari et al. 2020; Biernacki et al. 2020). These analogs were evaluated and prioritized according to predicted metabolic stability and other ADME properties (Table 10) (Schrödinger Release 2018-1: Maestro (2018), Schrödinger, LLC, New York, NY). A total of 19 compounds (FIG. 33) in this fourth-generation series were synthesized. All compounds were prepared following the strategies shown in Scheme 22.

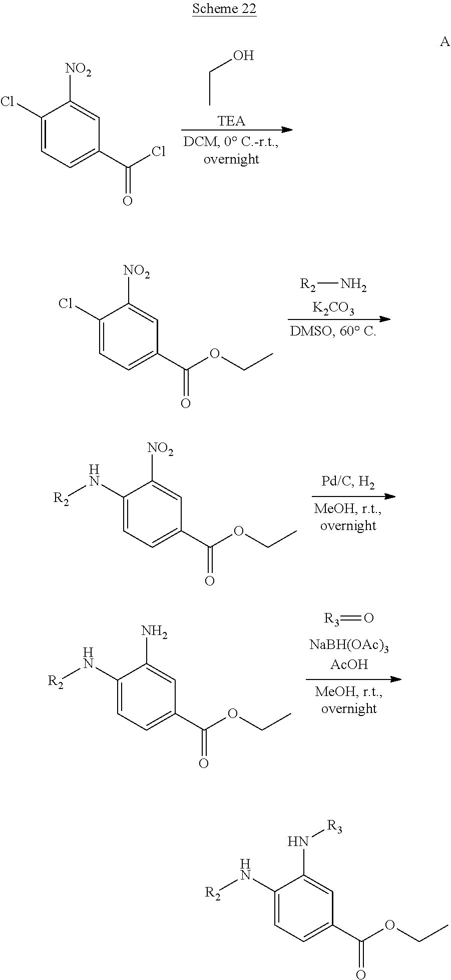

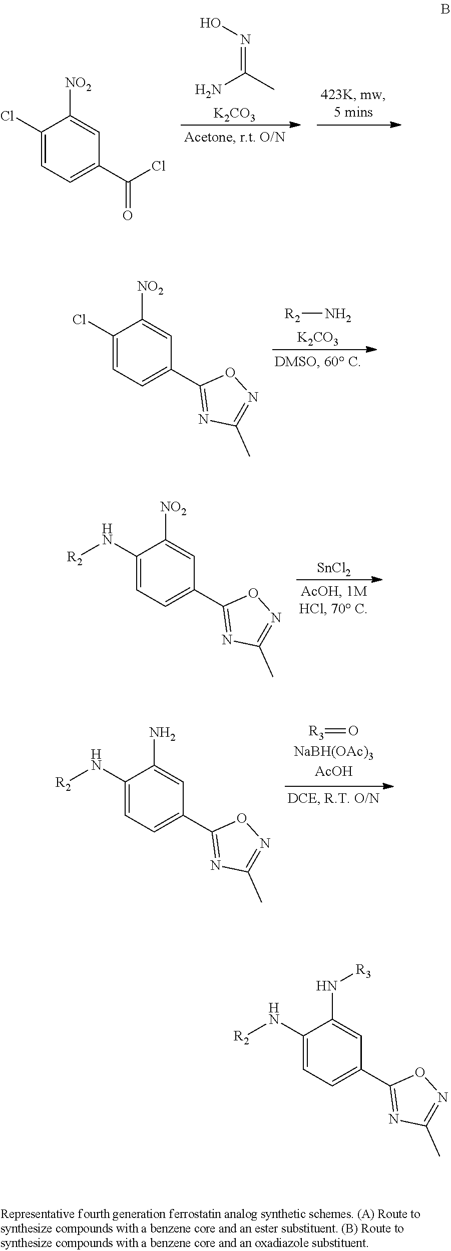

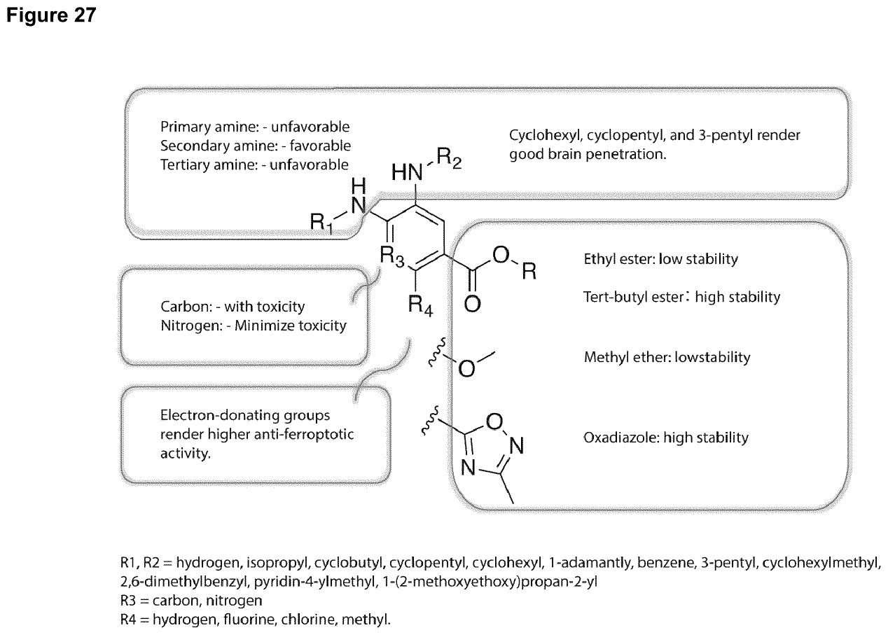

TABLE 10
Predicted ADME properties of the fourth and fifth generation,
related to FIG. 27

| molecule | mol_ | donor- | | QPlogPo/ | | |
|---|---|---|---|---|---|---|
| | MW | HB | accptHB | w | QPlogBB | PSA |
| AZII78 | 360.471 | 2.5 | 4 | 4.25 | −0.604 | 65.323 |
| AZIII11 | 459.07 | 2 | 4 | 6.84 | −0.115 | 49.781 |
| AZIII3 | 376.925 | 2.5 | 4 | 4.502 | −0.541 | 64.851 |
| AZIII4 | 356.507 | 2.5 | 4 | 4.318 | −0.629 | 63.253 |
| AZIII8 | 442.616 | 2 | 4 | 6.637 | −0.17 | 50.595 |
| AZIII9 | 438.652 | 2 | 4 | 6.644 | −0.226 | 48.253 |
| DL032 | 424.625 | 2 | 4 | 6.367 | −0.245 | 50.935 |
| DL034 | 424.625 | 2 | 4 | 6.299 | −0.233 | 50.977 |
| DL047 | 460.658 | 2 | 4 | 7.276 | −0.283 | 50.499 |
| PHB4051 | 257.335 | 2.5 | 4 | 2.66 | −0.375 | 56.646 |
| PHB4051 | 257.335 | 2.5 | 4 | 2.674 | −0.361 | 57.942 |
| PHB4066 | 324.425 | 2.5 | 5 | 2.86 | −0.805 | 74.45 |
| PHB4066 | 324.425 | 2.5 | 5 | 2.862 | −0.819 | 75.724 |
| PHB4078 | 272.349 | 2.5 | 5 | 2.2 | −0.842 | 75.441 |
| PHB4078 | 272.349 | 2.5 | 5 | 2.213 | −0.823 | 76.724 |
| PHB4081 | 366.502 | 2 | 3.5 | 5.794 | −0.309 | 52.097 |
| PHB4081 | 366.502 | 2 | 3.5 | 5.677 | −0.31 | 52.594 |
| PHB4082 | 354.494 | 2 | 5 | 4.46 | −0.467 | 60.533 |
| PHB4083 | 406.57 | 2 | 5 | 5.136 | −0.428 | 59.408 |
| PHB4083 | 406.57 | 2 | 5 | 5.005 | −0.491 | 59.228 |
| TH-1-45-1 | 340.467 | 2 | 5 | 4.151 | −0.444 | 60.796 |
| TH-1-45-2 | 314.43 | 2 | 5 | 3.719 | −0.447 | 60.754 |
| TH-1-45-3 | 342.483 | 2 | 5 | 4.44 | −0.507 | 59.626 |
| TH-1-53-2 | 362.474 | 2 | 5 | 4.753 | −0.574 | 61.457 |
| TH-1-53-3 | 368.521 | 2 | 5 | 4.864 | −0.545 | 60.762 |
| Th-1-73 | 291.392 | 1.5 | 3.5 | 3.738 | −0.725 | 74.766 |
| Th-1-73 | 291.392 | 1.5 | 3.5 | 3.752 | −0.733 | 75.94 |
| Th-1-75 | 373.537 | 1 | 3.5 | 6.034 | −0.301 | 61.175 |
| Th-1-75 | 373.537 | 1 | 3.5 | 6.077 | −0.313 | 60.417 |
| Th-1-78 | 343.468 | 1.5 | 3.5 | 4.46 | −0.627 | 71.303 |
| Th-1-78 | 343.468 | 1.5 | 3.5 | 4.471 | −0.653 | 71.94 |
| Th-1-79 | 425.613 | 1 | 3.5 | 6.755 | −0.214 | 60.383 |
| Th-1-79 | 425.613 | 1 | 3.5 | 6.753 | −0.21 | 58.511 |
| Th-2-31 | 355.482 | 2 | 5.5 | 4.168 | −0.569 | 71.286 |
| Th-2-31 | 355.482 | 2 | 5.5 | 4.149 | −0.568 | 70.881 |
| Th-2-37-1 | 355.482 | 2 | 5.5 | 4.19 | −0.508 | 70.525 |
| Th-2-37-1 | 355.482 | 2 | 5.5 | 4.171 | −0.493 | 69.737 |
| Th-2-37-2 | 315.417 | 2 | 5.5 | 3.417 | −0.474 | 68.71 |
| Th-2-37-2 | 315.417 | 2 | 5.5 | 3.447 | −0.495 | 70.775 |
| Th-2-5 | 221.302 | 2.5 | 3.25 | 2.319 | −0.299 | 55.019 |
| Th-2-64-1 | 333.473 | 1 | 3.5 | 5.304 | −0.312 | 60.221 |
| Th-2-64-1 | 333.473 | 1 | 3.5 | 5.313 | −0.317 | 60.85 |
| Th-2-7 | 221.302 | 2.5 | 3.25 | 2.132 | −0.488 | 57.053 |
| TH-2-8 | 240.732 | 1 | 2.75 | 3.395 | 0.296 | 31.36 |
| Th-2-9-1 | 303.447 | 2 | 3.25 | 4.602 | 0.093 | 39.867 |
| TH-2-9-2 | 303.447 | 2 | 3.25 | 4.423 | −0.128 | 42.142 |
| th-3-83-2 | 181.237 | 2.5 | 2.5 | 1.939 | −0.21 | 51.598 |
| th-3-86-r2 | 357.498 | 1 | 5.5 | 4.47 | −0.375 | 58.534 |
| th-3-86-r2 | 357.498 | 1 | 5.5 | 4.503 | −0.42 | 60.669 |
| th-3-86-r2 | 357.498 | 1 | 5.5 | 4.492 | −0.465 | 62.317 |
| TH-3- | 263.382 | 2 | 2.5 | 4.163 | 0.16 | 36.465 |
| 95-1-r1 | | | | | | |
| TH-3- | 263.382 | 2 | 2.5 | 4.137 | 0.113 | 39.308 |
| 95-1-r1 | | | | | | |
| th-3- | 223.317 | 2 | 2.5 | 3.45 | 0.15 | 36.83 |
| 95-2-r1 | | | | | | |
| TH-4- | 367.493 | 2 | 5.5 | 4.123 | −0.452 | 67.269 |
| 100-2 | | | | | | |
| TH-4- | 367.493 | 2 | 5.5 | 4.126 | −0.467 | 67.762 |
| 100-2 | | | | | | |
| TH-4- | 341.455 | 2 | 5.5 | 3.841 | −0.562 | 71.592 |
| 16-1 | | | | | | |
| TH-4- | 341.455 | 2 | 5.5 | 3.83 | −0.564 | 71.218 |
| 16-1 | | | | | | |
| TH-4-45 | 277.282 | 0 | 5.5 | 1.47 | −1.109 | 93.72 |
| TH-4- | 277.282 | 0 | 5.5 | 1.56 | −1.056 | 92.306 |
| 46-2 | | | | | | |
| th-4-48-2 | 303.32 | 1 | 5.5 | 2.118 | −1.237 | 102.827 |
| TH-4- | 303.32 | 1 | 5.5 | 2.146 | −1.228 | 102.246 |
| 50-2 | | | | | | |
| th-4-52-2 | 277.282 | 0 | 5.5 | 1.461 | −1.097 | 93.72 |
| th-4-53-1 | 343.471 | 2 | 5.5 | 4.111 | −0.599 | 68.522 |
| th-4-53-1 | 343.471 | 2 | 5.5 | 4.154 | −0.582 | 69.877 |
| th-4-53-2 | 341.455 | 2 | 5.5 | 3.856 | −0.518 | 70.431 |
| th-4-53-2 | 341.455 | 2 | 5.5 | 3.849 | −0.466 | 68.304 |
| th-4-55-1 | 327.428 | 2 | 5.5 | 3.548 | −0.559 | 71.748 |
| th-4-55-1 | 327.428 | 2 | 5.5 | 3.535 | −0.548 | 71.329 |
| TH-4-55-2 | 343.471 | 2 | 5.5 | 4.116 | −0.613 | 69.458 |
| TH-4-55-2 | 343.471 | 2 | 5.5 | 4.146 | −0.611 | 70.447 |
| TH-4-58-2 | 289.293 | 1 | 5.5 | 1.834 | −1.198 | 101.879 |
| th-4-62 | 313.402 | 2 | 5.5 | 3.24 | −0.56 | 71.643 |
| th-4-62 | 313.402 | 2 | 5.5 | 3.226 | −0.551 | 71.218 |
| th-4-66 | 341.455 | 2 | 5.5 | 3.857 | −0.558 | 70.98 |
| th-4-66 | 341.455 | 2 | 5.5 | 3.839 | −0.558 | 70.582 |
| TH-4-67 | 327.428 | 2 | 5.5 | 3.526 | −0.525 | 71.424 |
| TH-4-67 | 327.428 | 2 | 5.5 | 3.672 | −0.609 | 70.841 |
| th-4-68 | 329.444 | 2 | 5.5 | 3.799 | −0.606 | 69.631 |
| th-4-68 | 329.444 | 2 | 5.5 | 3.816 | −0.58 | 69.903 |
| TH-4-99- | 355.396 | 1 | 5.5 | 2.874 | −1.232 | 102.313 |
| 2-mw-r2 | | | | | | |

These efforts resulted in the development of a new series of analogs with increased structural diversity and potency that could be evaluated for their suitability for in vivo applications. The initial compounds were evaluated for in vitro stability in mouse liver microsomes over two hours. Compounds (20 μM) were incubated with mouse liver microsomes (20 mg/mL), a NAPDH-regenerating system (Corning), and PBS pH 7.4, at 37° C. with shaking. At selected time points, an aliquot from each reaction mixture was quenched by diluting 1:4 in cold methanol containing an appropriate internal standard. Samples were centrifuged and the supernatant was analyzed by HPLC-MS, with the relative amount of compound remaining at each time-point compared against the t=0 time point.

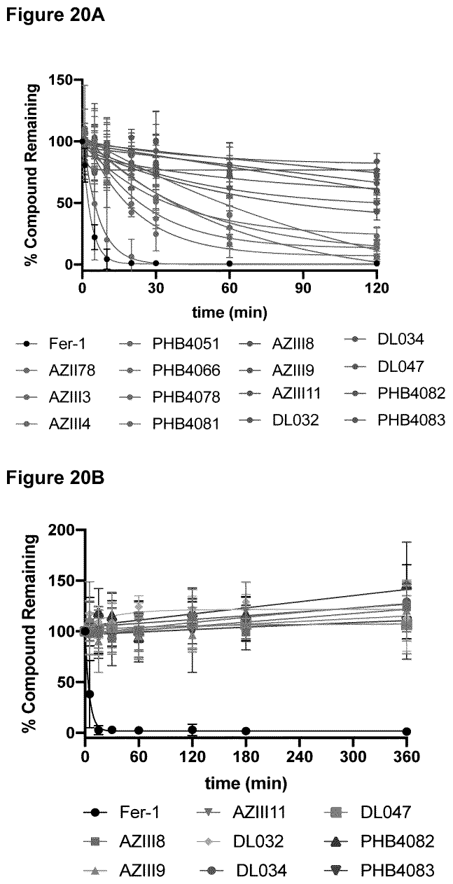

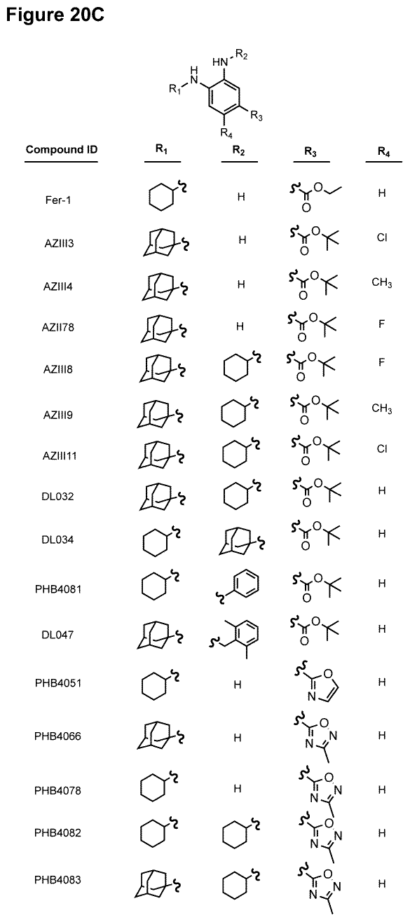

The tested compounds exhibited a range of stabilities in CD1 mouse liver microsomes (FIG. 20A). Consistent with previous reports, fer-1 was metabolically unstable, exhibiting a half-life of 2.4 min. The stability of each analog was compared to fer-1. Of the 15 tested compounds, seven (AZII78, AZIII3, AZIII4, PHB4051, PHB4066, PHB4078, and PHB4081) were found to have low-to-medium stability with a half-life less than 60 minutes. Structurally, all but PHB4081 contained an unsubstituted aniline group, suggesting that substitution on aniline is critical for minimizing CYP450-mediated metabolism. At other sites of substitution, the analogs were structurally diverse, containing both cyclohexyl and adamantyl groups at R₁, tert-butyl ester, oxazole, and 3-methyl-1,2,4-oxodiazole groups at R₃, and no substitution, or methyl, chloro, or fluoro groups at R₄, further highlighting the critical importance of aniline substitutions on microsomal stability in mouse liver microsomes.

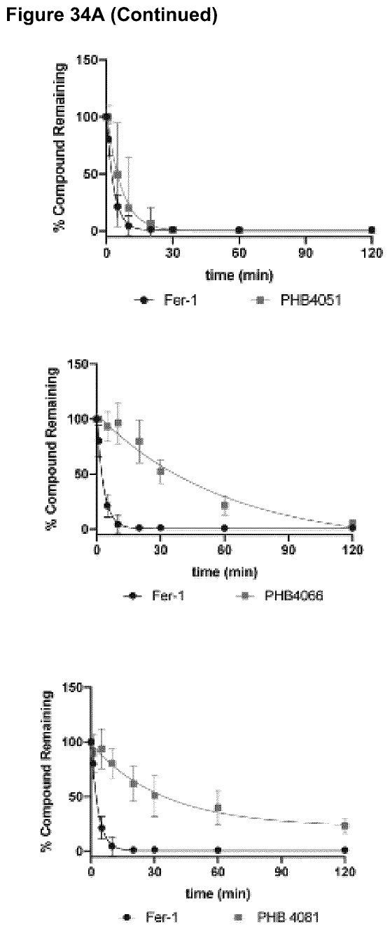

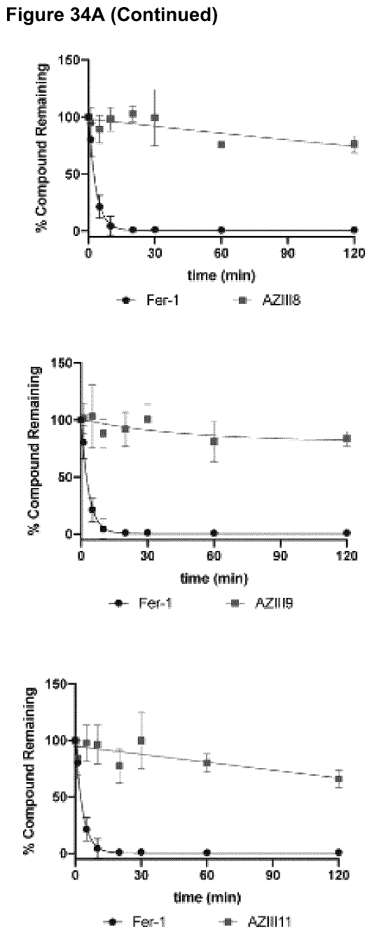

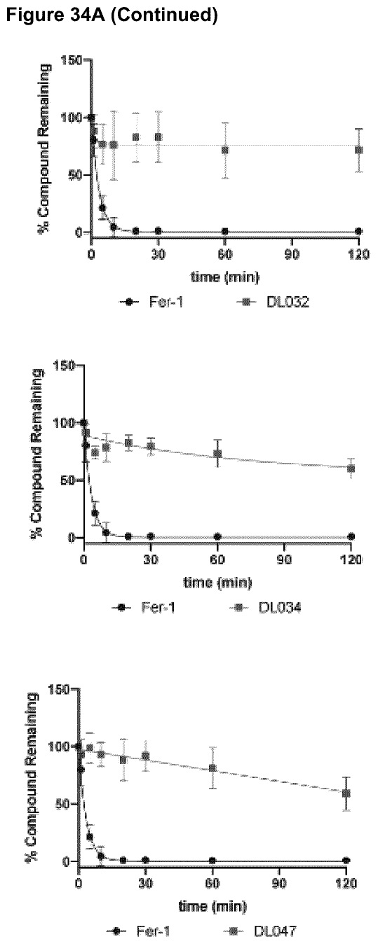

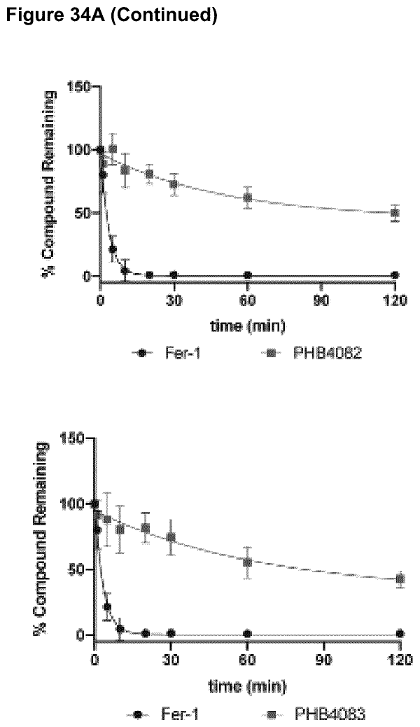

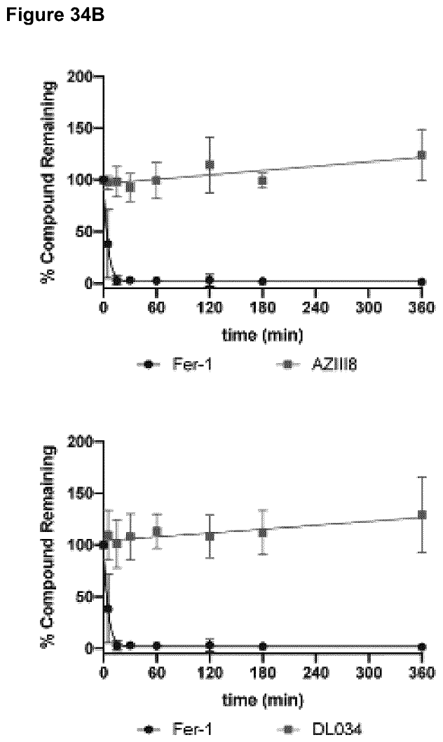

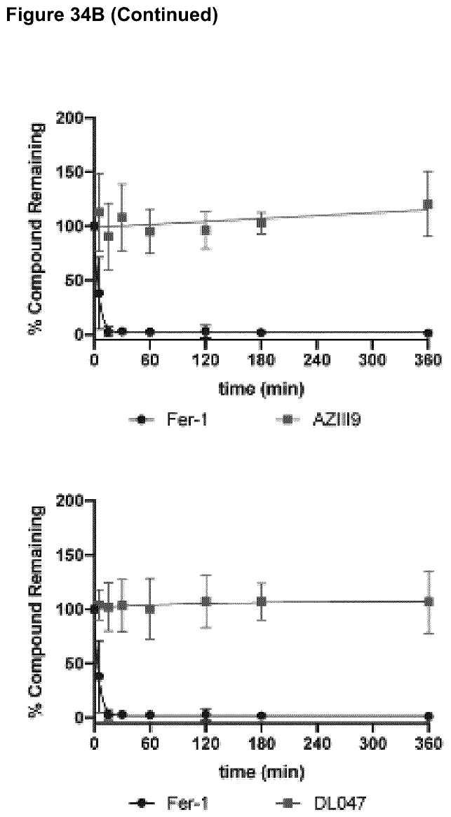

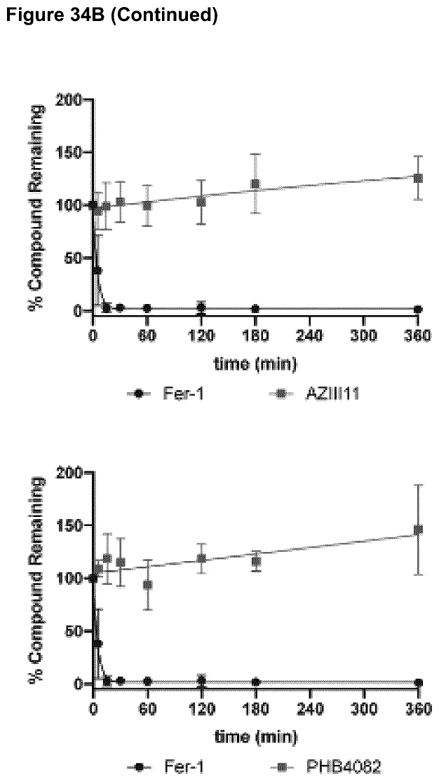

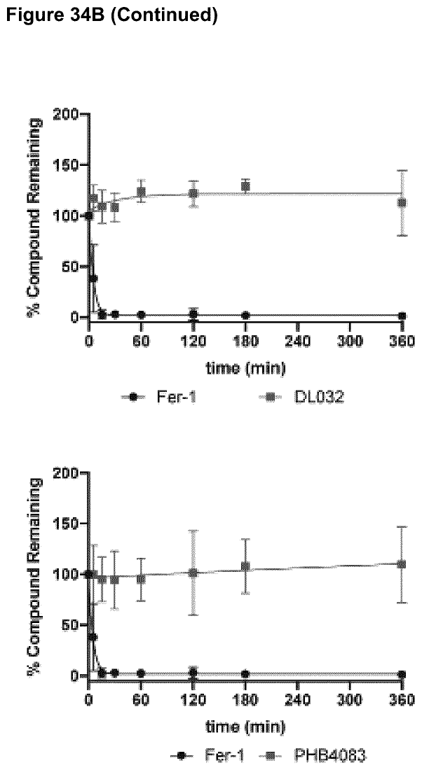

The eight analogs with high microsomal stability (AZIII8, AZIII9, AZIII11, DL032, DL034, DL047, PHB4082, and PHB4083) had half-lives greater than two hours. Please note that PHB4082 and PHB4083 are also named as CFI-4082 and CFI-4083, respectively, in the Examples above. All eight compounds contain a mono-substituted aniline group, with structural diversity at other substitution points. Of the analogs tested, no direct correlation was found between stability in mouse microsomes and potency. AZIII9, which was the most stable compound tested, was less potent than fer-1 at suppressing ferroptosis, while PHB4051, the least stable compound, was more potent than fer-1. Similarly, of the analogs with high stability, half (AZIII8, DL032, PHB4082, and PHB4083) were more potent than fer-1, while the other half (AZIII9, AZII11, DL034, and DL047) were less potent than fer-1 (FIGS. 34A-34B). This suggests that stability and potency can be optimized independently.

The eight analogs with high mouse microsomal stability were evaluated for their stability in mouse plasma. Each analog (20 μM) was incubated in mouse plasma diluted 1:1 in phosphate buffer pH 7.4. At selected time points, an aliquot of the reaction mixture was quenched by diluting in 1:4 in cold methanol containing an appropriate internal standard. Samples were centrifuged and the supernatant analyzed via HPLC-MS, with the relative amount of compound remaining at each time-point compared against the t=0 time-point. The stability of the compounds was compared to fer-1, which was rapidly degraded in mouse plasma. All compounds were found to be stable in mouse plasma, with no metabolism detected across six hours (FIG. 20B). Structurally, the compounds either contained a tert-butyl ester group or a 3-methyl-1,2,4 oxadiazole group at position R₃, validating this approach to compound design.

### PHB4082 has Potential for Preventing Ferroptosis in the Kidney

Of the eight compounds tested with high microsomal and plasma stability, PHB4082 was selected as a promising candidate due to its high potency in suppressing ferroptosis, with a 7-fold increase in potency in suppressing erastin-induced ferroptosis compared to fer-1. In fact, PHB4082 was the most potent of the eight analogs with high microsomal and plasma stability,

To evaluate whether PHB4082 was a suitable candidate for in vivo studies, we performed a pharmacokinetic study of PHB4082 in C57BL/6 mice at ˜8 weeks of age. To evaluate sex-specific effects on compound metabolism, two male and two female mice were used for each condition. Mice were dosed at 20 mg/kg via intraperitoneal (IP) injection and the injection volume ranged from 180 uL to 300 uL based on different body weights. At a pre-designated time-point following compound administration, mice were sacrificed via CO₂ asphyxiation, blood collected via cardiac puncture, and organs harvested. Plasma was separated from whole blood via centrifugation and organs homogenized prior to extracting with acetonitrile. Extracts were analyzed via UPLC-MS and the concentration of compound determined against a standard curve ran concurrently with samples.

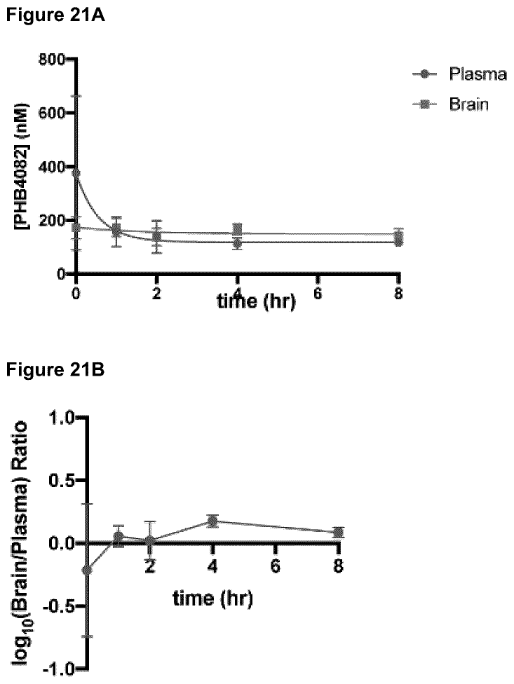

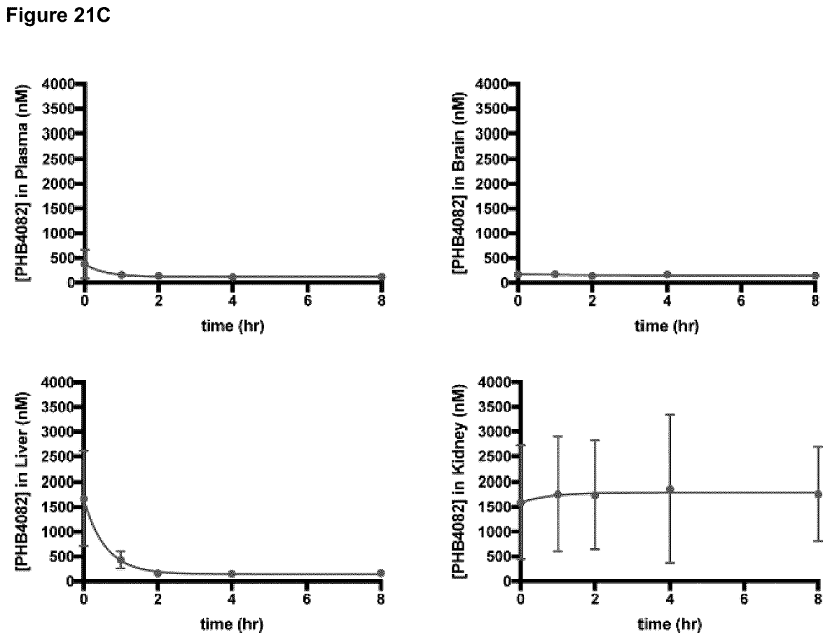

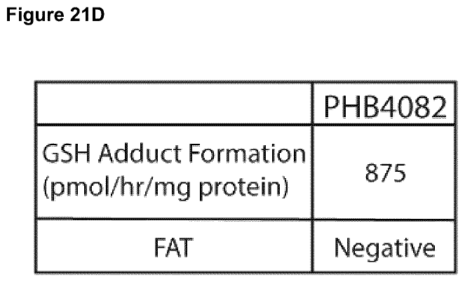

We first determined the concentration of PHB4082 in plasma and brain homogenates to determine whether PHB4082 was a suitable candidate for CNS disease applications. Despite the fact that we administered PHB4082 to mice at a dose of 20 mg/kg, we observed accumulation of PHB4082 in plasma and brain only to ˜100 nM (FIG. 21A). Notably, accumulation in brain appeared to be relatively stable across the eight-hour time period evaluated, with relatively minimal changes in brain concentration over time.

To determine whether PHB4082 accumulated in brain preferentially over plasma, the log₁₀(Brain/Plasma) ratio was calculated for each time-point with a log₁₀(Brain/Plasma) >0 indicating preferential accumulation in brain over plasma. We observed that PHB4082 did slightly accumulate in brain over plasma as time progressed (FIG. 21B); however, at the time-point where PHB4082 preferentially accumulated in brain, the difference in concentration in the brain compared to plasma was low, indicated by the near 0 log values. Notably, PHB4082 and SRS11-92 have similar potencies (Skouta et al. 2014), but PHB4082 accumulates to concentrations less than what is required to achieve efficacy in an HD brain slice model. The unfavorable brain pharmacokinetic profile was not due to vehicle composition, as mice dosed intraperitoneally and intravenously with 10 mg/kg PHB4082 in a vehicle composition used in later PK studies had comparable levels of compound accumulation in both brain and plasma, with log₁₀(Brain/Plasma) ratio values <0 (Table 11). Taken together, these findings suggest that PHB4082 is not a suitable candidate for CNS applications.

TABLE 11
PHB4082 has poor in vivo pharmacokinetics in cyclodextrin/PEG/tween
vehicle, related to Figures 21A-21D.

| | IP Administration | | | IV Administration | | |
|---|---|---|---|---|---|---|
| Time | | | log₁₀(Brain/ | | | log₁₀(Brain/ |
| (hr) | [PHB4082]ₚₗₐₛₘₐ | [PHB4082]_{brain} | Plasma) | [PHB4082]ₚₗₐₛₘₐ | [PHB4082]_{brain} | Plasma) |
| 2 | 957 ± 693 | 114 ± 21 | −0.84 ± 0.28 | 634 ± 359 | 557 ± 245 | −0.04 ± 0.12 |
| | nM | nM | | nM | nM | |
| 4 | 306 ± 379 | 99 ± 62 | −0.35 ± 0.42 | 281 ± 284 | 176 ± 76 | −0.08 ± 0.31 |
| | nM | nM | | nM | nM | |

The low concentration of PHB4082 observed in plasma and brain led us to question where the compound accumulated following administration. As the liver and kidney are primary routes of elimination, we evaluated the concentration of PHB4082 in liver and kidney homogenates (FIG. 21C). Immediately following administration, comparable levels of PHB4082 were observed in liver and kidney, but notably, the concentration of PHB4082 in the liver exponentially decreased thereafter, but the concentration of PHB4082 in the kidney stably accumulated, albeit with high intrasample variability over time, with an average kidney concentration of 1.8 μM PHB4082 at eight hours, which is far above its effective concentration in cell culture. This suggests that PHB4082 could be a candidate for testing the role of ferroptosis in kidney-related disease models, depending on the specific localization within the kidney relative to kidney pathologies in specific diseases.

We then tested PHB4082 in a GSH adduct formation assay and the Ames test. PHB4082 was positive in a GSH trapping assay (FIG. 21D). The result of the Ames test was negative, which suggested PHB4082 has minimal capacity to induce mutagenesis. However, the GSH trapping result suggested that PHB4082 has the potential to generate chemically reactive metabolites which can react with cellular molecules, such as proteins or DNA, leading to adverse reactions. The criterion for such reactivity is typically >200 pmol/hr/mg protein, while PHB4082 had a value of 875 pmol/hr/mg protein, indicating its potential reactivity.

Additionally, we evaluated PHB4082 and related fer-1 analogs with the Derek Nexus toxicity prediction software (Marchant, Briggs, and Long 2008), which revealed predicted toxicity due to the presence of the ortho-phenylenediamine moiety within the structure of PHB4082 and all previous fer-1 analogs. The o-phenylenediamine moiety has reported aquatic toxicity and skin sensitizing activity. As the o-phenylenediamine moiety is present in the structures of ferrostatins produced and tested to date, this necessitated the development of a new scaffold that could circumvent this toxicity.

However, PHB4082 is the first chemical probe useful for evaluating the in vivo relevance of ferroptosis in kidney-related diseases. This may aid in identifying kidney-related disease contexts where ferroptosis is the main driver.

### In Vivo Renal Tubular Ferroptosis can be Inhibited by PHB4082

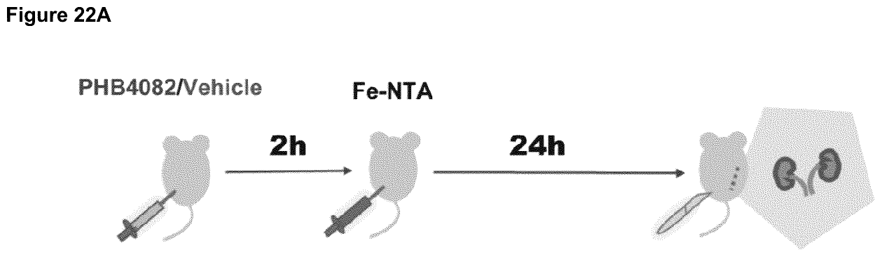

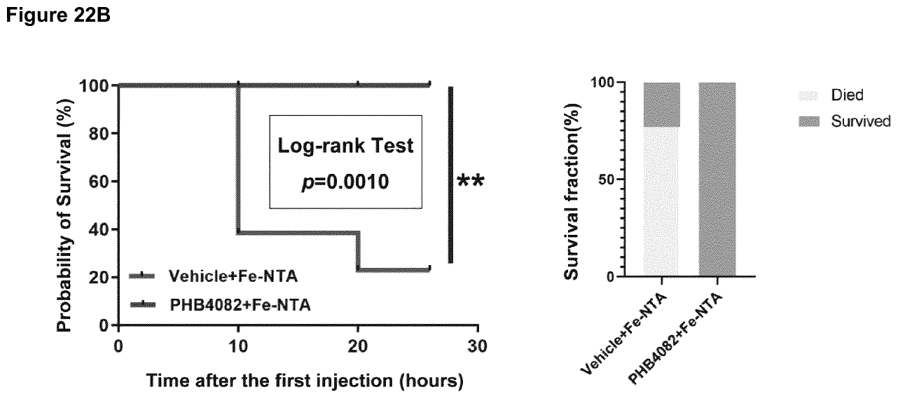

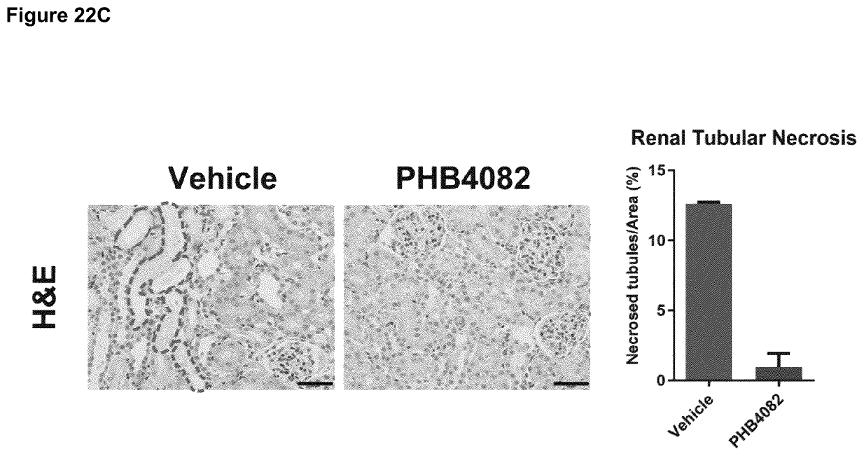

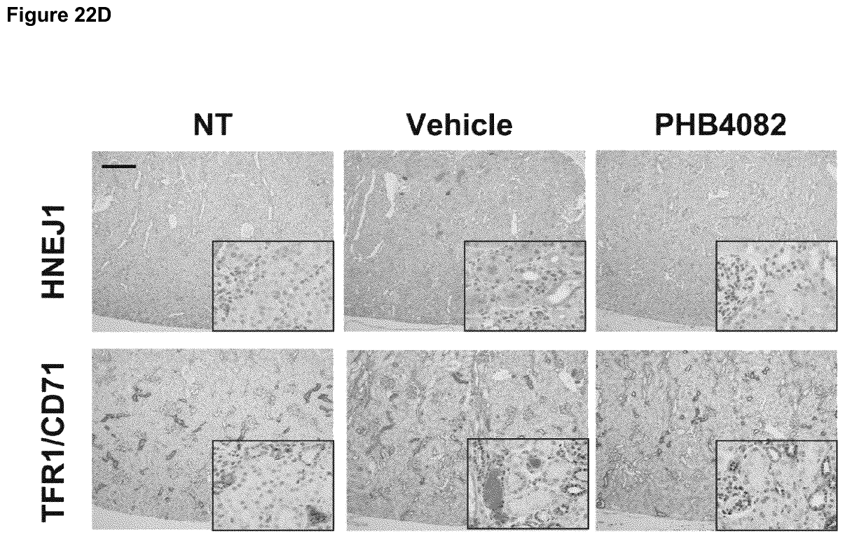

We evaluated the ferroptosis protection effect of PHB4082 in a renal tubular ferroptosis model. This model was previously established by Toyokuni lab (Kong et al. 2022; Toyokuni et al. 2022; Akatsuka et al. 2012). A Fenton-reaction based renal carcinogenesis in this model results from repeated intraperitoneal injections of ferric nitrilotriacetate (Fe-NTA). This iron chelate could specifically induce proximal epithelial ferroptosis via single IP administration. After two IP injections as described in methods (FIG. 22A), the rats with vehicle formulation died of renal failure with a high ratio (76.9%) after Fe-NTA injection, whereas all of those with PHB4082 pretreatment survived 24 h after Fe-NTA administration (FIG. 22B). We chose 24 h after Fe-NTA administration for evaluation of the effects of PHB4082 because this is the peak time for renal tubular necrosis. To search for the underlying pathological alterations, we performed renal tubular necrosis analysis and the results indicated that PHB4082 dramatically reduced the necrotized tubular area (FIG. 22C).

To further identify the microscopical necrotic change observed in the rat model, we performed immunostaining by several valid ferroptosis markers including HNEJ-1 (Zheng et al. 2021) and TRF1/CD71 (Stockwell, 2022). Regarding HNEJ-1, we found less amount of HNE adducts accumulated in a homogenous pattern in proximal tubules with the Fe-NTA-induced renal damage after the PHB4082 pretreatment in comparison to the vehicle control (FIG. 22D). Regarding TfR1 positive tubules, except that typically high staining intensity was shown in distal tubules including in the untreated control group, the PHB4082 pretreatment group showed low to moderate signals in the proximal tubules whereas moderate to high granular signals were detected in the cytoplasm of renal proximal tubular epithelium and luminal secretion in the vehicle group (FIG. 22D). Both biomarkers indicated that PHB4082 inhibits renal tubular ferroptosis, predominantly targeting proximal tubules.

### Development and Characterization of Fifth Generation Ferrostatin Analogs

To overcome the liabilities associated with PHB4082 and other 4^{th} generation ferrostatins, we sought to develop new compounds that circumvent the potential phenylenediamine toxicity in the ferrostatin scaffold. To this end, a pyridine core was employed, as pyridine has similar electronic properties to benzene and avoids the toxicity issues associated with phenylenediamines. The design was informed by the principles established with previous ferrostatins. The value of this design was confirmed with Derek Nexus toxicity prediction software, which indicated no predicted toxicities were associated with compounds employing a pyridine core.

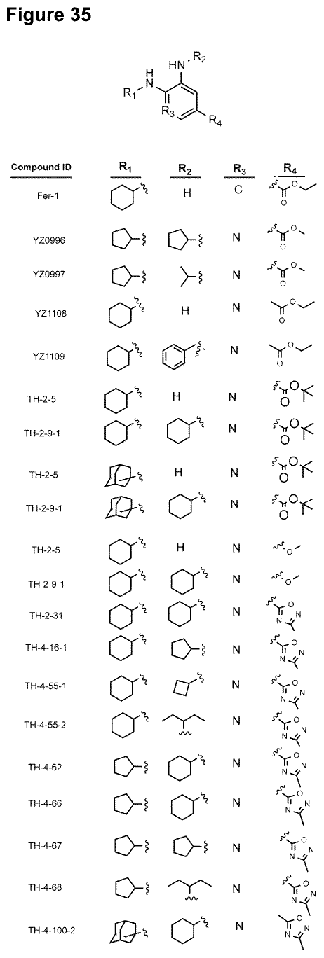

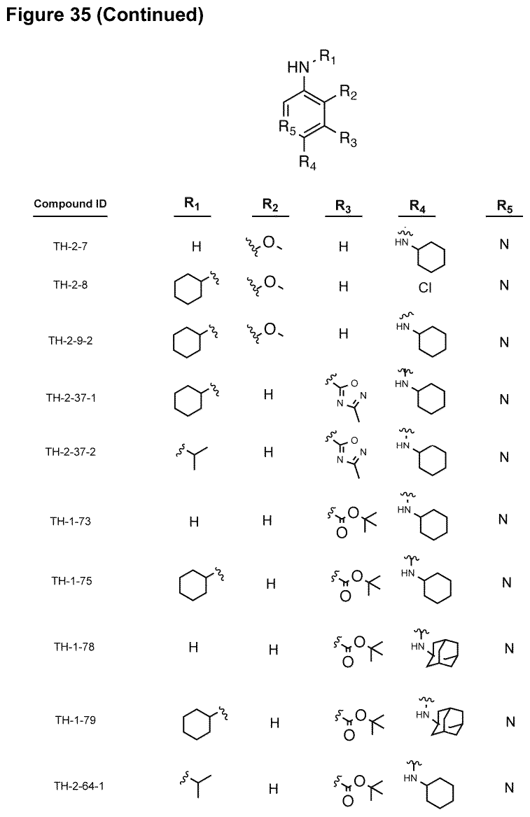

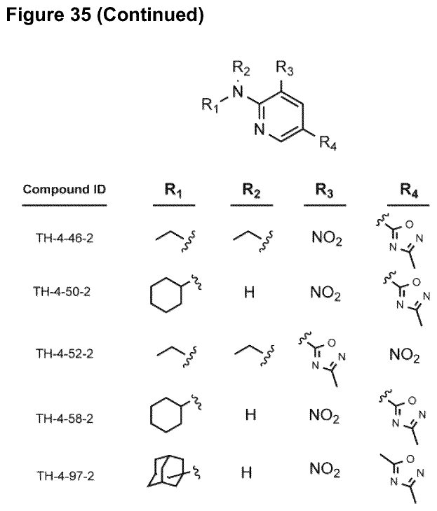

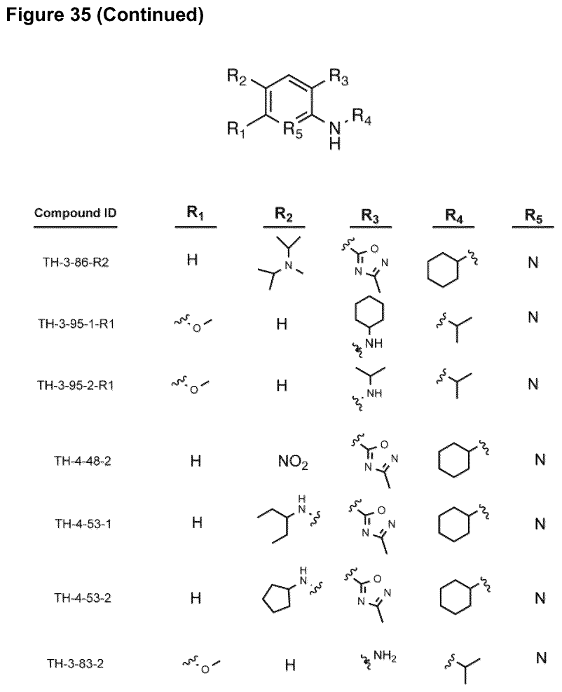

Additional compounds were designed to have increased brain penetration. Suitable lipophilicity is critical to the brain penetration of drugs. Lipophilicity can increase transport across the BBB, while it also favors uptake by peripheral tissues, which lowers the concentration in the blood (Banks 2009). We designed multiple compounds with different combinations of substituents to determine the most suitable balance of lipophilicity. Hydrogen-bonding capacity is another critical factor, which is associated with lower passive permeability, as desolvation of the associated hydrogen-bound water molecules is required for membrane permeability (Veber 2003). Polar surface area (PSA) and pKa are also common parameters considered in the BBB penetration improvement. Lower PSA and higher pKa are associated with higher BBB penetration in many cases (Rankovic 2015). We used Qikprop to predict clogP, H-bonding, PSA, and pKa to evaluate and prioritize compounds (Schrodinger Suite Qikprop). A total of 41 compounds (FIG. 35) in this 5th-generation series were synthesized. All compounds were prepared following the general synthetic schemes in Scheme 23.

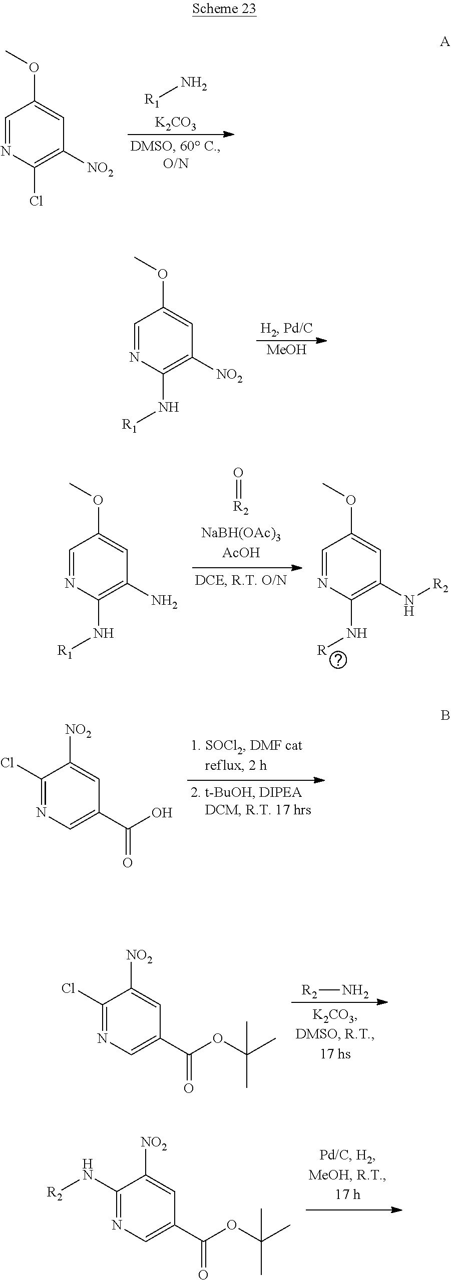

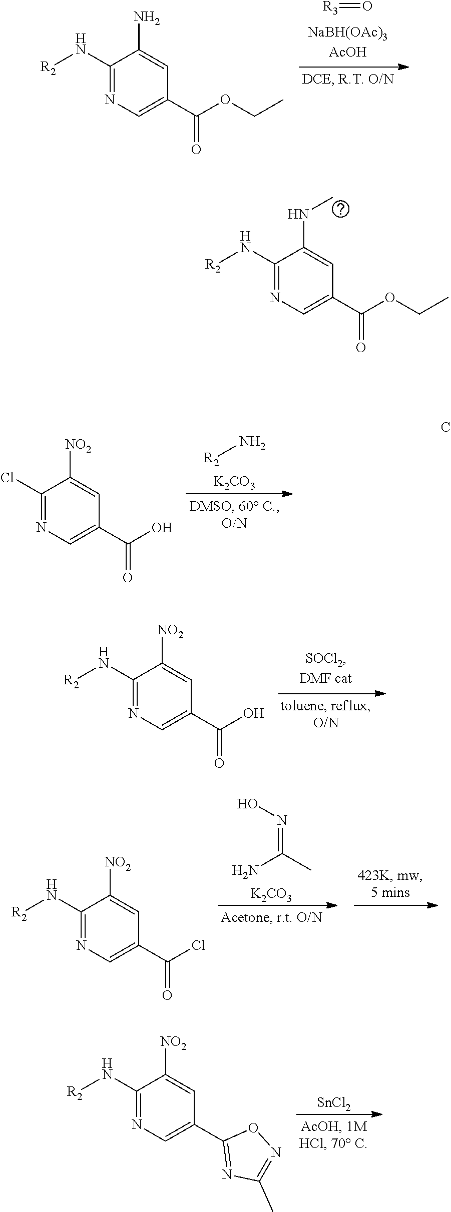

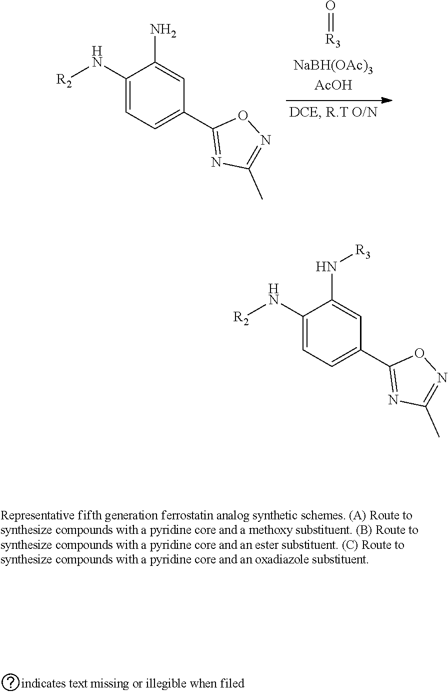

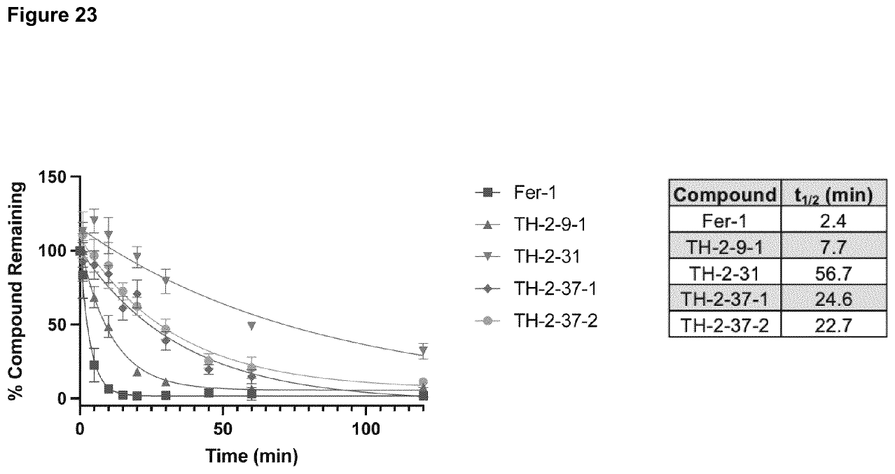

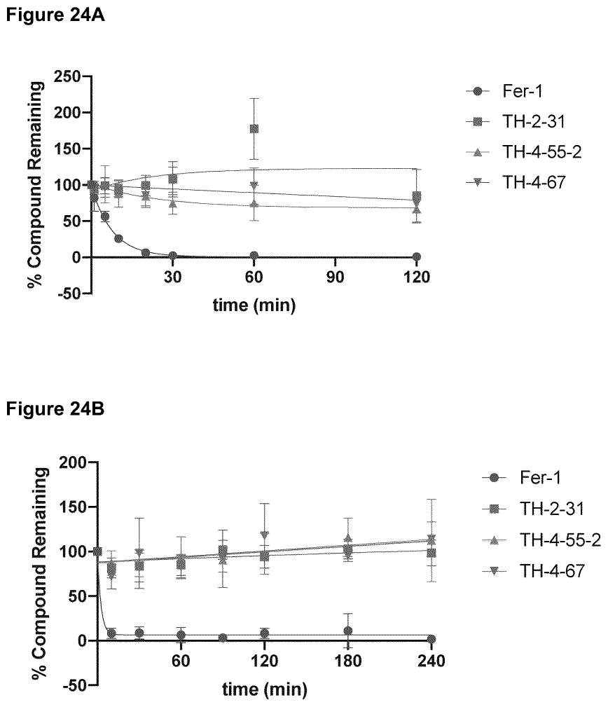

We first evaluated the efficacy of four pyridine analogs, TH-2-9-1, TH-2-31, TH-2-37-1, and TH-2-37-2, in suppressing ferroptosis induced by IKE and RSL3 in HT-1080 cells. All compounds except TH-2-9-1 inhibited ferroptosis at potencies greater than fer-1, while TH-2-9-1 was slightly less potent than fer-1 in inhibiting IKE-induced death (Table 12). This suggests that compounds using a pyridine core in place of an aniline core can be synthesized with no decrease in potency. We sought to determine whether incorporation of pyridine changed stability in mouse liver microsomes. All four compounds had decreased stability compared to the most stable fourth-generation analogs (FIG. 23). Of the four analogs tested, TH-2-31 was found to be the most stable, with a half-life between 30 and 60 minutes; compared to PHB4082, the fourth-generation counterpart, the stability was decreased, suggesting that incorporation of pyridine into the structure decreased stability.

TABLE 12
Potency of fifth generation ferrostation analogs. The potency of fifth
generation analogs in inhibiting ferroptosis was assessed in HT-1080 cells at 1,000
cells/well treated with ^{b}2 μM IKE, or ^{c}0.2 μM RSL3 treated for 48 hours.

| Compound ID | IC₅₀ (nM) | Structure |
|---|---|---|
| Fer-1 | 9.1^{b} 3.6^{c} | 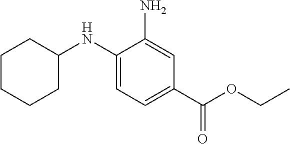 [SMILES: CCOC(=O)c1ccc(NC2CCCCC2)c(N)c1] |
| TH-2-5 | — | 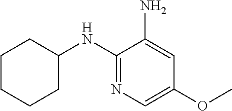 [SMILES: COc1cnc(NC2CCCCC2)c(N)c1] |
| TH-2-7 | — | 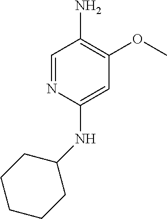 [SMILES: COc1cc(NC2CCCCC2)ncc1N] |
| TH-2-8 | — | 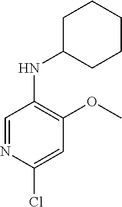 [SMILES: COc1cc(Cl)ncc1NC1CCCCC1] |
| TH-2-9-1 | 16.9^{b} 2.7^{c} | 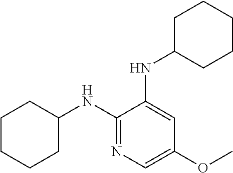 [SMILES: COc1cnc(NC2CCCCC2)c(NC2CCCCC2)c1] |
| TH-2-9-2 | — | 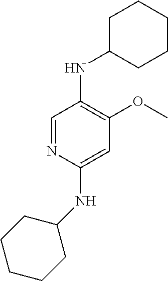 [SMILES: COc1cc(NC2CCCCC2)ncc1NC1CCCCC1] |
| TH-2-31 | 5.6^{b} 4.8^{c} | 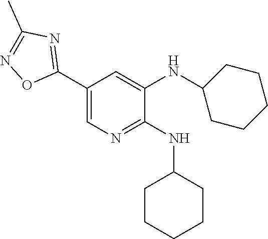 [SMILES: Cc1noc(-c2cnc(NC3CCCCC3)c(NC3CCCCC3)c2)n1] |
| TH-2-37-1 | 7.5^{b} 4.3^{c} | 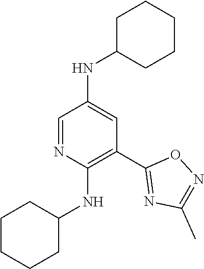 [SMILES: Cc1noc(-c2cc(NC3CCCCC3)cnc2NC2CCCCC2)n1] |
| TH-2-37-2 | 5.4^{b} 3.8^{c} | 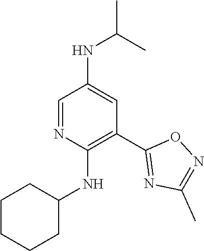 [SMILES: Cc1noc(-c2cc(NC(C)C)cnc2NC2CCCCC2)n1] |

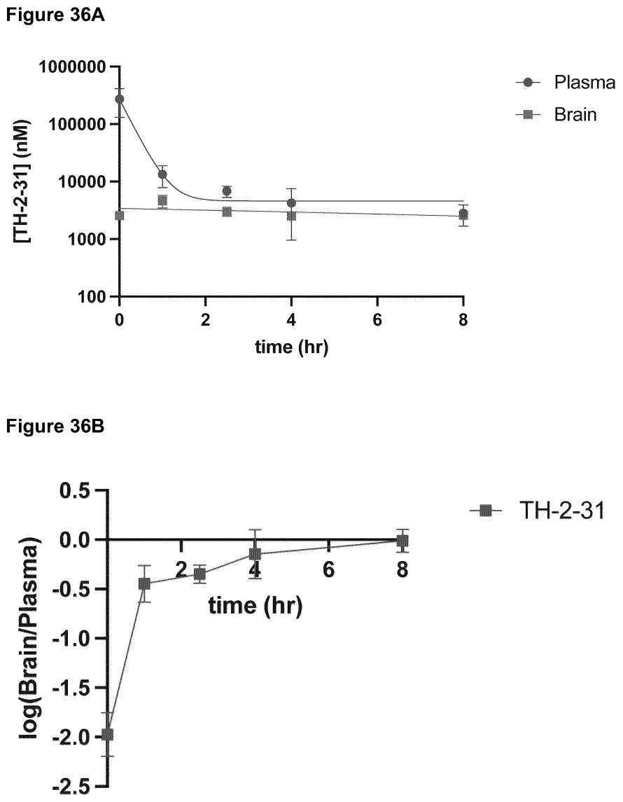

Given the limitations of PHB4082 for CNS applications, we evaluated whether TH-2-31 was stable and brain penetrant in vivo prior to proceeding with further development of the pyridine scaffold. C57BL/6 mice at 8 weeks of age were dosed intraperitoneally with TH-2-31 at a dose of 40 mg/kg in a vehicle consisting of 65% v/v of 25% w/v 2-hydroxypropyl-β-cyclodextrin, 30% v/v PEG-400, and 5% v/v Tween-80. Mice were dosed at a higher concentration of compound compared to previous studies to account for the observed decrease in stability compared to PHB4082. The injection volume ranged from 180 uL to 300 uL based on different body weights. At a pre-designated time-point following compound administration, mice were sacrificed via CO₂ asphyxiation, blood collected via cardiac puncture, and brain harvested. Plasma was separated from whole blood via centrifugation and brain homogenized prior to extracting with acetonitrile. Extracts were analyzed via UPLC-MS, and the concentration of compound determined against a standard curve that was evaluated concurrently with samples. TH-2-31 was found to stably accumulate at micromolar concentrations in plasma and brain even eight hours after following compound administration (FIGS. 36A-36B). As such, we were confident to proceed with further development and optimization of the pyridine scaffold.

### Development of Pyridine-Based Ferrostatins

Due to the high levels of TH-2-31 observed in brain, as well as our interest in developing ferroptosis inhibitors for neurodegenerative disease applications, we determined that it was relevant to examine the potency of compounds in a neuronal cell line. The potency of analogs in inhibiting ferroptotic death was examined using the N27 rat dopaminergic cell line treated with 20 nM RSL3, a concentration that was sufficient to induce death.

Most analogs were active in this cell model and able to inhibit ferroptosis; however, a wide range of IC₅₀ values was observed, with TH-3-86-R2 having an IC₅₀ of 1.0 μM while TH-4-67 had an IC₅₀ of 2.1 nM (Table 13). Th-3-86-R2 has a tertiary amine instead of a secondary amine, which matches our SAR result that the compounds with secondary amines have better potency. Five compounds (TH-4-55-1, TH-4-55-2, TH-4-66, TH-4-67, and TH-4-53-2) were found to be more potent than fer-1. Of these five potent analogs, we selected two: TH-4-55-2 and TH-4-67, along with TH-2-31 for further analysis. Although TH-2-31 was less potent than fer-1 in N27 cells, its favorable PK profile warranted its inclusion. TH-4-55-2 and TH-4-67 were chosen for their structural differences, with a branched alkyl group at R₃ for TH-4-55-2 and a cyclopentyl moiety at R₁ and R₃ for TH-4-67, compared to fer-1 and TH-2-31. While TH-4-53-2 was the most potent of the analogs tested, this analog utilized a different scaffold than TH-2-31, TH-4-55-2, and TH-4-67 and appeared less promising.

TABLE 13
Potency of fifth generation ferrostation analogs in N27 rat dopaminergic
cells. The potency of fifth generation analogs for inhibiting ferroptosis in rat
dopaminergic cells was assessed in N27 cells treated with 20 nM RSL3 with cells
seeded at 1,000 cells/well and treated for 24 hours^{d} or 48 hours^{e}.

| Compound ID | IC₅₀ (nM) | Structure |
|---|---|---|
| Fer-1 | 3.2^{d} 12.5^{e} | 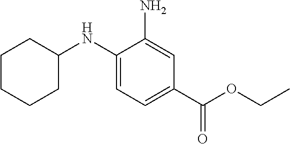 [SMILES: CCOC(=O)c1ccc(NC2CCCCC2)c(N)c1] |
| PHB4082 | 2.6^{d} | 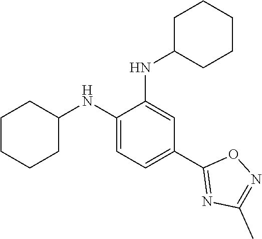 [SMILES: Cc1noc(-c2ccc(NC3CCCCC3)c(NC3CCCCC3)c2)n1] |
| TH-2-31 | 3.4^{d} | 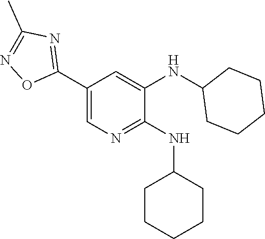 [SMILES: Cc1noc(-c2cnc(NC3CCCCC3)c(NC3CCCCC3)c2)n1] |
| TH-3-83-2 | 19.5^{d} 33.6^{e} | 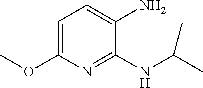 |
| TH-3-86-R2 | 1036.7^{e} | 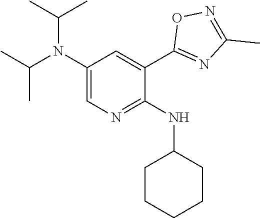 [SMILES: Cc1noc(-c2cc(N(C(C)C)C(C)C)cnc2NC2CCCCC2)n1] |
| TH-3-95-1-R1 | 30.5^{d} 22.4^{e} | 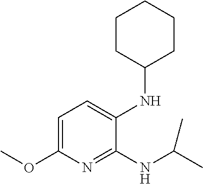 [SMILES: COc1ccc(NC2CCCCC2)c(NC(C)C)n1] |
| TH-3-95-2-R1 | 35.7^{d} 0.22^{e} | 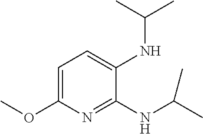 [SMILES: COc1ccc(NC(C)C)c(NC(C)C)n1] |
| TH-4-16-1 | 5.1^{d} 8.2^{e} | 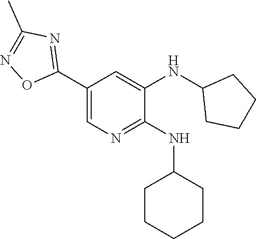 [SMILES: Cc1noc(-c2cnc(NC3CCCCC3)c(NC3CCCC3)c2)n1] |
| TH-4-53-1 | 32.7^{d} | 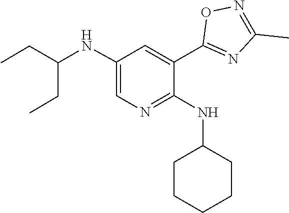 [SMILES: CCC(CC)Nc1cnc(NC2CCCCC2)c(-c2nc(C)no2)c1] |
| TH-4-53-2 | 1.8^{d} | 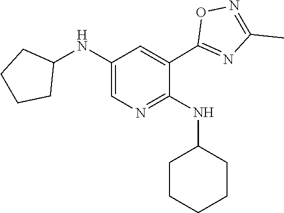 [SMILES: Cc1noc(-c2cc(NC3CCCC3)cnc2NC2CCCCC2)n1] |
| TH-4-55-1 | 2.2^{d} | 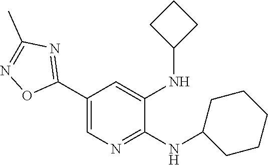 |
| TH-4-55-2 | 2.2^{d} | 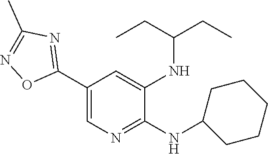 |
| TH-4-62 | 6.9^{d} | 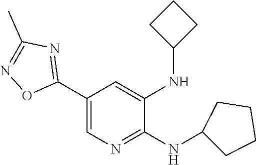 |
| TH-4-66 | 3.1^{d} | 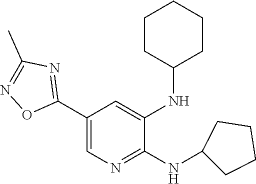 |
| TH-4-67 | 2.1^{d} | 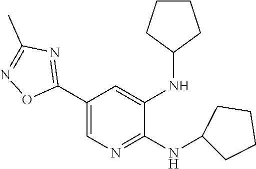 |
| TH-4-68 | 4.0^{d} | 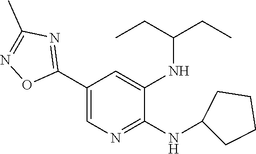 |

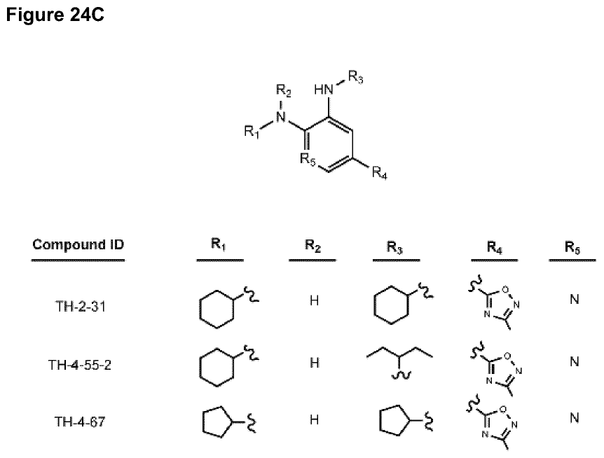

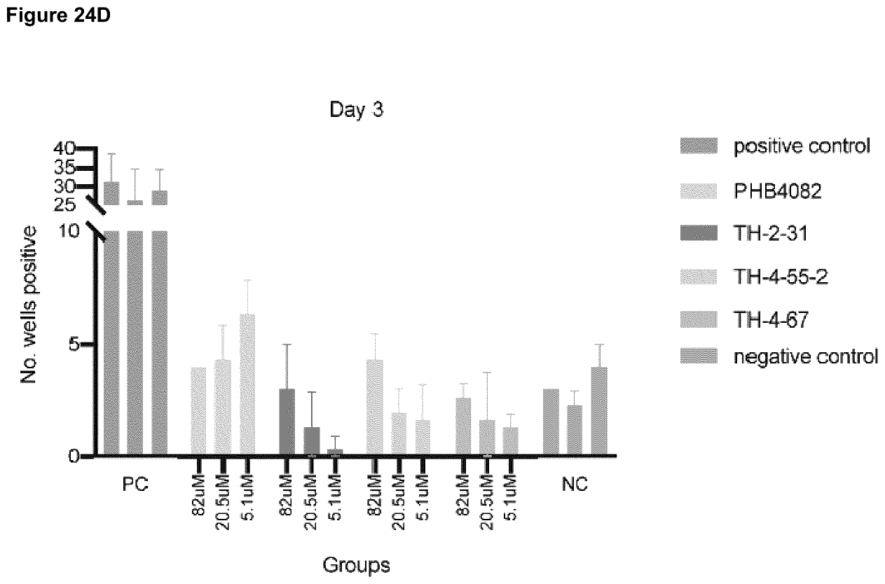

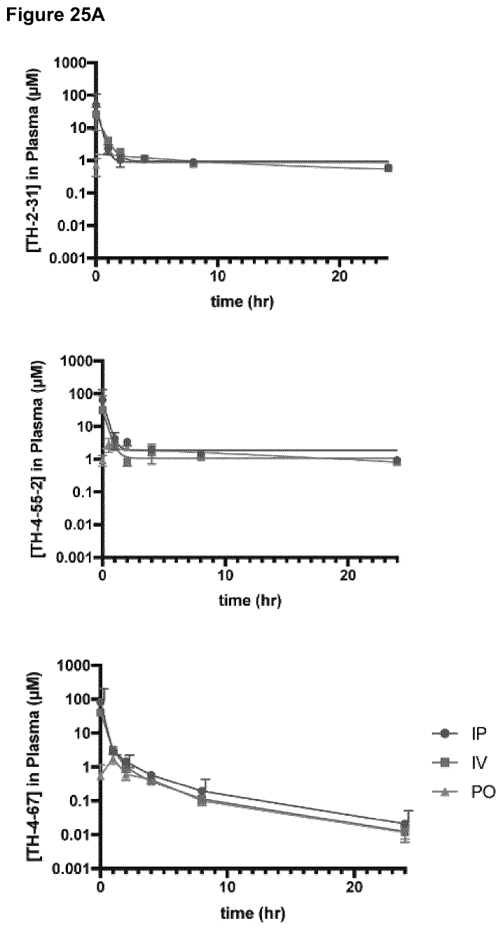

We sought to determine whether TH-2-31, TH-4-55-2, and TH-4-67 were stable in vitro. We assessed the stability of these compounds in both mouse liver microsomes and mouse plasma. We anticipated that the compounds would be stable in vitro, given the stability of TH-2-31 in vivo as well as the SAR derived from the fourth-generation analogs. We observed this to be the case, with all three compounds having half-lives greater than two hours in the microsomal stability assay and exhibiting no metabolism in mouse plasma during the time period assessed (FIGS. 24A-24D). These findings indicated that these compounds would be suitable to examine in mice.

In addition to the Derek Nexus toxicity prediction, we sought to confirm that these compounds did not have mutagenic potential in the Ames test (Zeiger 2019). The Ames test uses modified bacteria sensitive to mutagenic agents to assess a compound's ability to cause direct DNA mutations. If a tested compound can induce revert mutational events, it will cause bacteria to revert back to a prototrophic state and grow on media lacking selected nutrients. We evaluated TH-2-31, TH-4-55-2, and TH-4-67 in the Ames test. We also included PHB4082 in this test. Bacteria strains were incubated under exposure to different concentrations of tested compounds for three days and 144 data points were collected on mutation status across a concentration range from 5.1 μM to 82 μM, which was the highest local organ concentration observed in previous mouse studies. The result showed that none of the compounds had mutagenic potential and that the fifth-generation compounds had a lower ratio compared to the fourth-generation compound (FIGS. 24A-24D).

### In Vivo Pharmacokinetic Study of TH-2-31, TH-4-55-2, and TH-4-67

All three compounds, TH-2-31, TH-4-55-2, and TH-4-67 are potent and stable in vitro. To further characterize these compounds, we performed a pharmacokinetic study to assess stability in vivo and brain penetrance. While we previously found that TH-2-31 was stable and brain penetrant in vivo, mice in that study were only dosed IP. In addition, the stability and brain penetration of the compound was only assessed up to 8 hours post-administration, where high levels of TH-2-31 in both the plasma and brain were observed. As such, we sought to determine the stability of the compounds up to 24 hours post administration with mice dosed intravenously (IV), intraperitoneally (IP), or via oral gavage (PO) to provide us with a more comprehensive understanding of the pharmacokinetics of the compounds upon various routes of administration, and the compounds' suitability for further in vivo applications.

To detect sex-specific effects, two male and two female C57BL/6 mice at 8 weeks of age were used for each time-point and route of administration. Mice were dosed with 20 mg/kg compound in a vehicle consisting of 1:1 65% v/v of 25% w/v 2-hydroxypropyl-β-cyclodextrin, 30% v/v PEG-400, and 5% v/v Tween-80: milliQ H₂O. The viscosity of the undiluted solution prevented IV administration, necessitating dilution with milliQ water. The injection volume ranged from 180 uL to 300 uL based on different body weights. All three analogs were well-tolerated with no immediate toxicity observed following administration. However, IV administration resulted in the mice immediately fainting with a slow recovery, usually requiring an average of 15 minutes to become active and mobile again. Once recovered, however, no other issues were observed with the mice prior to CO₂ euthanasia. Plasma and brain homogenate were extracted with acetonitrile and analyzed via UPLC-MS.

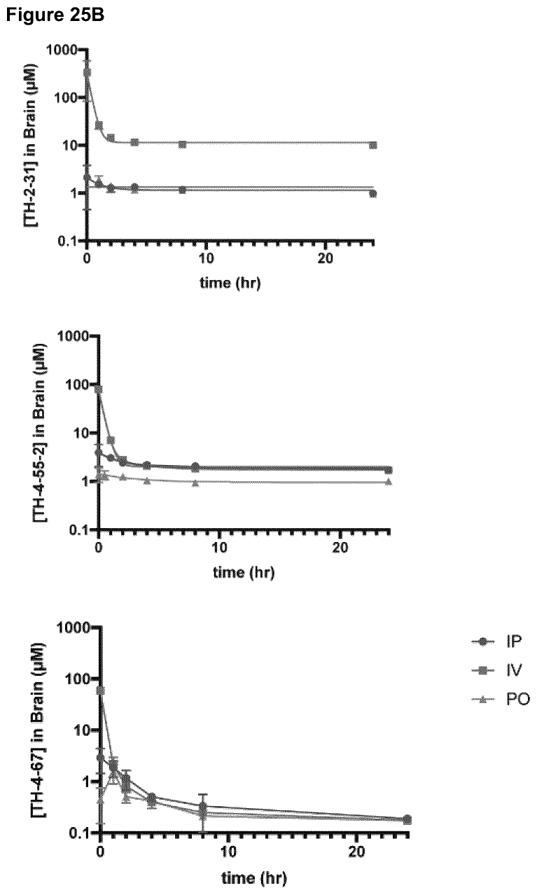

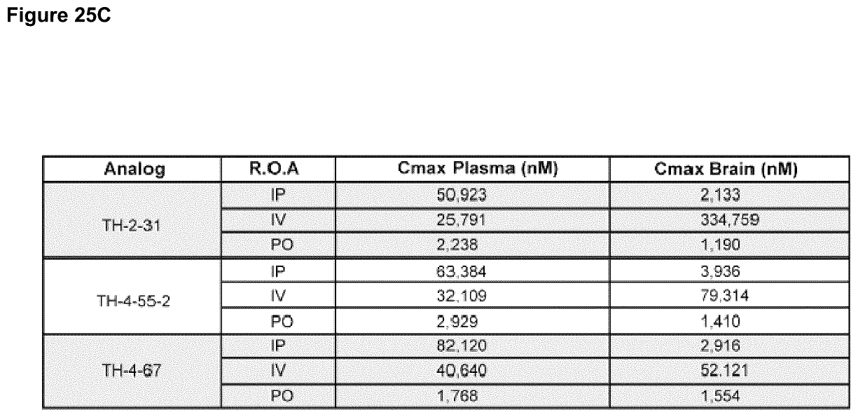

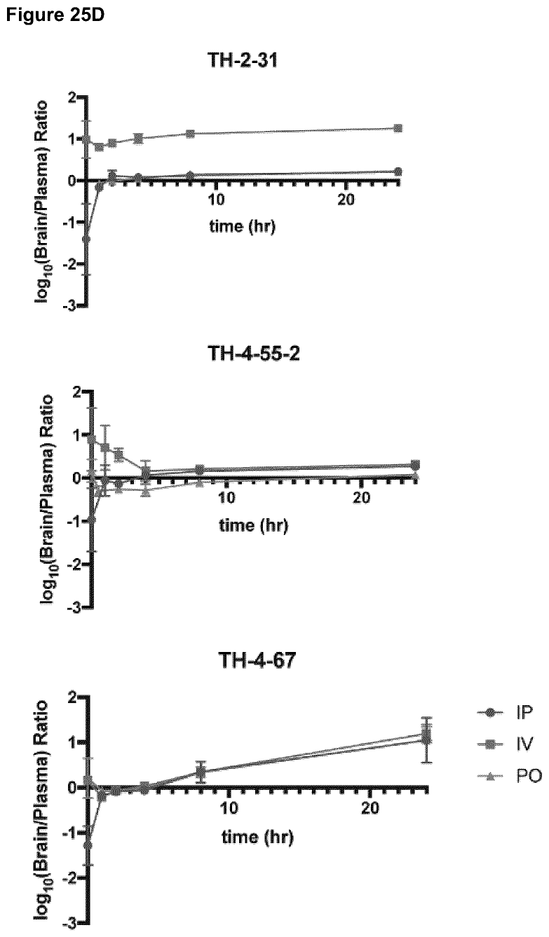

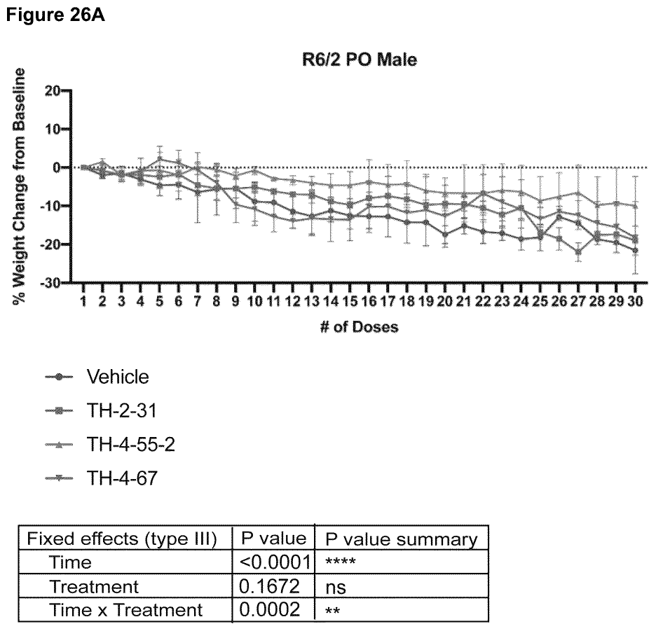

All three compounds were found to be stable in plasma for up to 24 hours, independent of the route of administration (FIG. 25A). Each compound was orally bioavailable and all three followed the expected PK trends: immediately following IP or IV administration, the concentration of each compound in plasma peaked and exponentially decreased thereafter, while with PO administration, the plasma concentration peaked after a delay and slowly decreased thereafter.

Comparing the concentration of each compound in plasma at 24 hours, we found that TH-4-55-2 accumulated the most, and TH-4-67 accumulated the least, for all routes of administration. TH-2-31 was present in plasma at micromolar concentrations for up to four hours post administration with a concentration >500 nM in plasma 24 hours after administration for all routes of administration. TH-4-55-2 was present in plasma at micromolar concentrations for up to 8 hours post administration for all routes of administration with a concentration >800 nM compound 24 hours after administration for all routes of administration. TH-4-67 had the highest initial concentrations in plasma following both IP and IV administration; however, it was only present in plasma at micromolar concentrations for up to 1 hour post administration for PO and IV administration, and up to 2 hours post IP administration. At 24 hours post administration, TH-4-67 was present in plasma at a concentration <25 nM, an order of magnitude lower than both TH-2-31 and TH-4-55-2 at the same time-point. Based on these data, TH-4-55-2 appeared to have the most favorable PK profile.

All three compounds were found to be brain penetrant following all routes of administration (FIG. 25B). IV administration of each compound resulted in rapid accumulation in brain, at concentrations greater than 300 μM, 75 μM, and 50 μM for TH-2-31, TH-4-55-2, and TH-4-67, respectively. We ruled out the possibility that high initial concentration in brain is due to residual blood, as we didn't observe a similar trend in the PK study of PHB4082. Instead, the concentration of PHB4082 in brain was lower than the concentration of it in plasma and it was around 200 nM. We reasoned that this rapid brain accumulation was the cause of the fainting phenotype noted above, and sought to test this idea by designing a less brain-penetrant analog (TH-4-100-2) that incorporated an adamantyl moiety at the R₁ position versus a cyclohexyl moiety in TH-2-31. Mice dosed with TH-4-100-2 were less impaired immediately following injection and recovered more quickly than mice dosed with an equivalent dose of TH-2-31, suggesting that decreasing the brain penetrance of ferrostatins can decrease this transient adverse effect following bolus IV injection. Among the three compounds of interest, TH-2-31 was the most brain penetrant when administered IV, accumulating in brain at a concentration of 10 μM even 24 hours after administration. TH-4-67 accumulated the least of the three compounds in brain with concentrations <200 nM for all routes of administration 24 hours post administration, while TH-4-55-2 was the most stable following IP and PO administration with concentrations >1 μM 24 hours post administration. Again, this suggested that TH-4-55-2 had the most favorable PK and biodistribution profile and might be most effective for CNS-linked ferroptosis applications.

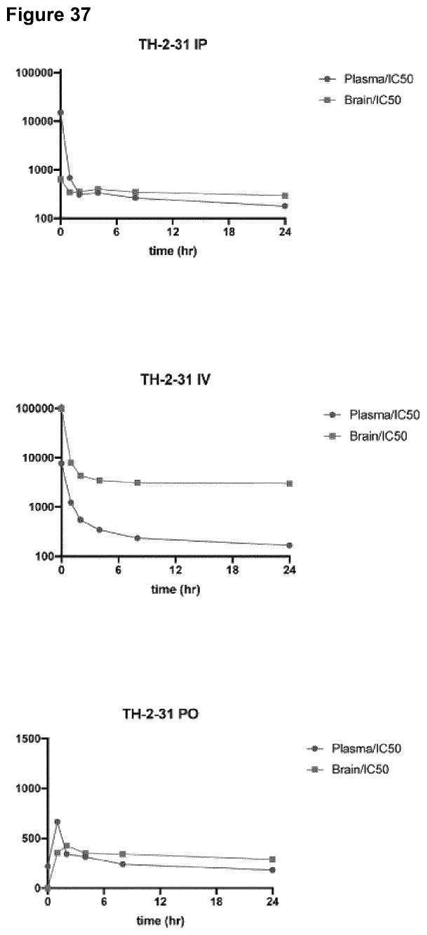

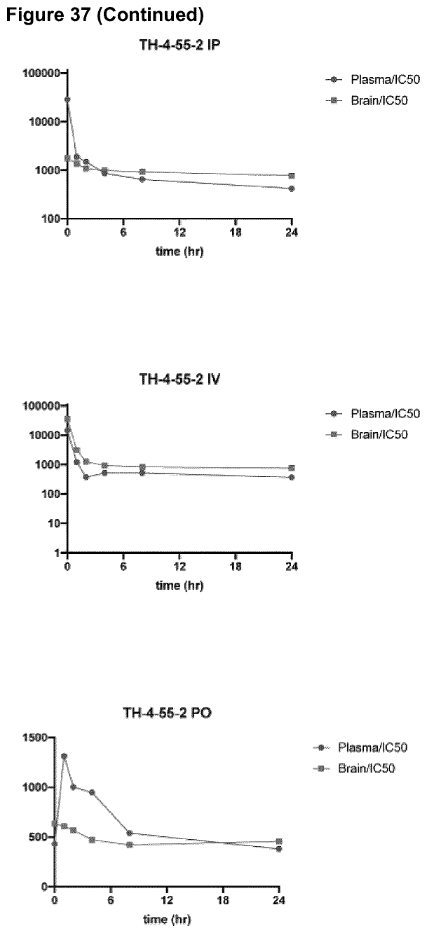

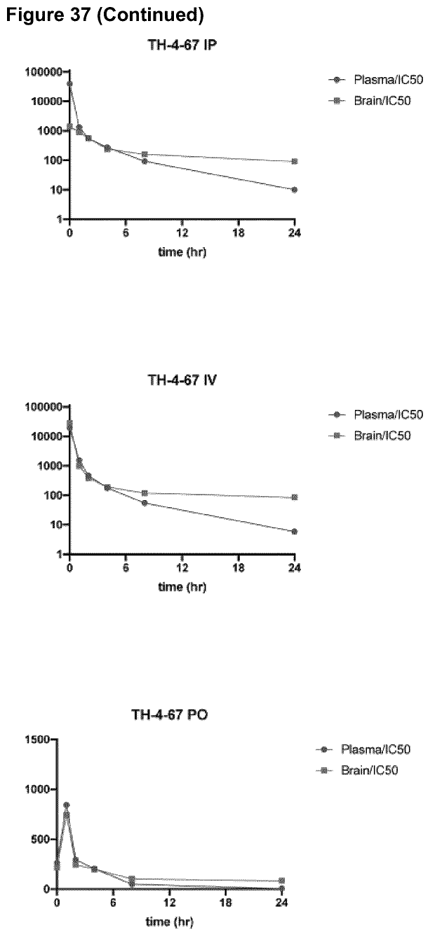

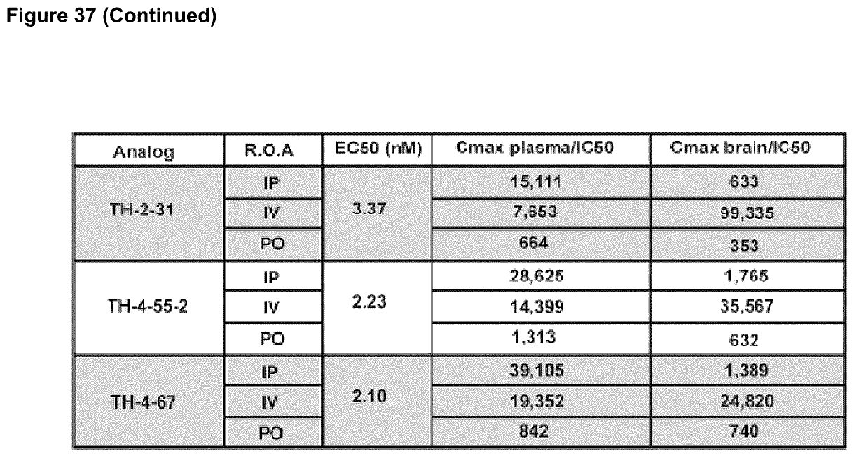

In plasma and brain, all three compounds had Cₘₐₓ values in the micromolar range for all routes of administration (FIG. 25C). As expected, oral administration resulted in the lowest Cₘₐₓ values in the single digit μM range, while IP and IV administration yielded Cₘₐₓ values in the double-to-triple digit μM range. For each compound, comparing the concentration in plasma and brain with the IC₅₀ value for cell culture efficacy revealed that each compound accumulated at concentrations at least five times greater than the IC₅₀ values for all routes of administration in plasma, and at concentrations greater than 50 times the IC₅₀ value in brain, even 24 hours post administration (FIG. 37).

While all three compounds were found to accumulate in brain, to be effective for neurodegenerative disease applications, they should preferentially accumulate in brain over plasma. The log ratio of the concentration of compound in brain over plasma, log₁₀(Brain/Plasma) was calculated for each time-point and route of administration and plotted for each compound (FIG. 25D). At 24 hours, all three compounds had a log₁₀(Brain/Plasma) value >0 for all routes of administration, indicating that they preferentially accumulate in brain over plasma. TH-2-31 and TH-4-55-2 preferentially accumulated in brain over plasma for all time-points following IV administration.

With IP and PO administration of all three compounds, the compounds initially accumulated in plasma and over time began to accumulate in brain. For all three routes of administration at 24 hours, TH-4-67 had the highest log₁₀(brain/plasma) values, other than TH-2-31 IV administration. This is likely due to the fact that both TH-2-31 and TH-4-55-2 stably accumulated at similar concentrations in both plasma and brain, while TH-4-67 was metabolized in plasma, but to a lesser extent in brain. The high retention of compounds in brain might be due to partitioning into the lipophilic region. Localization of compounds might slow down their metabolism and influence their function.

### In Vivo Safety Testing of Fifth-Generation Analogs in Disease Models

To determine whether these fifth-generation ferrostatins were suitable to probe whether ferroptosis is involved in the etiology of neurodegenerative diseases, we utilized two mouse models of degeneration: the phenotypic 3-nitropropionic acid (3-NP) model of striatal degeneration, which is not linked to ferroptosis, and the N-terminal transgenic R6/2 Huntington's mouse model, which has features of ferroptosis (Mangiarini et al. 1996; Tunez et al. 2010).

First, male C57BL/6 mice at ˜8 weeks of age were dosed with vehicle or each fifth-generation analog at 20 mg/kg IP daily for three days prior to, and in addition to, daily IP dosing with 3-NP in an escalating dose series over 5 days, with mice receiving a total of 360 mg/kg of 3-NP (Table 9). The injection volume ranged from 180 uL to 300 uL based on different body weights. The body weight of each mouse was recorded daily and the percentage weight change from baseline for each treatment group was plotted as a measure of overall health. Any mouse that lost more than 20% body weight or had a poor body condition was euthanized. Beginning on day three, all mice independent of treatment group steadily lost weight and mixed-effect analysis indicated a significant effect of time, but not treatment, on body weight. These three fifth generation ferrostatins thus did protect against the loss in body weight observed in the 3-NP model of striatal degeneration, which is not linked to ferroptosis.

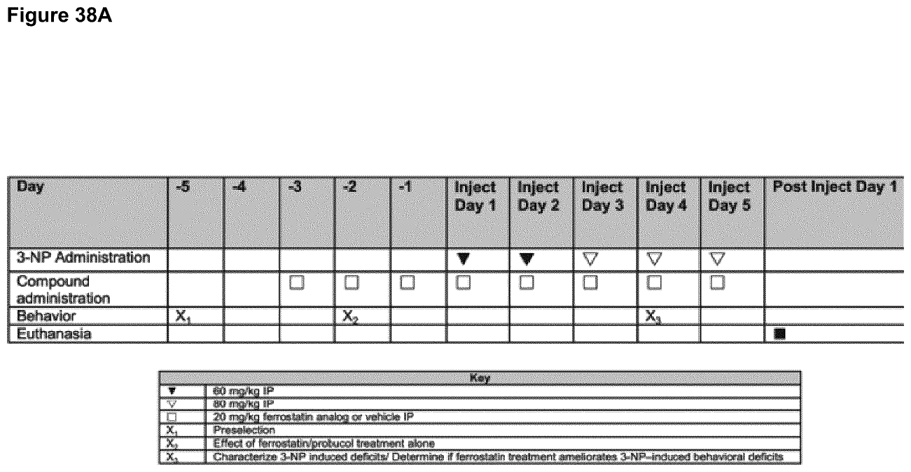

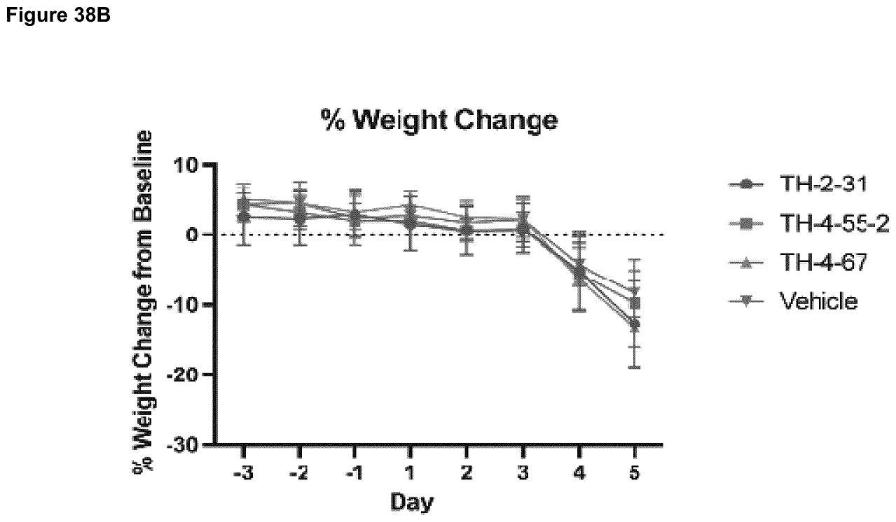

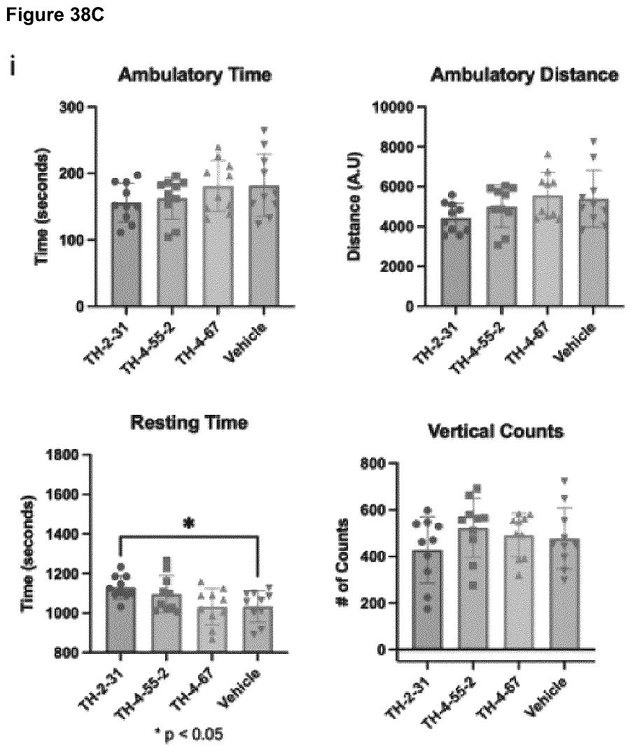

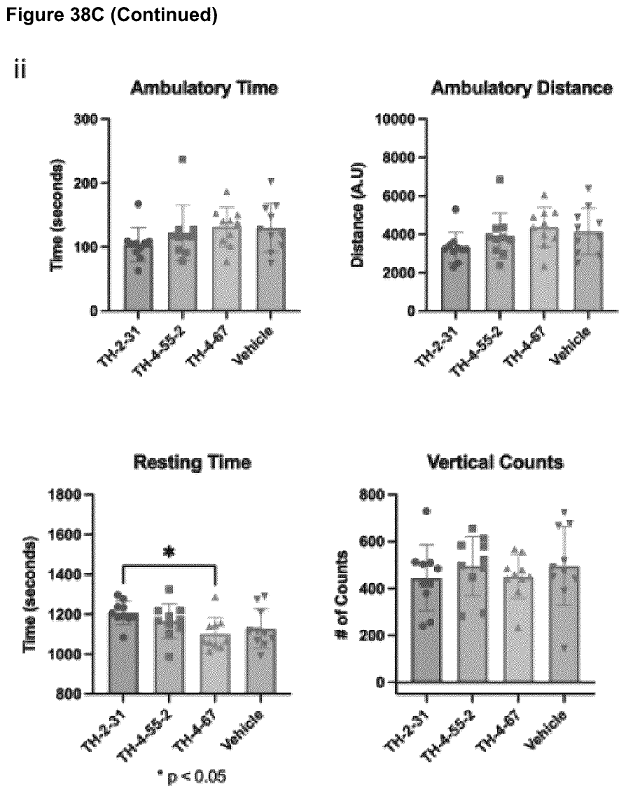

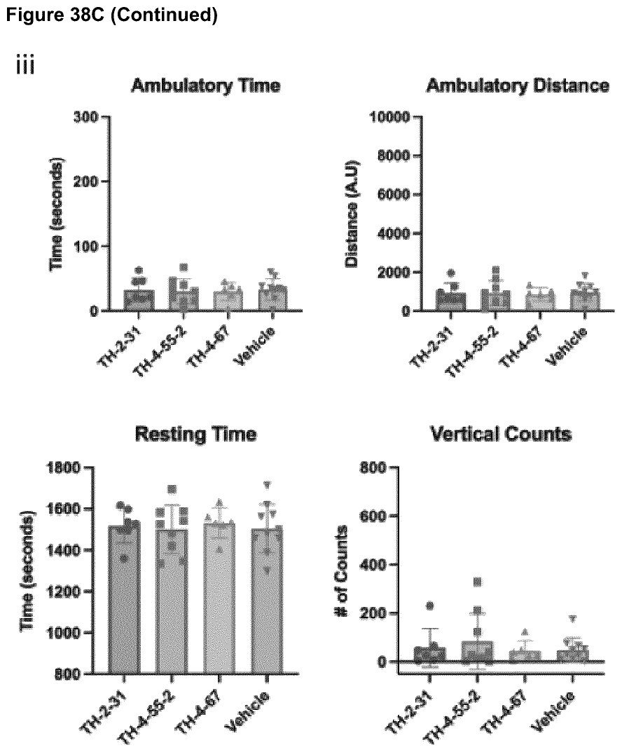

In addition to weighing the mice daily, open field behavior in a 30-minute bin was recorded and analyzed at three different points in the study: on day −5 to establish baseline behavior prior to both ferrostatin and 3-NP treatment (FIGS. 38A-38C), on day −2 to assess whether compound treatment had any effect on behavior (FIG. 38A), and on day +4 to determine whether compound treatment protected against open field deficits induced by 3-NP treatment (FIG. 38B). Open Field performance was assessed across 10 metrics, including time, distance, and vertical counts (i.e., how many times they stand). There was no difference in ambulatory time, distance, or vertical counts between vehicle and ferrostatin analogs on days −5 and −2, indicating that compound treatment alone had no impact on behavioral deficit caused by 3-NP. All mice displayed profound open field deficits on day 4 across all metrics with no significant difference between the treatment groups (FIG. 38C). Taken together, these findings suggest that treatment with ferrostatins is ineffective in preventing weight loss and behavioral deficits in the 3-NP model. Given that the 3-NP model is not linked to ferroptosis, this suggests that ferrostatins don't generally protect against neurodegeneration or ROS-based neuronal damage, which is evident in the 3-NP model. Thus, this study allowed us to rule out the possibility that these ferrostatin compounds have a general neuroprotective effect in mice.

In order to assess whether ferrostatins can be used for long-term efficacy treatments and are selectively protective in models with features of ferroptosis, we performed a toxicity study to determine whether symptomatic R6/2 Huntington Disease model mice could tolerate chronic administration of compounds. We focused on TH-4-55-2, given its promising PK profile. Symptomatic R6/2 and age-matched C57BL/6 wild type mice of both sexes at ˜10 weeks of age were dosed daily with 20 mg/kg of TH-4-55-2 via IP or PO for 30 days. Body weight was measured and recorded and the percent change in body weight from baseline calculated. Per IACUC guidelines, any mouse that lost more than 20% body weight for three days was euthanized prior to completion of the study.

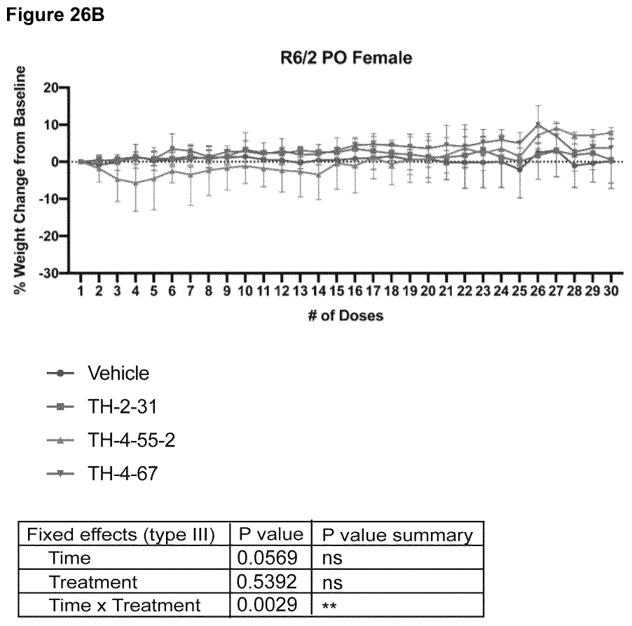

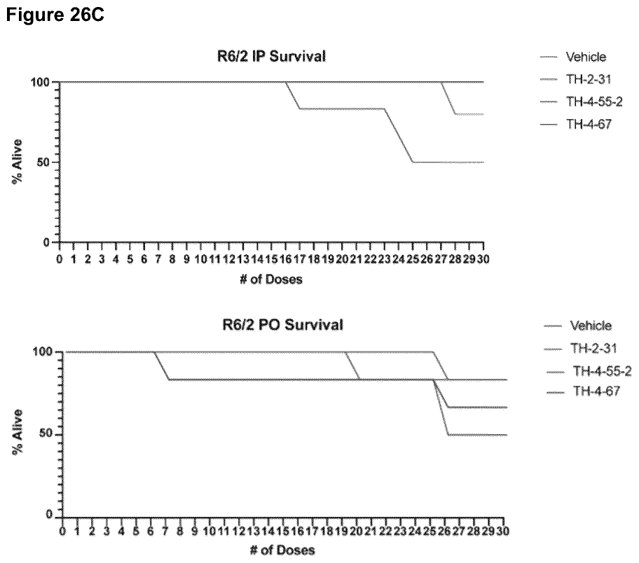

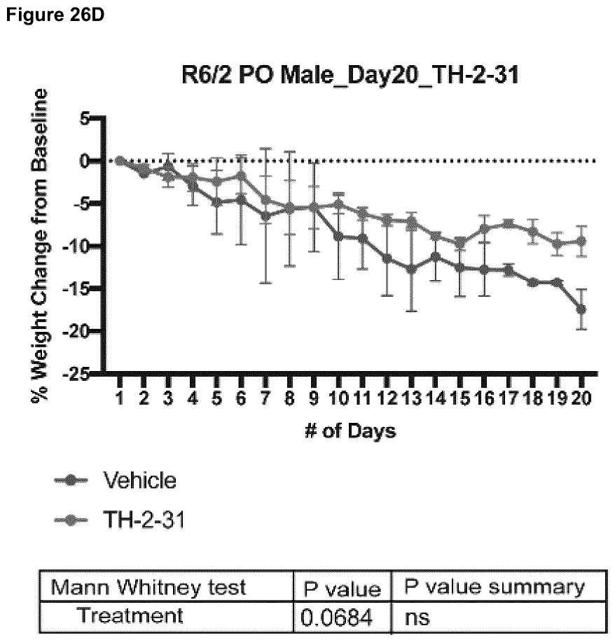

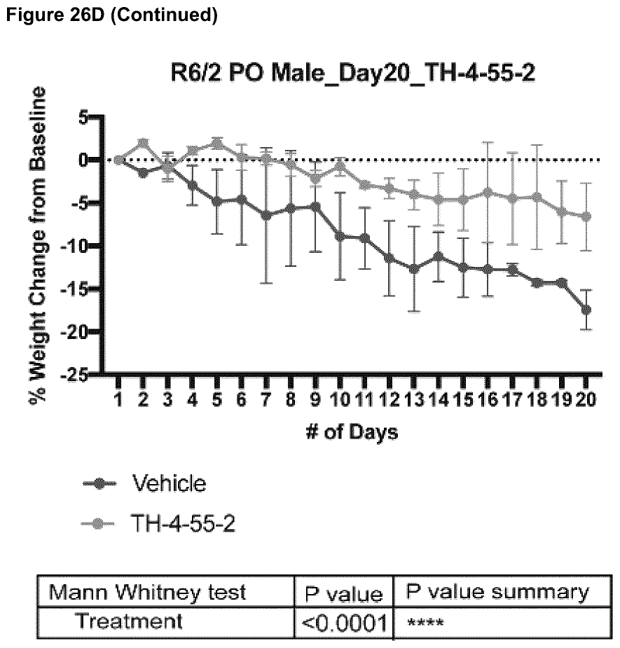

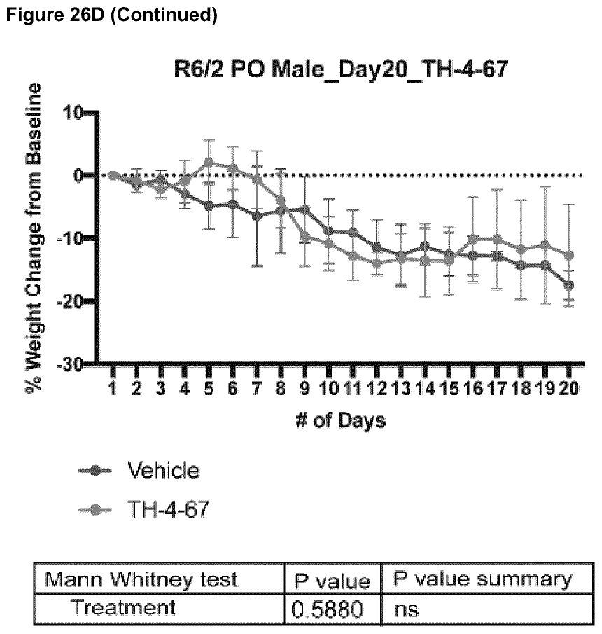

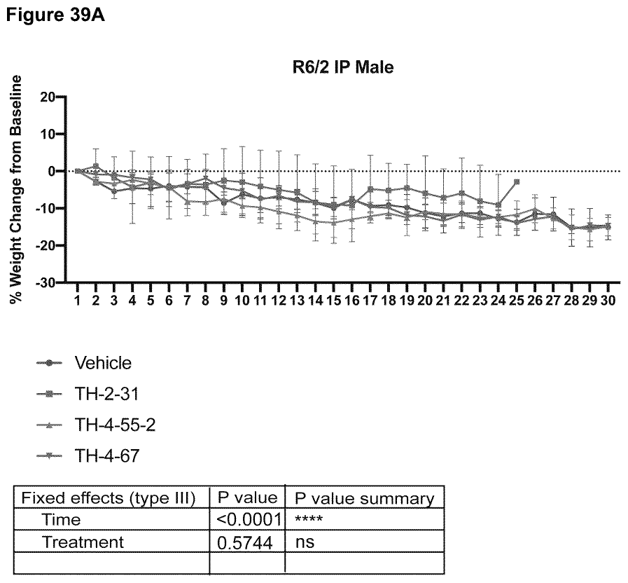

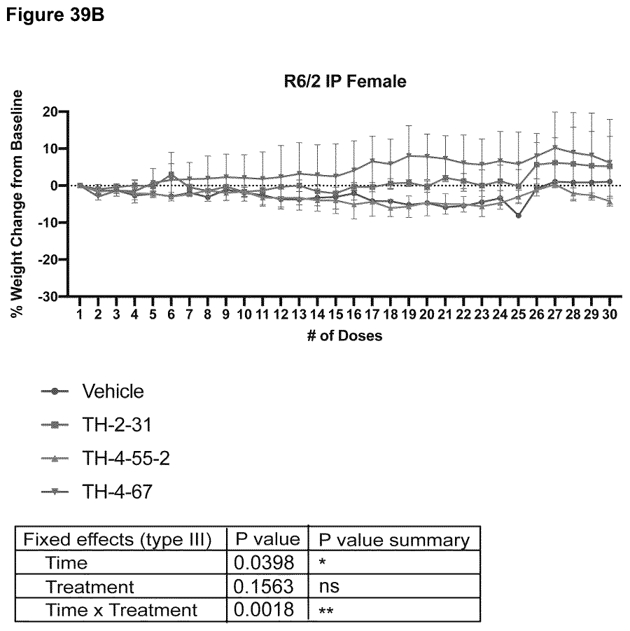

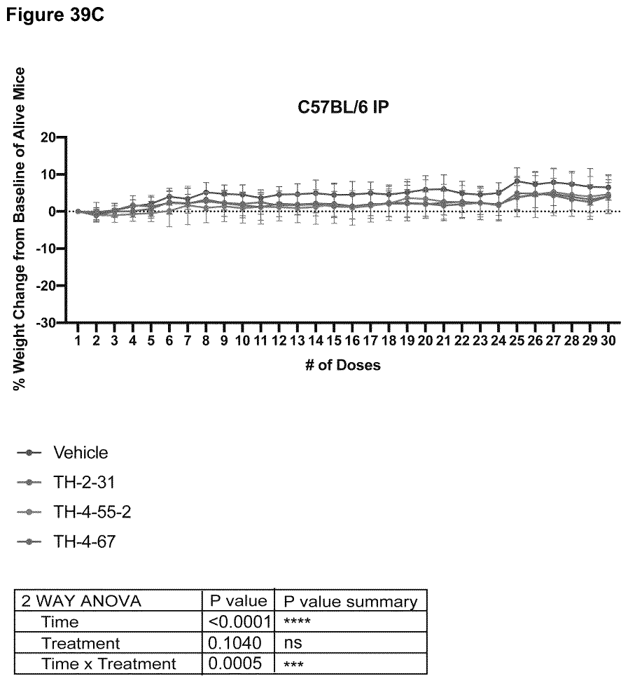

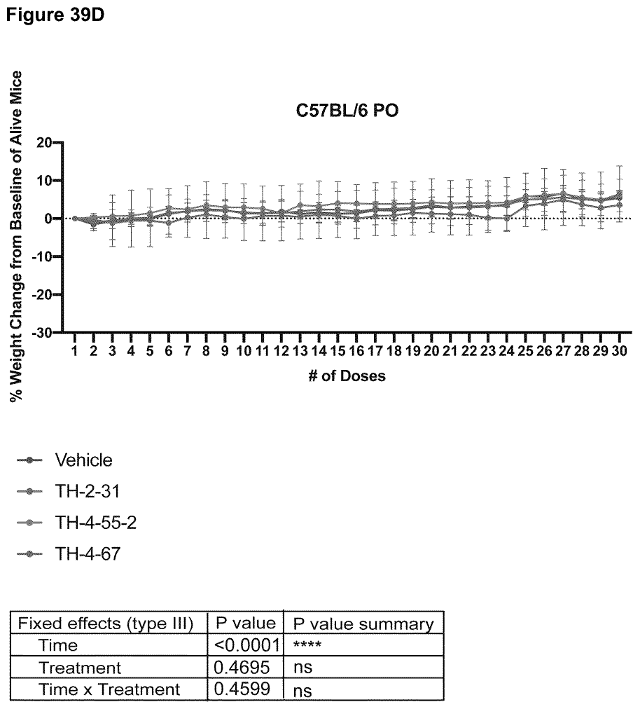

After 30 days of IP administration, no vehicle-treated mice and one TH-4-55-2-treated mouse died; after 30 days of PO administration, two vehicle-treated and one TH-4-55-2-treated mouse died (FIG. 26C). Analysis revealed no significant effect of treatment for PO-treated mice or IP-treated R6/2 mice (FIGS. 26A-26B and FIGS. 39A-39B). There is also no significant effect of treatment for PO-treated mice or IP-treated wild type mice. Thus, compound treatment by IP or PO routes did not cause a loss of body weight, which is a general marker of animal health. These results suggest that these lead compounds are well tolerated over 30 days in wild-type and R6/2 mice.

There was a significant weight loss trend over time in the R6/2 male group regardless of administration route or treatment group (FIG. 26A), as has been reported (van der Burg et al. 2008). We didn't observe such a trend with the female R6/2 mice. Notably, R6/2 male mice treated with TH-4-55-2 via PO administration were protected from body weight loss over time. This protective effect of TH-4-55-2 was confirmed to be statistically significant in the male group via PO administration over a 20-day course, as we sacrificed mice losing over 20% body weight due to their R6/2 genotype on the 21^{th} day. TH-2-31 and TH-4-67 didn't show this protective effect (FIG. 26D).

In summary, these fifth generation ferrostatins are well-tolerated by R6/2 mice, suggesting that they can be used in future studies requiring chronic administration regimens. Moreover, the protective effect of TH-4-55-2 against weight loss in the male R6/2 mice suggests that this compound may protect against deficits associated with Huntington Disease, and perhaps other neurodegenerative diseases linked to ferroptosis.

The results from the prior in vivo PK study indicate that all three compounds are brain penetrant and preferentially accumulate in the brain at concentrations greater than 50 times their IC₅₀ values. Additionally, these ferrostatins were demonstrated to be specific for ferroptotic-cell death, and the fifth-generation compound TH-4-55-2 was well-tolerated in a 30-day toxicity study in wild-type mice and symptomatic R6/2 HD mice, and showed protection against HD-related weight loss.

Taken together, these studies indicate that fifth generation ferrostatins can be utilized to probe the contribution of ferroptosis to neurodegenerative diseases. These studies also illuminated detailed SAR around the benzene and pyridine cores for ferrostatins (FIG. 27). We additionally tested the tolerability of TH-2-31 and TH-4-67 over 30 days using IP and PO administration and similarly found no detectable weight loss induced by either compound (FIGS. 39A-39D). Thus, all three ferrostatins were well tolerated using both routes of administration.

Formulation of Compounds in Water without Polyethylene Glycol (PEG)

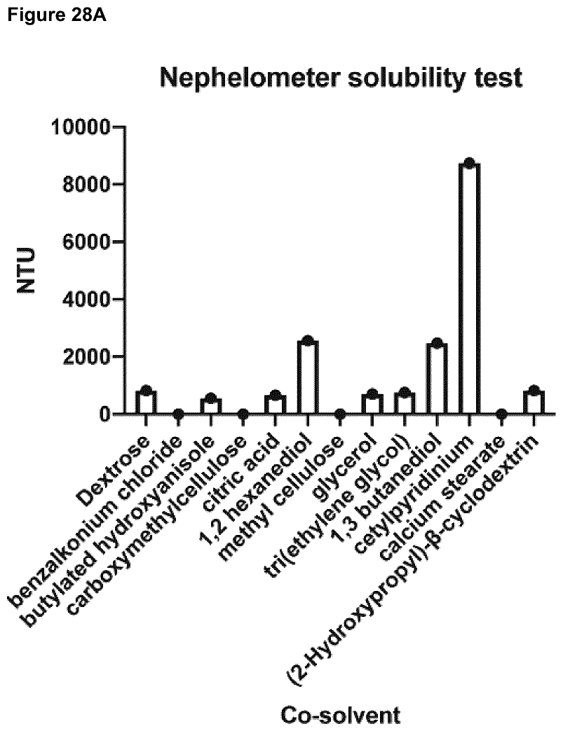

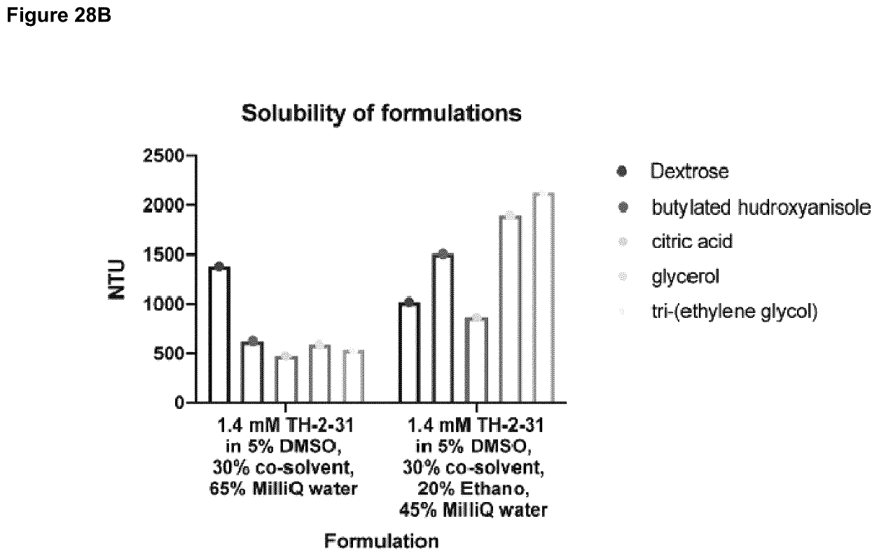

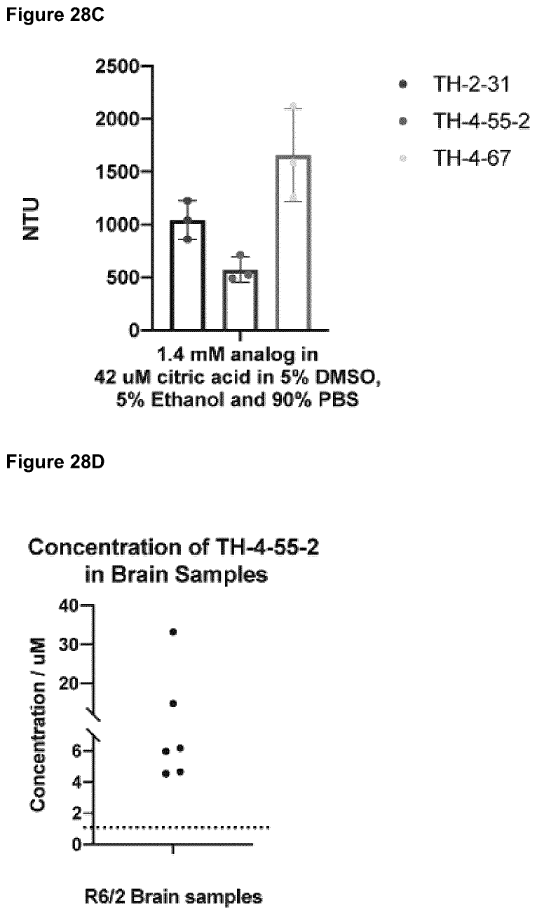

Evaluating extended studies in mouse models of neurodegenerative diseases will likely necessitate the compounds being formulated into drinking water. This is due to the logistical challenges and tolerability issues associated with a daily IP or PO administration, particularly for ailing mice. The original formulation employed in prior mouse studies involved dissolving the ferrostatin in 5% DMSO/95% of 1:1 [(65% v/v of 25% w/v 2-hydroxypropyl-β-cyclodextrin dissolved in 20% EtOH, 30% v/v Poly(ethylene glycol)-400, 5% v/v Tween 80):MilliQ water]. However, inclusion of PEG-400 in the previous formulation poses contamination concerns for LC-MS analyses. Thus, we took efforts to design a suitable formulation for administering these three compounds in drinking water. We first identified potential co-solvents using a nephelometer by measuring aerosol light scattering. Solutions were prepared using the existing formulation, substituting PEG-400 with various co-solvent candidates, and nephelometric turbidity was assessed (FIG. 28A). Dextrose, butylated hydroxyanisole, citric acid, glycerol, and tri(ethylene glycol) were identified as potential co-solvents for the next step. Then, we further tested potential co-solvents in small-scale experiments with analog TH-2-31. Formulations with or without ethanol were tested with different co-solvent candidates (FIG. 28B). Among these five co-solvents, citric acid had the lowest readings and was the most promising co-solvent. In further tests, PBS was included in the formulation to adjust the pH value. We tested this formulation with all three lead compounds, TH-2-31, TH-4-55-2, and TH-4-67 (FIG. 28C) and finalized the formation as 1.4 mM of analog and 42 μM citric acid in 5% DMSO, 5% ethanol, and 90% PBS. This formulation without PEG can be used in drinking water and in long-term efficacy studies and facilitates subsequent LC-MS analysis.

### In Vivo Pharmacokinetic Study of TH-4-55-2 in Drinking Water

Effectively formulating compounds suitable for administration through drinking water, we initiated an in vivo analysis of the pharmacokinetic properties of TH-4-55-2 due to its promising profile. Symptomatic R6/2 mice of both sexes at ˜8 weeks received daily doses of TH-4-55-2 via drinking water. Our observation indicated the average amount of water each mouse would drink is approximately 2 mL per day. We adjusted the concentration of the compound in the drinking water to have an expected dosage of 20-30 mg/kg/day. Mice were administered compound in drinking water for four days. Body weight was measured and recorded and the percent change in body weight from baseline was calculated. After 4-day treatment, mice were euthanized, with plasma and brain homogenate extracted with acetonitrile and analyzed via UPLC-MS.

The presence of TH-4-55-2 was detected in all brain samples of symptomatic R6/2 mice. The detected concentration varied from 1.5 μM to 33 μM (FIG. 28D). The variation may be due to different amounts of water drunk by different mice, and different timing of drinking. However, the concentrations of compound achieved in the brain suggest this formulation can be used in future long-term efficacy study.

### In Vivo Pharmacokinetic Study of TH-4-55-2, TH-4-67, TH-2-31 in Drinking Water and PO

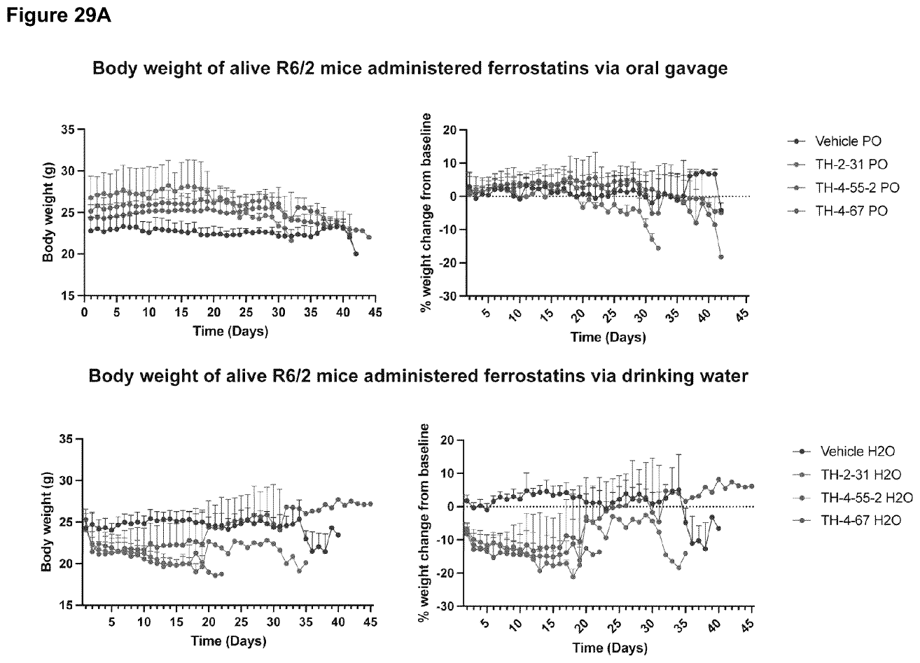

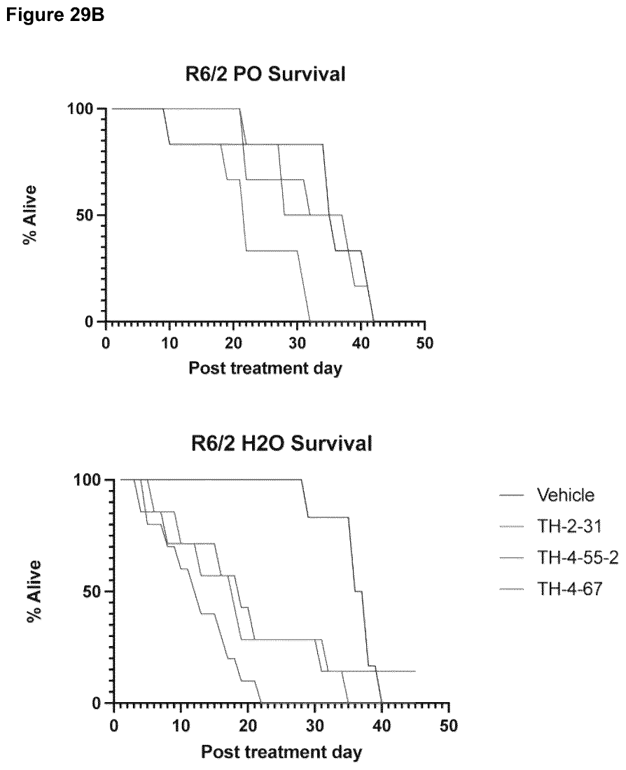

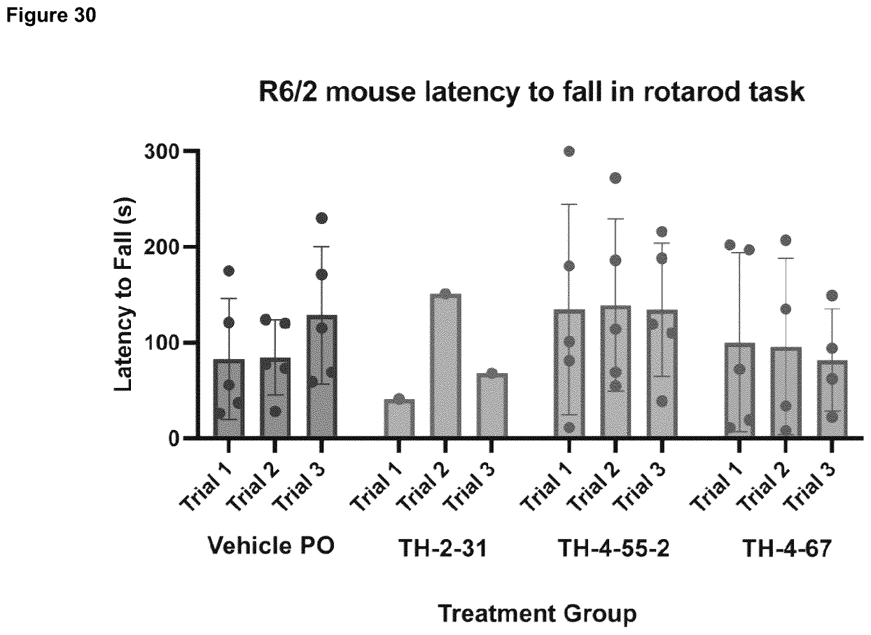

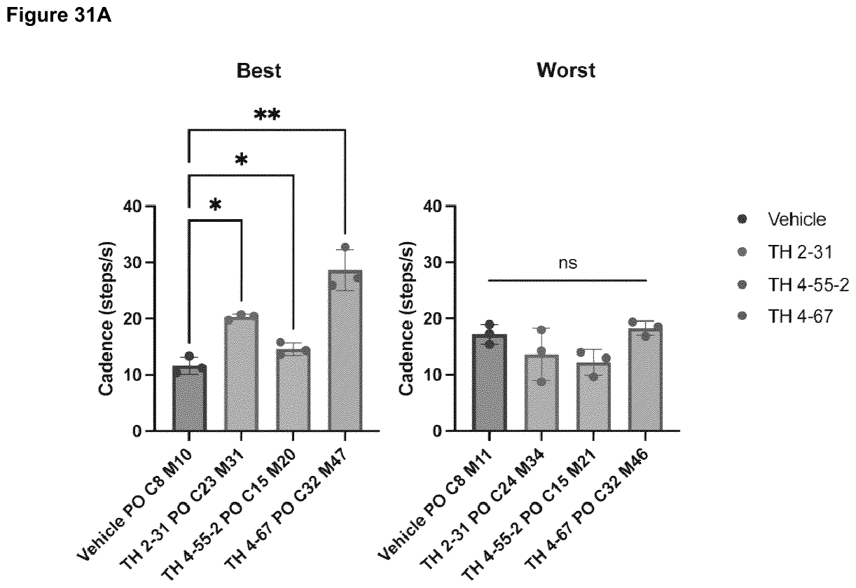

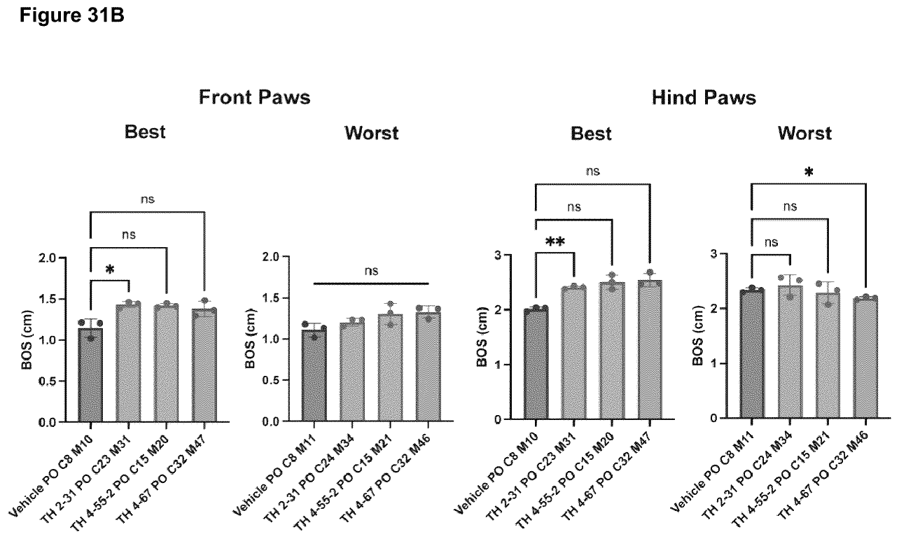

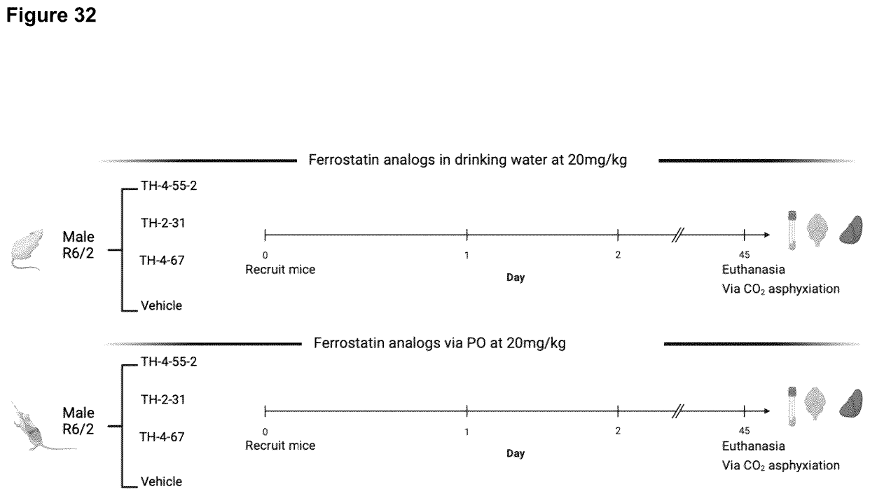

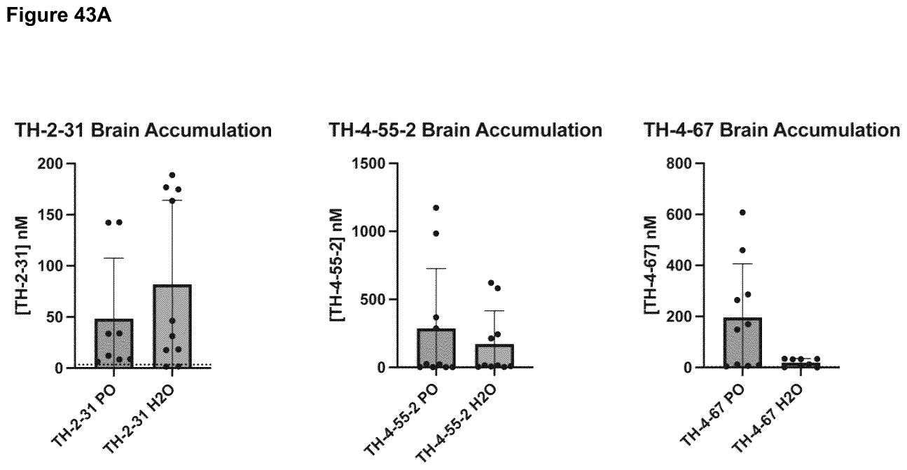

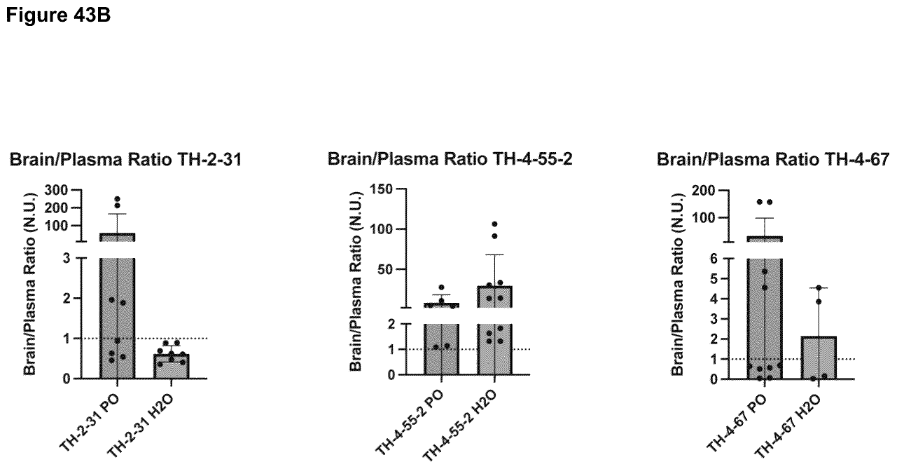

The pharmacokinetic properties of TH-4-55-2 were initially assessed in vivo, focusing on its BBB distribution. Subsequent investigations involved TH-4-67 and TH-2-31. Symptomatic R6/2 male mice, aged ˜6-7 weeks, dosed via PO and treated through drinking water with TH-4-55-2, TH-4-67, TH-2-31 and Vehicle. Due to previously observed weight loss trends in R6/2 males ROA, only male mice were employed. Daily consumption of approximately 2 mL of water delivered a dosage of 20 mg/kg/day of the respective compound, along with compounds administered orally (FIG. 32). This regimen continued for 45 days, during which changes in body weight were monitored (FIG. 29A). Mouse body weights that fell under 20% of baseline measurements were sacrificed, while spontaneous deaths over the course of the study were monitored (FIG. 29B). Motor coordination was assessed via Rotarod behavior measurements (FIG. 30), and gait analysis was conducted using CatWalk (FIGS. 31A-31B). At the end of the 45-day treatment, mice were euthanized via CO₂ asphyxiation, blood collected via cardiac puncture, brain and liver organs were harvested. Plasma and brain were subjected to UPLC-MS analysis, enabling determination of compound concentrations against concurrently run standard curves (FIGS. 43A-43B).

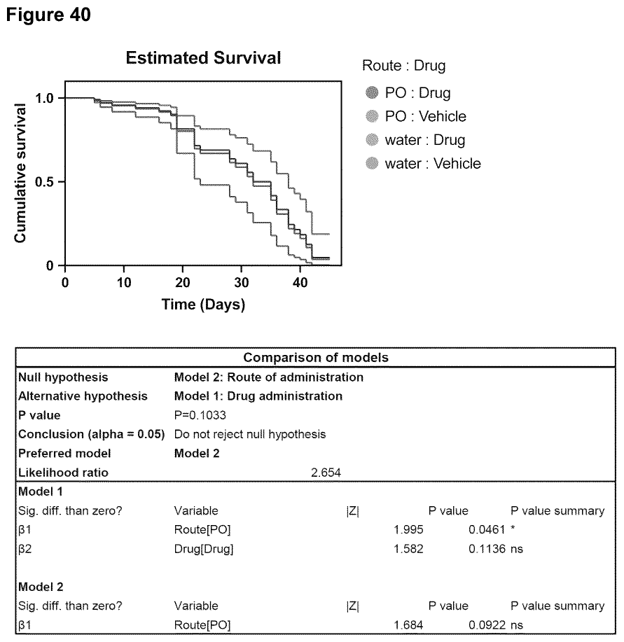

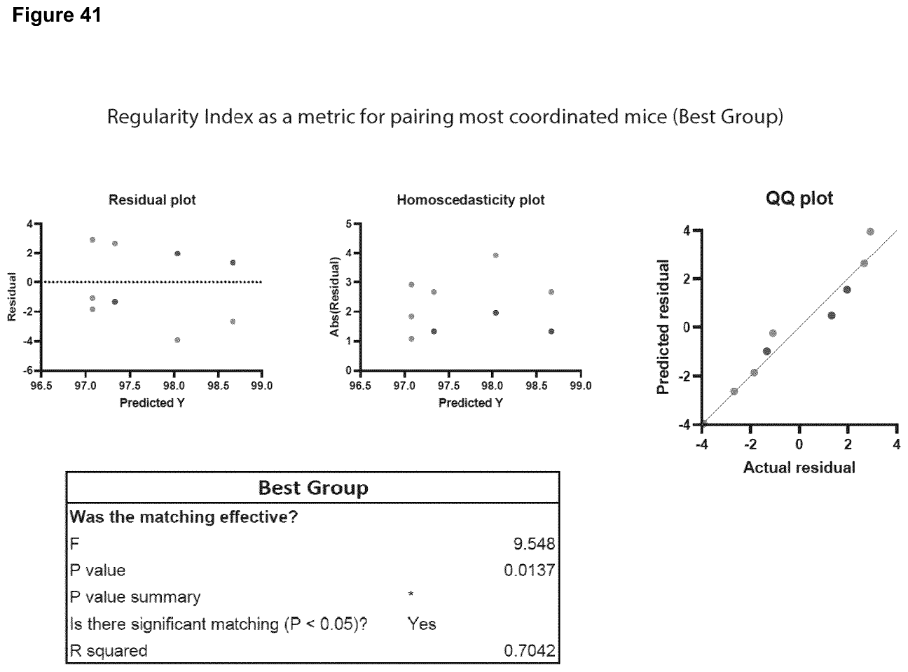

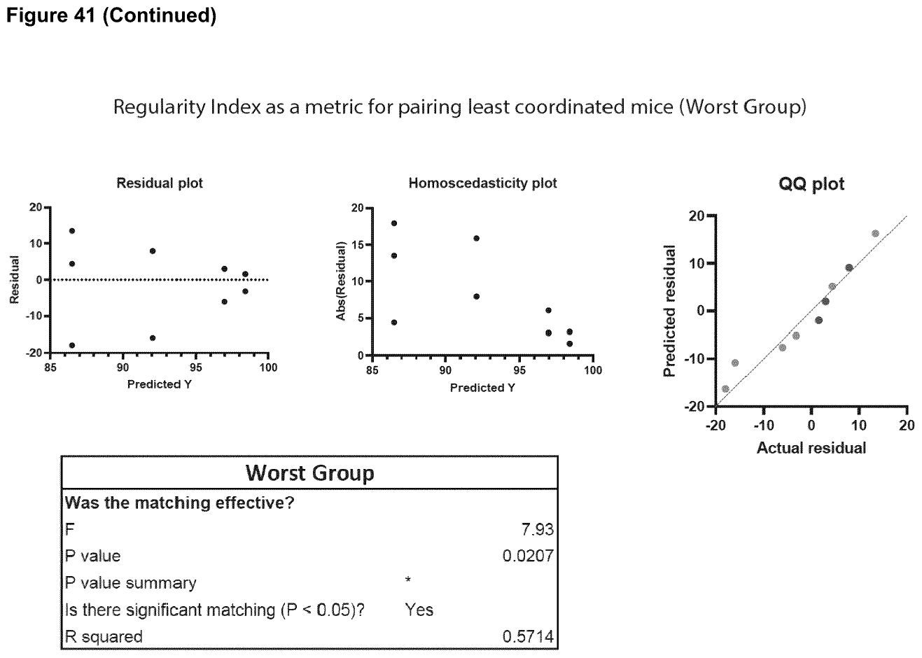

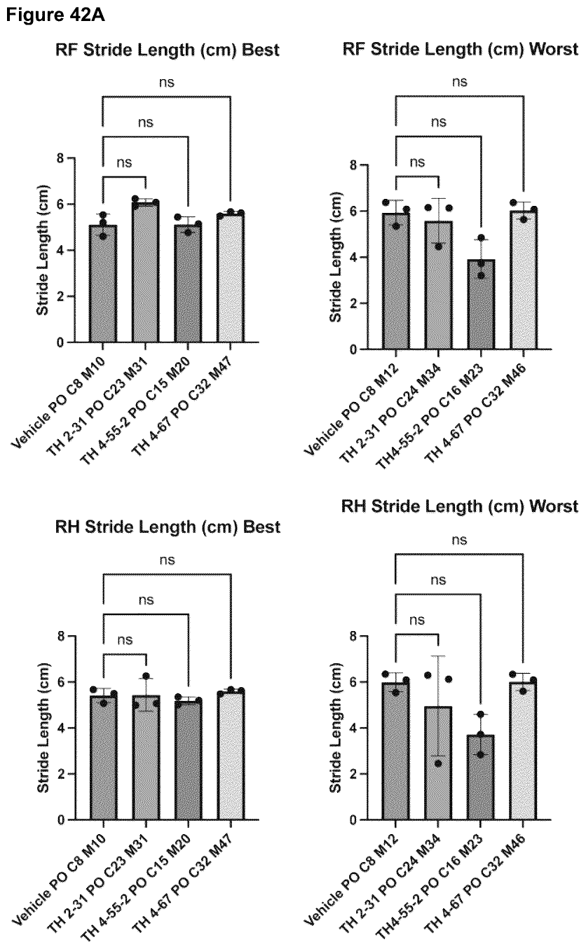

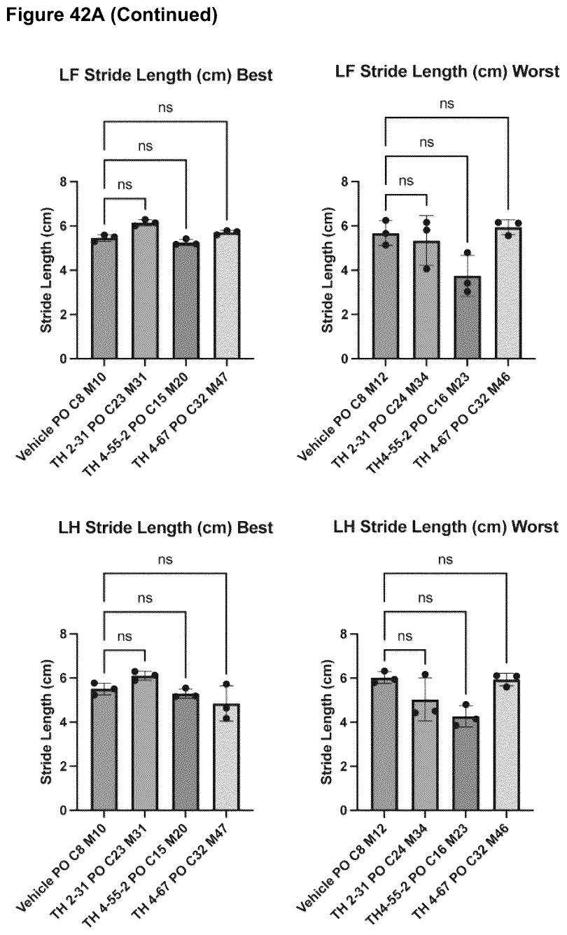

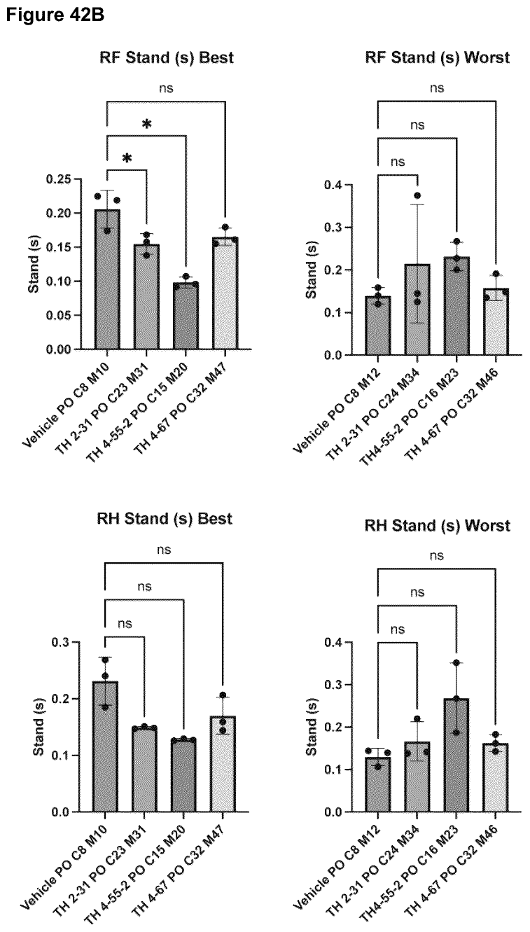

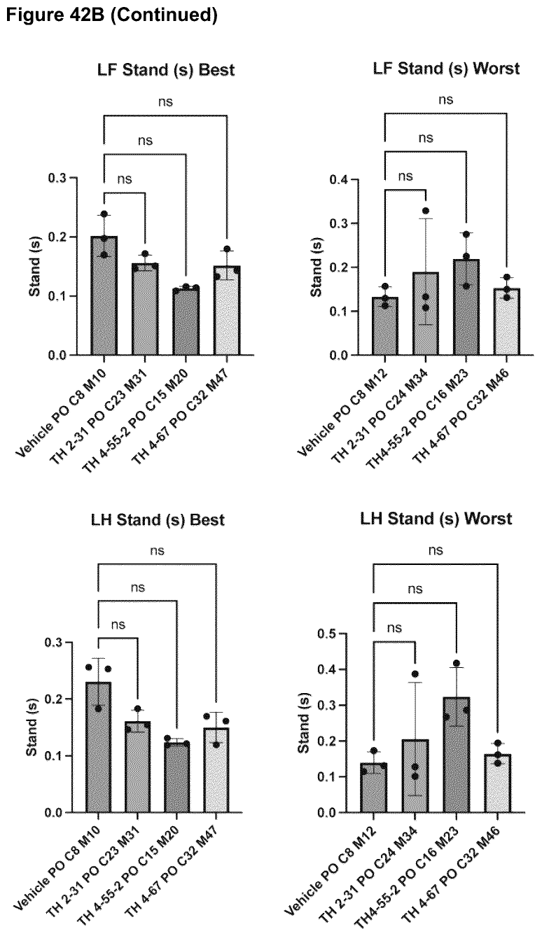

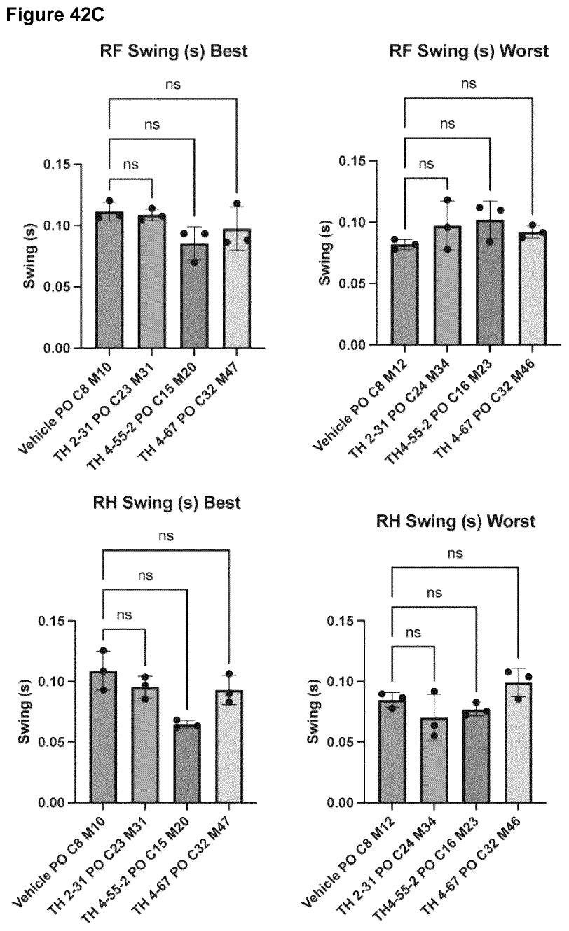

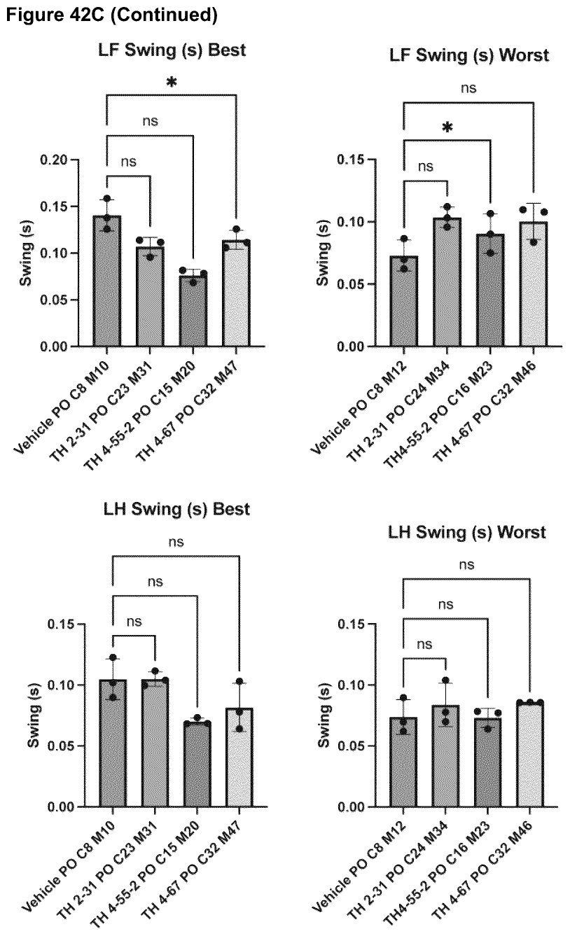

Treatment of R6/2 mice with ferrostatins yielded mixed results for each ferrostatin analog. Long-term administration of ferrostatins in drinking water had a significant effect of route of administration on mouse survival detected via Cox regression analysis (FIG. 40). We propose that the long-term administration of ferrostatins via drinking water may have caused dehydration in mice due to bitter flavor, contributing to their spontaneous death over the course of the study. Thus, further behavioral analysis was focused on mice dosed via PO. Rotarod behavioral data did not reveal significant differences between treatment groups, while the average performance of the TH-4-55-2 PO treatment group consistently scored higher compared to control (FIG. 30). In the Catwalk Gait Analysis task, individual run and mouse data was paired by regularity index, a measure of interlimb coordination, to compare runs and mice of most coordinated “Best” and least coordinated “Worst” groups (FIG. 41). Significant improvements in Cadence were observed among the Best performing mice for all ferrostatin analogs, with TH-4-67 as the leading compound among this group (FIG. 31A). Similarly, TH-4-67 showed a significant decrease in Base of Support (BOS) for the Worst performing mice in Hind paws compared to control, hinting at an improvement of gait under TH-4-67 treatment. Conversely, TH-2-31 showed significantly worse BOS compared to Vehicle (FIG. 31B). Other individual paw measures relevant to HD rodent models in the Catwalk task, such as stride length, stand time, and swing, either had isolated significance to one paw in one group, or did not yield any significance (FIGS. 42A-42C). Overall, behavioral data collected in this pharmacokinetic study warrants further in vivo investigation of TH-4-55-2 and TH-4-67. An efficacy study designed with greater statistical power and optimized long-term drug delivery may reveal prominent improvements to motor impairment in Huntington's Disease upon ferrostatin treatment.

### Discussion

Previous reports suggested that fer-1 is protective in a variety of disease-relevant contexts, including Huntington Disease, ischemia reperfusion injury in the kidney, Parkinson's disease, and ischemic stroke, among others (Li et al. 2017; Skouta et al. 2014). However, in vivo applications were limited, due to this original ferrostatin's low in vitro metabolic and plasma stability, lack of brain penetration, and the presence of aniline and phenylenediamine moieties. There is thus a need for potent, brain-penetrant, stable, and non-toxic ferrostatin analogs that can be used to evaluate the role of ferroptosis in neurodegenerative disease models.

In this study, we developed two new generations of ferrostatin analogs. Among the fourth generation, the lead compound PHB4082 stably accumulated in kidney over time, with an average concentration of 1.8 μM, which suggests a potential for PHB4082 to be used in kidney-related applications. PHB4082 significantly ameliorated renal tubular ferroptosis in an acute kidney injury (AKI) model by ferric nitrilotriacetate (Fe-NTA). Previous research suggested that pharmacological inhibitors of ferroptosis can be employed to alleviate ferroptosis in acute kidney injury (AKI) (Ni, Yuan, and Wu 2022), including renal ischaemia reperfusion injury (Chen et al. 2021), cisplatin-induced AKI (Deng et al. 2019), and folic acid-triggered AKI (Martin-Sanchez et al. 2017). PHB4082 can be a lead compound to develop new alternatives for the treatment of these diseases. Co-treatment of PHB4082 with cisplatin may improve the functional and histological deterioration in cisplatin-induced AKI, for example.

Among the fifth-generation compounds, the lead TH-4-55-2 was brain-penetrant, stable, and non-toxic, which is a candidate for future applications in neurodegenerative diseases, such as Huntington Disease, Parkinson's Disease, and in other ferroptosis-related diseases, including ischemic stroke, and traumatic brain injury. Previously, Do Van et al. demonstrated protection of fer-1 against the toxic effect of MPTP, a Parkinson's Disease model, on dopaminergic neurons (Do Van et al. 2016). Fer-1 has also been shown to significantly reduce functional deficits in ischemic stroke mouse models (Tuo et al. 2017). An increase in the ferroptosis phosphatidylethanolamine marker after traumatic brain injury was reported (Wu et al. 2019). However, fer-1 is not brain-penetrant; thus, in these mouse models, fer-1 was either directly injected into the brain (Parkinson's Disease) or by intranasal administration for acute treatment (ischemic stroke), which has limited translational potential. With the newly developed TH-4-55-2 compound having good brain penetration, it may be possible to evaluate the impact of inhibiting ferroptosis in a variety of neurodegenerative models, including Huntington Disease, Parkinson's Disease, ischemic stroke, and traumatic brain injury.

### Significance

Ferrostatin-1, while effective in vitro, is unsuitable for in vivo use. Previous efforts have demonstrated that fer-1 is protective in a variety of disease-relevant contexts, including Huntington disease. However, in order to examine the role of ferroptosis in vivo, a potent, brain-penetrant, and stable ferrostatin is required. Our efforts at developing fourth generation and fifth-generation ferrostatin analogs resulted in the identification of four lead candidates that can be utilized in vivo. The lead compound of fourth generation ferrostatins, PHB4082, has been proven to ameliorate renal tubular ferroptosis in an acute kidney injury (AKI) model, suggests a potential in AKI application. The development of fifth generation ferrostatin analogs represents a significant step forward in ferrostatin development efforts. For the first time, potent, stable, and brain penetrant ferrostatin analogs are available without a phenylenediamine toxic moiety associated with all previously developed ferrostatins. Additionally, the ferrostatin scaffold and SAR have been greatly expanded. In addition to the scaffold utilized by fer-1, a new fifth-generation scaffold was developed, with the most potent analog in N27 cells utilizing the new scaffold. Accordingly, this expands the pool of potential ferrostatin analogs that can be developed and evaluated in disease models. These studies also represent the first instance of fifth generation ferrostatin analogs being evaluated in vivo. The finding that both wild-type and symptomatic R6/2 mice can tolerate chronic daily dosing with ferrostatin analog allows for use in vivo in applications requiring chronic dosing regimens. Notably, the observed protection against weight loss in male R6/2 mice treated PO with TH-4-55-2 as well as conceivable motor improvements with TH-4-55-2 and TH-4-67 suggests that ferrostatins could have efficacy in HD animal models.

In summary, the findings described in this paper expand the knowledge, chemical space, and applicability of ferrostatins, and identify one lead compound that can be evaluated in kidney-related diseases and three lead compounds that can be evaluated in neurodegenerative disease models to determine in which contexts ferroptosis is involved in disease etiology and pathology.

### DOCUMENTS CITED

ABDEL-MAGID, A. F., et al. J. Org. Chem. 1996, 61, 3849-3862.
Akatsuka, S., et al. 2012. ‘Fenton reaction induced cancer in wild type rats recapitulates genomic alterations observed in human cancer’, PLoS One, 7: e43403.
ANITHA, M., et al. Targeting glutamate mediated excitotoxicity in Huntington's disease: neural progenitors and partial glutamate antagonist—emantine. Medical hypotheses 76, 138-140, doi:10.1016/j.mehy.2010.09.003 (2011).
BANJAC, A., et al. (2008). The cystine/cysteine cycle: a redox cycle regulating susceptibility versus resistance to cell death. Oncogene 27, 1618-1628.
Banks, W. A. 2009. ‘Characteristics of compounds that cross the blood-brain barrier’, Bmc Neurology, 9.
BARTZOKIS, G., et al. Increased basal ganglia iron levels in Huntington disease. Arch Neurol 56, 569-574 (1999).
BEAULIEU, P. L., et al. Synthesis, 2003, 11, 1683-1692.
BEHL, C. Alzheimer's disease and oxidative stress: implications for novel therapeutic approaches. Prog Neurobiol 57, 301-323 (1999).
BERGSBAKEN, T., et al. (2009). Pyroptosis: host cell death and inflammation. Nat Rev Microbiol 7, 99-109.
Biernacki, K., et al. 2020. ‘Novel 1,2,4-Oxadiazole Derivatives in Drug Discovery’, Pharmaceuticals (Basel), 13.
BLOIS, M. S. (1958). Antioxidant determinations by the use of a stable free radical. Nature 181, 1199-1200.
CATER, H. L., et al. (2007). Stretch-induced injury in organotypic hippocampal slice cultures reproduces in vivo post-traumatic neurodegeneration: role of glutamate receptors and voltage-dependent calcium channels. J Neurochem 101, 434-447.
CHA, J. H. et al. Altered brain neurotransmitter receptors in transgenic mice expressing a portion of an abnormal human huntington disease gene. Proc Natl Acad Sci USA 95, 6480-6485. (1998).
CHEAH, J. H., et al. (2006). NMDA receptor-nitric oxide transmission mediates neuronal iron homeostasis via the GTPase Dexras1. Neuron 51, 431-440.
CHEN, J. C. et al. MR of human postmortem brain tissue: correlative study between T2 and assays of iron and ferritin in Parkinson and Huntington disease. AJNR. American journal of neuroradiology 14, 275-281 (1993).
CHEN, J. et al. Iron accumulates in Huntington's disease neurons: protection by deferoxamine. PLoS One 8, e77023, doi:10.1371/journal.pone.0077023 (2013).
Chen, Y., et al. 2021. ‘Ferroptosis: A Novel Therapeutic Target for Ischemia-Reperfusion Injury’, Front Cell Dev Biol, 9: 688605.
CHOI, D. W. (1988). Glutamate neurotoxicity and diseases of the nervous system. Neuron 1, 623-634.
CHRISTOFFERSON, D. E., and Yuan, J. (2010). Necroptosis as an alternative form of programmed cell death. Current Opinion in Cell Biology 22, 263-268.
CHUNG, N., et al. (2008). Median absolute deviation to improve hit selection for genome-scale RNAi screens. J Biomol Screen 13, 149-158.
Conrad, M., and D. A. Pratt. 2019. ‘The chemical basis of ferroptosis’, Nat Chem Biol, 15: 1137-47.
CRUZ-AGUADO, R., et al. Nerve growth factor and striatal glutathione metabolism in a rat model of Huntington's disease. Restorative neurology and neuroscience 17, 217-221 (2000).
DEGTEREV A, et al. (2005) Chemical inhibitor of nonapoptotic cell death with therapeutic potential for ischemic brain injury. Nat Chem Biol 1(2):112-119.
Deng, F., et al. 2019. ‘Myo-inositol oxygenase expression profile modulates pathogenic ferroptosis in the renal proximal tubule’, J Clin Invest, 129: 5033-49.
DESILVA, T. M. et al. Glutamate transporter EAAT2 expression is up-regulated in reactive astrocytes in human periventricular leukomalacia. J Comp Neurol 508, 238-248, doi:10.1002/cne.21667 (2008).
Devisscher, L., et al. 2018. ‘Discovery of Novel, Drug-Like Ferroptosis Inhibitors with in Vivo Efficacy’, J Med Chem, 61: 10126-40.
DILLON C P, et al. (2012) Survival function of the FADD-CASPASE-8-cFLIP(L) complex. Cell Reports 1(5):401-407.
DILLON C P, et al. (2014) RIPK1 blocks early postnatal lethality mediated by caspase-8 and RIPK3. Cell 157(5):1189-1202.
DIXON, D. J. et al., (2012) Ferroptosis: An Iron-Dependent Form of Nonapoptotic Cell Death. Cell, Vol. 149, Issue 5, pp. 1060-1072.
Do Van, B., et al. 2016. ‘Ferroptosis, a newly characterized form of cell death in Parkinson's disease that is regulated by PKC’, Neurobiology of Disease, 94: 169-78.
Dolma, S., et al. 2003. ‘Identification of genotype-selective antitumor agents using synthetic lethal chemical screening in engineered human tumor cells’, Cancer Cell, 3: 285-96.
DOMMERGUES, M. A., et al. Iron supplementation aggravates periventricular cystic white matter lesions in newborn mice. European journal of paediatric neurology: EJPN: official journal of the European Paediatric Neurology Society 2, 313-318 (1998).
DUCE, J. A., et al. (2010). Iron-export ferroxidase activity of beta-amyloid precursor protein is inhibited by zinc in Alzheimer's disease. Cell 142, 857-867.
ESTRADA SANCHEZ, A. M., et al. Excitotoxic neuronal death and the pathogenesis of Huntington's disease. Archives of medical research 39, 265-276, doi:10.1016/j.arcmed.2007.11.011 (2008).
FOLKERTH, R. D. Periventricular leukomalacia: overview and recent findings. Pediatric and developmental pathology: the official journal of the Society for Pediatric Pathology and the Paediatric Pathology Society 9, 3-13, doi:10.2350/06-01-0024.1 (2006).
FOLLETT, P. L. et al. Glutamate receptor-mediated oligodendrocyte toxicity in periventricular leukomalacia: a protective role for topiramate. J Neurosci 24, 4412-4420, doi:10.1523/JNEUROSCI.0477-04.2004 (2004).
FUCHS, Y., and Steller, H. (2011). Programmed cell death in animal development and disease. Cell 147, 742-758.
GALLUZZI L, et al. (2014) Molecular mechanisms of regulated necrosis. Semin Cell Dev Biol 35C:24-32.
Galluzzi, L., et al. 2018. ‘Molecular mechanisms of cell death: recommendations of the Nomenclature Committee on Cell Death 2018’, Cell Death Differ, 25: 486-541.
GENNA, D. T. & Posner, G. H. Cyanocuprates convert carboxylic acids directly into ketones. Org Lett 13, 5358-5361, doi:10.1021/ol202237j (2011).
GOUT, P. W., et al. (2001). Sulfasalazine, a potent suppressor of lymphoma growth by inhibition of the x(c)-cystine transporter: a new action for an old drug. Leukemia 15, 1633-1640.
GUO, W., et al. (2008). Identification of a small molecule with synthetic lethality for K-ras and protein kinase C iota. Cancer Res 68, 7403-7408.
HAMADA, Y. & Kiso, Y. The application of bioisosteres in drug design for novel drug discovery: focusing on acid protease inhibitors. Expert opinion on drug discovery 7, 903-922, doi:10.1517/17460441.2012.712513 (2012).
Hatami, A., et al. 2018. ‘Deuterium-reinforced linoleic acid lowers lipid peroxidation and mitigates cognitive impairment in the Q140 knock in mouse model of Huntington's disease’, FEBS J, 285: 3002-12.
HAYNES, R. L. et al. Nitrosative and oxidative injury to premyelinating oligodendrocytes in periventricular leukomalacia. Journal of neuropathology and experimental neurology 62, 441-450 (2003).
HAYNES, R. L. et al. Oxidative and nitrative injury in periventricular leukomalacia: a review. Brain pathology 15, 225-233 (2005).
HE S, et al. (2009) Receptor interacting protein kinase-3 determines cellular necrotic response to TNF-alpha. Cell 137(6):1100-1111.
HOFFSTROM, B. G. et al. Inhibitors of protein disulfide isomerase suppress apoptosis induced by misfolded proteins. Nat Chem Biol 6, 900-906, doi:10.1038/nchembio.467 (2010).
Hofmans, S., et al. 2016. ‘Novel ferroptosis inhibitors with improved potency and ADME properties’, Journal of medicinal chemistry, 59: 2041-53.
HUANG, D., et al. The chemistry behind antioxidant capacity assays. J Agric Food Chem 53, 1841-1856, doi:10.1021/jf030723c (2005).
ISHIDA, T., et al. (2006). Benzimidazole inhibitors of hepatitis C virus NS5B polymerase: identification of 2-[(4-diarylmethoxy)phenyl]-benzimidazole. Bioorg Med Chem Lett 16, 1859-1863.
ISHII, T., et al. (1981). Mechanism of growth stimulation of L1210 cells by 2-mercaptoethanol in vitro. Role of the mixed disulfide of 2-mercaptoethanol and cysteine. The Journal of biological chemistry 256, 12387-12392.
JACOBSON, M. D., and Raff, M. C. (1995). Programmed cell death and Bcl-2 protection in very low oxygen. Nature 374, 814-816.
Ji, J., et al. 2012. ‘Lipidomics identifies cardiolipin oxidation as a mitochondrial target for redox therapy of brain injury’, Nature Neuroscience, 15: 1407-13.
Jiang, X., et al. 2021. ‘Ferroptosis: mechanisms, biology and role in disease’, Nat Rev Mol Cell Biol, 22: 266-82.
JOHRI, A. & Beal, M. F. Antioxidants in Huntington's disease. Biochim Biophys Acta 1822, 664-674, doi:10.1016/j.bbadis.2011.11.014 (2012).
KAMATA, T. (2009). Roles of Nox1 and other Nox isoforms in cancer development. Cancer Sci 100, 1382-1388.
KANAI, Y., and Endou, H. (2003). Functional properties of multispecific amino acid transporters and their implications to transporter-mediated toxicity. J Toxicol Sci 28, 1-17.
Kong, Y., et al. 2022. ‘BRCA1 haploinsufficiency promotes chromosomal amplification under Fenton reaction-based carcinogenesis through ferroptosis-resistance’, Redox Biol, 54: 102356.
Kumari, S., et al. 2020. ‘Amide Bond Bioisosteres: Strategies, Synthesis, and Successes’, J Med Chem, 63: 12290-358.
LALEU, B., et al. (2010). First in class, potent, and orally bioavailable NADPH oxidase isoform 4 (Nox4) inhibitors for the treatment of idiopathic pulmonary fibrosis. Journal of medicinal chemistry 53, 7715-7730.
LEI, P., et al. (2012). Tau deficiency induces parkinsonism with dementia by impairing APP-mediated iron export. Nature medicine 18, 291-295.
Li, J., et al. 2020. ‘Ferroptosis: past, present and future’, Cell Death Dis, 11: 88.
Li, Q., et al. 2017. ‘Inhibition of neuronal ferroptosis protects hemorrhagic brain’, JCI Insight, 2: e90777.
LI, Y., et al. (1997). A role for 12-lipoxygenase in nerve cell death caused by glutathione depletion. Neuron 19, 453-463.
Lin, C., et al. 2006. ‘Discovery and development of VX-950, a novel, covalent, and reversible inhibitor of hepatitis C virus NS3.4A serine protease’, Infect Disord Drug Targets, 6: 3-16.
LINKERMANN A, et al. (2012) Rip1 (receptor-interacting protein kinase 1) mediates necroptosis and contributes to renal ischemia/reperfusion injury. Kidney Int 81(8):751-761.
LINKERMANN A, et al. (2013A) The RIP1-kinase inhibitor necrostatin-1 prevents osmotic nephrosis and contrast-induced AKI in mice. J Am Soc Nephrol 24(10):1545-1557.
LINKERMANN A, et al. (2013B) Two independent pathways of regulated necrosis mediate ischemia-reperfusion injury. Proc Natl Acad Sci USA 110(29):12024-12029.
LINKERMANN A, et al. (2014B) Regulated Cell Death in AKI. J Am Soc Nephrol, ASN.2014030262.
LINKERMANN A, Green D R (2014A) Necroptosis. N Engl J Med 370(5):455-465.
LIPINSKI, C. A., et al. (2001). Experimental and computational approaches to estimate solubility and permeability in drug discovery and development settings. Advanced drug delivery reviews 46, 3-26.
LO, M., et al. (2008). The xc-cystine/glutamate antiporter: a mediator of pancreatic cancer growth with a role in drug resistance. British journal of cancer 99, 464-472.
Loscalzo, J. 2008. ‘Membrane redox state and apoptosis: death by peroxide’, Cell Metab, 8: 182-3.
LOSSI, L., Alasia, S., Salio, C., and Merighi, A. (2009). Cell death and proliferation in acute slices and organotypic cultures of mammalian CNS. Prog Neurobiol 88, 221-245.
LUEDDE M, et al. (2014) RIP3, a kinase promoting necroptotic cell death, mediates adverse remodelling after myocardial infarction. Cardiovasc Res 103(2):206-216.
MACARRON, R., et al. (2011). Impact of high-throughput screening in biomedical research. Nature reviews Drug discovery 10, 188-195.
Mangiarini, L., et al. 1996. ‘Exon 1 of the HD gene with an expanded CAG repeat is sufficient to cause a progressive neurological phenotype in transgenic mice’, Cell, 87: 493-506.
Marchant, C. A., et al. 2008. ‘In silico tools for sharing data and knowledge on toxicity and metabolism: derek for windows, meteor, and vitic’, Toxicol Mech Methods, 18: 177-87.
Martin-Sanchez, D., et al. 2017. ‘Ferroptosis, but Not Necroptosis, Is Important in Nephrotoxic Folic Acid-Induced AKI’, Journal of the American Society of Nephrology, 28: 218-29.
MASON, R. P. et al. Glutathione peroxidase activity is neuroprotective in models of Huntington's disease. Nat Genet 45, 1249-1254, doi:10.1038/ng.2732 (2013).
MILLER, B. R. & Bezprozvanny, I. Corticostriatal circuit dysfunction in Huntington's disease: intersection of glutamate, dopamine and calcium. Future neurology 5, 735-756, doi:10.2217/fnl.10.41 (2010).
MOFFAT, J., et al. (2006). A lentiviral RNAi library for human and mouse genes applied to an arrayed viral high-content screen. Cell 124, 1283-1298.
MORRISON, B., 3rd, et al. (2002). L-arginyl-3,4-spermidine is neuroprotective in several in vitro models of neurodegeneration and in vivo ischaemia without suppressing synaptic transmission. Br J Pharmacol 137, 1255-1268.
MORRISON, P. J. & Nevin, N. C. Serum iron, total iron binding capacity and ferritin in early Huntington disease patients. Irish journal of medical science 163, 236-237 (1994).
MULAY S R, et al. (2013) Calcium oxalate crystals induce renal inflammation by NLRP3-mediated IL-1β secretion. J Clin Invest 123(1):236-246.
MULLEN, A. R., et al. (2011). Reductive carboxylation supports growth in tumour cells with defective mitochondria. Nature.
MURPHY, T. H., et al. (1989). Glutamate toxicity in a neuronal cell line involves inhibition of cystine transport leading to oxidative stress. Neuron 2, 1547-1558.
NATIONAL RESEARCH COUNCIL (2011) Guide for the Care and Use of Laboratory Animals (National Academies Press, Washington, DC), 8th Ed.
NI CHONGHAILE, T., et al. (2011). Pretreatment mitochondrial priming correlates with clinical response to cytotoxic chemotherapy. Science 334, 1129-1133.
Ni, L., et al. 2022. ‘Targeting ferroptosis in acute kidney injury’, Cell Death Dis, 13: 182.
NORABERG, J., et al. (2005). Organotypic hippocampal slice cultures for studies of brain damage, neuroprotection and neurorepair. Curr Drug Targets CNS Neurol Disord 4, 435-452.
PAGLIARINI, D. J., et al. (2008). A mitochondrial protein compendium elucidates complex I disease biology. Cell 134, 112-123.
PARK, J. S., et al. Cyclophilin D and the mitochondrial permeability transition in kidney proximal tubules after hypoxic and ischemic injury. American journal of physiology. Renal physiology 301, F134-150, doi:10.1152/ajprenal.00033.2011 (2011).
PASSANITI, P. et al. Synthesis, spectroscopic and electrochemical properties of mononuclear and dinuclear bis(bipy)ruthenium(II) complexes containing dimethoxyphenyl(pyridin-2-yl)-1,2,4-triazole ligands J. Chem. Soc., Dalton Transactions 8, 1740-1746 (2002).
PETR, G. T. et al. Glutamate transporter expression and function in a striatal neuronal model of Huntington's disease. Neurochem Int 62, 973-981, doi:10.1016/j.neuint.2013.02.026 (2013).
PINNIX, Z. K., et al. (2010). Ferroportin and iron regulation in breast cancer progression and prognosis. Sci Transl Med 2, 43ra56.
PIPIK, B., et al. A preferred synthesis of 1,2,4-oxadiazoles. Synthetic Communications 34, 1863-1870 (2004).
RAJ, L., et al. (2011). Selective killing of cancer cells by a small molecule targeting the stress response to ROS. Nature 475, 231-234.
RAMANA, Kota V. et al., “Lipid Peroxidation Products in Human Health and Disease,” Oxidative Medicine and Cellular Longevity, vol. 2013, Article ID 583438, 3 pages, 2013. doi:10.1155/2013/583438.
RAMANATHAN, A., and Schreiber, S. L. (2009). Direct control of mitochondrial function by mTOR. Proc Natl Acad Sci USA 106, 22229-22232.
Rankovic, Z. 2015. ‘CNS drug design: balancing physicochemical properties for optimal brain exposure’, J Med Chem, 58: 2584-608.
RATAN, R. R., et al. (1994). Oxidative stress induces apoptosis in embryonic cortical neurons. J Neurochem 62, 376-379.
RIBEIRO, F. M., et al. Huntington's disease and Group I metabotropic glutamate receptors. Molecular neurobiology 43, 1-11, doi:10.1007/s12035-010-8153-1 (2011).
RIBEIRO, M. et al. Glutathione redox cycle dysregulation in Huntington's disease knock-in striatal cells. Free Radic Biol Med 53, 1857-1867, doi:10.1016/j.freeradbiomed.2012.09.004 (2012).
SAITOH, M. et al. Design, synthesis and structure-activity relationships of 1,3,4-oxadiazole derivatives as novel inhibitors of glycogen synthase kinase-3beta. Bioorg Med Chem 17, 2017-2029, doi:10.1016/j.bmc.2009.01.019 (2009).
SALAHUDEEN, A. A., et al. (2009). An E3 ligase possessing an iron-responsive hemerythrin domain is a regulator of iron homeostasis. Science 326, 722-726.
SANCHEZ, M., et al. (2011). Iron regulatory protein-1 and -2: transcriptome-wide definition of binding mRNAs and shaping of the cellular proteome by iron regulatory proteins. Blood 118, e168-179.
SATO, H., et al. (1999). Cloning and expression of a plasma membrane cystine/glutamate exchange transporter composed of two distinct proteins. The Journal of biological chemistry 274, 11455-11458.
Seiler, A., et al. 2008. ‘Glutathione peroxidase 4 senses and translates oxidative stress into 12/15-lipoxygenase dependent- and AIF-Mediated cell death’, Cell Metabolism, 8: 237-48.
SHAW, A. T., et al. (2011). Selective killing of K-ras mutant cancer cells by small molecule inducers of oxidative stress. Proc Natl Acad Sci USA.
SKOUTA R, et al. (2014) Ferrostatins inhibit oxidative lipid damage and cell death in diverse disease models. J Am Chem Soc 136(12):4551-4556.
SMITH C C, et al. (2007) Necrostatin: A potentially novel cardioprotective agent? Cardiovasc Drugs Ther 21(4):227-233.
SOGABE, K., et al. Differential cytoprotection by glycine against oxidant damage to proximal tubule cells. Kidney international 50, 845-854 (1996).
Stimac, A., et al. 2017. ‘Adamantane in Drug Delivery Systems and Surface Recognition’, Molecules, 22.
Stockwell, B. R. 2022. ‘Ferroptosis turns 10: Emerging mechanisms, physiological functions, and therapeutic applications’, Cell, 185: 2401-21.
SUNDSTROM, L., et al. (2005). Organotypic cultures as tools for functional screening in the CNS. Drug discovery today 10, 993-1000.
TAN, S., et al. (1998). The regulation of reactive oxygen species production during programmed cell death. The Journal of Cell Biology 141, 1423-1432.
THOMPSON, C. B. (1995). Apoptosis in the pathogenesis and treatment of disease. Science 267, 1456-1462.
Toyokuni, S., et al. 2022. ‘Iron as spirit of life to share under monopoly’, J Clin Biochem Nutr, 71: 78-88.
TRACHOOTHAM, D., et al. (2006). Selective killing of oncogenically transformed cells through a ROS-mediated mechanism by beta-phenylethyl isothiocyanate. Cancer Cell 10, 241-252.
TRAYKOVA-BRAUCH M, et al. (2008) An efficient and versatile system for acute and chronic modulation of renal tubular function in transgenic mice. Nat Med 14(9): 979-984.
Tunez, I., et al. 2010. ‘3-Nitropropionic acid as a tool to study the mechanisms involved in Huntington's disease: past, present and future’, Molecules, 15: 878-916.
Tuo, Q. Z., et al. 2017. ‘Tau-mediated iron export prevents ferroptotic damage after ischemic stroke’, Molecular Psychiatry, 22: 1520-30.
TURMAINE, M. et al. Nonapoptotic neurodegeneration in a transgenic mouse model of Huntington's disease. Proc Natl Acad Sci USA 97, 8093-8097. (2000).
van der Burg, J. M., et al. 2008. ‘Increased metabolism in the R6/2 mouse model of Huntington's disease’, Neurobiol Dis, 29: 41-51.
VARMA, H., et al. in Neurobiology of Huntington's Disease: Applications to Drug Discovery Frontiers in Neuroscience (eds D. C. Lo & R. E. Hughes) (2011).
VASHISHT, A. A., et al. (2009). Control of iron homeostasis by an iron-regulated ubiquitin ligase. Science 326, 718-721.
Veber, D. F. 2003. ‘Molecular properties that influence the oral bioavailability of drug candidates.’, Abstracts of Papers of the American Chemical Society, 225: U208-U08.
VIGIL, D., et al. (2010). Ras superfamily GEFs and GAPs: validated and tractable targets for cancer therapy? Nat Rev Cancer 10, 842-857.
WANG, Y., et al. (2009). Poly(ADP-ribose) signals to mitochondrial AIF: a key event in parthanatos. Exp Neurol 218, 193-202.
WATKINS, P. A., et al. (2007). Evidence for 26 distinct acyl-coenzyme A synthetase genes in the human genome. J Lipid Res 48, 2736-2750.
WISE, D. R., et al. (2008). Myc regulates a transcriptional program that stimulates mitochondrial glutaminolysis and leads to glutamine addiction. Proceedings of the National Academy of Sciences of the United States of America 105, 18782-18787.
Wolpaw, A. J., et al. 2011. ‘Modulatory profiling identifies mechanisms of small molecule-induced cell death’, Proc Nat/Acad Sci USA, 108: E771-80.
WU, C. et al. Discovery, modeling, and human pharmacokinetics of N-(2-acetyl-4,6-dimethylphenyl)-3-(3,4-dimethylisoxazol-5-ylsulfamoyl)thiophene-2-carboxamide (TBC3711), a second generation, ETA selective, and orally bioavailable endothelin antagonist. J Med Chem 47, 1969-1986, doi:10.1021/jm030528p (2004).
Wu, Y. N., et al. 2019. ‘The Potential Role of Ferroptosis in Neonatal Brain Injury’, Frontiers in Neuroscience, 13.
Xun, Z., et al. 2012. ‘Targeting of XJB-5-131 to mitochondria suppresses oxidative DNA damage and motor decline in a mouse model of Huntington's disease’, Cell Rep, 2: 1137-42.
YAGODA, N., et al. (2007). RAS-RAF-MEK-dependent oxidative cell death involving voltage-dependent anion channels. Nature 447, 864-868.
Yang, W. S., and B. R. Stockwell. 2016. ‘Ferroptosis: Death by Lipid Peroxidation’, Trends Cell Biol, 26: 165-76.
YANG, W. S., and Stockwell, B. R. (2008). Synthetic lethal screening identifies compounds activating iron-dependent, nonapoptotic cell death in oncogenic-RAS-harboring cancer cells. Chemistry & biology 15, 234-245.
YONEZAWA, M., et al. (1996). Cystine deprivation induces oligodendroglial death: rescue by free radical scavengers and by a diffusible glial factor. J Neurochem 67, 566-573.
ZEIGER, E. (2019). The test that changed the world: The AMES test and the regulation of chemicals. Mutat Res Genet Toxicol Environ Mutagen 841, 43-48.
ZERON, M. M. et al. Increased sensitivity to N-methyl-D-aspartate receptor-mediated excitotoxicity in a mouse model of Huntington's disease. Neuron 33, 849-860. (2002).
ZHANG D W, et al. (2009) RIP3, an energy metabolism regulator that switches TNFinduced cell death from apoptosis to necrosis. Science 325(5938):332-336.
Zilka, O., et al. 2017. ‘On the Mechanism of Cytoprotection by Ferrostatin-1 and Liproxstatin-1 and the Role of Lipid Peroxidation in Ferroptotic Cell Death’, ACS Cent Sci, 3: 232-43.

All documents cited in this application are hereby incorporated by reference as if recited in full herein.

Although illustrative embodiments of the present disclosure have been described herein, it should be understood that the disclosure is not limited to those described, and that various other changes or modifications may be made by one skilled in the art without departing from the scope or spirit of the disclosure.

What is claimed is:

1. A compound having the structure selected from the group consisting of: 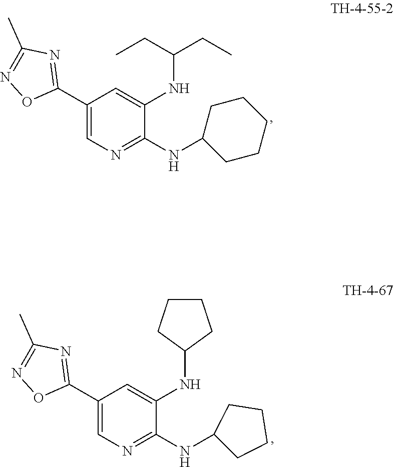 [SMILES: CCC(CC)Nc1cc(-c2nc(C)no2)cnc1NC1CCCCC1 ; Cc1noc(-c2cnc(NC3CCCC3)c(NC3CCCC3)c2)n1]
and combinations thereof, or an N-oxide, crystalline form, hydrate, or a pharmaceutically acceptable salt thereof.

2. A pharmaceutical composition comprising a pharmaceutically acceptable carrier or diluent and one or more compounds according to claim 1.

3. A kit comprising a compound according to claim 1 together with instructions for the use of the compound.

4. A kit comprising a pharmaceutical composition according to claim 2 together with instructions for the use of the pharmaceutical composition.

5. A method for treating or ameliorating the effects of a disorder in a subject in need thereof comprising administering to the subject an effective amount of one or more compounds according to claim 1.

6. The method according to claim 5, wherein the disorder is a degenerative disease that involves lipid peroxidation.

7. The method according to claim 5, wherein the disorder is an excitotoxic disease involving oxidative cell death.

8. The method according to claim 5, wherein the disorder is selected from the group consisting of epilepsy, kidney disease, stroke, myocardial infarction, type I diabetes, TBI, PVL, and neurodegenerative disease.

9. The method according to claim 8, wherein the neurodegenerative disease is selected from the group consisting of Alzheimer's, Parkinson's, Amyotrophic lateral sclerosis, Friedreich's ataxia, Multiple sclerosis, Huntington's Disease, Transmissible spongiform encephalopathy, Charcot-Marie-Tooth disease, Dementia with Lewy bodies, Corticobasal degeneration, Progressive supranuclear palsy, Chronic Traumatic Encephalopathy (CTE), and Hereditary spastic paraparesis.

10. The method according to claim 5 further comprising co-administering to the subject an effective amount of one or more additional therapeutic agents selected from the group consisting of 5-hydroxytryptophan, Activase, AFQ056 (Novartis), Aggrastat, Albendazole, alpha-lipoic acid/L-acetyl carnitine, Alteplase, Amantadine (Symmetrel), amlodipine, Ancrod, Apomorphine (Apokyn), Arimoclomol, Arixtra, Armodafinil, Ascorbic acid, Ascriptin, Aspirin, atenolol, Avonex, baclofen (Lioresal), Banzel, Benztropine (Cogentin), Betaseron, BGG492 (Novartis Corp.), Botulinum toxin, Bufferin, Carbatrol®, Carbidopa/levodopa immediate-release (Sinemet), Carbidopa/levodopa oral disintegrating (Parcopa), Carbidopa/levodopa/Entacapone (Stalevo), CERE-110: Adeno-Associated Virus Delivery of NGF (Ceregene), cerebrolysin, CinnoVex, citalopram, citicoline, Clobazam, Clonazepam, Clopidogrel, clozapine (Clozaril), Coenzyme Q, Creatine, dabigatran, dalteparin, Dapsone, Davunetide, Deferiprone, Depakene®, Depakote ER®, Depakote®, Desmoteplase, Diastat, Diazepam, Digoxin, Dilantin®, Dimebon, dipyridamole, divalproex (Depakote), Donepezil (Aricept), EGb 761, Eldepryl, ELND002 (Elan Pharmaceuticals), Enalapril, enoxaparin, Entacapone (Comtan), epoetin alfa, Eptifibatide, Erythropoietin, Escitalopram, Eslicarbazepine acetate, Esmolol, Ethosuximide, Ethyl-EPA (Miraxion™), Exenatide, Extavia, Ezogabine, Felbamate, Felbatol®, Fingolimod (Gilenya), fluoxetine (Prozac), fondaparinux, Fragmin, Frisium, Gabapentin, Gabitril®, Galantamine, Glatiramer (Copaxone), haloperidol (Haldol), Heparin, human chorionic gonadotropin (hCG), Idebenone, Inovelon®, insulin, Interferon beta 1a, Interferon beta 1b, ioflupane 123I (DATSCAN®), IPX066 (Impax Laboratories Inc.), JNJ-26489112 (Johnson and Johnson), Keppra®, Klonopin, Lacosamide, L-Alpha glycerylphosphorylcholine, Lamictal®, Lamotrigine, Levetiracetam, liraglutide, Lisinopril, Lithium carbonate, Lopressor, Lorazepam, losartan, Lovenox, Lu AA24493, Luminal, LY450139 (Eli Lilly), Lyrica, Masitinib, Mecobalamin, Memantine, methylprednisolone, metoprolol tartrate, Minitran, Minocycline, mirtazapine, Mitoxantrone (Novantrone), Mysoline®, Natalizumab (Tysabri), Neurontin®, Niacinamide, Nitro-Bid, Nitro-Dur, nitroglycerin, Nitrolingual, Nitromist, Nitrostat, Nitro-Time, Norepinephrine (NOR), Carbamazepine, octreotide, Onfi®, Oxcarbazepine, Oxybutinin chloride, PF-04360365 (Pfizer), Phenobarbital, Phenytek®, Phenytoin, piclozotan, Pioglitazone, Plavix, Potiga, Pramipexole (Mirapex), pramlintide, Prednisone, Primidone, Prinivil, probenecid, Propranolol, PRX-00023 (EPIX Pharmaceuticals Inc.), PXT3003, Quinacrine, Ramelteon, Rasagiline (Azilect), Rebif, ReciGen, remacemide, Resveratrol, Retavase, reteplase, riluzole (Rilutek), Rivastigmine (Exelon), Ropinirole (Requip), Rotigotine (Neupro), Rufinamide, Sabril, safinamide (EMD Serono), Salagen, Sarafem, Selegiline (I-deprenyl, Eldepryl), SEN0014196 (Siena Biotech), sertraline (Zoloft), Simvastatin, Sodium Nitroprussiate (NPS), sodium phenylbutyrate, Stanback Headache Powder, Tacrine (Cognex), Tamoxifen, tauroursodeoxycholic acid (TUDCA), Tegretol®, Tenecteplase, Tenormin, Tetrabenazine (Xenazine), THR-18 (Thrombotech Ltd.), Tiagabine, Tideglusib, tirofiban, tissue plasminogen activator (tPA), tizanidine (Zanaflex), TNKase, Tolcapone (Tasmar), Tolterodine, Topamax®, Topiramate, Trihexyphenidyl (formerly Artane), Trileptal®, ursodiol, Valproic Acid, valsartan, Varenicline (Pfizer), Vimpat, Vitamin E, Warfarin, Zarontin®, Zestril, Zonegran®, Zonisamide, Zydis selegiline HCL Oral disintegrating (Zelapar), and combinations thereof.

11. The method according to claim 5, wherein the subject is a mammal.

12. The method according to claim 11, wherein the mammal is selected from the group consisting of humans, veterinary animals, and agricultural animals.

13. The method according to claim 5, wherein the subject is a human.

14. A method for treating or ameliorating the effects of a disorder in a subject in need thereof comprising administering to the subject an effective amount of a pharmaceutical composition according to claim 2.

15. The method according to claim 14, wherein the disorder is a degenerative disease that involves lipid peroxidation.

16. The method according to claim 14, wherein the disorder is an excitotoxic disease involving oxidative cell death.

17. The method according to claim 14, wherein the disorder is selected from the group consisting of epilepsy, kidney disease, stroke, myocardial infarction, type I diabetes, TBI, PVL, and neurodegenerative disease.

18. The method according to claim 17, wherein the neurodegenerative disease is selected from the group consisting of Alzheimer's, Parkinson's, Amyotrophic lateral sclerosis, Friedreich's ataxia, Multiple sclerosis, Huntington's Disease, Transmissible spongiform encephalopathy, Charcot-Marie-Tooth disease, Dementia with Lewy bodies, Corticobasal degeneration, Progressive supranuclear palsy, Chronic Traumatic Encephalopathy (CTE), and Hereditary spastic paraparesis.

19. The method according to claim 14 further comprising co-administering to the subject an effective amount of one or more therapeutic agents selected from the group consisting of: 5-hydroxytryptophan, Activase, AFQ056 (Novartis), Aggrastat, Albendazole, alpha-lipoic acid/L-acetyl carnitine, Alteplase, Amantadine (Symmetrel), amlodipine, Ancrod, Apomorphine (Apokyn), Arimoclomol, Arixtra, Armodafinil, Ascorbic acid, Ascriptin, Aspirin, atenolol, Avonex, baclofen (Lioresal), Banzel, Benztropine (Cogentin), Betaseron, BGG492 (Novartis Corp.), Botulinum toxin, Bufferin, Carbatrol®, Carbidopa/levodopa immediate-release (Sinemet), Carbidopa/levodopa oral disintegrating (Parcopa), Carbidopa/levodopa/Entacapone (Stalevo), CERE-110: Adeno-Associated Virus Delivery of NGF (Ceregene), cerebrolysin, CinnoVex, citalopram, citicoline, Clobazam, Clonazepam, Clopidogrel, clozapine (Clozaril), Coenzyme Q, Creatine, dabigatran, dalteparin, Dapsone, Davunetide, Deferiprone, Depakene®, Depakote ER®, Depakote®, Desmoteplase, Diastat, Diazepam, Digoxin, Dilantin®, Dimebon, dipyridamole, divalproex (Depakote), Donepezil (Aricept), EGb 761, Eldepryl, ELND002 (Elan Pharmaceuticals), Enalapril, enoxaparin, Entacapone (Comtan), epoetin alfa, Eptifibatide, Erythropoietin, Escitalopram, Eslicarbazepine acetate, Esmolol, Ethosuximide, Ethyl-EPA (Miraxion™), Exenatide, Extavia, Ezogabine, Felbamate, Felbatol®, Fingolimod (Gilenya), fluoxetine (Prozac), fondaparinux, Fragmin, Frisium, Gabapentin, Gabitril®, Galantamine, Glatiramer (Copaxone), haloperidol (Haldol), Heparin, human chorionic gonadotropin (hCG), Idebenone, Inovelon®, insulin, Interferon beta 1a, Interferon beta 1b, ioflupane 123I (DATSCAN®), IPX066 (Impax Laboratories Inc.), JNJ-26489112 (Johnson and Johnson), Keppra®, Klonopin, Lacosamide, L-Alpha glycerylphosphorylcholine, Lamictal®, Lamotrigine, Levetiracetam, liraglutide, Lisinopril, Lithium carbonate, Lopressor, Lorazepam, losartan, Lovenox, Lu AA24493, Luminal, LY450139 (Eli Lilly), Lyrica, Masitinib, Mecobalamin, Memantine, methylprednisolone, metoprolol tartrate, Minitran, Minocycline, mirtazapine, Mitoxantrone (Novantrone), Mysoline®, Natalizumab (Tysabri), Neurontin®, Niacinamide, Nitro-Bid, Nitro-Dur, nitroglycerin, Nitrolingual, Nitromist, Nitrostat, Nitro-Time, Norepinephrine (NOR), Carbamazepine, octreotide, Onfi®, Oxcarbazepine, Oxybutinin chloride, PF-04360365 (Pfizer), Phenobarbital, Phenytek®, Phenytoin, piclozotan, Pioglitazone, Plavix, Potiga, Pramipexole (Mirapex), pramlintide, Prednisone, Primidone, Prinivil, probenecid, Propranolol, PRX-00023 (EPIX Pharmaceuticals Inc.), PXT3003, Quinacrine, Ramelteon, Rasagiline (Azilect), Rebif, ReciGen, remacemide, Resveratrol, Retavase, reteplase, riluzole (Rilutek), Rivastigmine (Exelon), Ropinirole (Requip), Rotigotine (Neupro), Rufinamide, Sabril, safinamide (EMD Serono), Salagen, Sarafem, Selegiline (1-deprenyl, Eldepryl), SEN0014196 (Siena Biotech), sertraline (Zoloft), Simvastatin, Sodium Nitroprussiate (NPS), sodium phenylbutyrate, Stanback Headache Powder, Tacrine (Cognex), Tamoxifen, tauroursodeoxycholic acid (TUDCA), Tegretol®, Tenecteplase, Tenormin, Tetrabenazine (Xenazine), THR-18 (Thrombotech Ltd.), Tiagabine, Tideglusib, tirofiban, tissue plasminogen activator (tPA), tizanidine (Zanaflex), TNKase, Tolcapone (Tasmar), Tolterodine, Topamax®, Topiramate, Trihexyphenidyl (formerly Artane), Trileptal®, ursodiol, Valproic Acid, valsartan, Varenicline (Pfizer), Vimpat, Vitamin E, Warfarin, Zarontin®, Zestril, Zonegran®, Zonisamide, Zydis selegiline HCL Oral disintegrating (Zelapar), and combinations thereof.

20. The method according to claim 14, wherein the subject is a mammal.

21. The method according to claim 20, wherein the mammal is selected from the group consisting of humans, veterinary animals, and agricultural animals.

22. The method according to claim 14, wherein the subject is a human.

23. A method of modulating ferroptosis in a subject in need thereof comprising administering to the subject an effective amount of a ferroptosis inhibitor, which comprises one or more compounds according to claim 1.

24. A method of reducing reactive oxygen species (ROS) in a cell comprising contacting a cell with a ferroptosis modulator, which comprises one or more compounds according to claim 1.

25. A method for treating or ameliorating the effects of a neurodegenerative disease in a subject in need thereof comprising administering to the subject an effective amount of one or more compounds according to claim 1.

26. The method according to claim 25, wherein the neurodegenerative disease is selected from the group consisting of Alzheimer's, Parkinson's, Amyotrophic lateral sclerosis, Friedreich's ataxia, Multiple sclerosis, Huntington's Disease, Transmissible spongiform encephalopathy, Charcot-Marie-Tooth disease, Dementia with Lewy bodies, Corticobasal degeneration, Progressive supranuclear palsy, Chronic Traumatic Encephalopathy (CTE), and Hereditary spastic paraparesis.

27. The method according to claim 25 further comprising co-administering to the subject an effective amount of one or more additional therapeutic agents selected from the group consisting of Donepezil (Aricept), Rivastigmine (Exelon), Galantamine (Razadyne), Tacrine (Cognex), Memantine (Namenda), Vitamin E, CERE-110: Adeno-Associated Virus Delivery of NGF (Ceregene), LY450139 (Eli Lilly), Exenatide, Varenicline (Pfizer), PF-04360365 (Pfizer), Resveratrol, Carbidopa/levodopa immediate-release (Sinemet), Carbidopa/levodopa oral disintegrating (Parcopa), Carbidopa/levodopa/Entacapone (Stalevo), Ropinirole (Requip), Pramipexole (Mirapex), Rotigotine (Neupro), Apomorphine (Apokyn), Selegiline (1-deprenyl, Eldepryl), Rasagiline (Azilect), Zydis selegiline HCL Oral disintegrating (Zelapar), Entacapone (Comtan), Tolcapone (Tasmar), Amantadine (Symmetrel), Trihexyphenidyl (formerly Artane), Benztropine (Cogentin), IPX066 (Impax Laboratories Inc.), ioflupane 123I (DATSCAN®), safinamide (EMD Serono), Pioglitazone, riluzole (Rilutek), Lithium carbonate, Arimoclomol, Creatine, Tamoxifen, Mecobalamin, tauroursodeoxycholic acid (TUDCA), Idebenone, Coenzyme Q, 5-hydroxytryptophan, Propranolol, Enalapril, Lisinopril, Digoxin, Erythropoietin, Lu AA24493, Deferiprone, IVIG, EGb 761, Avonex, Betaseron, Extavia, Rebif, Glatiramer (Copaxone), Fingolimod (Gilenya), Natalizumab (Tysabri), Mitoxantrone (Novantrone), baclofen (Lioresal), tizanidine (Zanaflex), methylprednisolone, CinnoVex, ReciGen, Masitinib, Prednisone, Interferon beta 1a, Interferon beta 1b, ELND002 (Elan Pharmaceuticals), Tetrabenazine (Xenazine), haloperidol (Haldol), clozapine (Clozaril), clonazepam(Klonopin), diazepam (Valium), escitalopram (Lexapro), fluoxetine (Prozac, Sarafem), sertraline (Zoloft), valproic acid (Depakene), divalproex (Depakote), lamotrigine (Lamictal), Dimebon, AFQ056 (Novartis), Ethyl-EPA (Miraxion™), SEN0014196 (Siena Biotech), sodium phenylbutyrate, citalopram, ursodiol, minocycline, remacemide, mirtazapine, Quinacrine, Ascorbic acid, PXT3003, Armodafinil, Ramelteon, Davunetide, Tideglusib, alpha-lipoic acid/L-acetyl carnitine, Niacinamide, Oxybutinin chloride, Tolterodine, Botulinum toxin, and combinations thereof.

28. The method according to claim 25, wherein the subject is a mammal.

29. The method according to claim 28, wherein the mammal is selected from the group consisting of humans, veterinary animals, and agricultural animals.

30. The method according to claim 25, wherein the subject is a human.

31. A method for alleviating side effects in a subject undergoing radiotherapy and/or immunotherapy, comprising administering to the subject an effective amount of one or more compounds according to claim 1.

32. A method for treating or ameliorating the effects of an infection associated with ferroptosis in a subject, comprising administering to the subject an effective amount of one or more compounds according to claim 1.

33. The method according to claim 32, wherein the infection is caused by Mycobacterium tuberculosis.
